SEQUENCE LISTING (86 sequences)

SEQ ID NO: 1 - DNA, 2368 bp, Homo sapiens
gcggtgccgcccgccgtggccgcctcagcccaccagccgggaccgcgagccatgctgtccgccgcccgcccccagggttgttaaagccagactgcgaactctcgccactgccgccaccgccgcgtcccgtcccaccgtcgcgggcaacaaccaaagtcgccgcaactgcagcacagagcgggcaaagccaggcaggccatggggctctgggcgctgttgcctggctgggtttctgctacgctgctgctggcgctggccgctctgcccgcagccctggctgccaacagcagtggccgatggtggggtattgtgaacgtagcctcctccacgaacctgcttacagactccaagagtctgcaactggtactcgagcccagtctgcagctgttgagccgcaaacagcggcgtctgatacgccaaaatccggggatcctgcacagcgtgagtggggggctgcagagtgccgtgcgcgagtgcaagtggcagttccggaatcgccgctggaactgtcccactgctccagggccccacctcttcggcaagatcgtcaaccgaggctgtcgagaaacggcgtttatcttcgctatcacctccgccggggtcacccattcggtggcgcgctcctgctcagaaggttccatcgaatcctgcacgtgtgactaccggcggcgcggccccgggggccccgactggcactgggggggctgcagcgacaacattgacttcggccgcctcttcggccgggagttcgtggactccggggagaaggggcgggacctgcgcttcctcatgaaccttcacaacaacgaggcaggccgtacgaccgtattctccgagatgcgccaggagtgcaagtgccacgggatgtccggctcatgcacggtgcgcacgtgctggatgcggctgcccacgctgcgcgccgtgggcgatgtgctgcgcgaccgcttcgacggcgcctcgcgcgtcctgtacggcaaccgcggcagcaaccgcgcttcgcgagcggagctgctgcgcctggagccggaagacccggcccacaaaccgccctccccccacgacctcgtctacttcgagaaatcgcccaacttctgcacgtacagcggacgcctgggcacagcaggcacggcagggcgcgcctgtaacagctcgtcgcccgcgctggacggctgcgagctgctctgctgcggcaggggccaccgcacgcgcacgcagcgcgtcaccgagcgctgcaactgcaccttccactggtgctgccacgtcagctgccgcaactgcacgcacacgcgcgtactgcacgagtgtctgtgaggcgctgcgcggactcgcccccaggaaacgctctcctcgagccctcccccaaacagactcgctagcactcaagacccggttattcgcccacccgagtacctccagtcacactccccgcggttcatacgcatcccatctctcccacttcctcctacctggggactcctcaaaccacttgcctggggcggcatgaaccctcttgccatcctgatggacctgccccggacctacctccctccctctccgcgggagaccccttgttgcactgccccctgcttggccaggaggtgagagaaggatgggtcccctccgccatggggtcggctcctgatggtgtcattctgcctgctccatcgcgccagcgacctctctgcctctcttcttcccctttgtcctgcgttttctccgggtcctcctaagtcccttcctattctcctgccatgggtgcagaccctgaacccacacctgggcatcagggcctttctcctccccacctgtagctgaagcaggaggttacagggcaaaagggcagctgtgatgatgtggaaatgaggttgggggaaccagcagaaatgcccccattctcccagtctctgtcgtggagccattgaacagctgtgagccatgcctccctgggccacctcctaccccttcctgtcctgcctcctcatcagtgtgtaaataatttgcactgaaacgtggatacagagccacgagtttggatgttgtaaataaaactatttattgtgctgggtcccagcctggtttgcaaagaccacctccaacccaacccaatccctctccactcttctctcctttctccctgcagccttttctggtccctcttctctcctcagtttctcaaagatgcgtttgcctcctggaatcagtatttccttccactgtagctattagcggctcctcgcccccaccagtgtagcatcttcctctgcagaataaaatctctatttttatcgatgacttggtggcttttccttgaatccagaacacaaccttgtttgtggtgtcccctatcctccccttttaccactcccag

SEQ ID NO: 2 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 3 - DNA, 2463 bp, Mouse
gccacagcttcgctcgccactcattgtctgtggccctgaccagtgcgccctggtgcttttagtgccgcccgggcccggaggggcagcctcttctcactgcagtcagcgccgcaactataagaggcggtgcctcccgcagtggctgcttcagcccagcagccaggacagcgaaccatgctgcctgcggcccgcctccagacttattagagccagcctgggaactcgcatcactgccctcaccgctgtgtccagtcccaccgtcgcggacagcaaccacagtcgtcagaaccccagcacagaaccagcaaggccaggcaggccatggggctctgggcgctgctgcccagctgggtttctactacgttgctactggcactgaccgctctgcccgcagccctagctgccaacagtagtggccgatggtggggcatcgtgaacatagcctcctccacgaacctgttgacggattccaagagcctgcagctggtgctcgagcccagtctgcagctgctgagccgcaagcagcggcgactgatccgacagaacccggggatcctgcacagcgtgagtggagggctccagagcgccgtgcgagagtgcaaatggcaattccgaaaccgccgctggaactgccccactgctccggggccccacctcttcggcaagatcgtcaaccgaggctgccgagaaacagcgttcatcttcgcaatcacctccgccggggtcacacattccgtggcgcgctcctgctccgaaggctccatcgagtcctgcacctgcgactaccggcggcgcggccctgggggccccgactggcactgggggggctgcagtgacaacatcgattttggtcgcctctttggccgagagttcgtggactccggggagaaggggcgggacctacgcttcctcatgaaccttcacaacaacgaggcagggcgaacgaccgtgttctctgagatgcgccaagagtgcaaatgccacgggatgtccggctcctgcacggtgcgcacgtgttggatgcggctgcccacgctgcgcgctgtgggcgacgtgctgcgcgaccgcttcgacggtgcctcccgcgtcctttacggcaacagaggcagcaaccgcgcctcgcgggcggagctgctgcgcctggagcccgaagaccccgcgcacaagcctccctcccctcacgacctcgtctacttcgagaaatcgcccaacttctgcacgtacagtggccgcctgggcacagctggcacagctggacgagcttgcaacagctcgtctcccgcgctggacggctgtgagctgctgtgctgtggccgaggccaccgcacgcgcacgcagcgcgtcacggagcgctgcaactgcaccttccactggtgctgccacgtcagctgccgcaactgcacgcacacgcgcgttctgcacgagtgtctatgaggtgccgcgcctccgggaacgggaacgctctcttccagttctcagacacactcactggtcctgatgtttgcccaccctaccgcgtccagccacagtcccagggttcatagcgatccatctctcccacctcctacctggggactcctgaaaccacttgcctgagtcggctcgaacccttttgccatcctgagggccctgacccagcctacctccctccctctttgagggagactccttttgcactgccccccaatttggccagagggtgagagaaagattcttcttctggggtgggggtggggaggtcaactcttgaaggtgttgcggttcctgatgtattttgcgctgtgacctctttgggtattatcacctttccttgtctctcaggtccctataggtcccttgagttctctaaccagcacctctgggcttcaaggcctttcccctcccacctgtagctgaagagtttccgagttgaaagggcacggaaagctaagtgggaaaggaggttgctggacccagcagcaaaaccctacattctccttgtctctgcctcggagccattgaacagctgtgaaccatgcctccctcagcctcctcccaccccttcctgtcctgcctcctcatcactgtgtaaataatttgcaccgaaatgtggccgcagagccacgcgttcggttatgtaaataaaactatttattgtgctgggttccagcctgggttgcagagaccaccctcaccccacctcactgctcctctgttctgcttgccagtccttttgttatccgaccttttttctcttttacccagcttctcataggcgcccttgcccaccggatcagtatttccttccactgtagctattagtggctcctcgcccccaccaatgtagtatcttcctctgaggaataaaatatctatttttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - protein, 370 aa, Mouse
MGLWALLPSWVSTTLLLALTALPAALAANSSGRWWGIVNIASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 5 - DNA, 2301 bp, Homo sapiens
agcagagcggacgggcgcgcgggaggcgcgcagagctttcgggctgcaggcgctcgctgccgctggggaattgggctgtgggcgaggcggtccgggctggcctttatcgctcgctgggcccatcgtttgaaactttatcagcgagtcgccactcgtcgcaggaccgagcggggggcgggggcgcggcgaggcggcggccgtgacgaggcgctcccggagctgagcgcttctgctctgggcacgcatggcgcccgcacacggagtctgacctgatgcagacgcaagggggttaatatgaacgcccctctcggtggaatctggctctggctccctctgctcttgacctggctcacccccgaggtcaactcttcatggtggtacatgagagctacaggtggctcctccagggtgatgtgcgataatgtgccaggcctggtgagcagccagcggcagctgtgtcaccgacatccagatgtgatgcgtgccattagccagggcgtggccgagtggacagcagaatgccagcaccagttccgccagcaccgctggaattgcaacaccctggacagggatcacagcctttttggcagggtcctactccgaagtagtcgggaatctgcctttgtttatgccatctcctcagctggagttgtatttgccatcaccagggcctgtagccaaggagaagtaaaatcctgttcctgtgatccaaagaagatgggaagcgccaaggacagcaaaggcatttttgattggggtggctgcagtgataacattgactatgggatcaaatttgcccgcgcatttgtggatgcaaaggaaaggaaaggaaaggatgccagagccctgatgaatcttcacaacaacagagctggcaggaaggctgtaaagcggttcttgaaacaagagtgcaagtgccacggggtgagcggctcatgtactctcaggacatgctggctggccatggccgacttcaggaaaacgggcgattatctctggaggaagtacaatggggccatccaggtggtcatgaaccaggatggcacaggtttcactgtggctaacgagaggtttaagaagccaacgaaaaatgacctcgtgtattttgagaattctccagactactgtatcagggaccgagaggcaggctccctgggtacagcaggccgtgtgtgcaacctgacttcccggggcatggacagctgtgaagtcatgtgctgtgggagaggctacgacacctcccatgtcacccggatgaccaagtgtgggtgtaagttccactggtgctgcgccgtgcgctgtcaggactgcctggaagctctggatgtgcacacatgcaaggcccccaagaacgctgactggacaaccgctacatgaccccagcaggcgtcaccatccaccttcccttctacaaggactccattggatctgcaagaacactggacctttgggttctttctggggggatatttcctaaggcatgtggcctttatctcaacggaagccccctcttcctccctgggggccccaggatggggggccacacgctgcacctaaagcctaccctattctatccatctcctggtgttctgcagtcatctcccctcctggcgagttctctttggaaatagcatgacaggctgttcagccgggagggtggtgggcccagaccactgtctccacccaccttgacgtttcttctttctagagcagttggccaagcagaaaaaaaagtgtctcaaaggagctttctcaatgtcttcccacaaatggtcccaattaagaaattccatacttctctcagatggaacagtaaagaaagcagaatcaactgcccctgacttaactttaacttttgaaaagaccaagacttttgtctgtacaagtggttttacagctaccacccttagggtaattggtaattacctggagaagaatggctttcaatacccttttaagtttaaaatgtgtatttttcaaggcatttattgccatattaaaatctgatgtaacaaggtggggacgtgtgtcctttggtactatggtgtgttgtatctttgtaagagcaaaagcctcagaaagggattgctttgcattactgtccccttgatataaaaaatctttagggaatgagagttccttctcacttagaatctgaagggaattaaaaagaagatgaatggtctggcaatattctgtaactattgggtgaatatggtggaaaataatttagtggatggaatatcagaagtatatctgtacagatcaagaaaaaaaggaagaataaaattcctatatcat

SEQ ID NO: 6 - protein, 360 aa, Homo sapiens
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 7 - DNA, 2027 bp, Mouse
ggcttctgctccgggcacgcatggcgcccgcacacggagtctgacctgatgtagacgcaagggggttaatatgaacgtccctctcggtggaatctggctctggctccctctgctcttgacctggctcacccctgaggtcagctcttcatggtggtacatgagagctacaggtggctcctccagggtgatgtgtgacaatgtgccaggcctggtgagccggcagcgtcagctgtgccaccgacacccagatgtgatgcgtgccattggcctgggtgtggctgagtggactgcagagtgccaacaccagttccgccagcatcgctggaactgcaacaccctggacagagatcacagcctctttggccgggtcctcctccgaagtagtcgggaatcggcctttgtttacgccatctcttcagctggcgttgtatttgccatcaccagggcctgtagccaaggagaattaaagtcctgctcctgtgatccaaagaagaaaggaagtgccaaggacagcaaaggcaccttcgactggggtggctgcagtgacaatattgactacgggatcaagtttgcccgtgcctttgtagatgccaaggagaggaaaggcaaggatgccagagccctgatgaaccttcacaacaacagagctggaaggaaggctgtaaagcgcttcttgaaacaagaatgcaagtgtcatggtgtgagtggctcctgtactctgaggacatgctggctggccatggctgacttcaggaaaacaggcgactatctctggaggaagtacaatggggccatccaggtagtcatgaaccagggtggcactggcttcactgtagccaataagaggtttaagaagccaacgaaaaatgacctcgtgtattttgagaattctccagactactgtatcagggaccgagaggcaggctccctgggtacagcgggccgtgtgtgcaacttgacttcccgaggcatggacagctgcgaagttatgtgttgtgggagaggctatgacacatcccacgtcacccggatgaccaagtgtgagtgtaaattccactggtgctgtgccgtgcgctgtcaggactgcctggaggccctggacgtgcacacatgcaaggcccccaagagtgccgactgggcgacgcctacatgacctcagcagaggtcatattcgccttttcttccctcaaggactccaattacatcttcaaggacactggacctctgggttgttttcaggggctctttcttaaggcatgaagccttcatctcaagagaaaccccctttcccctctctgggggccccaggactgggaaccacctgctgcacataagtacaccctattctgtctatcttgggcattctgatgtcacctctcttcctgctgatttctttttggaaatggcatgacaggctgttagaggaggagggtcatagccccccaccactgtcacctagacatttcctctttggctgcggggagaaacatcacatagcgaaggaacttcctctgtgttttcccagattccaacaacccagaaagtctgtgtttccctggggcgcggggtagggatggaaagcagaatgagctgacaccaaaatttcctcggatttttttaaaaaaagagtaagcaagggctttaactaagtgatagctgttgatagcatccttggtgactttctagagaaagatggcttccaataaacatcaggttaaaacatgtatgtcttcaaagaatttattggatatttattggctattggatataatagggtgagaatgtttgtcctttcagactgtgttatttttgaactttcctgtcagccaacaccttagaaagtgattgctattcctcactgtcccatcagtttaaggattcttaagagatgagacttctcagtgtgctctggagagaatctgaaaggggaatggatgatctagcaatattatttaactactgggtaaatatggtttaaaaataataataactttgtgagtggaatatcataaatgtgcttgtatggc

SEQ ID NO: 8 - protein, 360 aa, Mouse
MNVPLGGIWLWLPLLLTWLTPEVSSSWWYMRATGGSSRVMCDNVPGLVSRQRQLCHRHPDVMRAIGLGVAEWTAECQHQFRQHRWNCNTLDRDHSLFGRVLLRSSRESAFVYAISSAGVVFAITRACSQGELKSCSCDPKKKGSAKDSKGTFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQGGTGFTVANKRFKKPTKNDLVYFENSPDYCIRDREAGSLGTAGRVCNLTSRGMDSCEVMCCGRGYDTSHVTRMTKCECKFHWCCAVRCQDCLEALDVHTCKAPKSADWATPT

SEQ ID NO: 9 - DNA, 2014 bp, Homo sapiens
aaaccctgaagagcccaagcaatgtggttgtaaaatttgcaaaataagattaaatcttaactgcaatctgttaacactgctgtctcctttcactctttctcctatatcacactttcccacatgttggatggccttggagtggtagccataagcatttttggaattcaactaaaaactgaaggatccttgaggacggcagtacctggcatacctacacagtcagcgttcaacaagtgtttgcaaaggtacattggggcactgggggcacgagtgatctgtgacaatatccctggtttggtgagccggcagcggcagctgtgccagcgttacccagacatcatgcgttcagtgggcgagggtgcccgagaatggatccgagagtgtcagcaccaattccgccaccaccgctggaactgtaccaccctggaccgggaccacaccgtctttggccgtgtcatgctcagaagtagccgagaggcagcttttgtatatgccatctcatcagcaggggtagtccacgctattactcgcgcctgtagccagggtgaactgagtgtgtgcagctgtgacccctacacccgtggccgacaccatgaccagcgtggggactttgactggggtggctgcagtgacaacatccactacggtgtccgttttgccaaggccttcgtggatgccaaggagaagaggcttaaggatgcccgggccctcatgaacttacataataaccgctgtggtcgcacggctgtgcggcggtttctgaagctggagtgtaagtgccatggcgtgagtggttcctgtactctgcgcacctgctggcgtgcactctcagatttccgccgcacaggtgattacctgcggcgacgctatgatggggctgtgcaggtgatggccacccaagatggtgccaacttcaccgcagcccgccaaggctatcgccgtgccacccggactgatcttgtctactttgacaactctccagattactgtgtcttggacaaggctgcaggttccctaggcactgcaggccgtgtctgcagcaagacatcaaaaggaacagacggttgtgaaatcatgtgctgtggccgagggtacgacacaactcgagtcacccgtgttacccagtgtgagtgcaaattccactggtgctgtgctgtacggtgcaaggaatgcagaaatactgtggacgtccatacttgcaaagcccccaagaaggcagagtggctggaccagacctgaacacacagatacctcactcatccctccaattcaagcctctcaactcaaaagcacaagatccttgcatgcacaccttcctccaccctccaccctgggctgctaccgcttctatttaaggatgtagagagtaatccatagggaccatggtgtcctggctggttccttagccctgggaaggagttgtcaggggatataagaaactgtgcaagctccctgatttcccgctctggagatttgaagggagagtagaagagatagggggtctttagagtgaaatgagttgcactaaagtacgtagttgaggctccttttttctttcctttgcaccagcttcccgacacttcttggtgtgcaagaggaagggtacctgtagagagcttctttttgtttctacctggccaaagttagatgggacaaagatgaatggcatgtcccttctctgaagtccgtttgagcagaactacctggtaccccgaaagaaaaatcttaggctaccacattctattattgagagcctgagatgttagccatagtggacaaggttccattcacatgctcatatgtttataaactgtgttttgtagaagaaaaagaatcataacaatacaaacacacattcattctctctttttctctctaccattctcaacctgtattggacagcactgcctcttttgcttacttgctgcctgttcaaactgaggtggaatgcagtggttcccatgcttaacagatcattaaaacaccctagaacactcctaggatagattaatgt

SEQ ID NO: 10 - protein, 372 aa, Homo sapiens
MLDGLGVVAISIFGIQLKTEGSLRTAVPGIPTQSAFNKCLQRYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 11 - DNA, 1970 bp, Homo sapiens
cgggagtcttcggggagctatgctgagaccgggtggtgcggaggaagctgcgcagctcccgcttcggcgcgccagcgccccggtccctgtgccgtcgcccgcggcccccgacggctcccgggcttcggcccgcctaggtcttgcctgccttctgctcctgctgctgctgacgctgccggcccgcgtagacacgtcctggtggtacattggggcactgggggcacgagtgatctgtgacaatatccctggtttggtgagccggcagcggcagctgtgccagcgttacccagacatcatgcgttcagtgggcgagggtgcccgagaatggatccgagagtgtcagcaccaattccgccaccaccgctggaactgtaccaccctggaccgggaccacaccgtctttggccgtgtcatgctcagaagtagccgagaggcagcttttgtatatgccatctcatcagcaggggtagtccacgctattactcgcgcctgtagccagggtgaactgagtgtgtgcagctgtgacccctacacccgtggccgacaccatgaccagcgtggggactttgactggggtggctgcagtgacaacatccactacggtgtccgttttgccaaggccttcgtggatgccaaggagaagaggcttaaggatgcccgggccctcatgaacttacataataaccgctgtggtcgcacggctgtgcggcggtttctgaagctggagtgtaagtgccatggcgtgagtggttcctgtactctgcgcacctgctggcgtgcactctcagatttccgccgcacaggtgattacctgcggcgacgctatgatggggctgtgcaggtgatggccacccaagatggtgccaacttcaccgcagcccgccaaggctatcgccgtgccacccggactgatcttgtctactttgacaactctccagattactgtgtcttggacaaggctgcaggttccctaggcactgcaggccgtgtctgcagcaagacatcaaaaggaacagacggttgtgaaatcatgtgctgtggccgagggtacgacacaactcgagtcacccgtgttacccagtgtgagtgcaaattccactggtgctgtgctgtacggtgcaaggaatgcagaaatactgtggacgtccatacttgcaaagcccccaagaaggcagagtggctggaccagacctgaacacacagatacctcactcatccctccaattcaagcctctcaactcaaaagcacaagatccttgcatgcacaccttcctccaccctccaccctgggctgctaccgcttctatttaaggatgtagagagtaatccatagggaccatggtgtcctggctggttccttagccctgggaaggagttgtcaggggatataagaaactgtgcaagctccctgatttcccgctctggagatttgaagggagagtagaagagatagggggtctttagagtgaaatgagttgcactaaagtacgtagttgaggctccttttttctttcctttgcaccagcttcccgacacttcttggtgtgcaagaggaagggtacctgtagagagcttctttttgtttctacctggccaaagttagatgggacaaagatgaatggcatgtcccttctctgaagtccgtttgagcagaactacctggtaccccgaaagaaaaatcttaggctaccacattctattattgagagcctgagatgttagccatagtggacaaggttccattcacatgctcatatgtttataaactgtgttttgtagaagaaaaagaatcataacaatacaaacacacattcattctctctttttctctctaccattctcaacctgtattggacagcactgcctcttttgcttacttgctgcctgttcaaactgaggtggaatgcagtggttcccatgcttaacagatcattaaaacaccctagaacactcctaggatagattaatgt

SEQ ID NO: 12 - protein, 391 aa, Homo sapiens
MLRPGGAEEAAQLPLRRASAPVPVPSPAAPDGSRASARLGLACLLLLLLLTLPARVDTSWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 13 - DNA, 3576 bp, Mouse
ggagccactgacaccgcacccgaccgcccacacccggctcagcgctcgtcggtctcctggccctgcacgctcttgggaaccctgcgtctggctcccgggctccacgtgccttgaggtcctcggtcgcccatggtccccatggccactctgtggggcgatctaggagacgcctgagcgaagcccagacagtgcccgtccacggccctgcgggcttcggggcgggagtctgcggggagctatgctgaagctgcagggtgaggatgaagccgcgcagctcgcccctcggcgtgcccgcgtccccgtgcccagacccacggcccccgacgtgtccccatcttccgcccgcctgggtcttgcctgcctgctgctgctgctactcctgactctgccggcccgtgtagacacgtcctggtggtacataggggctctgggagcccgagtgatctgtgacaacatccccggtctggtgagccggcagcggcagttgtgtcaacgctacccagacatcatgcgctcagtaggtgagggtgcccgggaatggatccgagagtgccagcaccagttccgtcaccaccgctggaattgcaccacactggaccgggaccacactgtctttggccgcgccatgctcagaagcagccgggaggcagcgttcgtctatgctatctcgtcagcaggagtggtccacgctatcactcgggcctgcagccagggtgagctgagcgtgtgcagctgtgacccatatacccgcggtcggcaccatgatcaacgaggggactttgactggggtggctgtagtgacaacatccattacggtgttcgctttgccaaggcttttgtggatgccaaagagaagaggcttaaggatgcccgggccctcatgaacttacacaacaaccgctgtggtcgcacggctgttcggagattcctgaagctggagtgcaagtgtcacggtgtgagtggctcctgtactctgcgcacctgctggagagcactctcagacttccgacgcacaggtgactacctgaggaggcgatatgatggggctgtgcaggtgacggccacacaggatggggccaatttcacagcagcgcgccagggctatcgccacgccacccggactgatcttgtctactttgacaactcccctgactactgtgtcttggacaaggctgcaggttccctaggtaccgcaggccgcgtctgcagcaagacttctaaaggaacagatgggtgtgaaatcatgtgttgtggccgagggtatgacacaactcgggtcacccgcgtcacccagtgtgagtgcaaattccactggtgctgtgctgtgcggtgcaaggagtgcagaaacactgtggatgtccacacatgcaaggcccctaagaaggcagagtggctggaccagacctgaacacacagaaacctcattcttccctccacttcaagcctctgactcaaaagcacaagacccttgcatgcgcaccttcctctaccctcaatcctgggctgctatggcttctgtcacggacctggagagtgatccggagggaccccaatgtcccggccgcctggttccttagccctagggacgtgttgataggggatggatttaggaggctgagtgactccctgatggtccatctggaggtttgaagggagagtaggagaggtctgtcttcagagtgatttgagttgcactaagtcaaggctcatcctcccctttgcttgcactgacttctgatcctctttgggtatgcaacaggaagggaacctggaggtagcttccgtgtttgatgctactctgcctgaggataggacagagataaaactgcctgtccctttgctggagacagtacgggcagactatcttaggccatagtattctgctgagaccctgagatagctagatgggttagccacattgaacaaggctccacatcatgcttctacgcagcttataaagtagtggtttggtgaggaggaaaatcacaatgctctacagatacacattctctgtgcctccctttccacctacatcacacagcagcagcctgctcactggctgcctgttcagagtgaggcagcttgcagtgggtcaaattcttaccaggccattagaggcccggaacaggattgtgagagaatgacatagaaagcctggctaggccttgggacttcccccacatccactattccggagattcggtaggaagggaggtaactcatgggaagggtgagcgcacctgatctcaggggttccatgaggatcagtgtatactaggaaggcagagatctcgcattttgctagttcttgaggatcttcagctttgaagtaggaacaaaaggcagcagctatagagagagagctggtgctggagccgaggtggcaaacatcctataaggcctttctcatttacccagcaaatctttattttgtgattcaccaggtccaactgttaactactgcacgttccacgatcgacttaaacagggaaggttctctctgtgctactgaccgttgcctaacgagggtacacaggagtggagccttcaaagagagcaggcacagtgacatgggggttccaaaccttgatggtctagttttatgtgacctcgacaatggtcatcttcttccctattgataaacagaaatagtatagaaatccacagttagacttaggtctaatcccagctatttactctctattttttattttcagcagggtctttaaattctcctctcccattttcttatctgtaaagtgagggtgaaactgagatctaactgtgccccaaactgtagccgactgatagacgtcatcaacactctcactggtcaagtacttcctgcttctctgggaccttctgatttagggctgtctgggcagacaacagagtagattcaaagggctttcacaatgaattctggatatagctcctctctctcttctcagggttcctcttcatccaatcgtactctcagatgtttgtggagcaacctctttctgcccaggcagcaggaggctggggtggggtggggtgggggggcacagctctggccacagaggcagatttatttggatgataggactaatatttgtgtaacctgctgagacctgtgtgggagagtttagtatggtttttcttttggtgaggggatttgctccggtttcacatccattaacacaaaacatgagctagtcagggcccttgtggtctgtggtgaggggatgactggagaaacgggactgagtgagtcaggcggagggaatgtcttcctcgcagagtagagtcaacgggataactgatgagccagtggtggggtcacggagggggcggaggggaagagggacttctcttggaagagaggagttttgggggcaggggcgagaacatccaagttacggtatcagtgatggcattggccttcactggggagccagcctgaggtaaatctacttgtgctgtattctctttgagtttgggttcttagctgtggcagacatctgtgacatctcatattactccatgcctttgcctgggctccaaattctagctgataaagatatacaaccactt

SEQ ID NO: 14 - protein, 389 aa, Mouse
MLKLQGEDEAAQLAPRRARVPVPRPTAPDVSPSSARLGLACLLLLLLLTLPARVDTSWWYIGALGARVICDNIPGLVSRQRQLCQRYPDIMRSVGEGAREWIRECQHQFRHHRWNCTTLDRDHTVFGRAMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVTATQDGANFTAARQGYRHATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 15 - DNA, 1506 bp, Homo sapiens
gcgcttctgacaagcccgaaagtcatttccaatctcaagtggactttgttccaactattgggggcgtcgctccccctcttcatggtcgcgggcaaacttcctcctcggcgcctcttctaatggagccccacctgctcgggctgctcctcggcctcctgctcggtggcaccagggtcctcgctggctacccaatttggtggtccctggccctgggccagcagtacacatctctgggctcacagcccctgctctgcggctccatcccaggcctggtccccaagcaactgcgcttctgccgcaattacatcgagatcatgcccagcgtggccgagggcgtgaagctgggcatccaggagtgccagcaccagttccggggccgccgctggaactgcaccaccatagatgacagcctggccatctttgggcccgtcctcgacaaagccacccgcgagtcggccttcgttcacgccatcgcctcggccggcgtggccttcgccgtcacccgctcctgcgccgagggcacctccaccatttgcggctgtgactcgcatcataaggggccgcctggcgaaggctggaagtggggcggctgcagcgaggacgctgacttcggcgtgttagtgtccagggagttcgcggatgcgcgcgagaacaggccggacgcgcgctcggccatgaacaagcacaacaacgaggcgggccgcacgactatcctggaccacatgcacctcaaatgcaagtgccacgggctgtcgggcagctgtgaggtgaagacctgctggtgggcgcagcctgacttccgtgccatcggtgacttcctcaaggacaagtatgacagcgcctcggagatggtagtagagaagcaccgtgagtcccgaggctgggtggagaccctccgggccaagtactcgctcttcaagccacccacggagagggacctggtctactacgagaactcccccaacttttgtgagcccaacccagagacgggttcctttggcacaagggaccggacttgcaatgtcacctcccacggcatcgatggctgcgatctgctctgctgtggccggggccacaacacgaggacggagaagcggaaggaaaaatgccactgcatcttccactggtgctgctacgtcagctgccaggagtgtattcgcatctacgacgtgcacacctgcaagtagggcaccagggcgctgggaaggggtgaagtgtgtggctgggcggattcagcgaagtctcatgggaagcaggacctagagccgggcacagccctcagcgtcagacagcaaggaactgtcaccagccgcacgcgtggtaaatgacccagacccaactcgcctgtggacggggaggctctccctctctctcatcttacatttctcaccctactctggatggtgtgtggtttttaaagaagggggctttctttttagttctctagggtctgataggaacagacctgaggcttatctttgcacatgttaaagaaaaaaaaaaaaa

SEQ ID NO: 16 - protein, 355 aa, Homo sapiens
MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 17 - DNA, 3000 bp, Mouse
cctcttcatgatcgccggcaaacttcctcctcggcgctgcttctaatggagccccacctgctcgggctgctactcggcctcctgctcagtggcaccagggtcctcgctggctacccaatttggtggtccctggccctgggccagcagtacacatctctggcctcccagcctctgctctgcggctccatcccaggcctggtccccaagcaactgcgcttctgccgcaattacatcgagatcatgcccagcgtagcagaaggtgtgaagctgggcatccaggagtgccagcatcagttccggggccgccggtggaactgtaccaccatagatgacagcctggccatctttgggcctgtcttggacaaagccacccgtgaatcggccttcgtgcatgccatcgcctcggctggtgtcgccttcgcagtcacacgctcctgcgctgagggaacctccaccatctgcggctgtgactcacatcataaggggccacctggagaaggctggaagtggggcggctgcagcgaggacgccgacttcggggtgctggtgtcccgggaatttgcggatgcgcgggagaacaggccagatgcccgctcagctatgaacaagcacaacaatgaagcaggccgaacgaccatcctggaccacatgcacctaaagtgtaaatgccacgggttgtccggcagctgcgaggtgaagacctgctggtgggcccagcccgacttccgtgccattggcgacttcctcaaggacaagtacgacagtgcctccgagatggtggtggagaaacaccgtgagtcccgaggctgggtggagaccctgcgggctaagtacgcgctcttcaagccacccaccgagagggacctggtctactacgagaactcccccaacttttgtgagcccaacccagagacgggctcctttggtaccagggaccggacttgcaatgtcacctcccacggcatcgatggctgcgatctgctgtgctgtggccggggccacaacacgaggacggagaaacggaaggagaaatgccattgcgtcttccactggtgctgctatgtcagctgccaagagtgtattcgcatctacgatgtgcacacctgcaagtagtgagccagggcactgggaaggggtagattgtgcggctggatccattcatcgaagtcccatgagaagcaggatctagatccaggccagccttcggcactggccagcaaggagcatggactgttgccagctgcatgtgataaacgacctggacccagccggcctcggacggacgggcggcttctttctcaactaacgtctctccccctgctctggatggtgtacggctttacagaggggctttctttatggttttaccagggtctgctggggacagactcgaggcttacctttgcacatgttaaagaaaataaaaatgaaaaaaaaaaatctaccgcaacagaacaggctgggctagtgtgagctcttggcctggtgggaaggacaagaccatggcgagattctgtgtccaagctgcctctactcgtgacattccaagatgcctctgaggtgggaactgtgaagtaggacagagccccgcagtcccctcttgtccgtcgactcccatttaaattggacataccttgtcgttctgagaaaagccatagataggtgtagctgggatgtagtgatggggaggcccctggccaacagtgggagcaagatcttgagttttgaagacctcagagttctgggcggcctgggaagccatctgcagaacagagttccttgtgggctcctgttttcgctagccctgttctgccctggagcgacagtcagatctccacgcccctttctgttgttctacagtgtccacctttactacgcgttttttttttttttttcatgatgaccttgtaaataggtcagatgtggaggcaggtctcttctggctccatccaccacacccagaaagaatgggctgctctgcccttctcagccttgctaaccagcagacaccgaggagagcagcggggcaccttagagagcaatctaaacatggttggcaggtggggagggtaaagagtcccacttcctttgtgttagaaggcagactaccctgcgtccttttctcccattggctgaagtaaccagaaagacaagagatccttaacaagcccttcttcccacttgtaaaagggatagcctatctcagttcccaaggatctggattagatagatattcaaaagaggcaagcagcgaatggaggcagctcccagctctgttcccgacgcatgatggtactggctgggtttagtaaggtgggtggggctgcacggatcaatccatcaactccgtcttaaggagaatcagaaagaggagataaaatgggggaatggggcagaacaaagaatttgtcctttcccgcttctgtctagggtctgctaatgctggcttgacgaggggtcagccacttctttcctgttgtgcagttggcttgccaagcaggctccagtaggcccttgcctgcactctctaccatgtgaccatgagcactgctctagggacacctcccatcccttcctagcaccccaaatgccccttcccatctctccttccagaagttggaaatcaagtcaactggataacgcttgtgtgagacacttgagcagaacggatacaacaatttacaagtctcttcatatctatgtattctatattaaaagtgataaagtcatgtttccggggcgtattcaagtagctgacaagtaattatttaataatagtacatgagcgcattgtaattatcctcgccatagtcaggtaatagcatccaatgggaggtccctaccaacctgctgtatccaaagttttgtaaaaagttgtagaagttgttgatctttttgattttatattcaaaaagtctctttttataaatattatttattatacaatgtatatacctttgagttaactaagattatatattatataaatatatatatatt

SEQ ID NO: 18 - protein, 355 aa, Mouse
MEPHLLGLLLGLLLSGTRVLAGYPIWWSLALGQQYTSLASQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYALFKPPTERDLVYYENSPNFCEPNPETGSFGTRDRTCNVTSHGIDGCDLLCCGRGHNTRTEKRKEKCHCVFHWCCYVSCQECIRIYDVHTCK

SEQ ID NO: 19 - DNA, 2932 bp, Homo sapiens
agctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacgg

SEQ ID NO: 20 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 21 - DNA, 2814 bp, Mouse
gaattcatgtcttacggtcaaggcagagggcccagcgccactgcagccgcgccacctcccagggccgggccagcccaggcgtccgcgctctcggggtggactccccccgctgcgcgctcaagccggcgatggctcctctcggatacctcttagtgctctgcagcctgaagcaggctctgggcagctacccgatctggtggtccttggctgtgggaccccagtactcctctctgagcactcagcccattctctgtgccagcatcccaggcctggtaccgaagcagctgcgcttctgcaggaactacgtggagatcatgcccagcgtggctgagggtgtcaaagcgggcatccaggagtgccagcaccagttccgaggccggcgttggaactgcaccaccgtcagcaacagcctggccatctttggccctgttctggacaaagccacccgggagtcagcctttgtccatgccatcgcctccgctggagtagctttcgcagtgacacgctcctgtgcagagggatcagctgctatctgtgggtgcagcagccgcctccagggctccccaggcgagggctggaagtggggcggctgtagtgaggacattgaatttggaggaatggtctctcgggagtttgccgatgccagggagaaccggccggatgcccgctctgccatgaaccgtcacaacaatgaggctgggcgccaggccatcgccagtcacatgcacctcaagtgcaaatgccacgggctatctggcagctgtgaagtgaagacctgctggtggtcgcagccggacttccgcaccatcggggatttcctcaaggacaagtatgacagtgcctcggagatggtggtagagaaacaccgagagtctcgtggctgggtggagaccctgaggccacgttacacgtacttcaaggtgccgacagaacgcgacctggtctactacgaggcctcacccaacttctgcgaacctaaccccgaaaccggctccttcgggacgcgtgaccgcacctgcaatgtgagctcgcatggcatagatgggtgcgacctgttgtgctgcgggcgcgggcataacgcgcgcactgagcgacggagggagaaatgccactgtgttttccattggtgctgctacgtcagctgccaggagtgcacacgtgtctatgacgtgcacacctgcaagtaggagagctcctaacacgggagcagggttcattccgaggggcaaggttcctacctgggggcggggttcctacttggaggggtctcttacttggggactcggttcttacttgagggcggagatcctacctgtgagggtctcatacctaaggacccggtttctgccttcagcctgggctcctatttgggatctgggttcctttttaggggagaagctcctgtctgggatacgggtttctgcccgagggtggggctccacttggggatggaattccaatttgggccggaagtcctacctcaatggcttggactcctctcttgacccgacagggctcaaatggagacaggtaagctactccctcaactaggtggggttcgtgcggatgggtgggaggggagagattagggtccctcctcccagaggcactgctctatctagatacatgagagggtgcttcagggtgggccctatttgggcttgaggatcccgtgggggcggggcttcaccccgactgggtggaacttttggagacccccttccactggggcaaggcttcactgaagactcatgggatggagctccacggaaggaggagttcctgagcgagcctgggctctgagcaggccatccagctcccatctggcccctttccagtcctggtgtaaggttcaacctgcaagcctcatctgcgcagagcaggatctcctggcagaatgaggcatggagaagaactcaggggtgataccaagacctaacaaaccccgtgcctgggtacctcttttaaagctctgcaccccttcttcaagggctttcctagtctccttggcagagctttcctgaggaagatttgcagtcccccagagttcaagtgaacacccatagaacagaacagactctatcctgagtagagagggttctctaggaatctctatggggactgctaggaaggatcctgggcatgacagcctcgtatgatagcctgcatccgctctgacacttaatactcagatctcccgggaaacccagctcatccggtccgtgatgtccatgccccaaatgcctcagagatgttgcctcactttgagttgtatgaacttcggagacatggggacacagtcaagccgcagagccagggttgtttcaggacccatctgattccccagagcctgctgttgaggcaatggtcaccagatccgttggccaccaccctgtcccgagcttctctagtgtctgtctggcctggaagtgaggtgctacatacagcccatctgccacaagagcttcctgattggtaccactgtgaaccgtccctccccctccagacaggggaggggatgtggccatacaggagtgtgcccggagagcgcggaaagaggaagagaggctgcacacgcgtggtgactgactgtcttctgcctggaactttgcgttcgcgcttgtaactttattttcaatgctgctatatccacccaccactggatttagacaaaagtgattttctttttttttttttcttttctttctatgaaagaaattattttagtttatagtatgtttgtttcaaataatggggaaagtaaaaagagagaaaaaaaaaaaaaaaaaaaaaaaaaaaaaaaaaaaaaaaaaa

SEQ ID NO: 22 - protein, 352 aa, Mouse
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 23 - DNA, 1595 bp, Homo sapiens
ggctctggggcggcgctgacagtctggtccgcgccgggcagcgggcgcagcagcgggcaggctgccggcaggcacacggaggcagagccccgccgcgcgcgccccggcccgcccgcgggcgcccacctgcagccccgacgggaggccccccgcggccgcagccgctgccccgggccgggcgcccgcggcggcaccatgagtccccgctcgtgcctgcgttcgctgcgcctcctcgtcttcgccgtcttctcagccgccgcgagcaactggctgtacctggccaagctgtcgtcggtggggagcatctcagaggaggagacgtgcgagaaactcaagggcctgatccagaggcaggtgcagatgtgcaagcggaacctggaagtcatggactcggtgcgccgcggtgcccagctggccattgaggagtgccagtaccagttccggaaccggcgctggaactgctccacactcgactccttgcccgtcttcggcaaggtggtgacgcaagggactcgggaggcggccttcgtgtacgccatctcttcggcaggtgtggcctttgcagtgacgcgggcgtgcagcagtggggagctggagaagtgcggctgtgacaggacagtgcatggggtcagcccacagggcttccagtggtcaggatgctctgacaacatcgcctacggtgtggccttctcacagtcgtttgtggatgtgcgggagagaagcaagggggcctcgtccagcagagccctcatgaacctccacaacaatgaggccggcaggaaggccatcctgacacacatgcgggtggaatgcaagtgccacggggtgtcaggctcctgtgaggtaaagacgtgctggcgagccgtgccgcccttccgccaggtgggtcacgcactgaaggagaagtttgatggtgccactgaggtggagccacgccgcgtgggctcctccagggcactggtgccacgcaacgcacagttcaagccgcacacagatgaggacctggtgtacttggagcctagccccgacttctgtgagcaggacatgcgcagcggcgtgctgggcacgaggggccgcacatgcaacaagacgtccaaggccatcgacggctgtgagctgctgtgctgtggccgcggcttccacacggcgcaggtggagctggctgaacgctgcagctgcaaattccactggtgctgcttcgtcaagtgccggcagtgccagcggctcgtggagttgcacacgtgccgatgaccgcctgcctagccctgcgccggcaaccacctagtggcccagggaaggccgataatttaaacagtctcccaccacctaccccaagagatactggttgtattttttgttctggtttggtttttgggtcctcatgttatttattgccgaaaccaggcaggcaaccccaagggcaccaaccagggcctccccaaagcctgggcctttgtggctgccactgaccaaagggaccttgctcgtgccgctggctgcccgcatgtggctgccactgaccactcagttgttatctgtgtccgtttttctacttgcagacctaaggtggagtaacaaggagtattaccaccaca

SEQ ID NO: 24 - protein, 351 aa, Homo sapiens
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 25 - DNA, 1101 bp, Mouse
cgggagccttgcggccgctgccccgggctgggcgcgcacggcaccatgagcccccgttcgtgcctgcggtccctgcgactcctcgtcttcgccgtgttctcggccgccgcgagcaattggctgtacctggccaagctgtcatcggtgggcagcatctccgaagaggagacgtgcgagaaactcaaaggcctgatccagaggcaggtgcagatgtgcaaacggaaccttgaggtgatggactcagtgcgccgtggtgcccagctggccatcgaggagtgccaataccagttccggaaccggcgctggaactgttccacactggactccctccctgtctttgggaaggtggtgacacaagggacccgggaggcggcctttgtatacgccatctcttcagcaggtgtggcctttgcagtgacaagggcatgcagcagtggagaactggagaagtgtggctgtgaccggacagtgcacggggtcagcccacagggcttccagtggtcaggatgctcggacaacatcgcctatggcgtagccttctcacagtcctttgtggacgtccgggagaggagcaagggggcctcctccagccgggcactcatgaatcttcacaacaacgaggctggcaggaaggccatcttgacacacatgcgggtggagtgcaagtgtcacggggtgtcgggctcctgcgaggtaaagacgtgctggcgtgctgtaccgcccttccgccaggttggccacgcgctaaaggagaagtttgacggtgccacggaggtggagccacgacgcgtaggctcctcccgggcgctggtgcctcggaatgcacagttcaagccacatacagatgaggacctggtatacctggagcctagcccggacttctgtgagcaggacatccgcagtggcgtgctaggcacgaggggccgcacgtgcaacaagacatctaaagccattgacggctgcgagctactgtgctgtggccgcggcttccacacagcgcaagtggagctggccgagcgctgtggctgcaggttccactggtgctgcttcgtcaagtgccggcagtgccagcggctcgtggagatgcacacgtgccggtga

SEQ ID NO: 26 - protein, 351 aa, Mouse
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDIRSGVLGTRGRTCNKTSKAIDGCELLCCGRGFHTAQVELAERCGCRFHWCCFVKCRQCQRLVEMHTCR

SEQ ID NO: 27 - DNA, 5855 bp, Homo sapiens
agttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctgcacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatccaaaaaaaaaaaaaaaaaa

SEQ ID NO: 28 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 29 - DNA, 4273 bp, Mouse
agtccctggaagcagacgtttcggccacagacccagagaggaggagctgacaatcaggaggcgtgagccgcctggagtctgcagaattcgtggtgtgaatgaactgggggcatcttgggcacagggattgccccccctccttccccgcctcgggccacagttgagtagtggggcattttttttcaccttcttgtgaagaattttttttattatttgttgtaaagtcttttgcacaatcacgcccacatttggggttggaaagccctaattaccgccgtcgctgatggacgttagagagggagcgcctcgccgcggaacagtcgcctgcgcgccctcgtcggacccgcggctcctgcactgtgtccccgctcggccctgcgcttgctgctcgcccgcgcgcgccggcgccctctcggttcctgggcacatttccacgctataccaactcctctgcccgagtccgggcgccagtgctcgcttccgctccgggtcgctgcgcccacccgacgcgcccaggaggactccgcagccctgctttggattgtcccccaaggcttaaccccgacgcttcgcttgaattcctcggccgccttcgctcgggtggcgacttcctctccgtgccccctccccctcgccatgaagaagcccattggaatattaagcccgggagtggctttggggaccgctggaggtgccatgtcttccaagttcttcctaatggctttggccacgtttttctccttcgcccaggttgttatagaagctaattcttggtggtctctaggtatgaataaccctgttcagatgtcagaagtatatatcataggtgcacagcctctctgcagccaactggcaggactttctcaaggacagaagaaactctgccacttgtatcaggaccacatgcagtacattggagaaggtgcgaagacaggcatcaaggaatgccagtaccagttccggcatcggagatggaactgcagcacagtggacaatacttctgtctttggcagggtgatgcaaataggcagccgagagacggccttcacgtacgcggtgagcgcagctggggtggtgaacgccatgagccgagcatgccgggagggcgagctgtctacctgtggctgcagccgcgctgcgcgccccaaggacctgcctcgggactggttgtggggcggctgcggagacaacatcgactatggctaccgcttcgccaaggagttcgtggacgctagagaaagggaacgaatccacgctaagggttcctatgagagcgcacgcatcctcatgaacttacacaacaatgaagcaggccgtaggacagtatacaacctggcagatgtagcctgtaagtgtcatggagtgtctggctcctgtagcctcaagacgtgctggctgcagctggcggacttccggaaggtgggcgatgccctcaaggagaagtatgatagcgcggcggccatgaggctcaacagccggggcaagctggtgcaggtcaacagccgcttcaactccccgaccacgcaggacctggtctacatcgaccccagtccggactactgtgtgcgcaacgagagcactggctcgctgggcacgcagggacgcctgtgcaacaagacctcagaggggatggacggctgcgagctcatgtgctgtgggcgtggctatgaccagtttaagacagtgcagaccgaacgctgtcattgcaagtttcactggtgctgctatgtcaaatgcaagaagtgcacggagattgtggatcagttcgtgtgcaaatagtggtgtgcctgcccttcacccagtcccactcccaggacccacttatttatagaaagtacagtgcttctggttctttttatttctcccccaagaattgcagctggaaccatgtgttttgttttgttttattttgttttttcttttctgttaccatctaagaactctgtggtttattattaatattataattaatatttggcaatagtgggggaaactaagaaaaatatttattttgaggatctttgcaaagttagtacaaaatttctttcttctgatgctacaggataaaggggaaaaactatgtattcgaacttagctgtgcagttgggggttcacatctagaaggtgtaggagccattttcttctcaaacagagagtcctttgagatgggtggtatccaggtgaaggaggaggtacagacccatgaataacagttcctgtgaccaaaatgaattgcaggtgctctggtacaaaagatcttaaatatagatatattaaatatacatatatgccaaaaatacagaatatgagacactccctaacccagaggttaccagcctggttttgtgggttttttgttttgttttgttttttctttttttgggttttgtttgtttgtttgtttgtttgtatttttggtgtgtgtgtgtgtgtatttctagaatgatcttttagaaggtacaagcaagaatctcatatcttcagaagcaggcatatcatgtatgttactgtgtcccacctacagatactccattcatgaatgggcctttttctaacagttcatgaatattggggagccggtgggctgggggagggaggtccccagaaattagaaaacttgaagtttcctacattgaggccataatcttgtgttagcccagctgattcttaataccagacttttagatccataaaggaatttttgactaaaaaaaaaaaatcttgttttgaaagccatcttattttcttaaaaatgaaaaattacccatgaatcccatttgcaacccctcacccccacaggcaacaagaaagtcccatgtagttgagcactgcgaacacctctgtgaggagatgatggcagccatcttcctgcatgatcccatgccctttctggactctctgctggccatgcttccgaatggcagccctggtggacactcactgctggtagggcagaaaatgtacacgaggagccatgttcagaaccagccacttaggggttgttctctgaggcttttctttggaggtacggtaacttgatgtgttttgatgatatctcttggcccagggagtccacagaggtgttgcagctgtttggttgttatcctcctgcgtttagactttccatttgtgcttttcctattaccctgcaggtgtaccctaaaactgttcctagtgtacttgaacagttgcatttataaggggggatgtggtttaatggtgcctgatatctcagtttttttgtatataacatatatataaatatacatatataaatatagatataattatatctcagtgcagtctgggatttagacctacagttttctctgggcttgctctctgcctggagtatcgtccttcattgcagtccaattgggatttctttttttccaaaaattttgagtcttaacattgacctgtgacaggatcctaccacgaataccaggaagcaagctaagactcggaggaagctctcagggctcatgtcctgaatgtatgttggttagaaagtagcctttctgcttcctgcccatggccagttctccaccctctctttggtgttctttgtggggagggcactgtggtttgtcgcagccctggacttcgagaggctcccagaacccaggatcaccagcctcctgtctgtttgcttcactcctttcccagggaggacttgggactgtcctgtctgacaggacggatctgagttcccgaagcaaaccagctcaccacatagatagctagtttaaacaatgttttaaaataagggcacctctgtttcaaaagtgacatctgctgtgttgttttcgaggcctgatactcttacaaggtttgaaaaaaaatgtgtgtatccattcatgggcttggtagccttctggtcacctcagtcctgtggctcttaacttattgcccaacaatattcatttcccctcagctacaatgaattgcaagcaaaagatgttgaaaaaaagcactaatttagtttaaaatgtcactttttggtttttattctacaaaaaccatgaagttctctctctctctctctctctctcttatttgttaaatcagattatgttctttttttgtttttgtttttagtgattcatgtttatgagcagagtggagtttaacaatcctagctttaaaaaaaacctatttaatgtaagatattctacgcatccttcagatattttgtatatcccctatggcctttattctgtacttttaatgtacatatttctgtcttgtgtgatttgtatatttcactggt

SEQ ID NO: 30 - protein, 380 aa, Mouse
MKKPIGILSPGVALGTAGGAMSSKFFLMALATFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 31 - DNA, 2184 bp, Homo sapiens
tattcttccaaatggaaactgctaatttttgaagcagaaggttgacagcttcagtaagatctcaagagagcgagaagactggaatcagggaaccctactctggaaactgtcagtcccagggcactggggagggctgaggccgaccatgcccagcctgctgctgctgttcacggctgctctgctgtccagctgggctcagcttctgacagacgccaactcctggtggtcattagctttgaacccggtgcagagacccgagatgtttatcatcggtgcccagcccgtgtgcagtcagcttcccgggctctcccctggccagaggaagctgtgccaattgtaccaggagcacatggcctacataggggagggagccaagactggcatcaaggaatgccagcaccagttccggcagcggcggtggaattgcagcacagcggacaacgcatctgtctttgggagagtcatgcagataggcagccgagagaccgccttcacccacgcggtgagcgccgcgggcgtggtcaacgccatcagccgggcctgccgcgagggcgagctctccacctgcggctgcagccggacggcgcggcccaaggacctgccccgggactggctgtggggcggctgtggggacaacgtggagtacggctaccgcttcgccaaggagtttgtggatgcccgggagcgagagaagaactttgccaaaggatcagaggagcagggccgggtgctcatgaacctgcaaaacaacgaggccggtcgcagggctgtgtataagatggcagacgtagcctgcaaatgccacggcgtctcggggtcctgcagcctcaagacctgctggctgcagctggccgagttccgcaaggtcggggaccggctgaaggagaagtacgacagcgcggccgccatgcgcgtcacccgcaagggccggctggagctggtcaacagccgcttcacccagcccaccccggaggacctggtctatgtggaccccagccccgactactgcctgcgcaacgagagcacgggctccctgggcacgcagggccgcctctgcaacaagacctcggagggcatggatggctgtgagctcatgtgctgcgggcgtggctacaaccagttcaagagcgtgcaggtggagcgctgccactgcaagttccactggtgctgcttcgtcaggtgtaagaagtgcacggagatcgtggaccagtacatctgtaaatagcccggagggcctgctcccggcccccctgcactctgcctcacaaaggtctatattatataaatctatataaatctattttatatttgtataagtaaatgggtgggtgctatacaatggaaagatgaaaatggaaaggaagagcttatttaagagacgctggagatctctgaggagtggactttgctggttctctcctcttggtgggtgggagacagggctttttctctccctctggcgaggactctcaggatgtagggacttggaaatatttactgtctgtccaccacggcctggaggagggaggttgtggttggatggaggagatgatcttgtctggaagtctagagtctttgttggttagaggactgcctgtgatcctggccactaggccaagaggccctatgaaggtggcgggaactcagcttcaacctcgatgtcttcagggtcttgtccagaatgtagatgggttccgtaagaggcctggtgctctcttactctttcatccacgtgcacttgtgcggcatctgcagtttacaggaacggctccttccctaaaatgagaagtccaaggtcatctctggcccagtgaccacagagagatctgcacctcccggacttcaggcctgcctttccagcgagaattcttcatcctccacggttcactagctcctacctgaagaggaaagggggccatttgacctgacatgtcaggaaagccctaaactgaatgtttgcgcctgggctgcagaagccagggtgcatgaccaggctgcgtggacgttatactgtcttcccccacccccggggaggggaagcttgagctgctgctgtcactcctccaccgagggaggcctcacaaaccacaggacgctgcaacgggtcaggctggcgggcccggcgtgctcatcatctctgccccaggtgtacggtttctctctgacattaaatgcccttcatgg

SEQ ID NO: 32 - protein, 359 aa, Homo sapiens
MPSLLLLFTAALLSSWAQLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 33 - DNA, 2129 bp, Mouse
ccttgctgcttctcattccatgagctggggagagacagtgtggaagtcaaaccatgtgtttcttgagagcaggtgctgctggggctccctgaatggcggctaggtgccaagagggagctccgctttggaagatgttggtcccagggcattgggatgggttgaggccggccatgcccagcctgctgctggtggtcgtggcagctctgctctccagctgggcacagctgctgactgacgccaactcctggtggtcactagctctgaacccagtgcagagaccggagatgttcatcattggcgctcagcccgtgtgcagccaacttcctgggctttccccaggccagagaaagctgtgtcagttgtatcaggagcacatgtcctacatcggggagggagccaagacgggcatcagagagtgccaacaccagtttcgacagaggcgctggaactgcagcaccgtggacaacacatctgtctttggcagagttatgcagataggtagccgagagactgccttcacgtatgcagtgagcgccgctggcgtggtgaatgccatcagccgagcctgcagagagggtgagctgtccacctgtggctgcagccgtgctgcgaggcccaaggacctgcctcgggactggctgtggggtggctgtggagacaacgtggagtacggctaccgctttgccaaggagtttgtggatgcccgagagcgtgagaagaactttgccaagggatcggaggagcagggccgagctctcatgaacctacagaacaacgaggctggccgccgggccgtgtataagatggctgatgtcgcctgcaaatgtcacggagtctccgggtcctgcagcctcaagacctgctggctccagctggccgagttccgcaaggttggggaccgtttgaaggagaagtacgacagcgccgcggccatgcgcatcacccgccagggcaagctggagctggccaacagccgcttcaaccagcccaccccagaggacctggtctacgtggaccccagtcctgactactgcttgcgtaatgagaccacaggctccctgggcacccagggtcgcctctgcaacaagacctcagagggcatggacggctgcgagctcatgtgctgtggccgcggctatgaccgcttcaagagcgttcaggtggaacgctgccactgcaggttccactggtgttgctttgtcagatgcaaaaaatgcaccgaggttgtggaccagtatgtctgtaagtgactgcaccacacgggccttcaggccgctcctctccgccttacaaaagtctatattatataaatctatctaaatatattttatatttgtacaaatggatggatggatggatgatagataatcaagagaagaaagtggagaggaagagcttaggagatgctggccctctgtgaggactggattttgctggaaatccacaaccagtgggagagaaacgggcttttccccattttctggccaggacttttgggacatgggcttgagagtgtctgtgtgccatagcctccaggagtcaggtggggattagatgaaggaactggacttattccacatctacagtcctgtggggaagatgagtgtctgtgaccctggccaggagacccagaggccctgtggaaagacctgataactgggatggtagcctaggtcttcctgaaaatggagccagctttgggaaggggctctgtacttccttcttttctcatctgagtacacactgcaggaaagtcccctgccccaatatgggggagtggtctcaagtcactccaaccggtgaccgtaagagatctgggcctccctggaccctggctctgccttctgatgagaatgtcactagctcctgcctcaagctcttgtgccaagagaaagactgttccgtcacctgctacagccaggaagacgtggagcaaacctgggttttgactggggaccaagtgcctgttgcacaggacaggaatctgctgtcactctgtcaagggaggctttgagaatgacagggcatgctagcaggtcaggtcaactgcctgtgagactgtcatctctgcccacatgtacagcgtccctctgacattaaatatctttttactgaaaaaaaaaaaaaaa

SEQ ID NO: 34 - protein, 372 aa, Mouse
MLVPGHWDGLRPAMPSLLLVVVAALLSSWAQLLTDANSWWSLALNPVQRPEMFIIGAQPVCSQLPGLSPGQRKLCQLYQEHMSYIGEGAKTGIRECQHQFRQRRWNCSTVDNTSVFGRVMQIGSRETAFTYAVSAAGVVNAISRACREGELSTCGCSRAARPKDLPRDWLWGGCGDNVEYGYRFAKEFVDAREREKNFAKGSEEQGRALMNLQNNEAGRRAVYKMADVACKCHGVSGSCSLKTCWLQLAEFRKVGDRLKEKYDSAAAMRITRQGKLELANSRFNQPTPEDLVYVDPSPDYCLRNETTGSLGTQGRLCNKTSEGMDGCELMCCGRGYDRFKSVQVERCHCRFHWCCFVRCKKCTEVVDQYVCK

SEQ ID NO: 35 - DNA, 1726 bp, Homo sapiens
ggcacgagcgcaggagacacaggcgctggctgccccgtccgctctccgcctccgccgcgccctcctcgcccgggatgggcccccccgccgccgccggatccctcgcctcccggccgccgccgttgcgctcgccgcgctcgcactgaagcccgggccctcgcgcgccgcggttcgccccgcagcctcgccccctgcccacccgggcggccgtagggcggtcacgatgctgccgcccttaccctcccgcctcgggctgctgctgctgctgctcctgtgcccggcgcacgtcggcggactgtggtgggctgtgggcagccccttggttatggaccctaccagcatctgcaggaaggcacggcggctggccgggcggcaggccgagttgtgccaggctgagccggaagtggtggcagagctagctcggggcgcccggctcggggtgcgagagtgccagttccagttccgcttccgccgctggaattgctccagccacagcaaggcctttggacgcatcctgcaacaggacattcgggagacggccttcgtgttcgccatcactgcggccggcgccagccacgccgtcacgcaggcctgttctatgggcgagctgctgcagtgcggctgccaggcgccccgcgggcgggcccctccccggccctccggcctgcccggcacccccggaccccctggccccgcgggctccccggaaggcagcgccgcctgggagtggggaggctgcggcgacgacgtggacttcggggacgagaagtcgaggctctttatggacgcgcggcacaagcggggacgcggagacatccgcgcgttggtgcaactgcacaacaacgaggcgggcaggctggccgtgcggagccacacgcgcaccgagtgcaaatgccacgggctgtcgggatcatgcgcgctgcgcacctgctggcagaagctgcctccatttcgcgaggtgggcgcgcggctgctggagcgcttccacggcgcctcacgcgtcatgggcaccaacgacggcaaggccctgctgcccgccgtccgcacgctcaagccgccgggccgagcggacctcctctacgccgccgattcgcccgacttttgcgcccccaaccgacgcaccggctcccccggcacgcgcggtcgcgcctgcaatagcagcgccccggacctcagcggctgcgacctgctgtgctgcggccgcgggcaccgccaggagagcgtgcagctcgaagagaactgcctgtgccgcttccactggtgctgcgtagtacagtgccaccgttgccgtgtgcgcaaggagctcagcctctgcctgtgacccgccgcccggccgctagactgacttcgcgcagcggtggctcgcacctgtgggacctcagggcaccggcaccgggcgcctctcgccgctcgagcccagcctctccctgccaaagcccaactcccagggctctggaaatggtgaggcgaggggcttgagaggaacgcccacccacgaaggcccagggcgccagacggccccgaaaaggcgctcggggagcgtttaaaggacactgtacaggccctccctccccttggcctctaggaggaaacagttttttagactggaaaaaagccagtctaaaggcctctggatactgggctccccagaactgctggccacaggatggtgggtgaggttagtatcaataaagatatttaaaccaaaaaaaaaaaaaaaaaaaaa

SEQ ID NO: 36 - protein, 365 aa, Homo sapiens
MLPPLPSRLGLLLLLLLCPAHVGGLWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 37 - DNA, 1669 bp, Mouse
ccgccgcgccctcctcgcccgggatgggcccccccgccgccaccgccgccggagccctagtctccgggccgccgcctcggtcgccgcgtttgccctgaagcccggtgcccgcgcgccccggctcaccccgcagcttcactccccacccccagccgcctccccggccagactgcggtagagctctcaggatgctgccgccggtgccctcccgcctcggactgctgctgctgctcttgtgccccgcgcacgtcgatggactgtggtgggccgtgggcagccccttggtcatggatcctaccagcatctgcaggaaggccaggcggctggcaggaagacaggccgagctgtgccaggcggagccggaagtagtggcagagcttgcccgaggcgcaagactgggggttcgagaatgtcagttccagttccgtttccgacgctggaactgctccagccacagcaaggcctttgggcgcgtcctgcagcaggacatccgagagacagctttcgtgtttgcaatcaccgcagctggtgccagccacgcggtcactcaagcctgttccatgggagagctcctacagtgtggttgtcaggcaccccgcgggcgggcaccgcctaggccctccggccttctgggcactcctggacctccaggaccaactggctctccagatgctagcgcagcctgggagtggggaggctgcggagacgatgtggacttcggggatgagaagtcaagactctttatggatgcgcagcacaagcggggccgtggagatatccgtgcattggtgcaactgcacaacaacgaggcgggcaggctggcggtgcggagtcacacgcgcaccgagtgtaagtgccatgggctttcgggttcctgcgctctgcgcacctgctggcagaagctgcctccgttccgcgaggtgggcgcacggctgctggagcgcttccacggcgcctcgcgcgtcatgggcaccaacgacggcaaagctctgctgcctgcggtccgcacactcaagcctcccggacgagcggatctcctctacgcagccgattcacccgacttctgcgcccccaaccggcgcacgggttcgccgggcacgcgcggacgcgcctgcaacagcagtgccccggacctcagcggctgcgacctgttgtgctgcggtcgcgggcaccgccaggagagcgtacagctcgaggagaactgtctgtgccgcttccactggtgctgcgtggtgcaatgccaccgctgccgggtgcgcaaggaactcagcctgtgcctctgacccgtcgcctgcctcggaactgctggcagccacctctgggccatctacaggactattagattccagcagggggcgctgtctgagtccagcagctccctaggaaaagtacctatccaggccttgggaaattacaggggccagccaggaacttggggtttacaccagcccacgaaagcccgggggaacatacccctccagcattcccctgaaaggccctttgctagttcctgcaggagatcactccccttggccccccagatggaaataagaaagccagactctgccctctggaaataatattcctcagaattactgggatggatgggtgagtttagtatcaataaagacatttaaatccacaaaaaaaaaaaaaaaaaaaaaaaaaa

SEQ ID NO: 38 - protein, 364 aa, Mouse
MLPPVPSRLGLLLLLLCPAHVDGLWWAVGSPLVMDPTSICRKARRLAGRQAELCQAEPEVVAELARGARLGVRECQFQFRFRRWNCSSHSKAFGRVLQQDIRETAFVFAITAAGASHAVTQACSMGELLQCGCQAPRGRAPPRPSGLLGTPGPPGPTGSPDASAAWEWGGCGDDVDFGDEKSRLFMDAQHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 39 - DNA, 1732 bp, Homo sapiens
gaggggcgggggctggaggcagcagcgcccccgcactccccgcgtctcgcacacttgcaccggtcgctcgcgcgcagcccggcgtcgccccacgccgcgctcgctcctccctccctcctcccgctccgtggctcccgtgctcctggcgaggctcaggcgcggagcgcgcggacgggcgcaccgacagacggccccggggacgcctcggctcgcgcctcccgggcgggctatgttgattgccccgccggggccggcccgcgggatcagcacagcccggcccgcggccccggcggccaatcgggactatgaaccggaaagcgcggcgctgcctgggccacctctttctcagcctgggcatggtctacctccggatcggtggcttctcctcagtggtagctctgggcgcaagcatcatctgtaacaagatcccaggcctggctcccagacagcgggcgatctgccagagccggcccgacgccatcatcgtcataggagaaggctcacaaatgggcctggacgagtgtcagtttcagttccgcaatggccgctggaactgctctgcactgggagagcgcaccgtcttcgggaaggagctcaaagtggggagccgggaggctgcgttcacctacgccatcattgccgccggcgtggcccacgccatcacagctgcctgtacccagggcaacctgagcgactgtggctgcgacaaagagaagcaaggccagtaccaccgggacgagggctggaagtggggtggctgctctgccgacatccgctacggcatcggcttcgccaaggtctttgtggatgcccgggagatcaagcagaatgcccggactctcatgaacttgcacaacaacgaggcaggccgaaagatcctggaggagaacatgaagctggaatgtaagtgccacggcgtgtcaggctcgtgcaccaccaagacgtgctggaccacactgccacagtttcgggagctgggctacgtgctcaaggacaagtacaacgaggccgttcacgtggagcctgtgcgtgccagccgcaacaagcggcccaccttcctgaagatcaagaagccactgtcgtaccgcaagcccatggacacggacctggtgtacatcgagaagtcgcccaactactgcgaggaggacccggtgaccggcagtgtgggcacccagggccgcgcctgcaacaagacggctccccaggccagcggctgtgacctcatgtgctgtgggcgtggctacaacacccaccagtacgcccgcgtgtggcagtgcaactgtaagttccactggtgctgctatgtcaagtgcaacacgtgcagcgagcgcacggagatgtacacgtgcaagtgagccccgtgtgcacaccaccctcccgctgcaagtcagattgctgggaggactggaccgtttccaagctgcgggctccctggcaggatgctgagcttgtcttttctgctgaggagggtacttttcctgggtttcctgcaggcatccgtgggggaaaaaaaatctctcagagccctcaactattctgttccacacccaatgctgctccaccctcccccagacacagcccaggtccctccgcggctggagcgaagccttctgcagcaggaactctggacccctgggcctcatcacagcaatatttaacaatttattctgataaaaataatattaatttatttaattaaaaagaattcttccacaaaaaaaaaaaaaaaaaa

SEQ ID NO: 40 - protein, 349 aa, Homo sapiens
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 41 - DNA, 3177 bp, Mouse
ggcagtccccgcgcctcaaacacttgccgcgatcgctggcgcgcagcggcgccccttgttgcgcttgttctcccctcctctggctccgcggctcccgcgctctgggacagtctccagtgcctagcgcggaccgacgcaccgacggaccgcccagggagcctcggcccgcgccccctgcgcaggctatgtggattgccccgccgggcccggctggcgggatcagcacagcccggcccgtggcacccgccaccagcggggactatgacccggaaagcgcggcgctgcctgggccacctctttctcagcctgggcatagtctacctccggatcggtggcttctcttcggtggtagctctgggtgcgagcatcatctgtaacaagatcccaggcctggctcccagacagcgggcaatctgccagagccggccggacgccatcatcgtcataggagaaggctcccaaatgggcctggacgagtgtcagtttcagttccgaaatggccgttggaactgctcagcgctgggagagcgtactgtcttcgggaaggagctcaaagtggggagtcgggaggctgccttcacctatgcgattatcgctgcgggcgtggcccatgccatcactgctgcctgcacccagggcaacctgagcgactgtggctgcgacaaggagaagcaaggccagtaccaccgggacgagggctggaagtggggtggctgctctgccgacatccgctacggcatcggcttcgccaaggtcttcgtggatgcccgggagatcaagcagaatgcccggacgctcatgaacttacacaataacgaggcgggtcggaagatcctggaggagaacatgaagctggagtgtaagtgccatggtgtgtcaggctcctgtaccactaagacgtgctggaccacactgccacagttccgagagctaggctacgtgctcaaggacaaatacaacgaggccgtccacgtggagcctgtgcgtgccagtcgaaacaagcggcccacctttctgaagatcaagaagcccctgtcctaccgcaagcccatggacactgacctggtgtatatcgagaagtcacccaattactgtgaagaggacccagtgacaggcagcgtgggtacccagggccgagcctgcaataagacagcccctcaggccagtggctgtgacctcatgtgctgtggccgtggctacaacacacaccagtacgcccgggtgtggcagtgcaactgcaaattccactggtgctgctacgtcaagtgtaacacgtgcagcgagcgcacggagatgtatacgtgcaagtgaatgcggtcacaggtcagatcacaggcaggatacagtttccctgcaggccactgcctggatgctcacagggaaagaaccacagaagcactgtccttgtcttttctgctgaggggggaggggtattctgggtttcctgcagactcccgtgggaagcatctctcagaggcccgcccattcttctccacatggatgctgctcagccaccctcccccagacaccgcccgagcctctccagggctggaacaaagttttctacggcaggagctctggagcctcgggcctcgtcatagcaatatttaacagtttattctgatatgagataatattaatttatttaattaaagagaattcttccacttcgtcgggatccgtcttctgcaatcaaagtggactgcttgaggtcctggtgggatgacttgctaggactgggagctgagaacagctgtacataattattctttatgcagatgtttctactagttgatttcacaagtacccttctgcagcgctaggtgttaagtacaaagagaagacggtctttatacacatatagatatatatatgcatacacatttgtaactttgttttgttttgtttttgctgtttgctgctacctatccagactctaagctggtccagatctggaattgtttttctccaggacgtgctcctatccttttgccctttacagttcaaacctctccgttagaaaagttccattgggaatggcgtgtgtgtgatggggacgaggatcacaaattcccagcagtttccatcctgaaacgtgaaccactggataagaggctttctaagagactatttttctatggatattttatttatatggagtctgcctgcggtgccccatggcccatgcctcttcttaacactggtactcactcaggggcagaaggacaaggccaggtgtgtgggcaggtcccccggggaccctcacacagctggagcctggagttctatttgccaagggggccatagcagttaccagatgcctgggttgggtatcttctgtgttaaacaagagggaaccatcccctggctttagcctgctaagctcagggcttggaatggggtcactggatggttatcttgggagatgacctctggatgagcctcagcggtgggtcagtcagtgtctcacacactttgagaagcatgggacctggcattcatcatcaggcagaggccagctcagggatgccgctatcccatcaggacagcccaggcactgcctctaggtgaggtgtagtcctaagagaaggggtcaaggagggggaaggaggaagccaaggagtgttggccatcctcagtgaaagcgatgggagcgttctctcagcagcagagacacagctgtacctgtatctctccaatgggaaacccctccagaaggctggggatattttttatgtgtttccacatgcatttccacctgtgtgcatgtaagcacatgcgcacactcctgtgccagcactctgcggcacctccagggtgctcacgggtacatgtgcttacatgtatctctctgtgcttgggagatcagaccatgtgcatggagctgtatgcctgagcacttgtggtctcaggggttatttccaggtatctgcatttgtgggtggggtgcaaggtagacagcagggaactgatttgattgtgttgagccacagtgagactgcaactctgaactctgtctccacagctgctggtgaaactcagatgcctgtgagacaacagccctgagcctcatggcccacatgctgggagcccctcagtgtctaggtcatgtccagtcccccacctgggttacatcacgaccaataaacatggctgtatggctgatttcttcccttg

SEQ ID NO: 42 - protein, 349 aa, Mouse
MTRKARRCLGHLFLSLGIVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 43 - DNA, 2250 bp, Homo sapiens
gagtctgcccgcagccccctggcccctgcccggccctgcgtgcccgcgcgtccctccggccgcgctgtctatggcgcagcccccctccctggatcatgcacagaaactttcgcaagtggattttctacgtgtttctctgctttggcgtcctgtacgtgaagctcggagcactgtcatccgtggtggccctgggagccaacatcatctgcaacaagattcctggcctagccccgcggcagcgtgccatctgccagagtcggcccgatgccatcattgtgattggggagggggcgcagatgggcatcaacgagtgccagtaccagttccgcttcggacgctggaactgctctgccctcggcgagaagaccgtcttcgggcaagagctccgagtagggagccgtgaggctgccttcacgtacgccatcaccgcggctggcgtggcgcacgccgtcaccgctgcctgcagccaagggaacctgagcaactgcggctgcgaccgcgagaagcagggctactacaaccaagccgagggctggaagtggggcggctgctcggccgacgtgcgttacggcatcgacttctcccggcgcttcgtggacgctcgggagatcaagaagaacgcgcggcgcctcatgaacctgcataacaatgaggccggcaggaaggttctagaggaccggatgcagctggagtgcaagtgccacggcgtgtctggctcctgcaccaccaaaacctgctggaccacgctgcccaagttccgagaggtgggccacctgctgaaggagaagtacaacgcggccgtgcaggtggaggtggtgcgggccagccgtctgcggcagcccaccttcctgcgcatcaaacagctgcgcagctatcagaagcccatggagacagacctggtgtacattgagaagtcgcccaactactgcgaggaggacgcggccacgggcagcgtgggcacgcagggccgtctctgcaaccgcacgtcgcccggcgcggacggctgtgacaccatgtgctgcggccgaggctacaacacccaccagtacaccaaggtgtggcagtgcaactgcaaattccactggtgctgcttcgtcaagtgcaacacctgcagcgagcgcaccgaggtcttcacctgcaagtgaggccaggcccggaggcggccgcgggcaccctggaacccggcggcattttgcacatccactcctcaccttccctgccttggtgctgccagcagcagacatagacgggtgcagaagcggggagctccaggtgcaggagggcaccggccggggcccacgccctctgcccgcctccctggggctccttcctgccacctcctcccatcacctcctgcggcagaacagcacccgtgacccacccagagagcaaggccaggggtcttggtgctccccgacggggcccggcaagttctctttcttctctctgggaaaatgaacgtccaggacacacctgtatcccagagagcaaagtgatgaggagactgagcgtccccagccccacctggcggcatggacacagaaaagctacgccggctggcctctccagaccagttcccaggctgggtctgccgctgggccctggggcggtggggacagatgttgacacaaattatttatgttttcttagtatcagaagaggattctcggcactaacacatagccagtcctaactccgtactctgtgtcagcccatcccctagacaccctctgtttcctttcccggccccacctggccggccctctgcccctgcagagctgaggcagcctggggttgatggggaccacgcggtgcctgcaggtcctagaagtgagctgggcaggggctcttcagaccacacagccctgaccgggccttggaggagagccatggacaggctcctccatgccgtctttccttcttttgaaaatcctatcaatggctgggcgcggtggctcacacctgtaatcccagcactttgggagaccgaggcaggtggatcacctgaggtcaggagttcgagaccagcctggccaacgtggtgaaaccctgtctctactaaaaatacaaaaattagctgggcgtggtggcgtgcacctgtaatcccagctactcaggaggctgagacaggacacttgcttgaacccgggaggtggaggttgcaatgagccaagattgtgccactgtattccaacttgggtgacagagcacgactctgtctcaaaaaaaaaaaaaaaaaaaaaaaaaaaa

SEQ ID NO: 44 - protein, 349 aa, Homo sapiens
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 45 - DNA, 3154 bp, Mouse
cgcccgcctcccgagccgaagcgccggctgagcgtggtcctaccgcagctccctggctcctgcccggcccctgcccacccgcgcgtcccctccggccgcagctgtctatggcgcagcccccctccctggatcatgcacagaaactttcgaaagtggatcttttacgtgtttctctgctttggcgtcctctacgtgaagctcggagcattgtcatccgtggtggccctggtagccaacatcatctgcaacaagattcctggcctggccccacggcagcgtgccatctgccagagccgacccgatgccatcattgtgatcggggagggggcgcagatgggcatcgacgagtgccagcaccagttccgattcggccgctggaactgctccgccctgggcgagaagaccgtcttcgggcaagaactccgagtagggagtcgagaggctgccttcacctatgccatcacggcggcgggcgtggcgcatgctgtcaccgctgcctgcagccagggcaatctgagcaattgtggctgtgaccgggagaagcaaggctactacaaccaggcggaaggctggaagtgggggggctgctcagcggacgtccgctacggcatcgacttttctcgtcgctttgtggatgcccgtgagatcaaaaagaacgccaggcgcctcatgaaccttcacaacaatgaggcgggcagaaaggttctggaggaccgcatgaagctggaatgtaagtgtcacggtgtgtcaggctcctgtaccaccaaaacttgctggaccacgctacctaagttccgcgaggtgggccacctgctcaaggagaagtacaacgcagcggtgcaggtggaggtggtgcgagccagccgcctgcgccagcccaccttcctgcgcatcaagcagctacgcagctaccagaagcctatggagacggacctggtgtacatcgagaagtcgcccaactactgcgaggaggacgcggccacgggcagcgtgggcacgcagggccgtctgtgcaaccgcacctcgccgggggccgacggctgtgacaccatgtgctgcggccgcggctacaacacgcaccagtacaccaaggtgtggcagtgtaactgcaaattccactggtgttgcttcgtcaagtgcaacacgtgcagcgagcgcaccgaggtcttcacctgcaagtgaggctcccgcgcaggcgcgctcggcccctgccgaccctgcggccctcgccattattttgcacatccttctttgcttctggagctgccagctgcaggcacaggagggtggggatagaggtggggagctcgagatactccaggctccttcctactcgctctgtccccgcccagcatccaaggtcaacgcaatggtggtctggtacccaatggagacaaatccctttacttctctttgggaaagtgaaccacaaagggaccatgagactctgagggtcacctccctgcctgtgactggacacagaaaggccacacccaccagtcacactcaaaacggtttcctgggctgtttcctgccggccctgggcagtgtggatggatgttgacaaaattatttatgttttcttagcatcagatgaggactcagtactaacgactgggtagccagacctaaccctatttgaggacacccttccctcactcctcccggcccctccctgcagggtcctctgctccttgcagaactcgaggatgtcagaattggcacggaagctggctggtggggggactccttatcagcaccttgggaggggcttggtggccctacaaggcctgagatggccgcagaggacagccaatcttccattccatttggagactgtcatgcaaatcaaatgtcccttgtgtcaggctccaggcatgcctcgtcctctccctggtccttcaccctcccagcctgctgccaacctccacctccagtttacaaattctcttctcctctggagccaacctgacacccaggactgccccacaggttcaggagaggtcagggacagttgccccacatgacagatggacagagggcaatctgaagatttactggagaccccacggctctgtgaaataaatatactgacacagccccatccagcccaactctggaagttgccagggtgatgggaggctgcacccccttttcagtaccttgggttttgtccttcttctgtgatcctgatgccagagaactgacatccagaatttagggatgtattggtcaggccccctgcctagtgtccactgatacctgcttcagggtccttatattatgaggacatgggaccctcaaacaggggtccgtgggaagcttaatgtcccatttcctcaggcccttccagatggggacagaagaactcaggcctgggcatatcccaccctttcctccacaacacatggcagggtaagaaactgccagggctgataatacaactgcccacagcctaccccacactaaggtgtttcatagcagaagtccatggaaatgtggggtttggtggccaccaagccaggtggcctggacattgacctggggaaggtgacccttgtttgcccttgccttgcatccagctgtgtgtccctatcatgtcaggatgttccaagcctctgggccactggaaatgtcccaccctgatcctggccccatctcctcaccccaagtcctgggatacccacgtccgtcgcccagtgtcccctgtgaggagcctggttaacttatattgttatatagcgtcccctgtctgtcatgtctcttaagttattgtgacctacactgggtaccggaggggatgggggatggcttcagctgctgtcccccaagccaggctcctccttctgcttgaaacagaccctcgggggcccctgatgccaccgaggcaattcgcactgtccctgggctgccaggcacctgcgcctgcactcggtcagccgcagaccttgccttgggggagagaggtggttagtggacccaggcagggcactggctgtcccaatgctgtgtgctggggtggaggtggccgggcaccacatgtccttgaagtgccctacttctgatgggctgtgttcctgcctcctctggaggggagcacttagccccaataaaagctggaatcagaaaaaaaaaaaaaaaaaaaaaaaaaaaa

SEQ ID NO: 46 - protein, 349 aa, Mouse
MHRNFRKWIFYVFLCFGVLYVKLGALSSVVALVANIICNKIPGLAPRQRAICQSRPDAIIVIGEGAQMGIDECQHQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMKLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 47 - DNA, 1597 bp, Homo sapiens
cagaattttctcacataaatactgaggaagaccctgccctctcctcactcctctggacttggccctgagctggacctggtccactggggtaggcagggcgatggggaacctgtttatgctctgggcagctctgggcatatgctgtgctgcattcagtgcctctgcctggtcagtgaacaatttcctgataacaggtcccaaggcctatctgacctacacgactagtgtggccttgggtgcccagagtggcatcgaggagtgcaagttccagtttgcttgggaacgctggaactgccctgaaaatgctcttcagctctccacccacaacaggctgagaagtgctaccagagagacttccttcatacatgctatcagctctgctggagtcatgtacatcatcaccaagaactgtagcatgggtgacttcgaaaactgtggctgtgatgggtcaaacaatggaaaaacaggaggccatggctggatctggggaggctgcagcgacaatgtggaatttggggaaaggatctccaaactctttgtggacagtttggagaaggggaaggatgccagagccctgatgaatcttcacaacaacagggccggcagactggcagtgagagccaccatgaaaaggacatgcaaatgtcatggcatctctgggagctgcagcatacagacatgctggctgcagctggctgaattccgggagatgggagactacctaaaggccaagtatgaccaggcgctgaaaattgaaatggataagcggcagctgagagctgggaacagcgccgagggccactgggtgcccgctgaggccttccttcctagcgcagaggcggaactgatctttttagaggaatcaccagattactgtacctgcaattccagcctgggcatctatggcacagagggtcgtgagtgcctacagaacagccacaacacatccaggtgggagcgacgtagctgtgggcgcctgtgcactgagtgtgggctgcaggtggaagagaggaaaactgaggtcataagcagctgtaactgcaaattccagtggtgctgtacggtcaagtgtgaccagtgtaggcatgtggtgagcaagtattactgcgcacgctccccaggcagtgcccagtccctggggagagtttggtttggggtctatatctagagggaccttcaaagtatttgttcctttaaatttcagaccatgtccaacccagctgtgctgctgggaatcaggagaatagaagcaaaaaacgaaagagttctgttcagacttctgaagagcagcctgtggctacaaatctatgctgataaatgagattgagaactcaactgtattttgccataaatgcttctaagatatatccagctgggacttctattactccctttggaaaccttaagatcaaaaagggaataagaaacccttcttctgtatcccaataatccaccaggataaaggagaaactagaaatatgcaactcccttgatttcagtgtttggcaggtaacaaaaaattgagacccagacactggtcaacaggaaaacaatacagactcccagaattagaaagtgttattttaatgcaacctag

SEQ ID NO: 48 - protein, 355 aa, Homo sapiens
MGNLFMLWAALGICCAAFSASAWSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGRVWFGVYI

SEQ ID NO: 49 - DNA, 1747 bp, Mouse
gctctgccgaccttacttctctgcgcttggtcctggttcccggactgggcaggaccatgggacacttgttaatgctgtgggtggctgcgggcatgtgctatccagccctgggtgcttctgcctggtcagtgaacaacttcctgataaccggtcccaaggcctatctgacctacaccgccagtgtggccttgggagctcagattggcatcgaagagtgtaagttccagtttgcctgggaacggtggaattgtcctgagcatgcttttcagttttcaacccacaacaggctgcgagctgccacgagagagacatccttcattcatgccatccgctctgctgccatcatgtacgcagtcaccaagaactgcagcatgggtgacttggaaaactgcggctgtgacgagtcacaaaatggaaaaacaggtggccatggctggatctggggaggctgcagcgacaacgtggagttcggggaaaaaatctccagactcttcgtggacagtttggagaaagggaaggatgccagagccctggtgaaccttcacaacaacagggccggcagactggcagtgagggcctccacgaaaaggacctgcaagtgtcatggcatctcaggaagctgcagcatccagacgtgttggctgcagctggctgacttccggcagatgggaaattacctaaaggccaagtatgaccgcgcgctgaaaattgagatggacaagcgccagctaagggctggcaacagagccgagggccgctgggctctcacggaggccttccttcccagcacagaggctgagctgatcttcttagaggggtctcctgactactgcaaccgcaacgccagcctgagcatccagggcacagaggggagggagtgcctgcagaatgcccgcagtgcttcccggcgggagcagcgcagctgtgggcgcctgtgcacggagtgcgggctgcaggtggaggagaggagagcagaggccgtgagcagctgtgactgcaactttcagtggtgttgcactgtcaagtgtggccagtgcaggcgtgtggtgagcagatactactgcacacgccctgtaggtagtgccaggccccggggcaggggcaaggacagtgcctggtaacaccaccaccaaattcacgtgctgcctagttgcaggacagtggagatagagcctgaacttctggcctaggggacacagactggaaaacaattgggacatcacagggttggcctgtagaccttccacgataggtgggttagcctgtagaccttccacgataggcggggtagatggatgatctttaagcatcttcttcgcaggagtgaaatcggaaccttgttctcctggcttgtggacccagcctttcctgcgcagttactcttggacttaagcagcttgttaaagagggagtttgatttgggtgcacatccagaggagcctggaagaaccgtattccattaagtttcagataccgttccacccagctgtgctgctgggagtgcgagggaagagaagttaaaggaaaggaattctgggggcgggagagatggctcagtggttaagggccctggctggccctccagaggactggctcacttcacagcacccacttgatggctgtgaaccatctgtacttctagttccaggggatccaatgtccttgcctggtctctgtgaccaccaggcacaaatgtgcacagacagacatttatacatataaaataataaagtaaaaacttacattt

SEQ ID NO: 50 - protein, 354 aa, Mouse
MGHLLMLWVAAGMCYPALGASAWSVNNFLITGPKAYLTYTASVALGAQIGIEECKFQFAWERWNCPEHAFQFSTHNRLRAATRETSFIHAIRSAAIMYAVTKNCSMGDLENCGCDESQNGKTGGHGWIWGGCSDNVEFGEKISRLFVDSLEKGKDARALVNLHNNRAGRLAVRASTKRTCKCHGISGSCSIQTCWLQLADFRQMGNYLKAKYDRALKIEMDKRQLRAGNRAEGRWALTEAFLPSTEAELIFLEGSPDYCNRNASLSIQGTEGRECLQNARSASRREQRSCGRLCTECGLQVEERRAEAVSSCDCNFQWCCTVKCGQCRRVVSRYYCTRPVGSARPRGRGKDSAW

SEQ ID NO: 51 - DNA, 2117 bp, Homo sapiens
tccgcttacacaccaaggaaagttgggctttgaagaattccatccccatggccactggaggaagaatatttctccgtcttgcttacccatctcccagttttttggaattttctctagctgttactccagaggattatgtttctttcaaagccttctgtgtacatctgtcttttcacctgtgtcctccaactcagccacagctggtcggtgaacaatttcctgatgactggtccaaaggcttacctgatttactccagcagtgtggcagctggtgcccagagtggtattgaagaatgcaagtatcagtttgcctgggaccgctggaactgccctgagagagccctgcagctgtccagccatggtgggcttcgcagtgccaatcgggagacagcatttgtgcatgccatcagttctgctggagtcatgtacaccctgactagaaactgcagccttggagattttgataactgtggctgtgatgactcccgcaacgggcaactggggggacaaggctggctgtggggaggctgcagtgacaatgtgggcttcggagaggcgatttccaagcagtttgtcgatgccctggaaacaggacaggatgcacgggcagccatgaacctgcacaacaacgaggctggccgcaaggcggtgaagggcaccatgaaacgcacgtgtaagtgccatggcgtgtctggcagctgcaccacgcagacctgttggctgcagctgcccgagttccgcgaggtgggcgcgcacctgaaggagaagtaccacgcagcactcaaggtggacctgctgcagggtgctggcaacagcgcggccgcccgcggcgccatcgccgacacctttcgctccatctctacccgggagctggtgcacctggaggactccccggactactgcctggagaacaaaacgctagggctgctgggcaccgaaggccgagagtgcctaaggcgcgggcgggccctgggtcgctgggaactccgcagctgccgccggctctgcggggactgcgggctggcggtggaggagcgccgggccgagaccgtgtccagctgcaactgcaagttccactggtgctgtgcagtccgctgcgagcagtgccgccggagggtcaccaagtacttctgtagccgcgcagagcggccgcgggggggcgctgcgcacaaacccgggagaaaaccctaagggtttcctctgccccctccttttcccactggttcttggcttcctttagagaccccggtaattgtggaacctagggaatggggaacccgctctcccagacctagggatcctgaaagggaaaaactgcaatttctccaaagcttgccactttccagcctgtttccccaattcctctgtgctctcctaaagctctgtctgaatcctcgcagccacacctaggtctgaaaactcaggctttgagttactgatcttccttggattaggaaaacaggtgttcctcctcccctctcctatcagccctaatctctgacctagcctatcaacccttaggcgctggaaaaaccttctcatacacgcaggacccaggttaactcaaagctttgcccttttgcccactgtctgctaccaggggctcaccctctgctgcacctctcttctgcacagctcctcccctgctactgctgaccaaattcccaggaatcttgaatgctttctctcctcttctccctttcctttcccaaaaaaaactgaggaaactggccccggaaaagcatgtctttggggttggttcctagaggcagaggttgaagatggaagagggagctctggagtgctaacttgaacaccaagggtgctactcatccctatggtatcatatcatgaatggactttactagtggggcaatgactttcctagacaataacccgagggactccagatacataccccgaaggtctaggaaatacgttaagggcagattacagtcatttcctaccctttaaaggtaacttctcccttctcctgacctacttcctcctagcaaccaactttacctcttcttctccaaaggatctttgttcctctgagccaagactgaggtaaataaagccactttcctcttcagatcctggtctgcacctctaga

SEQ ID NO: 52 - protein, 351 aa, Homo sapiens
MFLSKPSVYICLFTCVLQLSHSWSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAARGAIADTFRSISTRELVHLEDSPDYCLENKTLGLLGTEGRECLRRGRALGRWELRSCRRLCGDCGLAVEERRAETVSSCNCKFHWCCAVRCEQCRRRVTKYFCSRAERPRGGAAHKPGRKP

SEQ ID NO: 53 - DNA, 1634 bp, Mouse
tccgcttcatttcaccaccccttaacactgtttgggatcgcttacacaccaaggtagccacccctctgcctccgaggagaatgcttcccatctctcaatgtttgagtcgctcaccctgcctttctccgaagaccatgtttcttatgaagcccgtgtgcgttcttctagtcacttgtgtccttcaccgcagccacgcctggtcagtgaacaattttctgatgaccggtccaaaggcttacctggtctactccagcagcgtggccgctggcgcccagagtggtattgaagaatgtaaataccagtttgcttgggaccgttggaattgccccgagagagctttacagctgtccagccatggtggacttcgaagcgctaaccgggagacagcatttgtgcacgccatcagctctgctggggttatgtacaccctgactagaaactgcagcctcggagactttgacaactgtggctgtgatgactcccgaaatggacaactggggggccaaggttggctctggggaggctgcagtgacaacgtgggcttcggagaggcaatttccaagcagtttgtggatgccctcgagacaggacaagatgcccgggcagccatgaatctgcacaacaatgaggctggccgcaaggcggtcaagggcaccatgaaacgcacgtgtaagtgccacggtgtgtccggcagctgcaccacgcagacctgctggttgcaactgccagagttccgggaggtaggcgcgcacttgaaggagaagtatcatgcggcgctcaaggtggacctgctgcaaggcgcgggcaacagcgcggcgggccgcggagccatcgccgacaccttccgctccatctccacccgcgagctggtgcatctggaggactccccagactactgcctggagaacaagaccctggggctgctgggcaccgagggccgagagtgtctgcggcgcgggcgcgccctgggtcgctgggagcgccgcagttgtcgccggctgtgcggggactgcgggctagcggtggaggagcgccgcgccgagacagtgtccagctgcaactgcaagtttcactggtgctgcgcggtccgctgcgagcagtgccgccggcgggtcaccaagtacttctgcagccgcgcagagcggccgcccagaggcgctgcgcacaaaccgggaaagaactcctaagggtatctatccctcccgcctccacccctgttcgtcctcggcttcctttagagacccccggaaatagaggaacccagaatgggggacctcgcactccctagcccagagattctgacaggaggaggctgcagtctctaccgagtgacactttgtagctcactcgtaggtctcaaaactgttataaaattctgcaagttgttcctgaaaagaggatgagaacaggcgagtctcctcaccccactttacctacttcggaccccaatggtcgctcaatgctggacctagcttatcaggcctaggaagggcccctctcagatattcagggtccagggaaagacgtggcccttctcttgctcgccatagcttcacctccctcctgtgagccagagcttctaggcctagactccccgctgttgattattcaagaatctaaaaaccttgaccgta

SEQ ID NO: 54 - protein, 350 aa, Mouse
MFLMKPVCVLLVTCVLHRSHAWSVNNFLMTGPKAYLVYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPPRGAAHKPGKNS

SEQ ID NO: 55 - DNA, 1631 bp, Homo sapiens
ggcgcggcaagatgctggatgggtccccgctggcgcgctggctggccgcggccttcgggctgacgctgctgctcgccgcgctgcgcccttcggccgcctacttcgggctgacgggcagcgagcccctgaccatcctcccgctgaccctggagccagaggcggccgcccaggcgcactacaaggcctgcgaccggctgaagctggagcggaagcagcggcgcatgtgccgccgggacccgggcgtggcagagacgctggtggaggccgtgagcatgagtgcgctcgagtgccagttccagttccgctttgagcgctggaactgcacgctggagggccgctaccgggccagcctgctcaagcgaggcttcaaggagactgccttcctctatgccatctcctcggctggcctgacgcacgcactggccaaggcgtgcagcgcgggccgcatggagcgctgtacctgcgatgaggcacccgacctggagaaccgtgaggcctggcagtgggggggctgcggagacaaccttaagtacagcagcaagttcgtcaaggaattcctgggcagacggtcaagcaaggatctgcgagcccgtgtggacttccacaacaacctcgtgggtgtgaaggtgatcaaggctggggtggagaccacctgcaagtgccacggcgtgtcaggctcatgcacggtgcggacctgctggcggcagttggcgcctttccatgaggtgggcaagcatctgaagcacaagtatgagacggcactcaaggtgggcagcaccaccaatgaagctgccggcgaggcaggtgccatctccccaccacggggccgtgcctcgggggcaggtggcagcgacccgctgccccgcactccagagctggtgcacctggatgactcgcctagcttctgcctggctggccgcttctccccgggcaccgctggccgtaggtgccaccgtgagaagaactgcgagagcatctgctgtggccgcggccataacacacagagccgggtggtgacaaggccctgccagtgccaggtgcgttggtgctgctatgtggagtgcaggcagtgcacgcagcgtgaggaggtctacacctgcaagggctgagttcccaggccctgccagccctgctgcacagggtgcaggcattgcacacggtgtgaagggtctacacctgcacaggctgagttcctgggctcgaccagcccagctgcgtggggtacaggcattgcacacagtgtgaatgggtctacacctgcatgggctgagtccctgggctcagacctagcagcgtggggtagtccctgggctcagtcctagctgcatggggtgcaggcattgcacagagcatgaatgggcctacacctgccaaggctgaatccctgggcccagccagccctgctgcacatggcacaggcattgcacacggtgtgaggagtgtacacctgcaagggctgaggccctgggcccagtcagccctgctgctcagagtgcaggcattgcacatggtgtgagaaggtctacacctgcaagggacgagtccccgggcctggccaaccctgctgtgcagggtgagggccatgcatgctagtatgaggggtctacacctgcaaggactgagaggctttt

SEQ ID NO: 56 - protein, 365 aa, Homo sapiens
MLDGSPLARWLAAAFGLTLLLAALRPSAAYFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 57 - DNA, 1106 bp, Mouse
cggcaagatgctggatgggtcccttctggcgcgctggctggccgcggccttcgggctgacgctgctgctcgccgcgctgcgcccttcggccgcctacttcgggctaacaggcagtgaacccctgactatcctccctctgaccctggagaccgaggctgcggcccaagcacactacaaggcctgcgacaggctgaagctggagcgcaagcagcgccgcatgtgccgcagggacccgggtgtggccgagacactggtggaggccgtaagcatgagtgccctggagtgccagtaccagttccgctttgagcgctggaactgcaccctggagggccgctaccgagccagcctgctcaagcgaggcttcaaggagactgctttcctctacgccatctcttctgccggcctgacgcatgcactggccaaggcctgcagtgcaggccgcatggagcgctgcacgtgtgatgaggcacccgacctggaaaaccgcgaggcctggcagtggggcggctgcggggacaacctcaagtacagcagcaagtttgtcaaggagttcctgggccggcgctctagcaaggatttgcgagcccgagtggacttccacaacaacctcgtgggtgtgaaggtgataaaggctggagtggaaaccacttgcaaatgccatggtgtgtctggctcctgcaccgtgcggacctgctggcggcagctagcacccttccacgaggtgggcaagcacctaaaacacaaatatgagacctcgctcaaggtgggcagcactaccaatgaagccactggagaggcaggtgccatctccccaccgcggggccgggcttctgggtcaggaggtggcgacccactgccccgaacaccagagcttgtacacctggacgactctcccagcttctgcctggctggccgcttttcccctggcacggcaggccgcaggtgtcaccgggagaagaactgtgagagtatttgttgtggccgaggccacaacacacagagtcgtgtggtgacaaggccctgccaatgccaggtccgctggtgctgctacgtggagtgcaggcagtgtacacagagagaggaggtctatacctgcaagggctgac

SEQ ID NO: 58 - protein, 365 aa, Mouse
MLDGSLLARWLAAAFGLTLLLAALRPSAAYFGLTGSEPLTILPLTLETEAAAQAHYKACDRLKLERKQRRMCRRDPGVAETLVEAVSMSALECQYQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETSLKVGSTTNEATGEAGAISPPRGRASGSGGGDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 59 - DNA, 1464 bp, Homo sapiens
gcgaggagatgctagagggcgcagcgccgccagcaccatgcgccccccgcccgcgctggccctggccgggctctgcctgctggcgctgcccgccgccgccgcctcctacttcggcctgaccgggcgggaagtcctgacgcccttcccaggattgggcactgcggcagccccggcacagggcggggcccacctgaagcagtgtgacctgctgaagctgtcccggcggcagaagcagctctgccggagggagcccggcctggctgagaccctgagggatgctgcgcacctcggcctgcttgagtgccagtttcagttccggcatgagcgctggaactgtagcctggagggcaggatgggcctgctcaagagaggcttcaaagagacagctttcctgtacgcggtgtcctctgccgccctcacccacaccctggcccgggcctgcagcgctgggcgcatggagcgctgcacctgtgatgactctccggggctggagagccggcaggcctggcagtggggcgtgtgcggtgacaacctcaagtacagcaccaagtttctgagcaacttcctggggtccaagagaggaaacaaggacctgcgggcacgggcagacgcccacaatacccacgtgggcatcaaggctgtgaagagtggcctcaggaccacgtgtaagtgccatggcgtatcaggctcctgtgccgtgcgcacctgctggaagcagctctccccgttccgtgagacgggccaggtgctgaaactgcgctatgactcggctgtcaaggtgtccagtgccaccaatgaggccttgggccgcctagagctgtgggcccctgccaggcagggcagcctcaccaaaggcctggccccaaggtctggggacctggtgtacatggaggactcacccagcttctgccggcccagcaagtactcacctggcacagcaggtagggtgtgctcccgggaggccagctgcagcagcctgtgctgcgggcggggctatgacacccagagccgcctggtggccttctcctgccactgccaggtgcagtggtgctgctacgtggagtgccagcaatgtgtgcaggaggagcttgtgtacacctgcaagcactaggcctactgcccagcaagccagtctggcactgccaggacctcctgtggcacccttcaagctgcccagccggccctctgggcagactgtcatcacatgcatgcataaaccggcatgtgtgccaatgcacacgagtgtgccactcaccaccattccttggccagccttttgcctccctcgatactcaacaaagagaagcaaagcctcctcccttaacccaagcatccccaaccttgttgaggacttggagaggagggcagagtgagaaagacatggagggaaataagggagaccaagagcacagcaggactgaaattttggacgggagagaggggctattccatcttgcttcctgg

SEQ ID NO: 60 - protein, 357 aa, Homo sapiens
MRPPPALALAGLCLLALPAAAASYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 61 - DNA, 4522 bp, Mouse
gacgagcgcctagtggcgcgaggagatgcgagagtgcaccggccgcctgcaccatgcgccccgcgcccgcgctggccctggctgcgctctgcctgctggtgctgcctgccgctgccgccgccgccgcctacttcggcctgaccggtcgtgaggtcctgacacccttcccaggcctgggtacggcagcagccccggcacaggctggtgctcacctgaagcagtgtgacctactgaagctgtccaggcggcagaagcagctctgcaggcgggagcccggcctggctgagaccctgagggatgctgcacacctggggctgctggaatgtcagttccagttcaggcaggagcgctggaactgcagcctggaggggaggactggcctgctccagagaggctttaaggagacggccttcctgtatgcagtgtctgcagctgccctcacgcatgcactggccagggcctgcagtgctgggcgcatggagcgctgtacttgtgacgactccccaggcctggagagccggcaggcctggcagtggggtgtgtgtggtgacaatctgaagtacagcaccaagttcctcagcaacttcctggggcccaagagaggaagcaaggacctgagggcgagggctgacgcccacaacacccacgtgggcatcaaggctgtgaagagcggcctgagaacaacctgcaagtgccatggtgtgtcaggctcctgtgctgttcgtacctgttggaagcagctctccccgtttcgcgagaccggccaggtgctgaagctacgctatgacacggctgtcaaggtgtccagtgccaccaacgaggccttgggtcgtctggagctatgggcccccgctaagccaggtggtaccgccaagggcctagcccctcgtcccggggacctggtctacatggaagattctcccagcttctgccggcccagcaagtactctccgggcacggcaggcagggtgtgttctcgagactccagttgcagcagcctatgctgtgggcgaggctacgacacccagagccgcatggtggttttctcctgccactgtcaggtgcagtggtgctgctacgtggagtgccagcagtgtgcacagcaggagctcgtgtatacctgcaagcgctaggcctccacagcgaatcccgcggaacagcgcgcaagcgcgcacctgtcgacgcacctgccgtgcacaagagtgtgcgactcatctctcttccccaacagatggttggccagcccttctgccttccccgacactcagcaaagagaaagaaagccctgcctcctagtcccaggatcaccaacctgctggaggacttggggccggagaacagactgagaaggggaatctttgaggaccagggtagggcaggaatgatgctgtgcgggaagagagaaacatcctcctatctcaaggccaaaaactgggaggatggggaagagggaggcggagccagctggagtgtggggtcagggcatccatctgggcgtggccgatctcttgtggtcccactctaatagcagagcgctctgggtgctgcatgcctaccctgctcttgtggcttcgtgcactggagacttcgaaatgtttattaggagcaagggaagcactttaggcttgggtggattgagtcgcagagcccatgccctgaagtcttacgtcctggcactcagggctgccaccttgtctccttgtcttgagatcccctgtcccccaaagccattgagctctgctcaacgagacccctaatatgtataagaagggtgcaggagccagtctcctcggtgagactcagataaacataactagggttgagcggggagacagtgaccctttctctttcctttggtccaaggaacctttaatcacagcccagaggtggagagaggcagggtccaaatgcctggaagagatatgacaggctctgtattgagataccactctggagtgtgtcctaccaattcctgtgaccagggacccccaagaaccgaggggcccccatccatgttagtgatacataagaacgagtgactcatgggccacacgtctgcttccaccccctgctctcaaagatgcttgtgcaggcttttttgccattgctaagtctttgccaagtctgcctcctcaatggtcttactcatttactaacgacctgtcacttgggctcccaccagaggaacaaaatgactgctggtgaatcctttggtcatttttaatgcccccatcaaggccctctgtgagaggagaggaagtagtgtacaggtacaggctcacacgtgcacacactcagcctagccaggcacagacatcccaaggagcagtgcggcgtctctccagcccagggcaaagacctcactggggtcacttctggaggctgtgagctactccagggcagggcccaaggccaaccaggaggaagtgacctcctttgggaagcctttggccatgtggctggctgtgctgcaccctcctgtgagcttccttccaccctgaaatctgttggggttactgtctctctaagggagcaggaagcttcggaatcagccggtactcagcactactggccctgccagctccaggaaagagacactgtggcggagaggtccgtggggcagaaggggctaccctttcttcagtgcctccgggcagcatgctgggaagatctttgatggtggaaagccccgaggcggagccaccgtgacctgagacccttctctgggacgactttgccacccacccgcagcttggcaggaggggtaaacagattgggagctgctttctacttccctgatgaagacagatgtgttccttggcaacccaaggcatccttctctatgaccctaatcctgctctggctcgagggtacaaggcaagaatggagcctggcaaaacttggggactagaacacctggacctacagccaaatcacctgtaccctgactctatggccaggagggccaggggtggaggagggttaaagatgaacttgaagttgagggctgaggctgaccaaccattaagactggtgccttaaggcaccctcagtcaggtcctctccctcccttctccattctttctccaaggccccgttccccctaaaatcccaccatagccatgctgggtccccccttcccccacactgggaactttaaggaagatattcacagggtatttctgcctacctcatacatgtaattttcaaaaaaaattaatttatatagttaagatatatgggaaagtatttatgttatttatatatcttctctatttcctgggcaccatatggggggttgtgtgtttacccagaagcctctgaggaaacatggctgggtctgtctggggcctcgcagagctggatgcgcatagctgagaggtcacagctcctgtgtctcactgtcttggagctcgggaagcacatgtacctcctgagataaaccccgtgacaccaagcagggccttccttgtgaagtctgtggattctctgcctctggccccagaggcctttctgctctggcccaagggttttgctcataaaggacaaaaagggtgagcagctctggatttgtaaagcactttccatcttcagaaacactcctctcttctctctccctcggttacccccggttccctatgaggtcatgccactgttaccacgttccaggcccagagacggaggcaggttggtcaaagccagtcactctctgaacccagaggttgaggaagagtgcatgctgcgtggaacgctggtcttcccccatggatggcatgctagtttctccagcaagctgagtctcatgtccccaaagacggggacttcctgagaagcctggagagacaagggctccgtggatgtcactcttagggagggtgtcctgcagccctcattgacctccacgactaggctatggtctccagcccctcacagctcgtggataatttgtgtttcttcgcttttgttttttgtcttttcaaagtgactttttccccactggatttctaagtttctctttgaaaatcagttcactggcaaatgggacctgcatcctgacctggctgcctgcatcaggagcgacaccaaacagagtgcgtggggatccccaattggcccagtgtcccccggcccttccttaagtcacacaagctcccgtgtggctttcgtgagcatggagaacctgtcccctggtcttagagaaagccagccattctgccaccctctgtttgtctggcagacagattaccacaccgtggctgtctttctagccaaagcttcctctctcaacacccatgaacgtccatgcttcctgtctgagcactgaggagaaccccagcggagctcattgttcagtgctggaatacccatcccccctcccgttgattatttagggagtgtctgataatgccaggggatactctgggtgctagggcgcagaagtacttaagagcaagtcccagcctcaggggacttatatgccggcgaggagaaagccaacaaaccaataaactatgcactggtt

SEQ ID NO: 62 - protein, 359 aa, Mouse
MRPAPALALAALCLLVLPAAAAAAAYFGLTGREVLTPFPGLGTAAAPAQAGAHLKQCDLLKLSRRQKQLCRREPGLAETLRDAAHLGLLECQFQFRQERWNCSLEGRTGLLQRGFKETAFLYAVSAAALTHALARACSAGRMERCTCDDSPGLESRQAWQWGVCGDNLKYSTKFLSNFLGPKRGSKDLRARADAHNTHVGIKAVKSGLRTTCKCHGVSGSCAVRTCWKQLSPFRETGQVLKLRYDTAVKVSSATNEALGRLELWAPAKPGGTAKGLAPRPGDLVYMEDSPSFCRPSKYSPGTAGRVCSRDSSCSSLCCGRGYDTQSRMVVFSCHCQVQWCCYVECQQCAQQELVYTCKR

SEQ ID NO: 63 - DNA, 2375 bp, Homo sapiens
acagtcacttactctacaggcagtggggcccgacacagacagcgccgcccccgccagccagcctcgcacgccctcggaagcgcaggctcccggcgctgcgctggagggttccccggcaccccagcctcccgtccccagcccgctgcacctccgggccccccttacccttgagaggcaccgggagttgtcgcgggggggcctcgggaaattccccggacccctgtgccaggaggtgcccggttcgcccgctcttcaccccccgccccccccgagggcggtgcccgggggtgctgccccatggagcggggaggcgggcgccgtctgctccgggagccctgacccgagtcggagctgtgtgtcgcagccgccccgaccccccgccgatcatgcgccggcgcccctggctctccagtcccactgggctgtgagccccccactcccagcccgtcagggcctgcgcgccatgggcagcgcccaccctcgcccctggctgcggctccgaccccagccccagccgcggccagcgctctgggtgctcctgttcttcctactgctgctggctgctgccatgcccaggtcagcacccaatgacattctggacctccgcctccccccggagcccgtgctcaatgccaacacagtgtgcctaacattgccaggcctgagccggcggcagatggaggtgtgtgtgcgtcaccctgatgtggctgcctcagccatacagggcatccagatcgccatccacgaatgccaacaccaattcagggaccagcgctggaactgctcaagcctggagactcgcaacaagatcccctatgagagtcccatcttcagcagaggtttccgagagagcgcttttgcctacgccatcgcagcagctggcgtggtgcacgccgtgtccaatgcgtgtgccctgggcaaactgaaggcctgtggctgtgatgcgtcccggcgaggggacgaggaggccttccgtaggaagctgcaccgcttacaactggatgcactgcagcgtggtaagggcctgagccatggggtcccggaacacccagccctgcccacagccagcccaggcctgcaggactcctgggagtggggcggctgcagccccgacatgggcttcggggagcgcttttctaaggactttctggactcccgggagcctcacagagacatccacgcgagaatgaggcttcacaacaaccgagttgggaggcaggcagtgatggagaacatgcggcggaagtgcaagtgccacggcacgtcaggcagctgccagctcaagacgtgctggcaggtgacgcccgagttccgcaccgtgggggcgctgctgcgcagccgcttccaccgcgccacgctcatccggccgcacaaccgcaacggcggccagctggagccgggcccagcgggggcaccctcgccggctccgggcgctcccgggccgcgccgacgggccagccccgccgacctggtctacttcgaaaagtctcccgacttctgcgagcgcgagccgcgcctggactcggcgggcaccgtgggccgcctgtgcaacaagagcagcgccggctcggatggctgcggcagcatgtgctgcggccgcggccacaacatcctgcgccagacgcgcagcgagcgctgccactgccgcttccactggtgctgtttcgtggtctgcgaagagtgccgcatcaccgagtgggtcagcgtctgcaagtgagcggcccggggtcccctgggccctgatcgaggtcccctcctggagcctggccctctgaggcttacggtcttggcaaggcagcatcgccttggctcttgggaagaggagattggaccacatgatcttataggaacccctcagctctgaggtctgtgatcgccggacagtccaggcctgtctgaaccccaccactcacttctgtgggctctaggactgactgggttcttcctccctccccgaagcccagacagttcagttgggctgggggttgctccacaccctaaaacaagcctcagccaggcaacccgtcagtctgtctccatcctttcaccccttccctggagatgggaggtggggaatgaatggaagctgacgggcagagagaggaggattaaaaaaaagaaatagacataactgagctgaagtaattccataaagggcccagacagcctcctccaccattcccttcatcattcatttaacaaatatttattttgcactctctttgcggcactctgggggcggtggggtgcgtgggggtggcaatgcaaggcactgaggccacagatgtgagtaagcgagacacaacacttgtcctcttggaggttacattcttgctggggggaggcatgggcaataaacaagtaaatatacaaac

SEQ ID NO: 64 - protein, 417 aa, Homo sapiens
MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 65 - DNA, 2487 bp, Mouse
ggcacgagttcaccctctgcatgcgttcccctcccccctctccagcaaacacggcgcgccagtccaaagcggactcagtggcctcggggacgggagcatgccacctcctgtggtgacgtcacttggggtagaacccttagacactacatggggggggggggtacagaactcccgagccaggacagtcactcactcttcaggcggtggggctgggccagacagtaccgcccccaccgcgcccgcctcgcacaccctcggaagcgcaggctcgcagcgcggcgctggggtggggggttgcgccccagaacttcggcctccagtccccagcccgctgcacctccttaccctctagaggccccctcccccttaccctctagaggcaccaggagttgtcgcaaggggcccttgggaaattccctggacccctgtgccaggaggtgcccggttcgcccgctccccatccacccccccgagggcggtgcccgggggcgctgccccatggagcggggaggcgggcgccgtctgctgcgggagctgtgacctgagtaggagctgtgtgtcgcagccgccccacccctgccgatcatgcgccggcgaccctggttcgccagtcccactgggctgtgagccccccactcctggcctgtcacggcccgcgcgccatgggcagcgcccaccctcgcccctggctgcggctcccacaagggccccagccgcggcctgagttctgggcgctcctgttcttcctactgctgctggctgccgctgtgcccaggtcagcacccaacgacatcctgggcctccgcctacccccagagcccgtgctcaacgccaacacagtgtgcctgacattgcccggcctgagccggcggcagatggaggtgtgtgtgcgtcaccctgacgtggccgcctctgctatccagggcatccagatcgccatccatgagtgccagcatcagttccgggaccagcgctggaactgctccagcctggagactcggaacaaagtcccctacgagagccccatcttcagccgaggttttcgagagagtgctttcgcctacgccatagcagctgccggggtggtgcacgcagtgtccaacgcgtgcgctctgggtaaactgaaggcttgcggttgcgacgcctccagacgtggggacgaagaagctttccgtcggaagctgcaccgcttgcagctggacgcgctgcagcgcggaaagggcttgagccacggggtccctgaacacccggccatacttcctgccagcccaggtctgcaggactcctgggagtggggtggctgcagtccggatgtgggcttcggagaacgcttctctaaggactttctggactcccgagagcctcacagagacatccatgctcgaatgagactccacaacaaccgtgtgggccggcaggcggtgatggagaacatgcggcgtaagtgcaaatgccacggcacctcaggcagctgccagctcaagacctgctggcaggtgacgcctgagttccgcacagtaggggcgctgctgcgcaaccgcttccaccgcgccacgctcatccggccgcacaaccgcaacggtggccagctggagcccggccccgcgggagcaccctcgccagcaccgggcactccagggctgcgccgcagggccagccactccgacctggtctactttgagaaatctcccgacttctgtgagcgcgagccgcgcctggactcggcaggcactgtgggccgcctgtgcaataagagcagcacgggtcccgatggctgcggcagcatgtgctgtggccgcggccacaacattctgcgccagacgcgcagcgagcgctgccactgccggttccactggtgctgcttcgtggtctgcgaagaatgccgcatcaccgagtgggtcagcgtctgcaagtgagcagacccaagctcctctgggtctcaagaatggttgtcctcttggtgcctggcttctgccgctagcggatctgagccaggcagcaagcagcagccttggctcctgagagaggtggttggctcttacagccccgagggtctacaatcaccagacagtccagatctgattgacattcctccgctcacctctgtaggttcccctctttctgttcctagctcagacagctgggggtgatagtggagactgttccacaccctaggacaggtcaccaaagcagcccagcctggcatgcctacctcctgtcatctcttcttcccttccccaggagtgataggcaatgcactgaagctgatgggcaccggggaagaaaactaaaaggcagaaatggccgtcatcgggctgaagtgactctaagggctccagacctctgctcctgtctttcacttaacagatatttatttttgcgctctctttgagacactctctggggaaaaagaagctccggagtctacaggctgattaagggacatggacaataaaccagtaaacacacaaaaaaaaaaaaaaaaaa

SEQ ID NO: 66 - protein, 417 aa, Mouse [sequence omitted]

SEQ ID NO: 67 - DNA, 2288 bp, Homo sapiens
ggggctgcagctccgtcagcccggcagagccaccctgagctcggtgagagcaaagccagagcccccagtcctttgctcgccggcttgctatctctctcgatcactccctcccttcctccctcccttcctcccggcggccgcggcggcgctggggaagcggtgaagaggagtggcccggccctggaagaatgcggctctgacaaggggacagaacccagcgcagtctccccacggtttaagcagcactagtgaagcccaggcaacccaaccgtgcctgtctcggaccccgcacccaaaccactggaggtcctgatcgatctgcccaccggagcctccgggcttcgacatgctggaggagccccggccgcggcctccgccctcgggcctcgcgggtctcctgttcctggcgttgtgcagtcgggctctaagcaatgagattctgggcctgaagttgcctggcgagccgccgctgacggccaacaccgtgtgcttgacgctgtccggcctgagcaagcggcagctaggcctgtgcctgcgcaaccccgacgtgacggcgtccgcgcttcagggtctgcacatcgcggtccacgagtgtcagcaccagctgcgcgaccagcgctggaactgctccgcgcttgagggcggcggccgcctgccgcaccacagcgccatcctcaagcgcggtttccgagaaagtgctttttccttctccatgctggctgctggggtcatgcacgcagtagccacggcctgcagcctgggcaagctggtgagctgtggctgtggctggaagggcagtggtgagcaggatcggctgagggccaaactgctgcagctgcaggcactgtcccgaggcaagagtttcccccactctctgcccagccctggccctggctcaagccccagccctggcccccaggacacatgggaatggggtggctgtaaccatgacatggactttggagagaagttctctcgggatttcttggattccagggaagctccccgggacatccaggcacgaatgcgaatccacaacaacagggtggggcgccaggtggtaactgaaaacctgaagcggaaatgcaagtgtcatggcacatcaggcagctgccagttcaagacatgctggagggcggccccagagttccgggcagtgggggcggcgttgagggagcggctgggccgggccatcttcattgatacccacaaccgcaattctggagccttccagccccgtctgcgtccccgtcgcctctcaggagagctggtctactttgagaagtctcctgacttctgtgagcgagaccccactatgggctccccagggacaaggggccgggcctgcaacaagaccagccgcctgttggatggctgtggcagcctgtgctgtggccgtgggcacaacgtgctccggcagacacgagttgagcgctgccattgccgcttccactggtgctgctatgtgctgtgtgatgagtgcaaggttacagagtgggtgaatgtgtgtaagtgagggtcagccttaccttggggctggggaagaggactgtgtgagaggggcgccttttcagccctttgctctgatttccttccaaggtcactcttggtccctggaagcttaaagtatctacctggaaacagctttaggggtggtgggggtcaggtggactctgggatgtgtagccttctccccaacaattggagggtcttgaggggaagctgccacccctcttctgctccttagacacctgaatggactaagatgaaatgcactgtattgctcctcccacttctcaactccagagcccctttaaccctgattcatactccttttggctggggagtccctatagtttcaccactcctctcccttgagggataaccccaggcactgtttggagccataagatctgtatctagaaagagatcacccactcctatgtactatccccaaactcctttactgcagcctgggctccctcttgtgggataatgggagacagtggtagagaggtttttcttgggaaagagacagagtgctgaggggcactctcccctgaatcctcagagagttgtctgtccaggcccttagggaagttgtctccttccattcagatgttaatggggaccctccaaaggaaggggttttcccatgactcttggagcctctttttccttcttcagcaggaagggtgggaagggataatttatcatactgagacttgttcttggttcctgtttgaaactaaaataaattaagttactggaaaaaaaaaaaaaaaaaa

SEQ ID NO: 68 - protein, 389 aa, Homo sapiens
MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 69 - DNA, 2215 bp, Mouse
ctcgagcagaaccacccgtgagttaggtcgagcagagccaaagcccccggtgcttcgtcgcgggttcgctcgctagctatctggatcactccctcccttttaccctcccttcctcccggcgggcggccgcggcgacgccggggaagcggcagagaggagtggctgggcgctgggagaatgctgctccgccgagggggctgaacccgacagtttccccacggtttaagccccaagagccgggcccgagtgactcaaccgcgagccttgtggatcctgcacctgaaccgctggaggctgactgactcgcccaccggagcctccgggcttcgacatgctggaggagccccggtctcggcctccgcccttaggcctcgcgggtctcctgttcttggctttgttcagtcgggctctaagcaatgagattctgggccttaaacttcccggtgagccgccgctgacggccaacaccgtgtgcttgaccctgtccggactgagtaagcgacagctggggctgtgcctgcgcagccccgacgtgacggcgtcggcgctccaggggctgcacatcgccgttcacgagtgtcagcaccagctgcgcgaccagcgctggaactgctcggcactggagggcggcggccggctgccgcaccacagcgccatcctcaagcgcggtttccgtgagagtgctttctccttctccatgctggctgctggggtcatgcatgctgttgccacagcctgcagcctgggcaagctggtgagctgcggctgcggatggaagggtagtggtgagcaagaccggcttagagccaagctgctgcagcttcaggcactgtctcggggcaagactttccccatctcccagcccagccctgttcctggctcagtccccagccccggcccccaggacacgtgggaatggggtggctgtaaccacgacatggacttcggagagaagttctctcgggatttcttggattccagggaggctccccgggacatccaggcgagaatgcggatccacaacaacagggtgggacgccaggtggtaacggaaaacctgaagcggaagtgcaaatgccatggaacgtcaggcagctgccaattcaagacctgttggagggcagcgccagagttccgggccatcggggcagcactgagggagcggctgagcagagccatctttatcgatacccacaaccgcaactctggagcgttccagccccgcctacgtccgcggcgcctctctggagagctggtttactttgagaagtctcctgacttctgcgagcgagaccctactctgggctccccaggcacgagaggccgggcttgcaacaagaccagccgcctcttggatggctgtggcagcctgtgctgtggccgtgggcacaacgtgctccggcagacgcgagtggagcgctgccactgtcgtttccactggtgctgttatgtgctgtgtgatgagtgtaaagtcacagagtgggtcaatgtgtgtaaatgaaggtgagcctcgcctaggcacgacgaggaggagaagcactgtgtgagggctgctctctttcagccctttgctcggatttctgtctagggtttatcgtggctcccggaagctcagagcatctgcctgagaacagctctgggggtgtagggtcaggtgaaatctgtaacgagcagccttttgtgggggaagtggccccacactctgttcttaaacactcgaatagactaagatgaaatgcactgtactgttagcgtcttctctacctacagctccctcgggctcaggttcctacttcctttggatagggagtctatcttttggccactcctcttcctcgaaggataatagcaggcattgtgtggagtcaataagacccgtatatatagcaagagaccacctcttcctatttgtggttctcaaactcctccactacagcccagaacctcctcttatgggacctcgggtgacaataatgagaggttttcggttggaaaaggacagagggcagggaagcctcagacagctgtcttgtcaggctcttgggaggcttctccttccgttcagttgttgaaagggtctctccaaaggaaaggttttagccataactcttggaggcccttttccttcttcagcaggaagggtgggaatggataatttattttactgagatgtgttcttggttcctgtttgaaactaaaataaattaagttactg

SEQ ID NO: 70 - protein, 389 aa, Mouse
MLEEPRSRPPPLGLAGLLFLALFSRALSNEILGLKLPGEPPLTANTVCLTLSGLSKRQLGLCLRSPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKTFPISQPSPVPGSVPSPGPQDTWEWGGCNHDMDFGEKFSRDFLDSREAPRDIQARMRIHNNRVGRQVVTENLKRKCKCHGTSGSCQFKTCWRAAPEFRAIGAALRERLSRAIFIDTHNRNSGAFQPRLRPRRLSGELVYFEKSPDFCERDPTLGSPGTRGRACNKTSRLLDGCGSLCCGRGHNVLRQTRVERCHCRFHWCCYVLCDECKVTEWVNVCK

SEQ ID NO: 71 - DNA, 1927 bp, Homo sapiens
taacccgccgcctccgctctccccggctgcaggcggcgtgcaggaccagcggcggccgtgcaggcggaggacttcggcgcggctcctcctgggtgtgaccccgggcgcgcccgccgcgcgacgatgagggcgcggccgcaggtctgcgaggcgctgctcttcgccctggcgctccagaccggcgtgtgctatggcatcaagtggctggcgctgtccaagacaccatcggccctggcactgaaccagacgcaacactgcaagcagctggagggtctggtgtctgcacaggtgcagctgtgccgcagcaacctggagctcatgcacacggtggtgcacgccgcccgcgaggtcatgaaggcctgtcgccgggcctttgccgacatgcgctggaactgctcctccattgagctcgcccccaactatttgcttgacctggagagagggacccgggagtcggccttcgtgtatgcgctgtcggccgccgccatcagccacgccatcgcccgggcctgcacctccggcgacctgcccggctgctcctgcggccccgtcccaggtgagccacccgggcccgggaaccgctggggaggatgtgcggacaacctcagctacgggctcctcatgggggccaagttttccgatgctcctatgaaggtgaaaaaaacaggatcccaagccaataaactgatgcgtctacacaacagtgaagtggggagacaggctctgcgcgcctctctggaaatgaagtgtaagtgccatggggtgtctggctcctgctccatccgcacctgctggaaggggctgcaggagctgcaggatgtggctgctgacctcaagacccgatacctgtcggccaccaaggtagtgcaccgacccatgggcacccgcaagcacctggtgcccaaggacctggatatccggcctgtgaaggactcggaactcgtctatctgcagagctcacctgacttctgcatgaagaatgagaaggtgggctcccacgggacacaagacaggcagtgcaacaagacatccaacggaagcgacagctgcgaccttatgtgctgcgggcgtggctacaacccctacacagaccgcgtggtcgagcggtgccactgtaagtaccactggtgctgctacgtcacctgccgcaggtgtgagcgtaccgtggagcgctatgtctgcaagtgaggccctgccctccgccccacgcaggagcgaggactctgctcaaggaccctcagcaactggggccaggggcctggagacactccatggagctctgcttgtgaattccagatgccaggcatgggaggcggcttgtgctttgccttcacttggaagccaccaggaacagaaggtctggccaccctggaaggagggcaggacatcaaaggaaaccgacaagattaaaaataacttggcagcctgaggctctggagtgcccacaggctggtgtaaggagcggggcttgggatcggtgagactgatacagacttgacctttcagggccacagagaccagcctccgggaaggggtctgcccgccttcttcagaatgttctgcgggaccccctggcccaccctggggtctgagcctgctgggcccaccacatggaatcactagcttgggttgtaaatgttttcttttgttttttgctttttcttcctttgggatgtggaagctacagaaatatttataaaacatagctttttctttggggtggcacttctcaattcctctttatatattttatatatataaatatatatgtatatatataatgatctctattttaaaactagctttttaagcagctgtatgaaataaatgctgagtgagccccagcccgcccctgcagttcccggcctcgtcaagtgaactcggcagaccctggggctggcagagggagctctccagtttccaggca

SEQ ID NO: 72 - protein, 354 aa, Homo sapiens
MRARPQVCEALLFALALQTGVCYGIKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 73 - DNA, 1821 bp, Mouse
gaattcgggcctaatccgagcctgacgccggcgggtctcgggcggttcggggagagagcggactccttcctcgctcagcctccccggcccgacccctcctttgtaatttgaataaaacgcctcccagcccgcgcgccgccttaacccgccgccctgttctccgtgattgcaggcggcgtgcgcgcaggaacagcagcggtggcctgcaggcggcggagttcggtgcggctcctgcagggtgcgacccccgggacgccgggccgcgcgacgatgagggcgcggccgcaggtctgcgaggctctgctctttgccttggcgctccacaccggcgtgtgctatggcatcaagtggctggcactgtccaagactccggcagccttggcactgaatcagacgcaacactgtaaacagctggagggcctggtgtctgcgcaggtgcagctctgccgcagcaacctggagctcatgcgcaccatcgtgcacgccgcccggggggccatgaaggcctgccgtagggccttcgctgacatgcgctggaactgctcctccatcgagctcgcccccaactacctgcttgacctggagagaggtacacgggagtcagccttcgtgtatgccctgtcggccgccaccatcagtcacaccatcgcccgggcctgcacctctggcgacctgcccggctgctcctgcggccccgtcccaggtgagccacccgggcccgggaaccgctggggaggatgtgcggacaacctcagctacgggctcctcatgggggccaagttttccgatgctcctatgaaggtgaaaaaaacaggatcccaagccaataaactgatgcgtctacacaacagtgaagtggggagacaggctctacgtgcctccctggaaacgaagtgtaaatgccatggggtgtctggctcctgctccatccgcacctgttggaaggggctgcaagagctccaggacgtggctgctgacctcaagacccgctacctgtcagccacgaaggtggtacaccggcctatgggcacccgcaaacacttggtgcccaaggacctggatatccggcctgtgaaggactcagaacttgtgtatctacagagctcccctgacttctgcatgaagaatgagaaggtgggatcccatgggacccaagacaggcagtgcaacaagacttccaacggcagtgacagctgcgacctcatgtgctgtgggcgcggctacaacccctacacggacagagtggtggagcgatgtcactgcaagtaccactggtgctgctacgtcacctgccgcaggtgtgagcgcacggtggagcgctacgtctgcaagtgagaccatatgccccacccctgaggaggggtgctgctcctctgaggacccactcaagggcctagagaccttggtggacttccctgcagatgccagatgccaggcgtgggaggcggcttgtgctgtgcctccacttggaagacaccacaccaggaggcctggtcgccctgggagagccggggcttcaaaggaaactgataggattaaaaataacctggcagcctggggcctgagtgccacatgttgccttccaggctgctccaagaagtcagggcagggatgggtaagactgtgcatttgacctttcaaggccagaaagaccggctttctggaatgttctttgggaccctgtgcccaccacatggaaccactaacttgggttgtaaatttttattttccttcccctctccgtgggatgtgggagttacagaaatatttataaaaatacagctttttcctttgggggtgaaaaaaaaaaaaaaagaattc

SEQ ID NO: 74 - protein, 354 aa, Mouse
MRARPQVCEALLFALALHTGVCYGIKWLALSKTPAALALNQTQHCKQLEGLVSAQVQLCRSNLELMRTIVHAARGAMKACRRAFADMRWNCSSIELAPNYLLDLERGTRESAFVYALSAATISHTIARACTSGDLPGCSCGPVPGEPPGPGNRWGGCADNLSYGLLMGAKFSDAPMKVKKTGSQANKLMRLHNSEVGRQALRASLETKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 75 - DNA, 3132 bp, Homo sapiens
cccgcatctcctgcacatctccacccctgcgcaggaggagatccccaggctgctctctccatctctcctacagctccctgcaaacgagggggaagctgctgagagtccctatcactgctggccttttaatgttgtatgcaaggaggaagagggcgagggataacttggtgctggacaactgacctgcggcccgaagggcctctggggagggggtgcaaaagaggagcggctgggctgggggactccatgcgggggcgatggacagggcggcgctcctgggactggcccgcttgtgcgcgctgtgggcagccctgctcgtgctgttcccctacggagcccaaggaaactggatgtggttgggcattgcctccttcggggttccagagaagctgggctgcgccaatttgccgctgaacagccgccagaaggagctgtgcaagaggaaaccgtacctgctgccgagcatccgagagggcgcccggctgggcattcaggagtgcgggagccagttcagacacgagagatggaactgcatgatcaccgccgccgccactaccgccccgatgggcgccagccccctctttggctacgagctgagcagcggcaccaaagagacagcatttatttatgctgtgatggctgcaggcctggtgcattctgtgaccaggtcatgcagtgcaggcaacatgacagagtgttcctgtgacaccaccttgcagaacggcggctcagcaagtgaaggctggcactgggggggctgctccgatgatgtccagtatggcatgtggttcagcagaaagttcctagatttccccatcggaaacaccacgggcaaagaaaacaaagtactattagcaatgaacctacataacaatgaagctggaaggcaggctgtcgccaagttgatgtcagtagactgccgctgccacggagtttccggctcctgtgctgtgaaaacatgctggaaaaccatgtcttcttttgaaaagattggccatttgttgaaggataaatatgaaaacagtatccagatatcagacaaaacaaagaggaaaatgcgcaggagagaaaaagatcagaggaaaataccaatccataaggatgatctgctctatgttaataagtctcccaactactgtgtagaagataagaaactgggaatcccagggacacaaggcagagaatgcaaccgtacatcagagggtgcagatggctgcaacctcctctgctgtggccgaggttacaacacccatgtggtcaggcacgtggagaggtgtgagtgtaagttcatctggtgctgctatgtccgttgcaggaggtgtgaaagcatgactgatgtccacacttgcaagtaaccactccatccagccttgggcaagatgcctcagcaatatacaatggcattgcaaccagagaggtgcccatccctgtgcagcgctagtaaagttgactcttgcagtggaatccctagaaccttggacctgagagtttcccttacctgatcgacatattttcctttatctgatcaacccatcaatcatgtggatttcttgggattctaatgttgaaaaggtttatattcaccttttgatgatttggggaatatatattgacatacaaggaagataatctgtttcctaagcaagaaataacaggaaagatcccttatgccaggaggcctgccatactcaggataagatccttgaatatggaacttagttacaggactcaataatggtgggtgaacattagtcatttttaaaagacacctcttatagcaataaggagacattaacatgaatctcatttattctctcagtattttaactgaagaaattatactgtttgtgtgtggatagaagatgttgaaaagttaacataagcattgggtgctgacttaccctttcatgtacttccaaagaaaggtaatcaaaaagaatcttcttaagtgatataatatccctaaaaaaatgatcattacagatgtttagtgacaaagaatcaatatgtaaaaagtataatgaatgatttagattttaagtgccttttcactgggagaatctggaaaaacctccataaggtatatagcaatctttgatctttagattcatacttttatcacagatcagtttcaactgttaaaaacccacctctgagatactggggggaggatcctgaaacatgcgggaaaaggagaggtaaacagtggaggtaaaaatataatttcatacattgtaaagaaaagcaccctttaaatgtgtaaagacagtgttttgtaaagaattttgtttaaaaagtttctattttgtaaatacagtacttaagttatatgatttatattaaaacatttattgacaaagcctaagagctaaggcagtaaaattatctcataaataatattagcttattttttttcatactattaatgctatttttttggacatcgaagagaatttaacttagcagttagttatatggatgtgtatttcttgctaaaatgacagttttatatgttatagattaaaatatgttgcaaaatatcaaaaatttgtgttatttcagcagtaagattaattgaattctcttttcacattagttatgcttaactcataaggttattataataaattatattagtaaaagtcttaactggaaaaaagaatctaaatcagaatagtgatcaatttgtggatttgatatcctggatatttattatattttatgtaatgctgcatttctatttgaatgttaagtggtctttcttgtttttaatattcatgcatgtatattcatcatattttacaaggttcctggtaaaaattacagggctctatttaaggatgtattttaatgtaaatgcttatgttttttatgaattgttaaatatttcagtattatatagaaaaaaatagatttttaaaattcagaatggacaaagagaatattcattttcttattaataagataaagaaatgtttccctgccccacagtcttcattctatttctctttaattttattcactgaggcagagaaacaatttttgaaaaagagcaaacccatggaaaatgtctcagatctaatattaaaatcaagactaagcatttaactgtgaaaaaaaaaaaaaaaaaaa

SEQ ID NO: 76 - protein, 365 aa, Homo sapiens
MDRAALLGLARLCALWAALLVLFPYGAQGNWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 77 - DNA, 2894 bp, Homo sapiens
agcctgcaaaaaccacagagggcaaagccagaaagatggaaaggcacccatgcagctcaccacttgcctcagggagaccctcttcacaggggcttctcaaaagacctccctatggtggttgggcattgcctccttcggggttccagagaagctgggctgcgccaatttgccgctgaacagccgccagaaggagctgtgcaagaggaaaccgtacctgctgccgagcatccgagagggcgcccggctgggcattcaggagtgcgggagccagttcagacacgagagatggaactgcatgatcaccgccgccgccactaccgccccgatgggcgccagccccctctttggctacgagctgagcagcggcaccaaagagacagcatttatttatgctgtgatggctgcaggcctggtgcattctgtgaccaggtcatgcagtgcaggcaacatgacagagtgttcctgtgacaccaccttgcagaacggcggctcagcaagtgaaggctggcactgggggggctgctccgatgatgtccagtatggcatgtggttcagcagaaagttcctagatttccccatcggaaacaccacgggcaaagaaaacaaagtactattagcaatgaacctacataacaatgaagctggaaggcaggctgtcgccaagttgatgtcagtagactgccgctgccacggagtttccggctcctgtgctgtgaaaacatgctggaaaaccatgtcttcttttgaaaagattggccatttgttgaaggataaatatgaaaacagtatccagatatcagacaaaacaaagaggaaaatgcgcaggagagaaaaagatcagaggaaaataccaatccataaggatgatctgctctatgttaataagtctcccaactactgtgtagaagataagaaactgggaatcccagggacacaaggcagagaatgcaaccgtacatcagagggtgcagatggctgcaacctcctctgctgtggccgaggttacaacacccatgtggtcaggcacgtggagaggtgtgagtgtaagttcatctggtgctgctatgtccgttgcaggaggtgtgaaagcatgactgatgtccacacttgcaagtaaccactccatccagccttgggcaagatgcctcagcaatatacaatggcattgcaaccagagaggtgcccatccctgtgcagcgctagtaaagttgactcttgcagtggaatccctagaaccttggacctgagagtttcccttacctgatcgacatattttcctttatctgatcaacccatcaatcatgtggatttcttgggattctaatgttgaaaaggtttatattcaccttttgatgatttggggaatatatattgacatacaaggaagataatctgtttcctaagcaagaaataacaggaaagatcccttatgccaggaggcctgccatactcaggataagatccttgaatatggaacttagttacaggactcaataatggtgggtgaacattagtcatttttaaaagacacctcttatagcaataaggagacattaacatgaatctcatttattctctcagtattttaactgaagaaattatactgtttgtgtgtggatagaagatgttgaaaagttaacataagcattgggtgctgacttaccctttcatgtacttccaaagaaaggtaatcaaaaagaatcttcttaagtgatataatatccctaaaaaaatgatcattacagatgtttagtgacaaagaatcaatatgtaaaaagtataatgaatgatttagattttaagtgccttttcactgggagaatctggaaaaacctccataaggtatatagcaatctttgatctttagattcatacttttatcacagatcagtttcaactgttaaaaacccacctctgagatactggggggaggatcctgaaacatgcgggaaaaggagaggtaaacagtggaggtaaaaatataatttcatacattgtaaagaaaagcaccctttaaatgtgtaaagacagtgttttgtaaagaattttgtttaaaaagtttctattttgtaaatacagtacttaagttatatgatttatattaaaacatttattgacaaagcctaagagctaaggcagtaaaattatctcataaataatattagcttattttttttcatactattaatgctatttttttggacatcgaagagaatttaacttagcagttagttatatggatgtgtatttcttgctaaaatgacagttttatatgttatagattaaaatatgttgcaaaatatcaaaaatttgtgttatttcagcagtaagattaattgaattctcttttcacattagttatgcttaactcataaggttattataataaattatattagtaaaagtcttaactggaaaaaagaatctaaatcagaatagtgatcaatttgtggatttgatatcctggatatttattatattttatgtaatgctgcatttctatttgaatgttaagtggtctttcttgtttttaatattcatgcatgtatattcatcatattttacaaggttcctggtaaaaattacagggctctatttaaggatgtattttaatgtaaatgcttatgttttttatgaattgttaaatatttcagtattatatagaaaaaaatagatttttaaaattcagaatggacaaagagaatattcattttcttattaataagataaagaaatgtttccctgccccacagtcttcattctatttctctttaattttattcactgaggcagagaaacaatttttgaaaaagagcaaacccatggaaaatgtctcagatctaatattaaaatcaagactaagcatttaactgtgaaaaaaaaaaaaaaaaaaa

SEQ ID NO: 78 - protein, 355 aa, Homo sapiens
MQLTTCLRETLFTGASQKTSLWWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 79 - DNA, 5100 bp, Homo sapiens
atggagcccgagtgagcgcggcgcgggcccgtccggccgccggacaacatggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagccgagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggactttaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacattttcgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatctactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaacagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtagacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgtgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctgacctcggccgggccactctggcttctctgtgcccctgtaaatagttttaaatatgaacaaagaaaaaaatatattttatgatttaaaaaataaatataattgggattttaaaaacatgagaaatgtgaactgtgatggggtgggcagggctgggagaactttgtacagtggaacaaatatttataaacttaattttgtaaaacag

SEQ ID NO: 80 - protein, 1615 aa, Homo sapiens
MEAAPPGPPWPLLLLLLLLLALCGCPAPAAASPLLLFANRRDVRLVDAGGVKLESTIVVSGLEDAAAVDFQFSKGAVYWTDVSEEAIKQTYLNQTGAAVQNVVISGLVSPDGLACDWVGKKLYWTDSETNRIEVANLNGTSRKVLFWQDLDQPRAIALDPAHGYMYWTDWGETPRIERAGMDGSTRKIIVDSDIYWPNGLTIDLEEQKLYWADAKLSFIHRANLDGSFRQKVVEGSLTHPFALTLSGDTLYWTDWQTRSIHACNKRTGGKRKEILSALYSPMDIQVLSQERQPFFHTRCEEDNGGCSHLCLLSPSEPFYTCACPTGVQLQDNGRTCKAGAEEVLLLARRTDLRRISLDTPDFTDIVLQVDDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIALHPVMGLMYWTDWGENPKIECANLDGQERRVLVNASLGWPNGLALDLQEGKLYWGDAKTDKIEVINVDGTKRRTLLEDKLPHIFGFTLLGDFIYWTDWQRRSIERVHKVKASRDVIIDQLPDLMGLKAVNVAKVVGTNPCADRNGGCSHLCFFTPHATRCGCPIGLELLSDMKTCIVPEAFLVFTSRAAIHRISLETNNNDVAIPLTGVKEASALDFDVSNNHIYWTDVSLKTISRAFMNGSSVEHVVEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERVVIADDLPHPFGLTQYSDYIYWTDWNLHSIERADKTSGRNRTLIQGHLDFVMDILVFHSSRQDGLNDCMHNNGQCGQLCLAIPGGHRCGCASHYTLDPSSRNCSPPTTFLLFSQKSAISRMIPDDQHSPDLILPLHGLRNVKAIDYDPLDKFIYWVDGRQNIKRAKDDGTQPFVLTSLSQGQNPDRQPHDLSIDIYSRTLFWTCEATNTINVHRLSGEAMGVVLRGDRDKPRAIVVNAERGYLYFTNMQDRAAKIERAALDGTEREVLFTTGLIRPVALVVDNTLGKLFWVDADLKRIESCDLSGANRLTLEDANIVQPLGLTILGKHLYWIDRQQQMIERVEKTTGDKRTRIQGRVAHLTGIHAVEEVSLEEFSAHPCARDNGGCSHICIAKGDGTPRCSCPVHLVLLQNLLTCGEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATVRPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 81 - DNA, 5119 bp, Mouse
gccgcggcgcccgaggcgggagcaagaggcgccgggagccgcgaggatccaccgccgccgcgcgcgccatggagcccgagtgagcgcgcggcgctcccggccgccggacgacatggaaacggcgccgacccgggcccctccgccgccgccgccgccgctgctgctgctggtgctgtactgcagcttggtccccgccgcggcctcaccgctcctgttgtttgccaaccgccgggatgtgcggctagtggatgccggcggagtgaagctggagtccaccattgtggccagtggcctggaggatgcagctgctgtagacttccagttctccaagggtgctgtgtactggacagatgtgagcgaggaggccatcaaacagacctacctgaaccagactggagctgctgcacagaacattgtcatctcgggcctcgtgtcacctgatggcctggcctgtgactgggttggcaagaagctgtactggacggactccgagaccaaccgcattgaggttgccaacctcaatgggacgtcccgtaaggttctcttctggcaggacctggaccagccaagggccattgccctggatcctgcacatgggtacatgtactggactgactggggggaagcaccccggatcgagcgggcagggatggatggcagtacccggaagatcattgtagactccgacatttactggcccaatgggctgaccatcgacctggaggaacagaagctgtactgggccgatgccaagctcagcttcatccaccgtgccaacctggacggctccttccggcagaaggtggtggagggcagcctcactcacccttttgccctgacactctctggggacacactctactggacagactggcagacccgctccatccacgcctgcaacaagtggacaggggagcagaggaaggagatccttagtgctctgtactcacccatggacatccaagtgctgagccaggagcggcagcctcccttccacacaccatgcgaggaggacaacggtggctgttcccacctgtgcctgctgtccccgagggagcctttctactcctgtgcctgccccactggtgtgcagttgcaggacaatggcaagacgtgcaagacaggggctgaggaagtgctgctgctggctcggaggacagacctgaggaggatctctctggacacccctgacttcacagacatagtgctgcaggtgggcgacatccggcatgccattgccattgactacgatcccctggagggctacgtgtactggaccgatgatgaggtgcgggctatccgcagggcgtacctagatggctcaggtgcgcagacacttgtgaacactgagatcaatgaccccgatggcattgctgtggactgggtcgcccggaacctctactggacagatacaggcactgacagaattgaggtgactcgcctcaacggcacctcccgaaagatcctggtatctgaggacctggacgaaccgcgagccattgtgttgcaccctgtgatgggcctcatgtactggacagactggggggagaaccccaaaatcgaatgcgccaacctagatgggagagatcggcatgtcctggtgaacacctcccttgggtggcccaatggactggccctggacctgcaggagggcaagctgtactggggggatgccaaaactgataaaatcgaggtgatcaacatagacgggacaaagcggaagaccctgcttgaggacaagctcccacacatttttgggttcacactgctgggggacttcatctactggaccgactggcagagacgcagtattgaaagggtccacaaggtcaaggccagccgggatgtcatcattgatcaactccccgacctgatgggactcaaagccgtgaatgtggccaaggttgtcggaaccaacccatgtgcggatggaaatggagggtgcagccatctgtgcttcttcaccccacgtgccaccaagtgtggctgccccattggcctggagctgttgagtgacatgaagacctgcataatccccgaggccttcctggtattcaccagcagagccaccatccacaggatctccctggagactaacaacaacgatgtggctatcccactcacgggtgtcaaagaggcctctgcactggactttgatgtgtccaacaatcacatctactggactgatgttagcctcaagacgatcagccgagccttcatgaatgggagctcagtggagcacgtgattgagtttggcctcgactaccctgaaggaatggctgtggactggatgggcaagaacctctattgggcggacacagggaccaacaggattgaggtggcccggctggatgggcagttccggcaggtgcttgtgtggagagaccttgacaaccccaggtctctggctctggatcctactaaaggctacatctactggactgagtggggtggcaagccaaggattgtgcgggccttcatggatgggaccaattgtatgacactggtagacaaggtgggccgggccaacgacctcaccattgattatgccgaccagcgactgtactggactgacctggacaccaacatgattgagtcttccaacatgctgggtcaggagcgcatggtgatagctgacgatctgccctacccgtttggcctgactcaatatagcgattacatctactggactgactggaacctgcatagcattgaacgggcggacaagaccagtgggcggaaccgcaccctcatccagggtcacctggacttcgtcatggacatcctggtgttccactcctcccgtcaggatggcctcaacgactgcgtgcacagcaatggccagtgtgggcagctgtgcctcgccatccccggaggccaccgctgtggctgtgcttcacactacacgctggaccccagcagccgcaactgcagcccgccctccaccttcttgctgttcagccagaaatttgccatcagccggatgatccccgatgaccagctcagcccggaccttgtcctaccccttcatgggctgaggaacgtcaaagccatcaactatgacccgctggacaagttcatctactgggtggacgggcgccagaacatcaagagggccaaggacgacggtacccagccctccatgctgacctctcccagccaaagcctgagcccagacagacagccacacgacctcagcattgacatctacagccggacactgttctggacctgtgaggccaccaacactatcaatgtccaccggctggatggggatgccatgggagtggtgcttcgaggggaccgtgacaagccaagggccattgctgtcaatgctgagcgagggtacatgtactttaccaacatgcaggaccatgctgccaagatcgagcgagcctccctggatggcacagagcgggaggtcctcttcaccacaggcctcatccgtcccgtggcccttgtggtggacaatgctctgggcaagctcttctgggtggatgccgacctaaagcgaatcgaaagctgtgacctctctggggccaaccgcctgaccctggaagatgccaacatcgtacagccagtaggtctgacagtgctgggcaggcacctctactggatcgaccgccagcagcagatgatcgagcgcgtggagaagaccactggggacaagcggactagggttcagggccgtgtcacccacctgacaggcatccatgccgtggaggaagtcagcctggaggagttctcagcccatccttgtgcccgagacaatggcggctgctcccacatctgtatcgccaagggtgatggaacaccgcgctgctcgtgccctgtccacctggtgctcctgcagaacctgctgacttgtggtgagcctcctacctgctcccctgatcagtttgcatgtaccactggtgagatcgactgcatccccggagcctggcgctgtgacggcttccctgagtgtgctgaccagagtgatgaagaaggctgcccagtgtgctccgcctctcagttcccctgcgctcgaggccagtgtgtggacctgcggttacgctgcgacggtgaggccgactgccaggatcgctctgatgaagctaactgcgatgctgtctgtctgcccaatcagttccggtgcaccagcggccagtgtgtcctcatcaagcaacagtgtgactccttccccgactgtgctgatgggtctgatgagctcatgtgtgaaatcaacaagccaccctctgatgacatcccagcccacagcagtgccattgggcccgtcattggtatcatcctctccctcttcgtcatgggcggggtctactttgtctgccagcgtgtgatgtgccagcgctacacaggggccagtgggccctttccccacgagtatgttggtggagcccctcatgtgcctctcaacttcatagccccaggtggctcacagcacggtcccttcccaggcatcccgtgcagcaagtccgtgatgagctccatgagcctggtgggggggcgcggcagcgtgcccctctatgaccggaatcacgtcactggggcctcatccagcagctcgtccagcacaaaggccacactatatccgccgatcctgaacccacccccgtccccggccacagacccctctctctacaacgtggacgtgttttattcttcaggcatcccggccaccgctagaccatacaggccctacgtcattcgaggtatggcacccccaacaacaccgtgcagcacagatgtgtgtgacagtgactacagcatcagtcgctggaagagcagcaaatactacctggacttgaattcggactcagacccctacccccccccgcccaccccccacagccagtacctatctgcagaggacagctgcccaccctcaccaggcactgagaggagttactgccacctcttcccgcccccaccgtccccctgcacggactcgtcctgacctcggccgtccacccggccctgctgcctccctgtaaatatttttaaatatgaacaaaggaaaaatatattttatgatttaaaaaataaatataattggggtttttaacaagtgagaaatgtgagcggtgaaggggtgggcagggctgggaaacttttctag

SEQ ID NO: 82 - protein, 1614 aa, Mouse
METAPTRAPPPPPPPLLLLVLYCSLVPAAASPLLLFANRRDVRLVDAGGVKLESTIVASGLEDAAAVDFQFSKGAVYWTDVSEEAIKQTYLNQTGAAAQNIVISGLVSPDGLACDWVGKKLYWTDSETNRIEVANLNGTSRKVLFWQDLDQPRAIALDPAHGYMYWTDWGEAPRIERAGMDGSTRKIIVDSDIYWPNGLTIDLEEQKLYWADAKLSFIHRANLDGSFRQKVVEGSLTHPFALTLSGDTLYWTDWQTRSIHACNKWTGEQRKEILSALYSPMDIQVLSQERQPPFHTPCEEDNGGCSHLCLLSPREPFYSCACPTGVQLQDNGKTCKTGAEEVLLLARRTDLRRISLDTPDFTDIVLQVGDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIVLHPVMGLMYWTDWGENPKIECANLDGRDRHVLVNTSLGWPNGLALDLQEGKLYWGDAKTDKIEVINIDGTKRKTLLEDKLPHIFGFTLLGDFIYWTDWQRRSIERVHKVKASRDVIIDQLPDLMGLKAVNVAKVVGTNPCADGNGGCSHLCFFTPRATKCGCPIGLELLSDMKTCIIPEAFLVFTSRATIHRISLETNNNDVAIPLTGVKEASALDFDVSNNHIYWTDVSLKTISRAFMNGSSVEHVIEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERMVIADDLPYPFGLTQYSDYIYWTDWNLHSIERADKTSGRNRTLIQGHLDFVMDILVFHSSRQDGLNDCVHSNGQCGQLCLAIPGGHRCGCASHYTLDPSSRNCSPPSTFLLFSQKFAISRMIPDDQLSPDLVLPLHGLRNVKAINYDPLDKFIYWVDGRQNIKRAKDDGTQPSMLTSPSQSLSPDRQPHDLSIDIYSRTLFWTCEATNTINVHRLDGDAMGVVLRGDRDKPRAIAVNAERGYMYFTNMQDHAAKIERASLDGTEREVLFTTGLIRPVALVVDNALGKLFWVDADLKRIESCDLSGANRLTLEDANIVQPVGLTVLGRHLYWIDRQQQMIERVEKTTGDKRTRVQGRVTHLTGIHAVEEVSLEEFSAHPCARDNGGCSHICIAKGDGTPRCSCPVHLVLLQNLLTCGEPPTCSPDQFACTTGEIDCIPGAWRCDGFPECADQSDEEGCPVCSASQFPCARGQCVDLRLRCDGEADCQDRSDEANCDAVCLPNQFRCTSGQCVLIKQQCDSFPDCADGSDELMCEINKPPSDDIPAHSSAIGPVIGIILSLFVMGGVYFVCQRVMCQRYTGASGPFPHEYVGGAPHVPLNFIAPGGSQHGPFPGIPCSKSVMSSMSLVGGRGSVPLYDRNHVTGASSSSSSSTKATLYPPILNPPPSPATDPSLYNVDVFYSSGIPATARPYRPYVIRGMAPPTTPCSTDVCDSDYSISRWKSSKYYLDLNSDSDPYPPPPTPHSQYLSAEDSCPPSPGTERSYCHLFPPPPSPCTDSS

SEQ ID NO: 83 - DNA, 5301 bp, Homo sapiens
gcggccgccccggctcctcgcctcccccacttctggccacccctcgccggtgagagaagagaacgcgagaagggaagatgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccattgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctgaggaggggccctcctcctctgactgcctccaacgtaaaaatgtaaatataaatttggttgagatctggagggggggagggagctattagagaaggatgaggcagaccatgtacagttaaaattataaaatggggtagggaatactggagatatttgtacagaagaaaaggatatttatatattttcttaaaacagcagatttgctgcttgtgccataaaagtttgtataaaaaaaatttgtactaaaagttttatttttgcaaactaaatacacaaagcatgccttaaacccagtgaagcaactgagtacaaaggaaacaggaataataaaggcatcactgaccaggaatatctgggctttattgataccaaaaaaaaaaaaa

SEQ ID NO: 84 - protein, 1613 aa, Homo sapiens
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAIVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 85 - DNA, 5521 bp, Mouse
gcggccgccccggctcctcgcccgcgcgtcccctggccgccgggcgccggtgagggccgcgagcgcgcgcggtggtggggggaagatgggggccgtgctgaggagcctcctggcctgcagcttctgcgtgctgctgagagcggcccctttgttgctttatgcaaacagacgggacttgagattggttgatgctacaaatggcaaagagaatgcaacgattgtagttggaggcttggaggatgcagctgcggtggactttgtgtttggtcatggcttgatatactggagtgatgtcagcgaagaagccattaaacgaacagaatttaacaaaagtgaaagtgtacagaatgttgttgtttctggattattgtccccggatgggctggcatgtgattggcttggagaaaaattgtactggacagattctgaaactaatcgtattgaagtttctaatttagatggatctttacgaaaagttttattttggcaagagttggatcaacccagagctattgccttagatccatcaagtgggttcatgtactggacagactggggagaagtgccaaagatagaacgggctgggatggatggctcaagtcgcttcgttataataaacacggagatttactggccaaacggactgactctggattatcaggagcggaagctttactgggccgatgcaaaacttaatttcatccataaatcaaacctggatggaacaaaccggcaggcagtggttaaaggttcccttccacatccttttgccttgacgttatttgaggacacattgtactggactgactggaatacacactctattttggcttgcaacaaatatactggcgagggtctgcgtgaaattcattctaacatcttctctcccatggatatacatgctttcagccaacagaggcagccaaatgctacaaatccatgtggaattgataatggtggttgttcccatttgtgtttgatgtctccagtcaagcctttttatcagtgtgcttgcccaactggggtcaagctgatggagaatggaaagacctgcaaagatggtgccactgaactattgctgttagcccgacggacagacttgaggcgaatttctttggatacacccgattttactgacattgttctgcagttagaagatatccggcatgccattgccatagactatgaccctgtagaaggctacatatactggacagatgacgaagtgagggctatccgtcgctccttcatagatggatctggcagtcagtttgtggtcacggcccagattgctcatcctgatggtattgctgttgactgggttgcaaggaacctgtactggacagacactggcacggatcgtatagaagtgacaaggctcaatgggaccatgaggaagatcttgatttcagaggacttagaggagccccgggctatcgtgttagatcccatggttgggtacatgtattggacagactggggagaaatcccaaaaatagagcgagctgctctggacggatctgaccgagtagttcttgtcaacacttcccttggttggccaaacggcttagccctggattatgatgaaggcacaatatactggggagatgccaaaacagacaaaattgaggttatgaataccgatggcaccgggaggcgagtgctggtggaagacaagatccctcacatatttgggtttaccttgctgggtgactatgtttactggactgactggcagaggcggagcatcgagagagtacacaaacggagcgcagagagggaagtcatcatagaccagctgccagacctcatgggactgaaggccacaagtgttcacagagtcattggttctaacccctgtgctgaggacaatggaggatgtagccatctttgcctgtacaggcctcaggggcttcgatgcgcctgtcccattggctttgagctcatcggtgacatgaagacatgcattgtccccgaggctttccttctgttctcgaggagagcggatatcagacgcatatctttggaaacaaacaacaacaatgtggccattcctctcactggtgtcaaagaagcctctgctttggattttgatgtcacagacaacaggatttactggactgatatatcactgaagactattagcagagcctttatgaatggcagtgcactggaacatgtggtagagtttggcttagattatccagaaggcatggcagtggactggcttgggaagaacttatactgggcagacacaggaacaaatcgcattgaggtatcaaagttggacggacagcaccgacaggttttggtatggaaagaccttgacagtcctcgagctctggcactggatcctgctgaagggtttatgtattggactgagtggggaggcaagcctaagattgacagggctgctatggatggaagtgaacgcactacattagttccaaatgtaggccgagcaaatggtctcaccatcgactatgctaaaaggcggctttactggacagacctggacactaacctaatagaatcctcagatatgctcggactcaaccgtgaagttatagcagatgacttgcctcatccttttggcttaactcagtaccaagattacatctactggacagactggagccgacgcagcattgaacgtgccaacaaaaccagtggccaaaaccgcaccatcatccagggccatttggactatgtgatggacatcctggtcttccactcttcccggcaggcagggtggaatgagtgtgcctccagcaacgggcactgctcccacctctgcttggctgtgcccgtcggaggttttgtgtgtggatgccctgcccactactccctgaatgctgacaacaggacctgcagtgctcccagcaccttcctgctcttcagtcagaagagcgccatcaaccgcatggtgattgatgaacaacagagccctgacatcatccttcctatccacagccttcggaacgtccgggccattgactatgaccctttggacaagcagctctactggattgactctcgacaaaactccatacgaaaggcacatgaagatggtggccagggttttaatgtagttgcaaactcggtcgcaaatcagaaccttgaaatacagccctatgatctcagcattgatatttatagccgttacatctactggacctgtgaagctaccaatgtcattgatgtgacgagattagatggacgatcagttggagtggttctaaaaggcgagcaagacagacctcgagccattgtggtaaaccccgagaaagggtatatgtattttaccaatcttcaggaaagatctcctaaaattgaacgggctgcattggatggtacagaacgagaggtcctctttttcagtggcttaagtaaaccaattgctttggctcttgatagcaagctgggcaagctcttctgggctgactcagatctccggcgaattgaaagcagtgatctctcaggtgccaacaggatcgtgctagaagactctaatatattacagcctgtgggcctgaccgtgtttgaaaactggctctattggattgataaacagcagcagatgattgaaaaaattgacatgactggtcgagaaggaagaaccaaggtccaggctcgaattgctcagctgagtgacatccatgcagtaaaggagctgaaccttcaggagtacagacagcacccttgtgcccaggataatggtggctgttcacatatctgccttgtaaaaggagatggtacgacaagatgctcctgccccatgcacttagttctgcttcaggatgagctgtcctgtggagagcctccaacgtgttctcctcagcagtttacctgcttcactggggacattgactgcatccctgtggcttggcggtgtgatgggttcactgagtgcgaagaccacagcgatgaactcaattgtcccgtgtgctcagagtctcagttccagtgtgccagcgggcagtgcattgatggtgcccttcgatgcaatggcgatgcgaactgccaggacaaatcagatgagaagaactgtgaagtgctttgtttaattgatcagttccgctgtgccaatggtcagtgcgttggaaagcacaagaaatgtgaccacagtgtggactgcagtgacagatctgacgagctggactgttatccaactgaggagccagcaccacaagccaccaacacagttggttccgttattggagtaattgtcaccatttttgtgtctggaaccatatactttatctgccagaggatgctgtgtcctcgtatgaagggagacggggagaccatgactaacgactatgtggttcacagcccggcgtctgtgccccttggttatgttcctcacccaagctctctctctggatctcttccaggaatgtctcgaggcaaatcaatgatcagttccctcagtatcatggggggaagcagtgggcccccctatgatcgagcgcacgtcacgggagcctcctcaagcagttcttccagtaccaaaggcacttatttccctgcaattttgaacccaccaccatcccctgccacagaaagatcccattataccatggaatttggttattcttccaacagtccttccacacataggtcctacagctataggccgtacagctaccggcactttgcaccgcccaccacaccctgcagcactgatgtctgtgacagtgactatgctcctagccggaggatgacctcggtggcaacagccaagggctacaccagtgacgtgaactatgactcagaacctgtgcccccaccgcccacaccccgaagccagtacttgtcagcggaggagaactatgaaagctgccccccttccccatacacggagaggagttactcccaccacctctacccgccaccaccctccccctgcacggactcctcctgaggagggcccctcctcctctgactgcctccaccgggacatgtaaatacacatctggttgagatctggagggggggagggagctatagagaagagtgaggcagactctgtacagttaacattataaagtgggtgggtgctggagatatttgtacagaagaaaaggatatttatatattttcttaaaacagcagttttgctgcttgtgccataaaagtttgtataaaaaataaatttgtactaaaagttttatttttgcaaactaaatacacagagcatgccttaagcccagtgaagagactgagtacaaaggaaacaggaaaagaaaggcatcactgaccaggagtgtctgggttttaatgataccaaaaaataaaaaggagaaagtaattccatcttagagtagcaaacttggaagtagccaaattgcctttgggttaactaacatttgagggccgacaagtcgagaacccatgaaagaagaaataatagtactaactttggacacagggctttgttttctccaggaatccagcaattttcacgaggaaaatagatagttgtgagatccgttttgagtaaaaaaaaaaaaaaaaa

SEQ ID NO: 86 - protein, 1613 aa, Mouse
MGAVLRSLLACSFCVLLRAAPLLLYANRRDLRLVDATNGKENATIVVGGLEDAAAVDFVFGHGLIYWSDVSEEAIKRTEFNKSESVQNVVVSGLLSPDGLACDWLGEKLYWTDSETNRIEVSNLDGSLRKVLFWQELDQPRAIALDPSSGFMYWTDWGEVPKIERAGMDGSSRFVIINTEIYWPNGLTLDYQERKLYWADAKLNFIHKSNLDGTNRQAVVKGSLPHPFALTLFEDTLYWTDWNTHSILACNKYTGEGLREIHSNIFSPMDIHAFSQQRQPNATNPCGIDNGGCSHLCLMSPVKPFYQCACPTGVKLMENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGTIYWGDAKTDKIEVMNTDGTGRRVLVEDKIPHIFGFTLLGDYVYWTDWQRRSIERVHKRSAEREVIIDQLPDLMGLKATSVHRVIGSNPCAEDNGGCSHLCLYRPQGLRCACPIGFELIGDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSDMLGLNREVIADDLPHPFGLTQYQDYIYWTDWSRRSIERANKTSGQNRTIIQGHLDYVMDILVFHSSRQAGWNECASSNGHCSHLCLAVPVGGFVCGCPAHYSLNADNRTCSAPSTFLLFSQKSAINRMVIDEQQSPDIILPIHSLRNVRAIDYDPLDKQLYWIDSRQNSIRKAHEDGGQGFNVVANSVANQNLEIQPYDLSIDIYSRYIYWTCEATNVIDVTRLDGRSVGVVLKGEQDRPRAIVVNPEKGYMYFTNLQERSPKIERAALDGTEREVLFFSGLSKPIALALDSKLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGDIDCIPVAWRCDGFTECEDHSDELNCPVCSESQFQCASGQCIDGALRCNGDANCQDKSDEKNCEVLCLIDQFRCANGQCVGKHKKCDHSVDCSDRSDELDCYPTEEPAPQATNTVGSVIGVIVTIFVSGTIYFICQRMLCPRMKGDGETMTNDYVVHSPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDVNYDSEPVPPPPTPRSQYLSAEENYESCPPSPYTERSYSHHLYPPPPSPCTDSS

(19) United States
(12) Patent Application Publication
(10) Pub. No.: US 2005/0261189 A1
(43) Pub. Date: Nov. 24, 2005

(54) Methods of promoting cardiac cell proliferation

(75) Inventors: Glenn Larsen, Sudbury, MA (US); Martha Marvin, Williamstown, MA (US); Dean Y. Li, Salt Lake City, UT (US); Elizabeth Wang, Carlisle, MA (US); C. M. Amy Chen, Belmont, MA (US); Steven M. Shamah, Acton, MA (US)

Correspondence Address: FISH & NEAVE IP GROUP;ROPES & GRAY LLP, ONE INTERNATIONAL PLACE, BOSTON, MA, 02110-2624, US

(73) Assignee: Hydra Biosciences, Inc.

(21) Appl. No.: 11/108,528

(22) Filed: Apr. 18, 2005

Related U.S. Application Data
(60) Provisional application No. 60/563,137, filed on Apr. 16, 2004.
(60) Provisional application No. 60/598,368, filed on Aug. 2, 2004.

(51) Int. Cl. A61K 38/17; C12N 5/08

(52) U.S. Cl. 514/12; 435/366

(57) ABSTRACT

The present invention provides novel methods and compositions for promoting proliferation and/or regeneration.

### RELATED APPLICATIONS

This application claims priority to U.S. provisional application Ser. No. 60/598,368, filed Aug. 2, 2004, and U.S. provisional application Ser. No. 60/563,137, filed Apr. 16, 2004. The disclosures of each of the foregoing applications are hereby incorporated by reference in their entirety.

### BACKGROUND OF THE INVENTION

Injuries and diseases of the cardiovascular system exact a dramatic personal and financial toll both in this country and throughout the world. Scientific advances have resulted in a variety of medical and surgical therapies to decrease mortality following a serious cardiovascular incident, as well as to improve the quality of life for survivors of such diseases and injuries. However, each of the available medical and surgical therapies has significant limitations. Most notably, since the term “cardiovascular disease and injury” encompasses a wide range of conditions, individual medical and/or surgical therapies must be developed to treat each indication. Accordingly, there exists a substantial need in the art for improved methods and compositions for treating a wide range of cardiac diseases and injuries.

Mammals typically heal an injury, whether induced from trauma or disease, by replacing the missing tissue with scar tissue. In the case of cardiac tissue, events such as a myocardial infarction result in substantial damage and even death to cardiomyocytes and other cardiac cells and tissues. However, instead of replacing the damaged cardiac muscle with functional cardiomyocytes, formation of scar tissue further strains and compromises the functional performance of the surviving cardiac tissue. This model, whereby diseased or damaged cardiomyocytes are replaced by scar tissue which further impedes the functional performance of the already compromised cardiovascular system, is recapitulated in a wide range of disease states including congenital cardiovascular disease states.

The loss of cardiac function resulting from injury or disease could be prevented if, as in other non-mammalian species, mammalian fetal, neonatal and adult cardiomyocytes and other cardiac cells regenerated following injury. In contrast to the tissue produced by scarring, regeneration would replace damaged or dead cardiac cells with functional cardiac cells, such as cardiomyocytes, thereby restoring functional performance following cardiac disease or injury. Furthermore, regeneration would replace cardiac cells, such as cardiomyocytes, damaged due to ischemia or other interruption of blood to cardiac tissue due to cardiovascular injury or disease. The present invention provides methods and compositions to promote cardiac cell proliferation, including mammalian fetal, neonatal and adult cardiac cell proliferation. The present invention further provides compositions and methods for promoting regeneration of cardiac cells, such as cardiomyocytes, following injury or disease. In contrast to currently available treatments designed for particular cardiac indications, the methods and compositions of the present invention can be used to treat a wide range of diseases and injuries characterized by damage to cardiac cells, including cardiomyocytes, and/or a decrease in cardiac function.

### BRIEF SUMMARY OF THE INVENTION

The present invention is based on the finding that particular polypeptides, particular modified polypeptides, and particular bioactive fragments promote cardiac cell proliferation. Such cardiac cell proliferation may include, but is not limited to, cardiomyocyte proliferation. Furthermore, such cardiac cell proliferation, for example cardiomyocyte proliferation, includes proliferation of mammalian fetal, neonatal and adult cardiac cells. These polypeptides can be used in methods for promoting cardiac regeneration, as well as methods of treating a wide range of injuries and diseases characterized by injury to cardiomyocytes and/or a decrease in cardiac function.

In a first aspect, the present invention provides methods of promoting cardiac cell proliferation. The method comprises administering a composition comprising a Wnt-related composition in an amount effective to promote proliferation. The Wnt-related compositions according to the invention promote Wnt signaling, specifically, the composition promotes signaling via the canonical Wnt signaling pathway mediated by β-catenin. Exemplary Wnt-related compositions for use in the methods of the present invention modulate Wnt signaling via the canonical Wnt signaling pathway and include Wnt-related compositions, modified Wnt related compositions, and bioactive fragments thereof. In one embodiment, the method promotes cardiomyocyte proliferation.

In one embodiment, the Wnt-related composition may comprise a Wnt polypeptide that may be selected from Wnt1, Wnt2, Wnt2B/Wnt13, Wnt3, Wnt3A, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment thereof, and which Wnt polypeptide promotes Wnt signaling via the canonical Wnt signaling pathway. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway. In another embodiment, the Wnt polypeptide that promotes Wnt signaling via the canonical wnt signaling pathway is selected from Wnt1, Wnt2, Wnt2B, Wnt3, Wnt3A, Wnt6, Wnt7A, Wnt8a, Wnt8b, Wnt9a, Wnt9b, Wnt10a, Wnt10b, and Wnt16. In another embodiment, the Wnt polypeptide is a Wnt polypeptide that promotes wnt signaling via the canonical wnt signaling pathway, and which is not a Wnt3 and/or Wnt3A polypeptide.

In another embodiment, the Wnt-related composition comprises a polypeptide comprising an amino acid sequence at least 80% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In another embodiment, the Wnt-related composition comprises a polypeptide comprising an amino acid sequence at least 90%, 95%, 98%, or 100% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In still another embodiment, the Wnt-related composition comprises a polypeptide encodable by a nucleic acid that hybridizes under stringent conditions, including a wash step of 0.2×SSC at 65° C., to a nucleic acid represented in any of SEQ ID NO: 1, SEQ ID NO:3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO:35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

In any of the foregoing, a Wnt-related polypeptide for use in the methods of the present invention promotes Wnt signaling via the canonical wnt signaling pathway in a cardiac cell type. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway.

In one embodiment, the cardiac cell is an adult cardiac cell. In another embodiment, the cardiac cell is a fetal or neonatal cardiac cell. Exemplary cardiac cells include mammalian cardiomyocytes. Such mammalian cardiomyocytes include, but are not limited to, human, non-human primate, mouse, rat, horse, cow, pig, rabbit, sheep, goat, dog, cat, or hamster. When the cardiomyocyte is a fetal cardiomyocyte, the present invention contemplates methods of promoting fetal cardiomyocyte proliferation in utero.

In any of the foregoing, the Wnt-related composition may further comprise one or more agents that promote binding of the Wnt-related composition to a Wnt-related receptor. In one embodiment, the Wnt-related composition comprises a Wnt3A polypeptide, or bioactive fragment thereof, and the composition further comprises one or more agents that promote binding of the Wnt3A polypeptide to a Wnt3A receptor. Such an agent can be a nucleic acid, peptide, polypeptide, or small organic molecule. By way of example, in one embodiment, the agent is selected from heparin or heparin sulfate. In another embodiment, the Wnt-related composition comprises a Wnt polypeptide selected from Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment of any of the foregoing.

In any of the foregoing, the Wnt-related composition may further comprise one or more agents that promote cardiomyocyte proliferation. Such an agent can be a nucleic acid, peptide, polypeptide, or small organic molecule. By way of example, in one embodiment, the agent is selected from insulin, insulin-like growth factor-1, insulin-like growth factor-2, or a member of the fibroblast growth factor (FGF) family. Exemplary FGF family members include, without limitation, FGF-1, FGF-2, FGF-3, FGF-4, FGF-8, FGF-10, FGF-17, and FGF-18.

In any of the foregoing, the Wnt-related composition may further comprise one or more agents that inhibit cardiomyocyte differentiation. Such an agent can be a nucleic acid, peptide, polypeptide, or small organic molecule. By way of example, in one embodiment, the agent is a p38 inhibitor.

In any of the foregoing, the Wnt-related composition can comprise a modified Wnt polypeptide, or bioactive fragment thereof. Modified Wnt-related compositions comprise a Wnt-related polypeptide appended with one or more moieties. In one embodiment, the Wnt-related composition comprises a modified Wnt3A polypeptide, or bioactive fragment thereof. In another embodiment, the Wnt-related compositions comprises a modified Wnt polypeptide selected from any of Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment of any of the foregoing. Modified polypeptides for use in the present methods retain the ability to promote Wnt signaling via the canonical Wnt signaling pathway (e.g., via the stablization of β-catenin). In certain embodiments, modified Wnt polypeptides retain the ability to promote Wnt signaling via the canonical Wnt signaling pathway and further possess one or more advantageous physiochemical properties in comparison to the corresponding native and/or un-modified Wnt polypeptide.

Modified polypeptides can be modified one, two, three, four, five, or more than five times. Furthermore, modified polypeptides can be modified on the N-terminal amino acid residue, the C-terminal amino acid residue, and/or on an internal amino acid residue. In one embodiment, the modified amino acid reside is a cysteine. In another embodiment, the modified amino acid residue is not a cysteine.

In one embodiment of any of the foregoing, the modified Wnt-related compositions comprise a Wnt-related polypeptide appended with one or more hydrophobic moieties. Exemplary hydrophobic moieties include, but are not limited to, sterols, fatty acids, hydrophobic amino acid residues, and hydrophobic peptides. When a Wnt polypeptide is appended with more than one hydrophobic moiety, each hydrophobic moiety is independently selected. The independently selected moieties can be the same or different. Furthermore, when a polypeptide is appended with more than one moiety, the moieties may include hydrophobic moieties and non-hydrophobic moieties.

In another embodiment of any of the foregoing, the modified Wnt-related compositions comprise a Wnt-related polypeptide appended with one or more hydrophilic moieties. Exemplary hydrophilic moieties include, but are not limited to, PEG containing moieties, cyclodextran, or albumin. When a Wnt-related polypeptide is appended with more than one hydrophilic moiety, each hydrophilic moiety is independently selected. The independently selected moieties can be the same or different. Furthermore, when a polypeptide is appended with more than one moiety, the moieties may include hydrophilic moieties and non-hydrophilic moieties.

In another embodiment of any of the foregoing, the Wnt-related compositions, modified Wnt-related compositions, and/or bioactive fragments thereof, are administered systemically. In yet another embodiment of any of the foregoing, the Wnt-related compositions, modified Wnt-related compositions, and/or bioactive fragments thereof, are administered locally to the myocardium, pericardium, or endocardium.

Furthermore, this aspect of the invention contemplates administration of a Wnt-related compositions alone, in combination with particular agents (e.g., such agents as described in detail herein), or in combination with one or more additional Wnt-related compositions. By way of example, one or more Wnt-related compositions can be administered together with one or more modified Wnt-related compositions, or one or more Wnt-related compositions can be administered with one or more bioactive fragments of a Wnt-related composition.

In any of the foregoing, the invention contemplates that Wnt-related compositions can promote proliferation and/or regeneration of cardiac cells. Such cardiac cells include, but are not limited to, cardiomyocytes. Furthermore, and in any of the foregoing, the invention recognizes that the promotion and/or regeneration of cardiac cells may be accompanied by an increase in cell survival.

In a second aspect, the present invention provides methods of promoting cardiac cell regeneration. The method comprises administering a composition comprising a Wnt-related composition in an amount effective to promote regeneration. The Wnt-related compositions according to the invention promote Wnt signaling, specifically, the composition promotes signaling via the canonical Wnt signaling pathway mediated by β-catenin. In one embodiment, the method promotes cardiomyocyte regeneration.

In one embodiment, the Wnt-related composition may comprise a Wnt polypeptide that may be selected from Wnt1, Wnt2, Wnt2B/Wnt13, Wnt3, Wnt3A, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment thereof, and which Wnt polypeptide promotes Wnt signaling via the canonical Wnt signaling pathway. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway. In another embodiment, the Wnt polypeptide that promotes Wnt signaling via the canonical wnt signaling pathway is selected from Wnt1, Wnt2, Wnt2B, Wnt3, Wnt3A, Wnt6, Wnt7A, Wnt8a, Wnt8b, Wnt9a, Wnt9b, Wnt10a, Wnt10b, and Wnt16. In another embodiment, the Wnt polypeptide is a Wnt polypeptide that promotes wnt signaling via the canonical wnt signaling pathway, and which is not a Wnt3 and/or Wnt3A polypeptide.

In another embodiment, the Wnt-related composition comprises a polypeptide comprising an amino acid sequence at least 80% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID No: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In another embodiment, the Wnt-related composition comprises a polypeptide comprising an amino acid sequence at least 90%, 95%, 98%, or 100% identical to any of SEQ ID NO: 2, SEQ ID NO: 4, SEQ ID NQ: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NQ: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In still another embodiment, the Wnt-related composition comprises a polypeptide encodable by a nucleic acid that hybridizes under stringent conditions, including a wash step of 0.2×SSC at 65° C., to a nucleic acid represented in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

In any of the foregoing, a Wnt-related polypeptide for use in the methods of the present invention promotes Wnt signaling via the canonical wnt signaling pathway in a cardiac cell type. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway.

In one embodiment, the cardiac cell is an adult cardiac cell. In another embodiment, the cardiac cell is a fetal or neonatal cardiac cell. Exemplary cardiac cells include mammalian cardiomyocytes. Such mammalian cardiomyocytes include, but are not limited to, human, non-human primate, mouse, rat, horse, cow, pig, rabbit, sheep, goat, dog, cat, or hamster. When the cardiomyocyte is a fetal cardiomyocyte, the present invention contemplates methods of promoting fetal cardiomyocyte regeneration in utero.

In any of the foregoing, the Wnt-related composition may further comprise one or more agents that promote binding of the Wnt-related composition to a Wnt-related receptor. In one embodiment, the Wnt-related composition comprises a Wnt3A polypeptide, or bioactive fragment thereof, and the composition further comprises one or more agents that promote binding of the Wnt3A polypeptide to a Wnt3A receptor. Such an agent can be a nucleic acid, peptide, polypeptide, or small organic molecule. By way of example, in one embodiment, the agent is selected from heparin or heparin sulfate. In another embodiment, the Wnt-related composition comprises a Wnt polypeptide selected from Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment of any of the foregoing.

In any of the foregoing, the Wnt-related composition may further comprise one or more agents that promote proliferation of cardiomyocytes. Such an agent can be a nucleic acid, peptide, polypeptide, or small organic molecule. By way of example, in one embodiment, the agent is selected from insulin, insulin-like growth factor-1, insulin-like growth factor-2, or a member of the fibroblast growth factor (FGF) family. Exemplary FGF family members include, without limitation, FGF-1, FGF-2, FGF-3, FGF-4, FGF-8, FGF-10, FGF-17, and FGF-18.

In any of the foregoing, the Wnt-related composition may further comprise one or more agents that inhibit cardiomyocyte differentiation. Such an agent can be a nucleic acid, peptide, polypeptide, or small organic molecule. By way of example, in one embodiment, the agent is a p38 inhibitor.

In any of the foregoing, the Wnt-related composition can comprise a modified Wnt polypeptide, or bioactive fragment thereof. Modified Wnt-related compositions comprise a Wnt polypeptide appended with one or more moieties. In one embodiment, the Wnt-related composition comprises a modified Wnt3A polypeptide, or bioactive fragment thereof. In another embodiment, the Wnt-related composition comprises a Wnt polypeptide selected from Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment of any of the foregoing. Modified polypeptides for use in the present method retain the ability to promote Wnt signaling via the canonical Wnt signaling pathway. In certain embodiments, modified Wnt polypeptides retain the ability to promote Wnt signaling and further possess one or more advantageous physiochemical properties in comparison to the corresponding native and/or un-modified Wnt polypeptide.

Modified polypeptides can be modified one, two, three, four, five, or more than five times. Furthermore, modified polypeptides can be modified on the N-terminal amino acid residue, the C-terminal amino acid residue, and/or on an internal amino acid residue. In one embodiment, the modified amino acid reside is a cysteine. In another embodiment, the modified amino acid residue is not a cysteine.

In one embodiment of any of the foregoing, the modified Wnt-related compositions comprise a Wnt polypeptide appended with one or more hydrophobic moieties. Exemplary hydrophobic moieties include, but are not limited to, sterols, fatty acids, hydrophobic amino acid residues, and hydrophobic peptides. When a Wnt polypeptide is appended with more than one hydrophobic moiety, each hydrophobic moiety is independently selected. The independently selected moieties can be the same or different. Furthermore, when a polypeptide is appended with more than one moiety, the moieties may include hydrophobic moieties and non-hydrophobic moieties.

In another embodiment of any of the foregoing, the modified Wnt-related compositions comprise a Wnt polypeptide appended with one or more hydrophilic moieties. Exemplary hydrophilic moieties include, but are not limited to, PEG containing moieties, cyclodextran, or albumin. When a Wnt polypeptide is appended with more than one hydrophilic moiety, each hydrophilic moiety is independently selected. The independently selected moieties can be the same or different. Furthermore, when a polypeptide is appended with more than one moiety, the moieties may include hydrophilic moieties and non-hydrophilic moieties.

In another embodiment of any of the foregoing, the Wnt-related compositions, modified Wnt-related compositions, and/or bioactive fragments thereof, are administered systemically. In yet another embodiment of any of the foregoing, the Wnt-related compositions, modified Wnt-related compositions, and/or bioactive fragments thereof, are administered locally to the myocardium, pericardium, or endocardium.

Furthermore, this aspect of the invention contemplates administration of a Wnt-related compositions alone, in combination with particular agents (e.g., such agents as described in detail herein), or in combination with one or more additional Wnt-related compositions. By way of example, one or more Wnt-related compositions can be administered together with one or more modified Wnt-related compositions, or one or more Wnt-related compositions can be administered with one or more bioactive fragments of a Wnt-related composition.

In any of the foregoing, the invention contemplates that Wnt-related compositions can promote proliferation and/or regeneration of cardiac cells. Such cardiac cells include, but are not limited to, cardiomyocytes. Furthermore, and in any of the foregoing, the invention recognizes that the promotion and/or regeneration of cardiac cells may be accompanied by an increase in cell survival.

In a third aspect, the present invention provides methods of treating a condition characterized by cardiac cell injury or death, for example, cardiomyocyte injury or death. The method comprises administering a composition comprising a Wnt-related composition in an amount effective to treat a condition characterized by cardiac cell injury or death. The Wnt-related compositions according to the invention promote Wnt signaling, specifically, the composition promotes signaling via the canonical Wnt signaling pathway mediated by β-catenin.

In one embodiment, the condition characterized by cardiomyocyte injury or death is selected from any of myocardial infarction; atherosclerosis, coronary artery disease; obstructive vascular disease; dilated cardiomyopathy; heart failure; myocardial necrosis; valvular heart disease; non-compaction of the ventricular myocardium; hypertrophic cardiomyopathy; cancer or cancer-related conditions such as structural defects resulting from cancer or cancer treatments.

In one embodiment, the injury to cardiomyocytes results from myocarditis, exposure to toxin, exposure to an infectious agent, or from a mineral deficiency.

In one embodiment, the Wnt-related composition may comprise a Wnt polypeptide that may be selected from Wnt1, Wnt2, Wnt2B/Wnt13, Wnt3, Wnt3A, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment thereof, and which Wnt polypeptide promotes Wnt signaling via the canonical Wnt signaling pathway. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway. In another embodiment, the Wnt polypeptide that promotes Wnt signaling via the canonical wnt signaling pathway is selected from Wnt1, Wnt2, Wnt2B, Wnt3, Wnt3A, Wnt6, Wnt7A, Wnt8a, Wnt8b, Wnt9a, Wnt9b, Wnt10a, Wnt10b, and Wnt16. In another embodiment, the Wnt polypeptide is a Wnt polypeptide that promotes wnt signaling via the canonical wnt signaling pathway, and which is not a Wnt3 and/or Wnt3A polypeptide.

In another embodiment, the Wnt-related composition comprises a polypeptide comprising an amino acid sequence at least 80% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In another embodiment, the Wnt-related composition comprises a polypeptide comprising an amino acid sequence at least 90%, 95%, 98%, or 100% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In still another embodiment, the Wnt-related composition comprises a polypeptide encodable by a nucleic acid that hybridizes under stringent conditions, including a wash step of 0.2×SSC at 65° C., to a nucleic acid represented in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

In any of the foregoing, a Wnt-related polypeptide for use in the methods of the present invention promotes Wnt signaling via the canonical wnt signaling pathway in a cardiac cell type. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway.

In one embodiment, the cardiac cell is an adult cardiac cell. In another embodiment, the cardiac cell is a fetal or neonatal cardiac cell. Exemplary cardiac cells include mammalian cardiomyocytes. Such mammalian cardiomyocytes include, but are not limited to, human, non-human primate, mouse, rat, horse, cow, pig, rabbit, sheep, goat, dog, cat, or hamster. When the cardiomyocyte is a fetal cardiomyocyte, the present invention contemplates methods of in utero administration.

In any of the foregoing, the Wnt-related composition may further comprise one or more agents that promote binding of the Wnt-related composition to a Wnt-related receptor. In one embodiment, the Wnt-related composition comprises a Wnt3A polypeptide, or bioactive fragment thereof, and the composition further comprises one or more agents that promote binding of the Wnt3A polypeptide to a Wnt3A receptor. Such an agent can be a nucleic acid, peptide, polypeptide, or small organic molecule. By way of example, in one embodiment, the agent is selected from heparin or heparin sulfate. In another embodiment, the Wnt-related composition comprises a Wnt polypeptide selected from Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment of any of the foregoing.

In any of the foregoing, the Wnt-related composition may further comprise one or more agents that promote proliferation of cardiomyocytes. Such an agent can be a nucleic acid, peptide, polypeptide, or small organic molecule. By way of example, in one embodiment, the agent is selected from insulin, insulin-like growth factor-1, insulin-like growth factor-2, or a member of the fibroblast growth factor (FGF) family. Exemplary FGF family members include, without limitation, FGF-1, FGF-2, FGF-3, FGF-4, FGF-8, FGF-10, FGF-17, and FGF-18.

In any of the foregoing, the Wnt-related composition may further comprise one or more agents that inhibit cardiomyocyte differentiation. Such an agent can be a nucleic acid, peptide, polypeptide, or small organic molecule. By way of example, in one embodiment, the agent is a p38 inhibitor.

In any of the foregoing, the Wnt-related composition can comprise a modified Wnt polypeptide, or bioactive fragment thereof. Modified Wnt-related compositions comprise a Wnt polypeptide appended with one or more moieties. In one embodiment, the Wnt-related composition comprises a modified Wnt3A polypeptide, or bioactive fragment thereof. In another embodiment, the Wnt-related composition comprises a Wnt polypeptide selected from Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment of any of the foregoing. Modified polypeptides for use in the present method retain the ability to promote Wnt signaling via the canonical Wnt signaling pathway. In certain embodiments, modified Wnt polypeptides retain the ability to promote Wnt signaling via the canonical Wnt signaling pathway and further possess one or more advantageous physiochemical properties in comparison to the corresponding native and/or unmodified Wnt polypeptide.

Modified polypeptides can be modified one, two, three, four, five, or more than five times. Furthermore, modified polypeptides can be modified on the N-terminal amino acid residue, the C-terminal amino acid residue, and/or on an internal amino acid residue. In one embodiment, the modified amino acid reside is a cysteine. In another embodiment, the modified amino acid residue is not a cysteine.

In one embodiment of any of the foregoing, the modified Wnt-related compositions comprise a Wnt polypeptide appended with one or more hydrophobic moieties. Exemplary hydrophobic moieties include, but are not limited to, sterols, fatty acids, hydrophobic amino acid residues, and hydrophobic peptides. When a Wnt polypeptide is appended with more than one hydrophobic moiety, each hydrophobic moiety is independently selected. The independently selected moieties can be the same or different. Furthermore, when a polypeptide is appended with more than one moiety, the moieties may include hydrophobic moieties and non-hydrophobic moieties.

In another embodiment of any of the foregoing, the modified Wnt-related compositions comprise a Wnt polypeptide appended with one or more hydrophilic moieties. Exemplary hydrophilic moieties include, but are not limited to, PEG containing moieties, cyclodextran, or albumin. When a Wnt polypeptide is appended with more than one hydrophilic moiety, each hydrophilic moiety is independently selected. The independently selected moieties can be the same or different. Furthermore, when a polypeptide is appended with more than one moiety, the moieties may include hydrophilic moieties and non-hydrophilic moieties.

In another embodiment of any of the foregoing, the Wnt-related compositions, modified Wnt-related compositions, and/or bioactive fragments thereof, are administered systemically. In yet another embodiment of any of the foregoing, the Wnt-related compositions, modified Wnt-related compositions, and/or bioactive fragments thereof, are administered locally to the myocardium, pericardium, or endocardium.

Furthermore, this aspect of the invention contemplates administration of a Wnt-related compositions alone, in combination with particular agents (e.g., such agents as described in detail herein), or in combination with one or more additional Wnt-related compositions. By way of example, one or more Wnt-related compositions can be administered together with one or more modified Wnt-related compositions, or one or more Wnt-related compositions can be administered with one or more bioactive fragments of a Wnt-related composition.

In a fourth aspect, the present invention provides methods of treating myocardial damage from myocardial infarction. The method comprises administering a composition comprising a Wnt-related composition in an amount effective to treat myocardial damage from myocardial infarction. The Wnt-related compositions according to the invention promote Wnt signaling, specifically, the composition promotes signaling via the canonical Wnt signaling pathway mediated by β-catenin.

In one embodiment, the Wnt-related composition may comprise a Wnt polypeptide that may be selected from Wnt1, Wnt2, Wnt2B/Wnt13, Wnt3, Wnt3A, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment thereof, and which Wnt polypeptide promotes Wnt signaling via the canonical Wnt signaling pathway. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway. In another embodiment, the Wnt polypeptide that promotes Wnt signaling via the canonical wnt signaling pathway is selected from Wnt1, Wnt2, Wnt2B, Wnt3, Wnt3A, Wnt6, Wnt7A, Wnt8a, Wnt8b, Wnt9a, Wnt9b, Wnt10a, Wnt10b, and Wnt16. In another embodiment, the Wnt polypeptide is a Wnt polypeptide that promotes wnt signaling via the canonical wnt signaling pathway, and which is not a Wnt3 and/or Wnt3A polypeptide.

In another embodiment, the Wnt-related composition comprises a polypeptide comprising an amino acid sequence at least 80% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In another embodiment, the Wnt-related composition comprises a polypeptide comprising an amino acid sequence at least 90%, 95%, 98%, or 100% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In still another embodiment, the Wnt-related composition comprises a polypeptide encodable by a nucleic acid that hybridizes under stringent conditions, including a wash step of 0.2×SSC at 65° C., to a nucleic acid represented in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

In any of the foregoing, a Wnt-related polypeptide for use in the methods of the present invention promotes Wnt signaling via the canonical wnt signaling pathway in a cardiac cell type. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway.

In one embodiment, the cardiomyocyte is an adult cardiomyocyte. In another embodiment, the cardiomyocyte is a fetal or neonatal cardiomyocyte. Exemplary cardiomyocytes include mammalian cardiomyocytes. Such mammalian cardiomyocytes include, but are not limited to, human, non-human primate, mouse, rat, horse, cow, pig, rabbit, sheep, goat, dog, cat, or hamster. When the cardiomyocyte is a fetal cardiomyocyte, the present invention contemplates methods of treating fetal cardiomyocytes in utero.

In any of the foregoing, the Wnt-related composition may further comprise one or more agents that promote binding of the Wnt-related composition to a Wnt-related receptor. In one embodiment, the Wnt-related composition comprises a Wnt3A polypeptide, or bioactive fragment thereof, and the composition further comprises one or more agents that promote binding of the Wnt3A polypeptide to a Wnt3A receptor. Such an agent can be a nucleic acid, peptide, polypeptide, or small organic molecule. By way of example, in one embodiment, the agent is selected from heparin or heparin sulfate. In another embodiment, the Wnt-related composition comprises a Wnt polypeptide selected from Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment of any of the foregoing.

In any of the foregoing, the Wnt-related composition may further comprise one or more agents that promote proliferation of cardiomyocytes. Such an agent can be a nucleic acid, peptide, polypeptide, or small organic molecule. By way of example, in one embodiment, the agent is selected from insulin, insulin-like growth factor-1, insulin-like growth factor-2, or a member of the fibroblast growth factor (FGF) family. Exemplary FGF family members include, without limitation, FGF-1, FGF-2, FGF-3, FGF-4, FGF-8, FGF-10, FGF-17, and FGF-18.

In any of the foregoing, the Wnt-related composition may further comprise one or more agents that inhibit cardiomyocyte differentiation. Such an agent can be a nucleic acid, peptide, polypeptide, or small organic molecule. By way of example, in one embodiment, the agent is a p38 inhibitor.

In any of the foregoing, the Wnt-related composition can comprise a modified Wnt polypeptide, or bioactive fragment thereof. Modified Wnt-related compositions comprise a Wnt polypeptide appended with one or more moieties. In one embodiment, the Wnt-related composition comprises a modified Wnt3A polypeptide, or bioactive fragment thereof. In another embodiment, the Wnt-related composition comprises a Wnt polypeptide selected from Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment of any of the foregoing. Modified polypeptides for use in the present method retain the ability to promote Wnt signaling via the canonical Wnt signaling pathway. In certain embodiments, modified Wnt polypeptides retain the ability to promote Wnt signaling and further possess one or more advantageous physiochemical properties in comparison to the corresponding native and/or un-modified Wnt polypeptide.

Modified polypeptides can be modified one, two, three, four, five, or more than five times. Furthermore, modified polypeptides can be modified on the N-terminal amino acid residue, the C-terminal amino acid residue, and/or on an internal amino acid residue. In one embodiment, the modified amino acid reside is a cysteine. In another embodiment, the modified amino acid residue is not a cysteine.

In one embodiment of any of the foregoing, the modified Wnt-related compositions comprise a Wnt polypeptide appended with one or more hydrophobic moieties. Exemplary hydrophobic moieties include, but are not limited to, sterols, fatty acids, hydrophobic amino acid residues, and hydrophobic peptides. When a Wnt polypeptide is appended with more than one hydrophobic moiety, each hydrophobic moiety is independently selected. The independently selected moieties can be the same or different. Furthermore, when a polypeptide is appended with more than one moiety, the moieties may include hydrophobic moieties and non-hydrophobic moieties.

In another embodiment of any of the foregoing, the modified Wnt-related compositions comprise a Wnt polypeptide appended with one or more hydrophilic moieties. Exemplary hydrophilic moieties include, but are not limited to, PEG containing moieties, cyclodextran, or albumin. When a Wnt polypeptide is appended with more than one hydrophilic moiety, each hydrophilic moiety is independently selected. The independently selected moieties can be the same or different. Furthermore, when a polypeptide is appended with more than one moiety, the moieties may include hydrophilic moieties and non-hydrophilic moieties.

In another embodiment of any of the foregoing, the Wnt-related compositions, modified Wnt-related compositions, and/or bioactive fragments thereof, are administered systemically. In yet another embodiment of any of the foregoing, the Wnt-related compositions, modified Wnt-related compositions, and/or bioactive fragments thereof, are administered locally to the myocardium, pericardium, or endocardium.

Furthermore, this aspect of the invention contemplates administration of a Wnt-related compositions alone, in combination with particular agents (e.g., such agents as described in detail herein), or in combination with one or more additional Wnt-related compositions. By way of example, one or more Wnt-related compositions can be administered together with one or more modified Wnt-related compositions, or one or more Wnt-related compositions can be administered with one or more bioactive fragments of a Wnt-related composition.

In a fifth aspect, the present invention provides methods of treating a developmental disorder of cardiac cells, for example, of cardiomyocytes. The method comprises administering a composition comprising a Wnt-related composition in an amount effective to promote proliferation, regeneration, or survival of cardiomyocytes, thereby treating the developmental disorder. The Wnt-related compositions according to the invention promote Wnt signaling, specifically, the composition promotes signaling via the canonical Wnt signaling pathway mediated by β-catenin. Exemplary Wnt-related compositions for use in the methods of the present invention include Wnt3 related compositions, modified Wnt3 related compositions, and bioactive fragments thereof. Further exemplary Wnt-related compositions include compositions comprising Wnt polypeptides or modified Wnt polypeptides selected from Wnt1, Wnt2, Wnt2B/Wnt13, Wnt3, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or bioactive fragments of any of the foregoing.

In one embodiment, the developmental disorder is selected from any of non-compaction of the ventricular myocardium, congenital heart disease, DiGeorge syndrome, or hypoplastic left heart syndrome.

In one embodiment, the Wnt-related composition comprises a Wnt3A polypeptide, or a bioactive fragment thereof. In one embodiment, the Wnt-related composition may comprise a Wnt polypeptide that may be selected from Wnt1, Wnt2, Wnt2B/Wnt13, Wnt3, Wnt3A, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, Wnt16, or a bioactive fragment thereof, and which Wnt polypeptide promotes Wnt signaling via the canonical Wnt signaling pathway. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway. In another embodiment, the Wnt polypeptide that promotes Wnt signaling via the canonical wnt signaling pathway is selected from Wnt1, Wnt2, Wnt2B, Wnt3, Wnt3A, Wnt6, Wnt7A, Wnt8a, Wnt8b, Wnt9a, Wnt9b, Wnt10a, Wnt10b, and Wnt16. In another embodiment, the Wnt polypeptide is a Wnt polypeptide that promotes wnt signaling via the canonical wnt signaling pathway, and which is not a Wnt3 and/or Wnt3A polypeptide.

In another embodiment, the Wnt-related composition comprises a polypeptide comprising an amino acid sequence at least 80% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In another embodiment, the Wnt-related composition comprises a polypeptide comprising an amino acid sequence at least 90%, 95%, 98%, or 100% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In still another embodiment, the Wnt-related composition comprises a polypeptide encodable by a nucleic acid that hybridizes under stringent conditions, including a wash step of 0.2×SSC at 65° C., to a nucleic acid represented in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO:31, SEQ ID NO:33, SEQ ID NO:35, SEQ ID NO:37, SEQ ID NO:39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

In any of the foregoing, a Wnt-related polypeptide for use in the methods of the present invention promotes Wnt signaling via the canonical wnt signaling pathway in a cardiac cell type. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway.

In one embodiment, the cardiac cell is an adult cardiac cell. In another embodiment, the cardiac cell is a fetal or neonatal cardiac cell. Exemplary cardiac cells include mammalian cardiomyocytes. Such mammalian cardiomyocytes include, but are not limited to, human, non-human primate, mouse, rat, horse, cow, pig, rabbit, sheep, goat, dog, cat, or hamster. When the cardiomyocyte is a fetal cardiomyocyte, the present invention contemplates methods of in utero administration.

In any of the foregoing, the Wnt-related composition may further comprise one or more agents that promote binding of the Wnt-related composition to a Wnt-related receptor. In one embodiment, the Wnt-related composition comprises a Wnt3A polypeptide, or bioactive fragment thereof, and the composition further comprises one or more agents that promote binding of the Wnt3A polypeptide to a Wnt3A receptor. Such an agent can be a nucleic acid, peptide, polypeptide, or small organic molecule. By way of example, in one embodiment, the agent is selected from heparin or heparin sulfate.

In any of the foregoing, the Wnt-related composition may further comprise one or more agents that promote proliferation of cardiomyocytes. Such an agent can be a nucleic acid, peptide, polypeptide, or small organic molecule. By way of example, in one embodiment, the agent is selected from insulin, insulin-like growth factor-1, insulin-like growth factor-2, or a member of the fibroblast growth factor (FGF) family. Exemplary FGF family members include, without limitation, FGF-1, FGF-2, FGF-3, FGF-4, FGF-8, FGF-10, FGF-17, and FGF-18.

In any of the foregoing, the Wnt-related composition may further comprise one or more agents that inhibit cardiomyocyte differentiation. Such an agent can be a nucleic acid, peptide, polypeptide, or small organic molecule. By way of example, in one embodiment, the agent is a p38 inhibitor.

In any of the foregoing, the Wnt-related composition can comprise a modified Wnt polypeptide, or bioactive fragment thereof. Modified Wnt-related compositions comprise a Wnt polypeptide appended with one or more moieties. In one embodiment, the Wnt-related composition comprises a modified Wnt3A polypeptide, or bioactive fragment thereof. Modified polypeptides for use in the present method retain the ability to promote Wnt signaling. In certain embodiments, modified Wnt polypeptides retain the ability to promote Wnt signaling and further possess one or more advantageous physiochemical properties in comparison to the corresponding native and/or un-modified Wnt polypeptide.

Modified polypeptides can be modified one, two, three, four, five, or more than five times. Furthermore, modified polypeptides can be modified on the N-terminal amino acid residue, the C-terminal amino acid residue, and/or on an internal amino acid residue. In one embodiment, the modified amino acid reside is a cysteine. In another embodiment, the modified amino acid residue is not a cysteine.

In one embodiment of any of the foregoing, the modified Wnt-related compositions comprise a Wnt polypeptide appended with one or more hydrophobic moieties. Exemplary hydrophobic moieties include, but are not limited to, sterols, fatty acids, hydrophobic amino acid residues, and hydrophobic peptides. When a Wnt polypeptide is appended with more than one hydrophobic moiety, each hydrophobic moiety is independently selected. The independently selected moieties can be the same or different. Furthermore, when a polypeptide is appended with more than one moiety, the moieties may include hydrophobic moieties and non-hydrophobic moieties.

In another embodiment of any of the foregoing, the modified Wnt-related compositions comprise a Wnt polypeptide appended with one or more hydrophilic moieties. Exemplary hydrophilic moieties include, but are not limited to, PEG containing moieties, cyclodextran, or albumin. When a Wnt polypeptide is appended with more than one hydrophilic moiety, each hydrophilic moiety is independently selected. The independently selected moieties can be the same or different. Furthermore, when a polypeptide is appended with more than one moiety, the moieties may include hydrophilic moieties and non-hydrophilic moieties.

In another embodiment of any of the foregoing, the Wnt-related compositions, modified Wnt-related compositions, and/or bioactive fragments thereof, are administered systemically. In yet another embodiment of any of the foregoing, the Wnt-related compositions, modified Wnt-related compositions, and/or bioactive fragments thereof, are administered locally to the myocardium, pericardium, or endocardium.

Furthermore, this aspect of the invention contemplates administration of a Wnt-related compositions alone, in combination with particular agents (e.g., such agents as described in detail herein), or in combination with one or more additional Wnt-related compositions. By way of example, one or more Wnt-related compositions can be administered together with one or more modified Wnt-related compositions, or one or more Wnt-related compositions can be administered with one or more bioactive fragments of a Wnt-related composition.

In a sixth aspect, the present invention provides use of a Wnt-related polypeptide, modified Wnt-related polypeptide, or bioactive fragment thereof, in the manufacture of a medicament for promoting, for example, cardiomyocyte proliferation. In one embodiment, the Wnt-related polypeptide, modified Wnt-related polypeptide, or bioactive fragment thereof is Wnt3A.

In a seventh aspect, the present invention provides use of a Wnt-related polypeptide, modified Wnt-related polypeptide, or bioactive fragment thereof, in the manufacture of a medicament for promoting, for example, cardiomyocyte regeneration. In one embodiment, the Wnt-related polypeptide, modified Wnt-related polypeptide, or bioactive fragment thereof is Wnt3A. In another embodiment, the Wnt-related polypeptide, modified Wnt-related polypeptide, or bioactive fragment thereof is selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, or Wnt16. In any of the foregoing, the Wnt-related polypeptide for use in the manufacture of a medicament for promoting cardiac cell (e.g., cardiomyocyte) regeneration promotes Wnt signaling via the canonical Wnt signaling pathway (e.g., the canonical β-catenin-mediated Wnt signaling pathway, the canonical β-catenin-dependent Wnt signaling pathway).

For any of the foregoing aspects, the invention contemplates administering a composition comprising a nucleic acid sequence encoding a Wnt-related polypeptide. In one embodiment, the nucleic acid sequence encodes a Wnt-related polypeptide selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, Wnt16. In another embodiment, the nucleic acid sequence encodes a polypeptide at least 80%, 85%, 90%, 95%, 96%, 97%, 98%, 99%, or 100% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or to a bioactive fragment thereof. In another embodiment, the nucleic acid sequence hybridizes under stringent conditions, including a wash step of 0.2×SSC at 65° C., to a sequence represented in SEQ ID NO: 1, SEQ ID NO: 3, SEQ ID NO:5, SEQ ID NO: 7, SEQ ID NO:9, SEQ ID NO: 11, SEQ ID NO: 13, SEQ ID NO: 15, SEQ ID NO:17, SEQ ID NO:19, SEQ ID NO:21, SEQ ID NO:23, SEQ ID NO:25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77. In still another embodiment, the composition comprises a nucleic acid sequence at least 80%, 85%, 90%, 95%, 96%, 97%, 98%, 99%, or 100% identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO:21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NQ: 65, SEQ ID NO: 67, SEQ ID NO: 69, SEQ ID NO: 71, SEQ ID NO: 73, SEQ ID NO: 75, or SEQ ID NO: 77, or a bioactive fragment thereof.

In any of the foregoing methods directed to administration of compositions comprising nucleic acids, the compositions can be formulated and administered using appropriate methodologies outlined for administration of polypeptides.

In an eighth aspect, the present invention provides modified Wnt polypeptides, and bioactive fragments thereof. The modified polypeptide comprises a Wnt polypeptide, or bioactive fragment thereof, appended with one or more moieties to produce a modified Wnt polypeptide, or bioactive fragment thereof. The modified polypeptide retains one or more biological activities of native and/or un-modified Wnt (e.g., promotes Wnt signaling, promotes expression, activity, and/or stability of β-catenin, and/or binds a frizzled receptor), and in the context of the present invention, the one or more biological activities retained by the modified polypeptides include the ability to promote Wnt signaling via the canonical Wnt signaling pathway. In one embodiment, the modified Wnt polypeptide retains a biological activity of native and/or unmodified Wnt and also possesses one or more advantageous physiochemical properties in comparison to native and/or unmodified Wnt.

In one embodiment, the modified Wnt polypeptide, or bioactive fragment thereof, is appended with two or more moieties.

In one embodiment, the Wnt-related composition comprises a Wnt polypeptide selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, Wnt16, or a bioactive fragment thereof. In another embodiment, the Wnt-related composition comprises a polypeptide comprising an amino acid sequence at least 80% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In another embodiment, the Wnt-related composition comprises a polypeptide comprising an amino acid sequence at least 90%, 95%, 98%, or 100% identical to any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In still another embodiment, the Wnt-related composition comprises a polypeptide encodable by a nucleic acid that hybridizes under stringent conditions, including a wash step of 0.2×SSC at 65° C., to a nucleic acid represented in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

Modified polypeptides can be modified one, two, three, four, five, or more than five times. Furthermore, modified polypeptides can be modified on the N-terminal amino acid residue, the C-terminal amino acid residue, and/or on an internal amino acid residue. In one embodiment, the modified amino acid reside is a cysteine. In another embodiment, the modified amino acid residue is not a cysteine.

In one embodiment of any of the foregoing, the modified Wnt-related compositions comprise a Wnt polypeptide appended with one or more hydrophobic moieties. Exemplary hydrophobic moieties include, but are not limited to, sterols, fatty acids, hydrophobic amino acid residues, and hydrophobic peptides. When a Wnt polypeptide is appended with more than one hydrophobic moiety, each hydrophobic moiety is independently selected. The independently selected moieties can be the same or different. Furthermore, when a polypeptide is appended with more than one moiety, the moieties may include hydrophobic moieties and non-hydrophobic moieties.

In another embodiment of any of the foregoing, the modified Wnt-related compositions comprise a Wnt polypeptide appended with one or more hydrophilic moieties. Exemplary hydrophilic moieties include, but are not limited to, PEG containing moieties, cyclodextran, or albumin. When a Wnt polypeptide is appended with more than one hydrophilic moiety, each hydrophilic moiety is independently selected. The independently selected moieties can be the same or different. Furthermore, when a polypeptide is appended with more than one moiety, the moieties may include hydrophilic moieties and non-hydrophilic moieties.

In another embodiment of any of the foregoing, the modified Wnt-related polypeptide, or bioactive fragment thereof, is formulated in a pharmaceutically acceptable carrier. In another embodiment of any of the foregoing, the modified Wnt-related polypeptide, or bioactive fragment thereof, is attached to a biocompatible device or dissolved in a biocompatible matrix.

In a ninth aspect, the present invention provides biocompatible devices comprising one or more (i) Wnt-related polypeptides, (ii) modified Wnt-related polypeptides, or (iii) bioactive fragments of Wnt-related or modified Wnt-related polypeptides. In another embodiment, the present invention provides biocompatible devices comprising a composition comprising (i) a Wnt-related polypeptide, (ii) a modified Wnt-related polypeptide, or (iii) a bioactive fragment of a Wnt-related or modified Wnt-related polypeptide.

In one embodiment, the biocompatible device is selected from a catheter, stent, wire, suture, or other intraluminal device.

In a tenth aspect, the present invention provides method of screening to identify, characterize, and/or optimize a modified Wnt-related polypeptide. The method comprises modifying a Wnt-related polypeptide or bioactive fragment thereof by attachment of one or more moieties (e.g., one, two, three, four, five, or more than five moieties) to an N-terminal amino acid residue, a C-terminal amino acid residue, and/or an internal amino acid residue and measuring the activity of said modified Wnt-related polypeptide to confirm that said modified polypeptide retains at least one biological activity of the corresponding native or un-modified Wnt polypeptide. In the context of the present invention, the at least one biological activity retained by the modified polypeptide includes the ability to promote Wnt signaling via the canonical Wnt signaling pathway.

In one embodiment, the Wnt-related polypeptide is a Wnt3-related polypeptide.

In another embodiment, the Wnt-related polypeptide is a Wnt polypeptide selected from Wnt1, Wnt2, Wnt2B/Wnt13, Wnt3, Wnt3A, Wnt4, Wnt,5A, Wnt5B, Wnt6, Wnt7A, Wnt7B, Wnt8A, Wnt8B, Wnt9A/Wnt14, Wnt9B, Wnt15, Wnt10A, Wnt10B, Wnt11, or Wnt16.

In one embodiment, the method further comprises measuring one or more physiochemical properties, and selecting modified Wnt-related polypeptides that retain one or more biological activities of the corresponding native and/or unmodified Wnt-related polypeptide and possess one or more advantageous physiochemical properties in comparison to the corresponding native and/or unmodified Wnt polypeptide. Exemplary modified Wnt-related polypeptides for use in the methods of the present invention retain the ability to promote Wnt signaling via the canonical Wnt signaling pathway.

In one embodiment, the method further comprises formulating the modified Wnt-related polypeptide so identified in a pharmaceutically acceptable carrier.

In addition to the foregoing aspects of the invention directed to Wnt-related methods and compositions, the present invention more generally provides methods and compositions for promoting cardiac cell proliferation and/or regeneration by promoting canonical Wnt signaling at the cell surface (e.g., promoting Wnt signaling via the canonical Wnt signaling pathway using agents that act at the cell surface). For example, the present invention provides methods and composition for promoting cardiomyocyte proliferation and/or regeneration by promoting canonical Wnt signaling at the cell surface (e.g., promoting Wnt signaling via the canonical Wnt signaling pathway using agents that act at the cell surface). Exemplary agents that act at the cell surface to promote Wnt signaling via the canonical Wnt signaling pathway include, but are not limited to, Wnt-related polypeptides, modified Wnt-related polypeptides, bioactive fragments of Wnt-related polypeptides, Wnt-related nucleic acids, LRP-related nucleic acids, LRP-related polypeptides, soluble extracellular fragments of LRP-related polypeptides, modified LRP-related polypeptides, modified soluble extracellular fragments of LRP-related polypeptides, N-terminal deletions of LRP-related polypeptide, and anti-LRP-related antibodies. The foregoing class of agents for use in the methods and compositions of the present invention will be referred to herein as agents that act at the cell surface to promote signaling via the canonical Wnt signaling pathway.

In one embodiment, the invention provides a method of promoting cardiac cell proliferation, for example, cardiomyocyte proliferation by administering an agent that acts at the cell surface to promote signaling via the canonical Wnt signaling pathway. Exemplary agents include one or more of the following: Wnt-related polypeptides, modified Wnt-related polypeptides, bioactive fragments of Wnt-related polypeptides, Wnt-related nucleic acids, LRP-related nucleic acids, LRP-related polypeptides, fragments of LRP-related polypeptides comprising N-terminal deletions of the LRP-related polypeptides, nucleic acids encoding fragments of LRP-related polypeptides comprising N-terminal deletions, soluble extracellular fragments of LRP-related polypeptides, modified soluble extracellular fragments of LRP-related polypeptides, or anti-LRP-related antibodies.

In another embodiment, the invention provides a method of promoting cardiac cell regeneration, for example, cardiomyocyte regeneration by administering an agent that acts at the cell surface to promote signaling via the canonical Wnt signaling pathway. Exemplary agents include one or more of the following: Wnt-related polypeptides, modified Wnt-related polypeptides, bioactive fragments of Wnt-related polypeptides, Wnt-related nucleic acids, LRP-related nucleic acids, LRP-related polypeptides, fragments of LRP-related polypeptides comprising N-terminal deletions of the LRP-related polypeptides, nucleic acids encoding fragments of LRP-related polypeptides comprising N-terminal deletions, soluble extracellular fragments of LRP-related polypeptides, modified soluble extracellular fragments of LRP-related polypeptides, or anti-LRP-related antibodies.

In another embodiment, the invention provides a method of treating a condition characterized by cardiac cell (e.g., cardiomyocyte) injury or death by administering an agent that acts at the cell surface to promote signaling via the canonical Wnt signaling pathway. Exemplary agents include one or more of the following: Wnt-related polypeptides, modified Wnt-related polypeptides, bioactive fragments of Wnt-related polypeptides, Wnt-related nucleic acids, LRP-related nucleic acids, LRP-related polypeptides, fragments of LRP-related polypeptides comprising N-terminal deletions of the LRP-related polypeptides, nucleic acids encoding fragments of LRP-related polypeptides comprising N-terminal deletions, soluble extracellular fragments of LRP-related polypeptides, modified soluble extracellular fragments of LRP-related polypeptides, or anti-LRP-related antibodies.

In still another embodiment, the invention provides a method of treating myocardial damage resulting from myocardial infarction by administering an agent that acts at the cell surface to promote signaling via the canonical Wnt signaling pathway. Exemplary agents include one or more of the following: Wnt-related polypeptides, modified Wnt-related polypeptides, bioactive fragments of Wnt-related polypeptides, Wnt-related nucleic acids, LRP-related nucleic acids, LRP-related polypeptides, fragments of LRP-related polypeptides comprising N-terminal deletions of the LRP-related polypeptides, nucleic acids encoding fragments of LRP-related polypeptides comprising N-terminal deletions, soluble extracellular fragments of LRP-related polypeptides, modified soluble extracellular fragments of LRP-related polypeptides, or anti-LRP-related antibodies.

In still another embodiment, the invention provides a method of treating a developmental disorder of cardiac cells, such as cardiomyocytes, by administering an agent that acts at the cell surface to promote signaling via the canonical Wnt signaling pathway. Exemplary agents include one or more of the following: Wnt-related polypeptides, modified Wnt-related polypeptides, bioactive fragments of Wnt-related polypeptides, Wnt-related nucleic acids, LRP-related nucleic acids, LRP-related polypeptides, fragments of LRP-related polypeptides comprising N-terminal deletions of the LRP-related polypeptides, nucleic acids encoding fragments of LRP-related polypeptides comprising N-terminal deletions, soluble extracellular fragments of LRP-related polypeptides, modified soluble extracellular fragments of LRP-related polypeptides, or anti-LRP-related antibodies.

In yet another embodiment, the invention provides the use of an agent that acts at the cell surface to promote Wnt signaling in the manufacture of a medicament to promote cardiomyocyte proliferation and/or to promote cardiomyocyte regeneration. In one embodiment, the cardiomyocyte may be an adult cardiomyocyte.

In any of the foregoing embodiments of this aspect of the invention, the cardiomyocyte may be a fetal or neonatal cardiomyocyte. Exemplary cardiomyocytes include mammalian cardiomyocytes. Such mammalian cardiomyocytes include, but are not limited to, human, non-human primate, mouse, rat, horse, cow, pig, rabbit, sheep, goat, dog, cat, or hamster. When the cardiomyocyte is a fetal cardiomyocyte, the present invention contemplates methods of promoting fetal cardiomyocyte proliferation in utero.

In any of the foregoing, the composition comprising an agent that acts at the cell surface to promote Wnt signaling via the canonical Wnt signaling pathway may further comprise one or more agents that promote binding of a Wnt-related composition to a Wnt-related receptor. Such an agent can be a nucleic acid, peptide, polypeptide, or small organic molecule. By way of example, in one embodiment, the agent is selected from heparin or heparin sulfate.

In any of the foregoing, the composition comprising an agent that acts at the cell surface to promote Wnt signaling via the canonical Wnt signaling pathway may further comprise one or more agents that promote cardiomyocyte proliferation. Such an agent can be a nucleic acid, peptide, polypeptide, or small organic molecule. By way of example, in one embodiment, the agent is selected from insulin, insulin-like growth factor-1, insulin-like growth factor-2, or a member of the fibroblast growth factor (FGF) family. Exemplary FGF family members include, without limitation, FGF-1, FGF-2, FGF-3, FGF-4, FGF-8, FGF-10, FGF-17, and FGF-18.

In any of the foregoing, the composition comprising an agent that acts at the cell surface to promote Wnt signaling via the canonical Wnt signaling pathway may further comprise one or more agents that inhibit cardiomyocyte differentiation. Such an agent can be a nucleic acid, peptide, polypeptide, or small organic molecule. By way of example, in one embodiment, the agent is a p38 inhibitor.

In a twelfth aspect, the invention provides compositions and pharmaceutical compositions comprising an agent that acts at the cell surface to promote signaling via the canonical Wnt signaling pathway.

In one embodiment, the agent is attached to or otherwise formulated on a biocompatible device.

For each of the above aspects of this invention, it is contemplated that any one of the embodiments may be combined with any other embodiments wherever applicable.

The methods and compositions described herein will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### DETAILED DESCRIPTION OF THE DRAWINGS

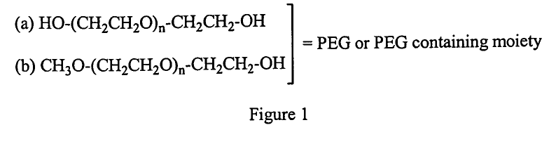

FIG. 1 shows a representation of a PEG containing moiety. Note that the PEG containing moiety may comprise any number of PEG moieties.

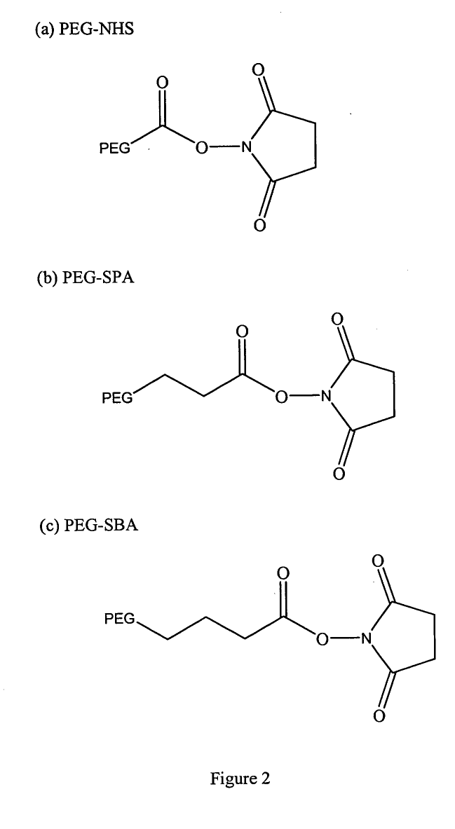

FIG. 2 shows a representation of a PEG containing moiety comprising a reactive group, wherein the PEG containing moiety is a PEG-ester (PEG-NHS, PEG-SPA, PEG-SBA).

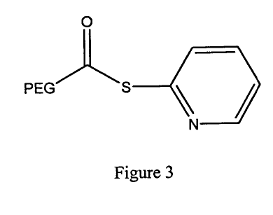

FIG. 3 shows a representation of a PEG containing moiety comprising a reactive group, wherein the PEG containing moiety is a PEG-thioester (PEG-OPTE).

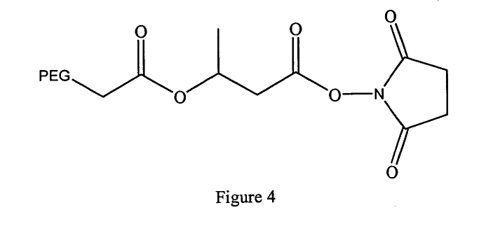

FIG. 4 shows a representation of a PEG containing moiety comprising a reactive group, wherein the PEG containing moiety is a PEG-double ester.

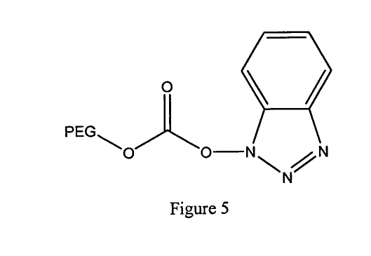

FIG. 5 shows a representation of a PEG containing moiety comprising a reactive group, wherein the PEG containing moiety is a PEG-benzotriazole carbonate (PEG-BTC).

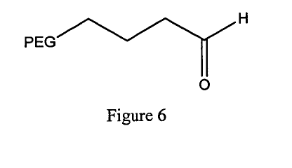

FIG. 6 shows a representation of a PEG containing moiety comprising a reactive group, wherein the PEG containing moiety is a PEG-butyrAld.

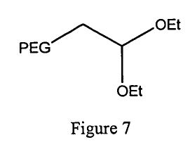

FIG. 7 shows a representation of a PEG containing moiety comprising a reactive group, wherein the PEG containing moiety is a PEG-acetaldehyde diethyl acetal (PEG-ACET).

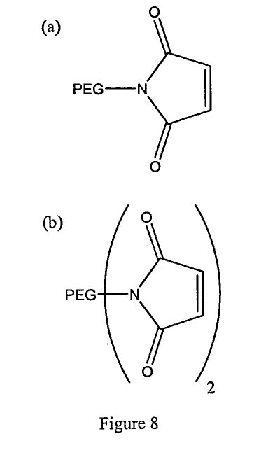

FIG. 8 shows a representation of a PEG containing moiety comprising a reactive group, wherein the PEG containing moiety is a sulfhydryl selective PEG [PEG-maleimide (PEG-MAL)].

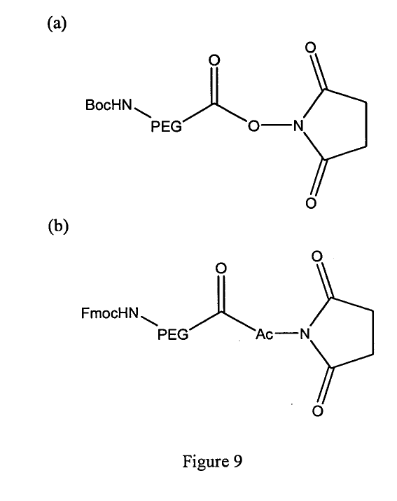

FIG. 9 shows a representation of a PEG containing moiety comprising a reactive group, wherein the PEG containing moiety further contains a Boc or Fmoc protecting group.

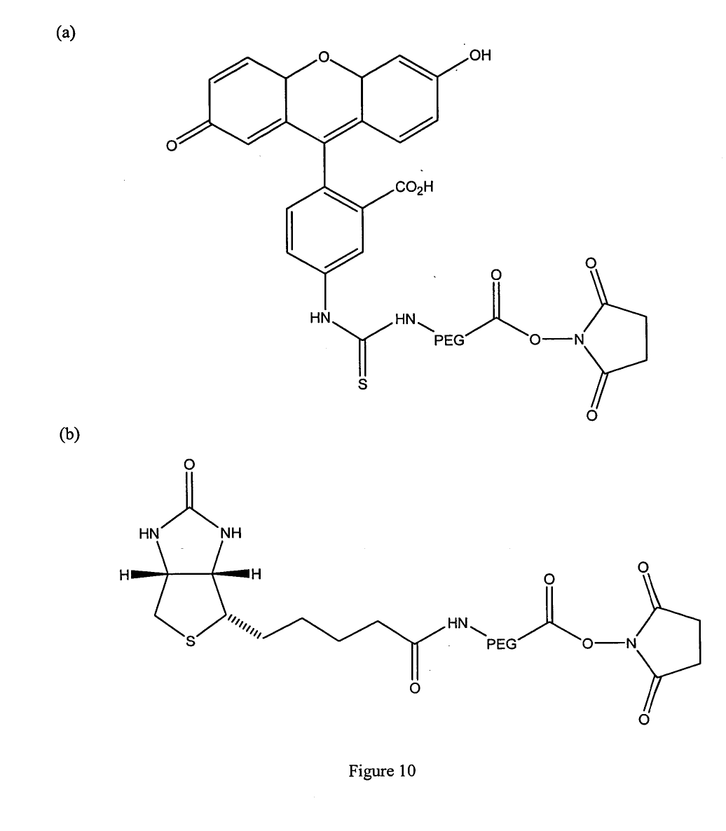

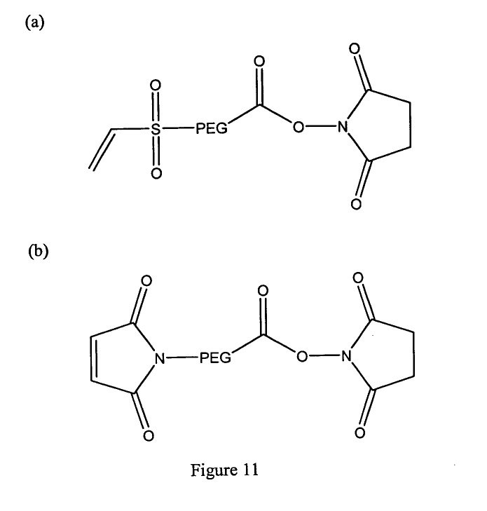

FIG. 10 shows a representation of a PEG containing moiety comprising a reactive group, wherein the PEG containing moiety further contains a detectable moiety for monitoring or otherwise detecting. FIG. 10 shows two such PEG containing moieties: fluorescein-PEG-NHS and Biotin-PEG-NHS FIG. 11 shows a representation of a PEG containing moiety comprising a reactive group, wherein the PEG containing moiety is further modified to promote vinyl polymerization.

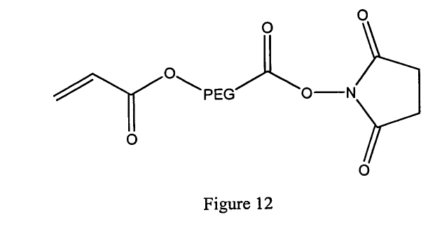

FIG. 12 shows a representation of a PEG containing moiety comprising a reactive group, wherein the PEG containing moiety is further modified to promote vinyl polymerization or co-polymerization.

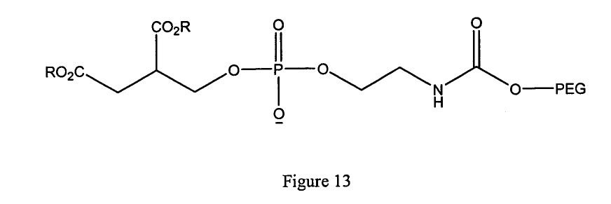

FIG. 13 shows a representation of a PEG containing moiety comprising a reactive group, wherein the PEG containing moiety is further modified with a phospholipid to promote incorporation of the PEG containing moiety into liposomes or other lipid membranes.

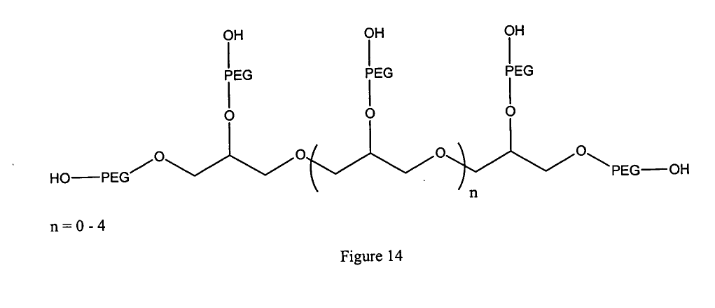

FIG. 14 shows a representation of multifunctional PEG containing moiety.

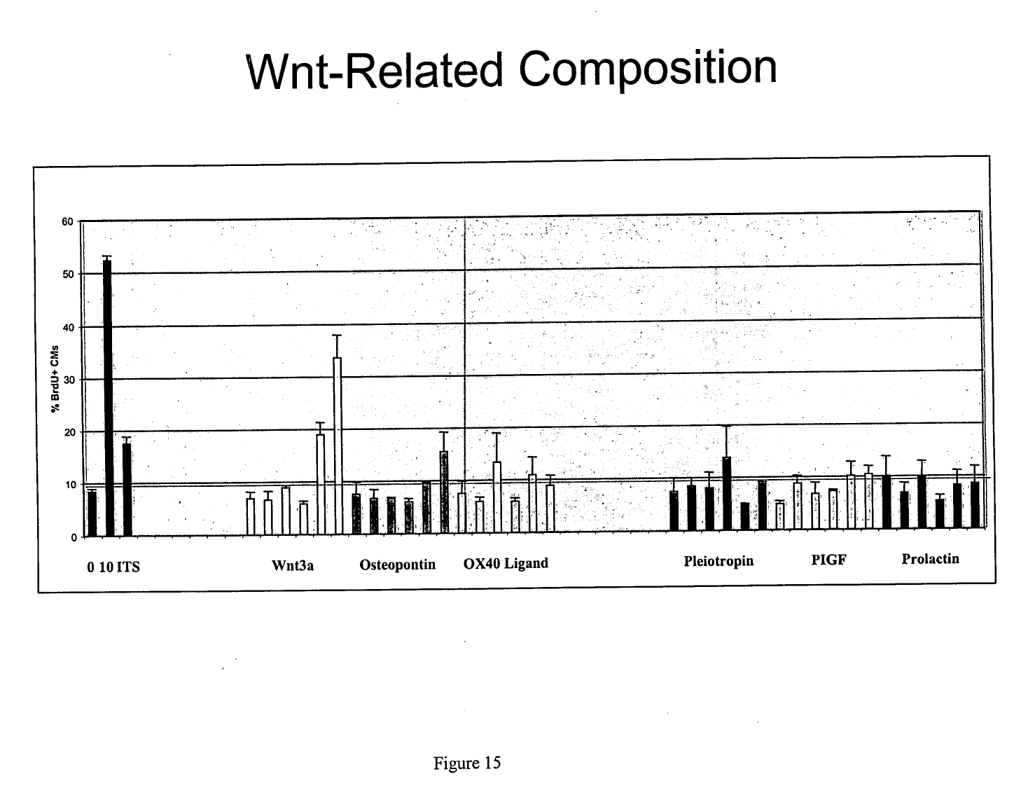

FIG. 15 shows that a Wnt-related composition promoted cardiomyocyte proliferation in neonatal rat cardiomyocytes. Specifically, administration of a recombinant Wnt3A polypeptide to neonatal rat cardiomyocytes resulted in an increase in DNA synthesis. This increase in DNA synthesis was assayed by BrdU incorporation.

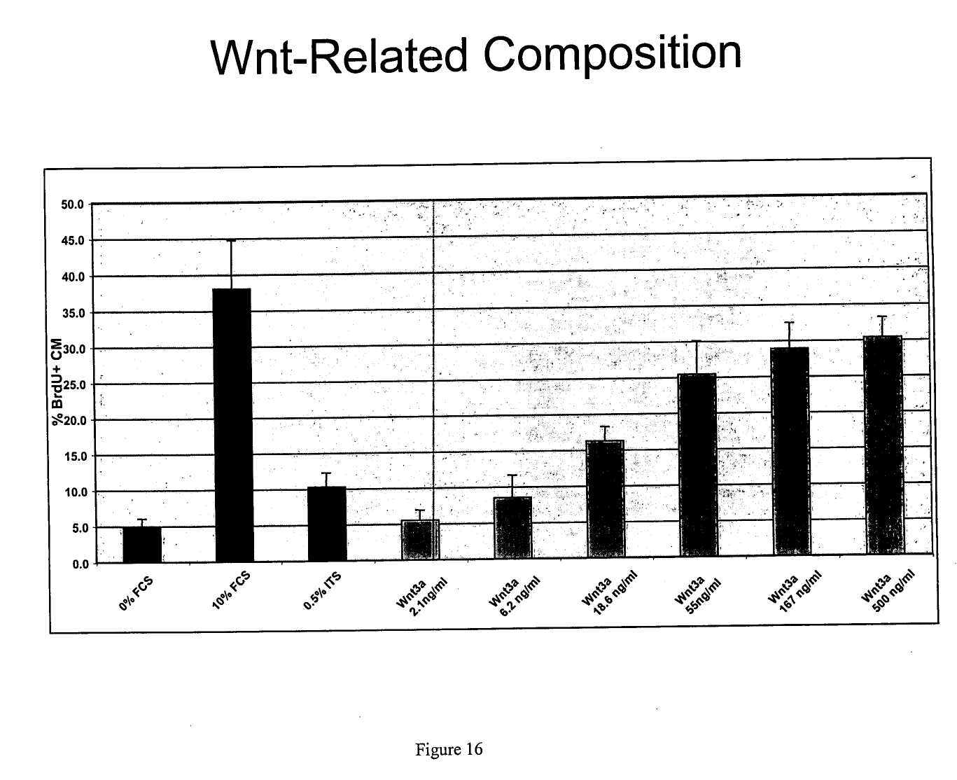

FIG. 16 shows a dilution series of a recombinant Wnt3A polypeptide administered to neonatal rat cardiomyocytes. Administration of a recombinant Wnt3A polypeptide to neonatal rat cardiomyocytes resulted in a dose dependent increase in DNA synthesis, as assayed by BrdU incorporation.

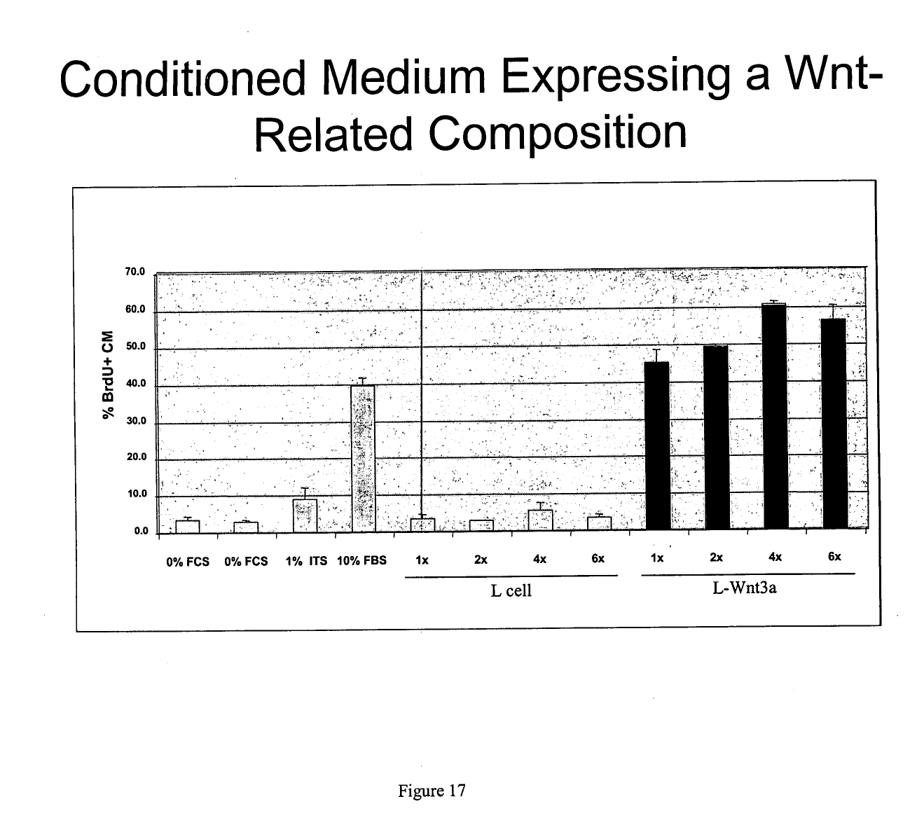

FIG. 17 shows that a Wnt-related composition promoted cardiomyocyte proliferation in neonatal rat cardiomyocytes. Specifically, administration of conditioned medium from Wnt3A expressing cells resulted in an increase in DNA synthesis, as assayed by BrdU incorporation. The increase in proliferation was comparable to the increase in proliferation observed when cells are contacted with 10% fetal bovine serum (FBS). In contrast, administration of conditioned medium from cells that do not express Wnt3A had no effect on proliferation of neonatal rat cardiomyocytes.

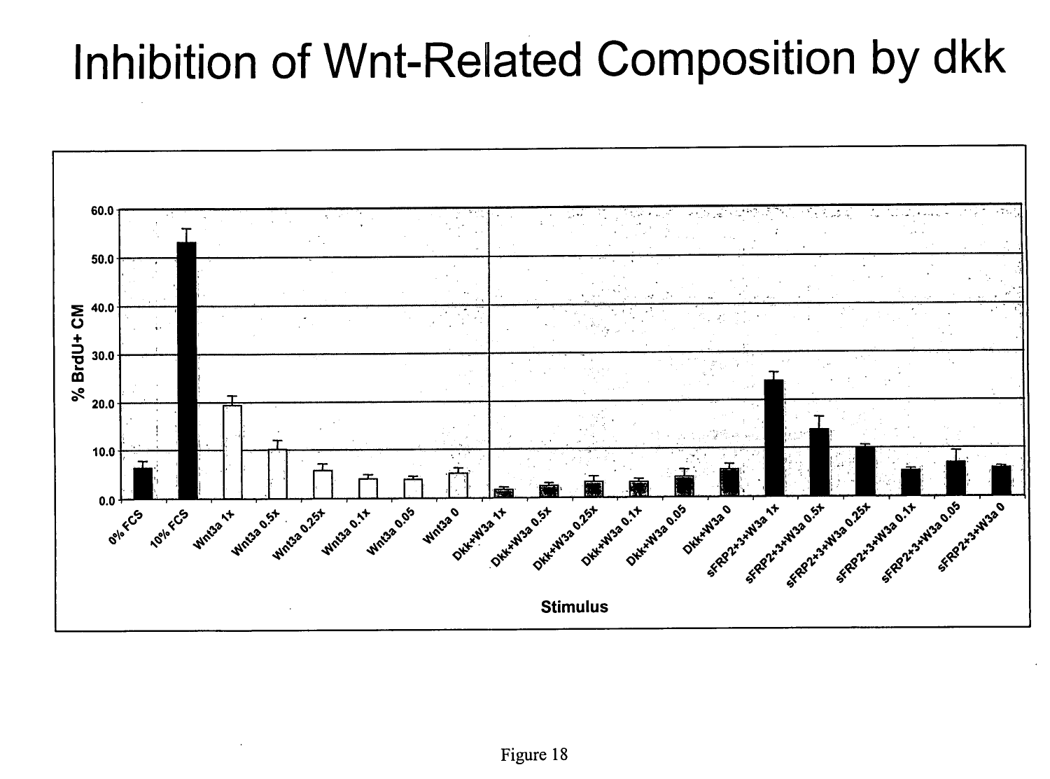

FIG. 18 shows that dkk blocked the proliferative affect of conditioned medium from Wnt3A expressing cells. This indicated that Wnt3A was the active component of the conditioned medium responsible for the increase in proliferation in neonatal cardiomyocytes.

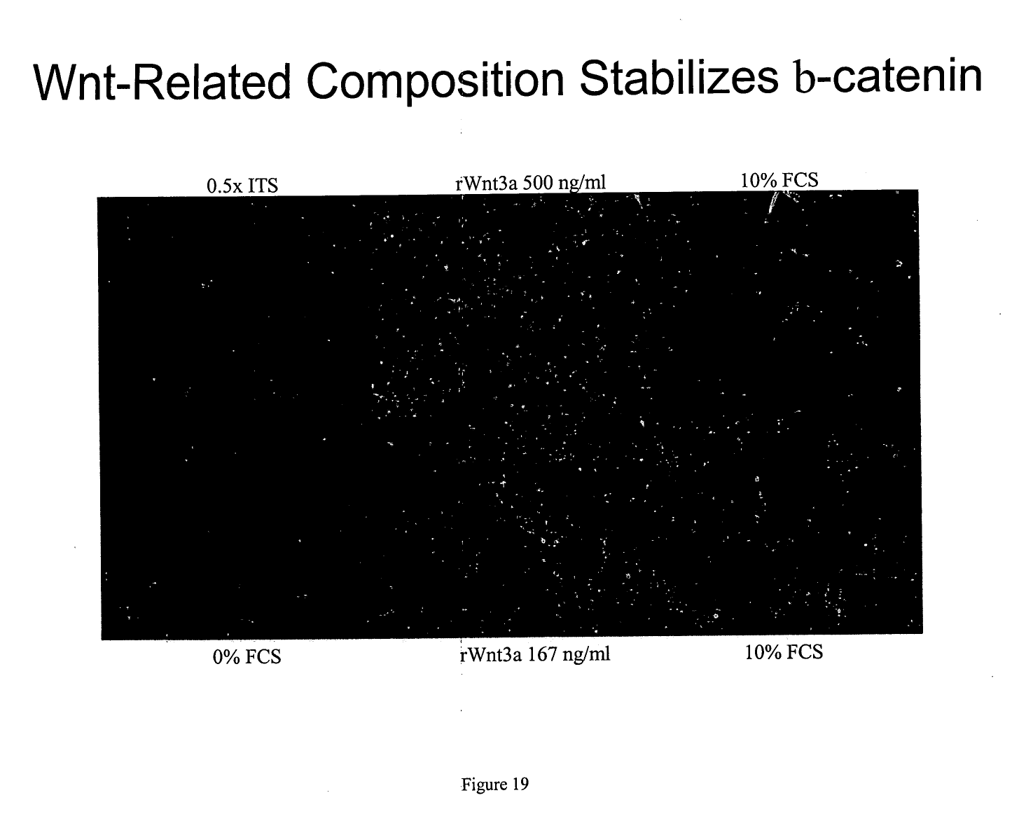

FIG. 19 shows that administration of recombinant Wnt3A to neonatal rat cardiomyocytes promoted Wnt signaling via the canonical β-catenin signaling pathway. Specifically, administration of recombinant Wnt3A stabilized β-catenin.

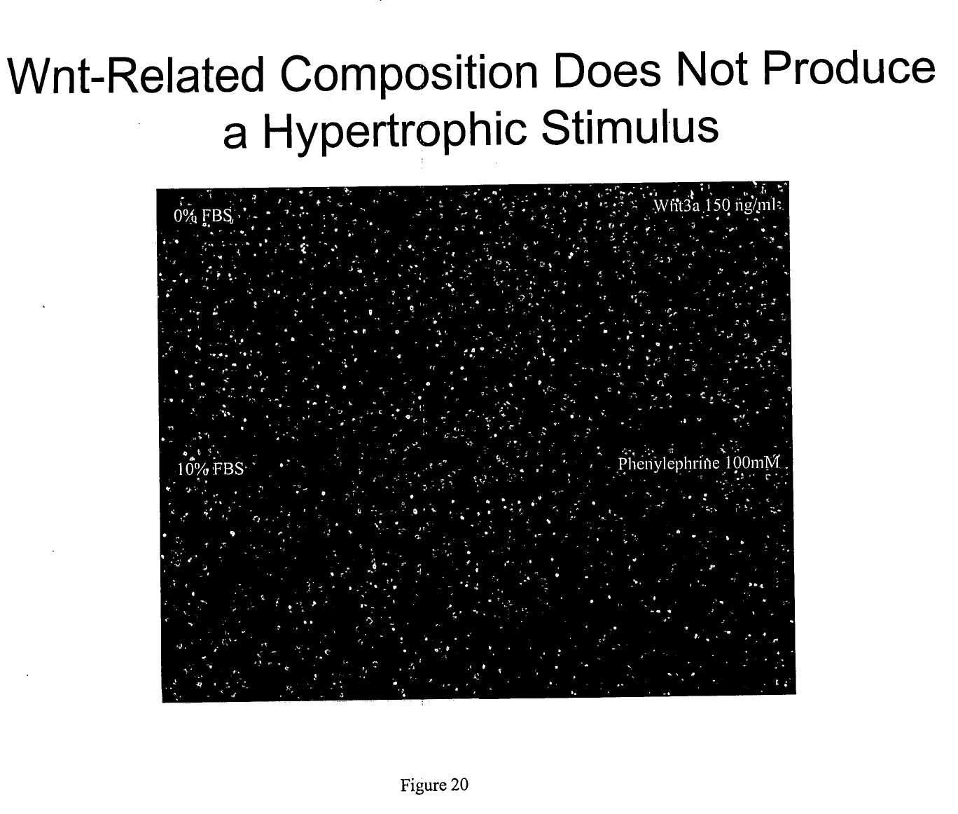

FIG. 20 shows that Wnt-related compositions did not produce a hypertrophic stimulus in cardiomyocytes. Specifically, cell shape and expression of ANF increased in cardiomyocytes exposed to a hypertrophic stimulus such as phenylephrine, and this response did not occur in cardiomyocytes exposed to Wnt3A.

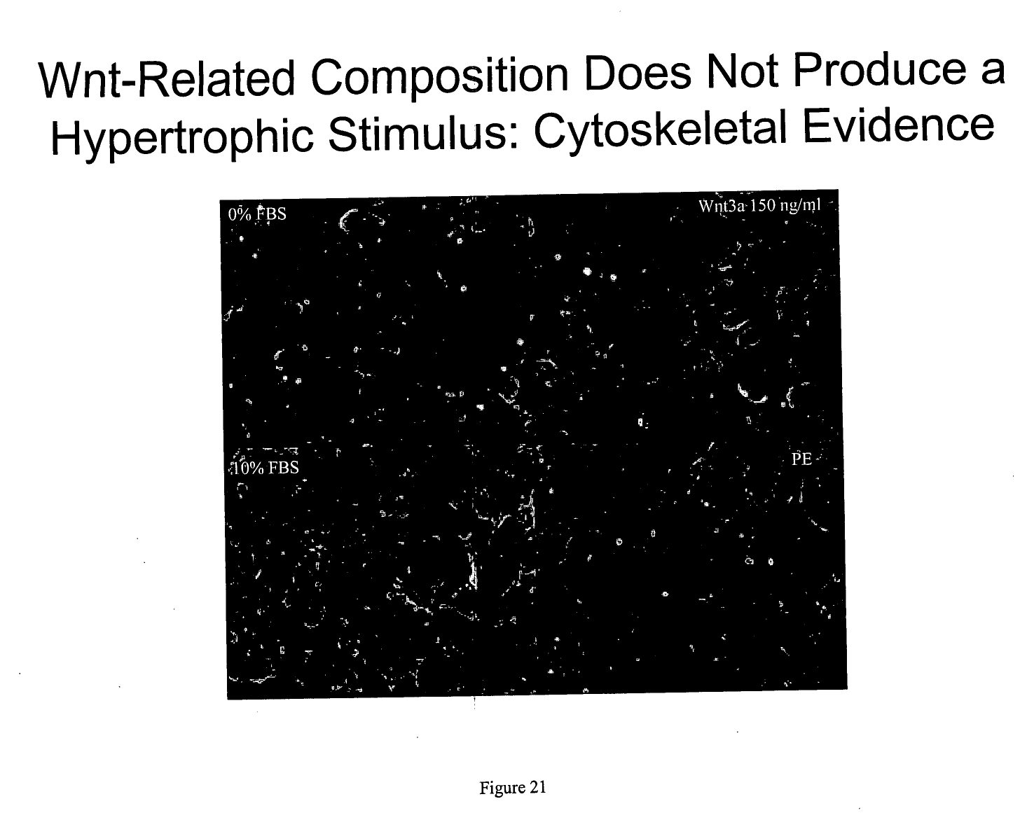

FIG. 21 shows that Wnt-related compositions did not produce a hypertrophic stimulus in cardiomyocytes. Specifically, note the vastly different cell shape, size, and morphology in cardiomyocytes exposed to a hypertrophic stimulus such as phenylephrine, in comparison to cells exposed to Wnt3A.

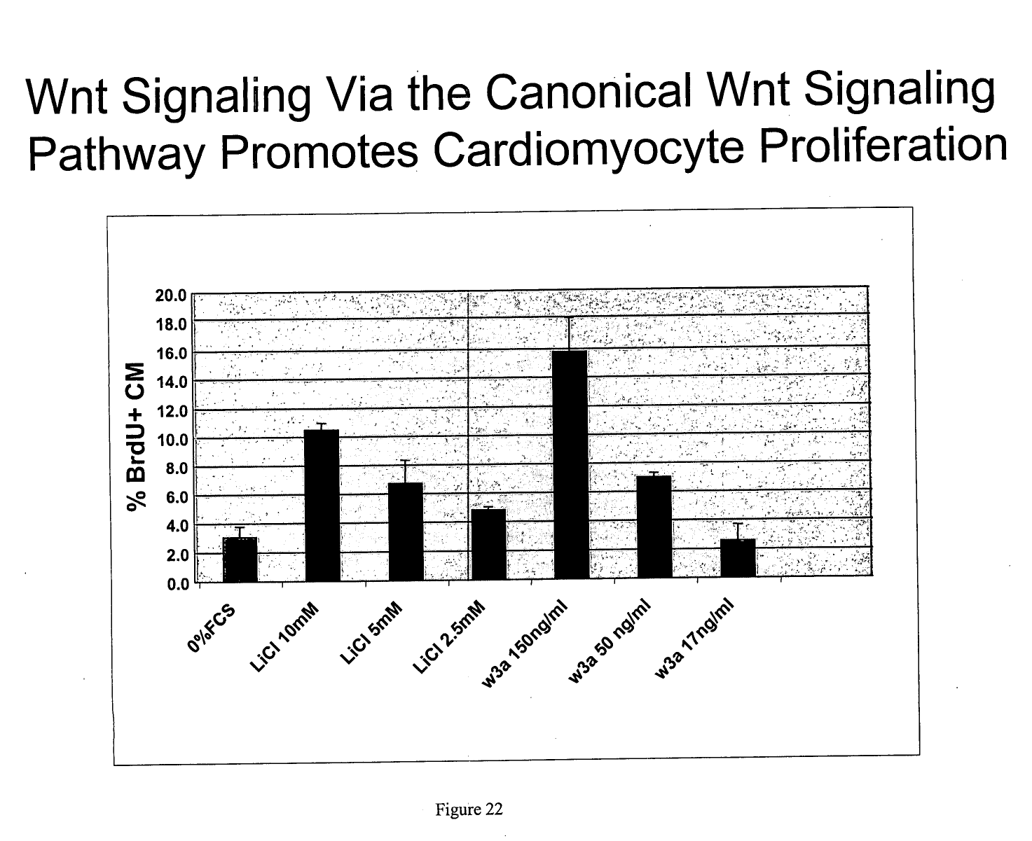

FIG. 22 shows that administration of recombinant Wnt3A to neonatal rat cardiomyocytes promoted cardiomyocyte proliferation. The cardiomyocyte promoting activity of Wnt3A is mimicked by administration of LiCl, a known activator of the canonical Wnt signaling pathway.

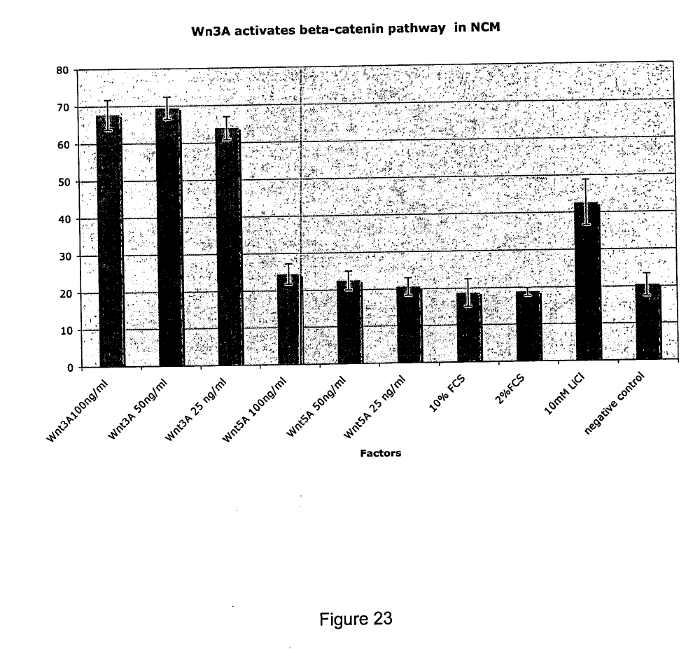

FIG. 23 provides the results of a beta-catenin nuclear localization assay that can be used to identify agents that promote Wnt signaling via the canonical Wnt signaling pathway in cardiac cells. As assessed by detecting nuclear localization of beta catenin, Wnt3A and LiCl promote Wnt signaling via the canonical Wnt signaling pathway in neonatal cardiomyocytes. However, Wnt5A does not promote Wnt signaling via the canonical Wnt signaling pathway in neonatal cardiomyocytes.

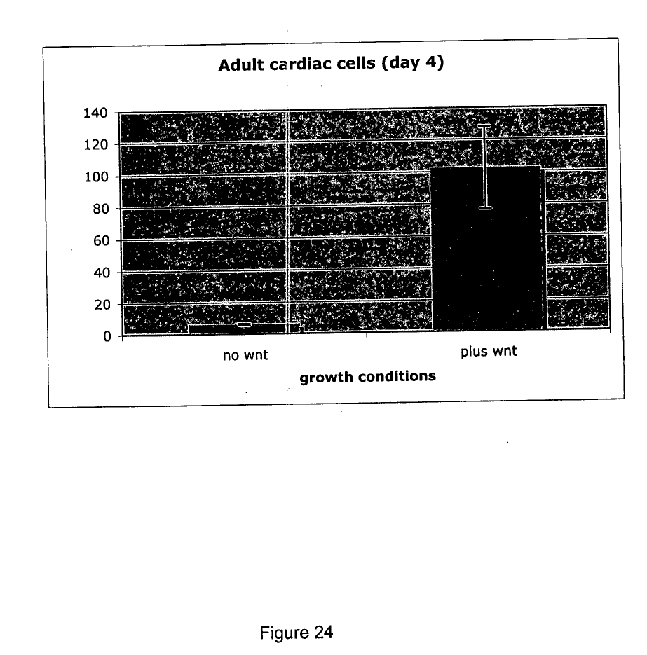

FIG. 24 shows the effect of Wnt-related compositions on adult cardiac cells. Unfractionated adult cardiac cells cultured in the presence of LWW60 conditioned medium have increased cell viability in comparison to cells cultured in the absence of LWW60 conditioned medium.

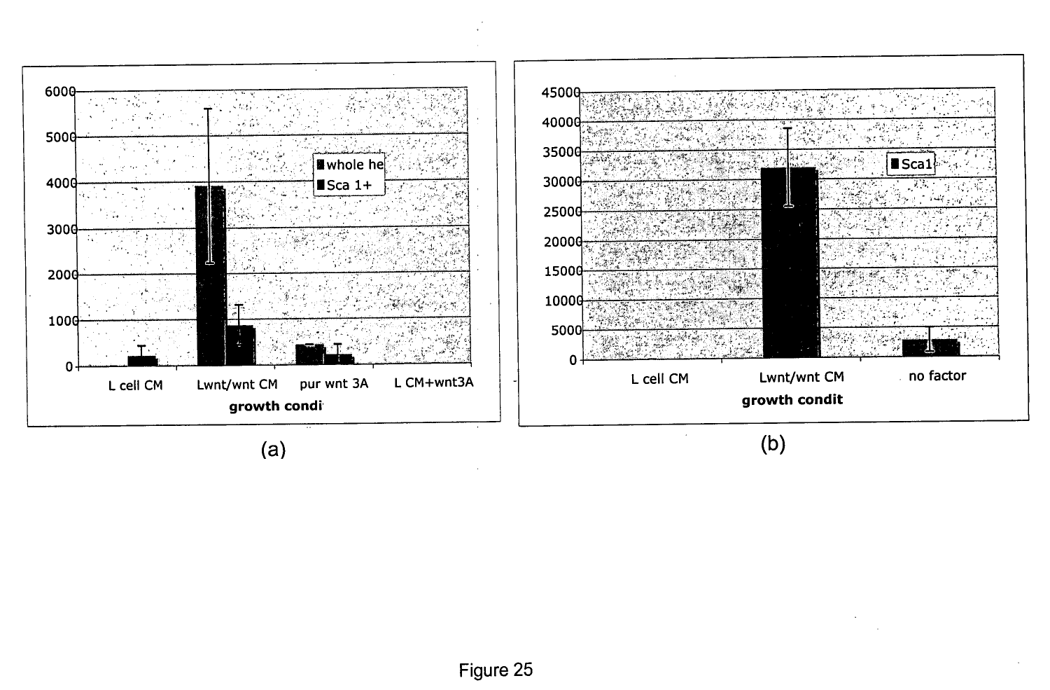

FIG. 25 shows the effect of Wnt-related compositions on various adult cardiac cell populations. FIGS. 25a and 25b show that both unfractionated and fractionated adult cardiac cells cultured in the presence of either LWW60 conditioned medium or recombinant Wnt3A protein have increased cell viability in comparison to controls.

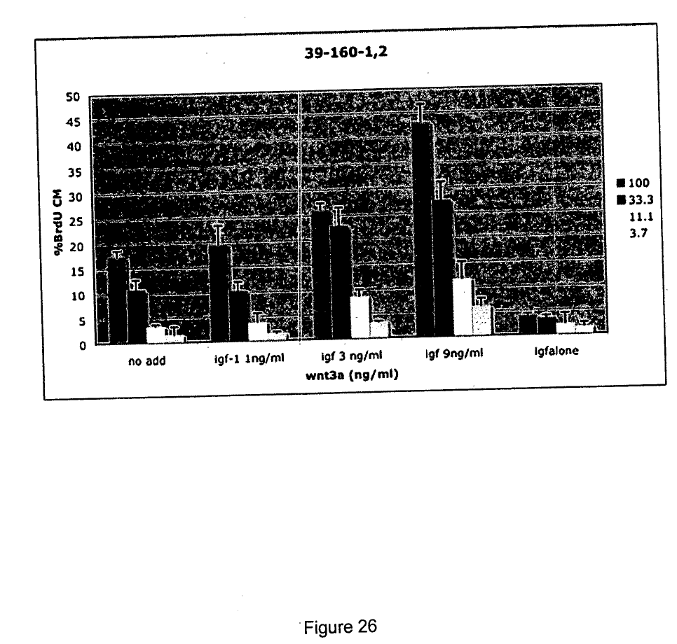

FIG. 26 shows the effect on cardiomyocyte proliferation of administering a combination of a Wnt-related composition (e.g., recombinant Wnt3A) and an agent that activates Akt/PI3 kinase signaling (e.g., IGF-1).

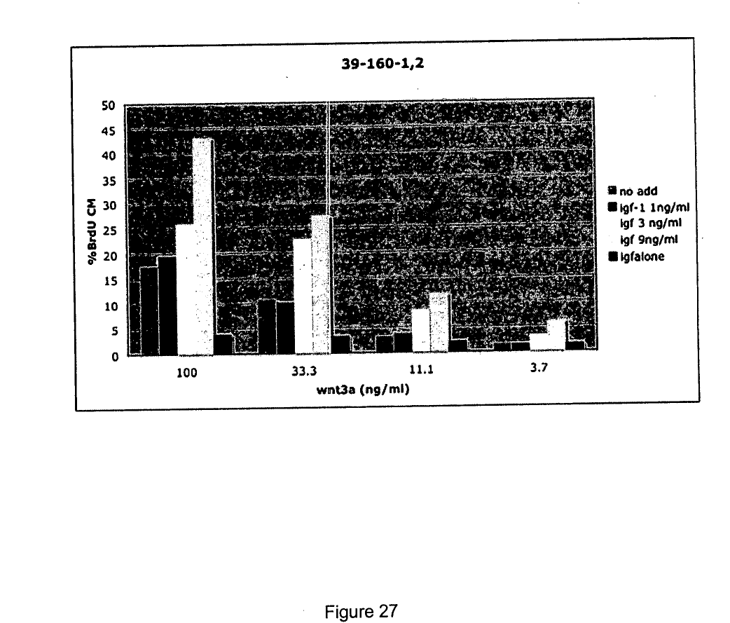

FIG. 27 shows the effect on cardiomyocyte proliferation of administering a combination of a Wnt-related composition (e.g., recombinant Wnt3A) and an agent that activates Akt/PI3 kinase signaling (e.g., IGF-1).

Table 1 provides the sequence identifiers and GenBank accession numbers for the Wnt and LRP nucleic acid and amino acid sequences referenced in the present application.

### DETAILED DESCRIPTION OF THE INVENTION

(i) Overview

The present invention provides methods and compositions with broad implications in the area of cardiovascular disease and treatment. By promoting regeneration of cardiac cells, instead of the scarring that typically results following disease or injury, the present invention provides methods and compositions with a range of important applications including: promoting cardiomyocyte proliferation, promoting regeneration of cardiomyocytes, and treating a range of cardiovascular conditions. Additionally, the compositions of the present invention are particular useful for promoting cardiomyocyte proliferation and/or regeneration without producing a hypertrophic response. This provides a substantial benefit over other agents that increase proliferation, but also induce cardiomyocyte hypertrophy. More generally, the invention provides methods and compositions for promoting proliferation and/or regeneration of cardiac cells and tissues

Wnt Signaling

Wnt proteins are secreted polypeptides with multiple roles in development. The nineteen vertebrate Wnt proteins and their cognate receptors signal through at least two distinct intracellular pathways. The “canonical” Wnt signaling pathway signals via β-catenin to activate transcription through TCF-related proteins (van de Wetering et al. (2002) Cell 109 Suppl: S13-9; Moon et al. (2002) Science 296(5573): 1644-6). There also exists at least one alternative pathway, in which Wnt polypeptides activate protein kinase C (PKC), calcium/calmodulin-dependent kinase II (CaMKII), JNK and Rho-GTPases (Veeman et al. (2003) Dev Cell 5(3): 367-77). This “non-canonical” signaling pathway is often involved in the control of cell polarity. Particular Wnt proteins tend to signal through one of these pathways (He et al. (1997) Science 275(5306): 1652-4; Gazit et al. (1999) Oncogene 18(44): 5959-66). For example, the compositions of the present invention promote canonical Wnt signaling, as measured by an increase of β-catenin expression, activity, and/or stabilization.

Wnt proteins bind to frizzled cell surface receptors, which are seven-transmembrane proteins with an extracellular N-terminal cysteine-rich domain (CRD) that is responsible for ligand binding (Bhanot et al. (1996) Nature 382(6588): 225-30). There are two Drosophila frizzleds and seven currently identified vertebrate homologs, which have different affinities and signaling properties with respect to the 19 vertebrate Wnt family members. Also required for Wnt signal transduction are low-density lipoprotein receptor related proteins (LRP5, LRP6), called arrow in Drosophila, which act as co-receptors, forming a complex together with frizzled and Wnt (Wehrli et al. (2000) Nature 407(6803): 527-30; Tamai et al. (2000) Nature 407(6803): 530-5; Pinson et al. (2000) Nature 407(6803): 535-8). LRP exists as a dimer in the absence of a Wnt signal, and Wnt binding disrupts this dimer to reveal a cytoplasmic domain that binds to the cytoplasmic protein Axin, which is involved in the regulation of β-catenin (Mao et al. (2001) Mol Cell 7(4): 801-9; Tolwinski et al. (2003) Dev Cell 4(3): 407-18). Overexpression of LRP or of various N-terminal deletions of LRP (e.g., various N-terminal deletions that retain the transmembrane and intracellular domains of LRP) activates signaling via the canonical Wnt signaling pathway (Liu et al. (2003) Molecular and Cellular Biology 23: 5825-5835; Brennan et al. (2004) Oncogene 23: 4873-84). The binding of Wnt polypeptides to frizzled receptors is further regulated by heparan sulfate proteoglycans (HSPGs) on the surface and in the extracellular matrix (ECM) (Baeg et al. (2001) Development 128(1): 87-94).

Wnt signaling can be inhibited in the extracellular space. The secreted frizzled-related protein (sFRP) class of Wnt inhibitors are homologous to the CRD of frizzled proteins, but lack the transmembrane domains, and can compete with Frizzled receptors for Wnt binding (Rattner et al. (1997) Proc Natl Acad Sci USA 94(7): 2859-63; Hoang et al. (1996) J Biol Chem 271(42): 26131-7; Leyns et al. (1997) Cell 88(6): 747-56). This competition likely involves some specificity, as all FRP family members do not inhibit signaling by all Wnt proteins. Another class of inhibitors are the unrelated Wnt-binding proteins Cerberus and WIF-1. These proteins interfere with Wnt signaling by directly associating with a Wnt protein, and thus preventing the binding of Wnt to its receptor (Hsieh et al. (1999) Nature 398(6726): 431-6; Piccolo et al. (1999) Nature 397(6721): 707-10). Another unrelated inhibitor class is represented by dkk, which binds to the LRP co-receptor, blocking Wnt binding, as well as inducing endocytosis of LRP in cooperation with Kremen (Glinka et al. (1998) Nature 391(6665): 357-62). Wise is yet another secreted Wnt inhibitor that binds to LRP, but depending on context can either augment or inhibit Wnt signaling (Itasaki et al. (2003) Development 130(18): 4295-305).

Although the mechanisms are, as yet, unclear, frizzled proteins transduce signal to the cytoplasmic protein dishevelled (dsh). Frizzled signals are blocked by pertussis toxin, suggesting a G protein-coupled signaling mechanism (Malbon et al. (2001) Biochem Biophys Res Commun 287(3): 589-93). At Dishevelled, the Wnt signaling pathway diverges, and different conserved domains of the dsh protein are required for the two Wnt signaling pathways (e.g., the canonical, β-catenin-dependent versus the non-canonical, planar cell-polarity-related). Canonical Wnt signaling requires the DIX domain of dsh; deletion of this domain strongly inhibits Wnt signals via β-catenin (Tada and Smith. (2000) Development 127(10): 2227-38). However, non-canonical Wnt signaling requires the DEP domain of dishevelled to alter cell movements (Axelrod et al. (1998) Genes Dev. 12(16): 2610-22; Boutros et al. (1998) Cell 94(1): 109-18). The mechanism of how different Wnts signal through distinct domains of the same dishevelled protein has not been determined.

In the absence of Wnt signals, β-catenin serves as an adaptor protein that links cadherins to the submembrane actin cytoskeleton. Excess free β-catenin in the cytosol is phosphorylated on Ser45 by casein kinase Iα(CKIα) or casein kinase ε (CKIε), permitting subsequent phosphorylations at serine/threonine 41, 37 and 33 by glycogen synthase kinase-3β (GSK-3β) (Peters et al. (1999) Nature 401(6751): 345-50; Sakanaka et al. (1999) Proc Natl Acad Sci USA, 96(22): 12548-52; Sakanaka. (2002) J Biochem (Tokyo) 132(5): 697-703; Song et al. (2003) J Biol Chem 278(26): 24018-25; Willert et al. (1997) EMBO J. 16(11): 3089-96). Phosphorylation at site 37 and 33 allows ubiquitination by βTrCP and proteasome degradation (Liu et al. (2002) Cell 108(6): 837-47; Amit et al. (2002) Genes Dev 16(9): 1066-76). CKIα, CKIε and GSK-3β are part of a multi-protein destruction complex, containing APC and the scaffold protein Axin. Dishevelled also interacts directly with CKIε, GBP/Frat1 and Axin. GBP/Frat1 inhibits phosphorylation of β-catenin by GSK-3β by dislodging GSK-3β from Axin (Li et al. (1999) EMBO J. 18(15): 4233-40; Salic (2000) Mol Cell 5(3): 523-32; Farr et al. (2000) J Cell Biol 148(4): 691-702). The kinase PAR1 interacts with dishevelled and is a positive regulator of Wnt/β-catenin signaling, while at the same time it inhibits Wnt/JNK signaling (Sun et al. (2001) Nat Cell Biol 3(7): 628-36). Dsh furthermore binds to protein phosphatase 2c (PP2C), which can dephosphorylate Axin (Strovel et al. (2000). J Biol Chem 275(4): 2399-403). The dishevelled binding protein Frodo is also an essential positive regulator of Wnt/β-catenin signals (Gloy et al. (2002) Nat Cell Biol 4(5): 351-7).

Drosophila Dsh is negatively regulated by naked cuticle (naked), which directly binds to Dsh (Zeng et al. (2000) Nature 403(6771): 789-95; Rousset et al. (2001) Genes Dev 15(6): 658-71). Dapper also negatively regulates Dsh (Cheyette et al. (2002) Dev Cell 2(4): 449-61). Disabled-2 (dab-2) interacts with both Dvl and Axin, and functions as a negative regulator of Wnt/β-catenin signaling (Hocevar et al. (2003) EMBO J. 22(12): 3084-94). LKB1/XEEK1 binds to GSK-3β and is required for β-catenin signaling (Ossipova et al. (2003) Nat Cell Biol 5(10): 889-94).

The β-catenin destruction box is composed of many proteins. Key components include GSK-3β, Axin, APC and CKIα (Behrens et al. (1998) Science 280(5363): 596-9; Itoh et al. (1998) Curr Biol 8(10): 591-4). Axin serves a scaffolding function, binding many of the components of the destruction complex. Both Axin and Dsh contain a DIX domain that promotes interactions between these two proteins (Hsu et al. (1999) J Biol Chem 274(6): 3439-45; Smalley et al. (1999) EMBO J. 18(10): 2823-35). Axin also binds protein phosphatase 2A (PP2A), which inhibits Wnt signaling (Hsu et al. (1999) J Biol Chem 274(6): 3439-45). When Dsh binds to Axin, GSK-3β dissociates from the complex and β-catenin phosphorylation is blocked (Salic (2000) Mol Cell 5(3): 523-32). Axin interaction with the cytoplasmic tail of the LRP co-receptor may also promote dissociation of GSK-3β. APC is also an essential component of the destruction complex; in its absence, β-catenin is stabilized and goes to the nucleus (Rosin-Arbesfeld et al. (2000) Nature 406(6799): 1009-12; Henderson. (2000) Nat Cell Biol 2(9): 653-60).

When Wnt signaling is silent, the transcription factor TCF is bound to the transcriptional repressor Groucho (grg) which interacts with histone deacetylases (HDAC) to inhibit transcription of target genes (Riese et al. (1997) Cell 88(6): 777-87; Brannon et al. (1997) Genes Dev 11(18): 2359-70; Roose et al. (1998) Nature 395(6702): 608-12; Cavallo et al. (1998) Nature 395(6702): 604-8). When β-catenin is stabilized and goes to the nucleus it binds TCF, displaces Groucho, and converts TCF into a transcriptional activator of the same target genes. Legless and pygopos (Bcl9) also are involved in this complex (Thompson et al. (2002) Nat Cell Biol 4(5): 367-73; Kramps et al. (2002) Cell 109(1): 47-60). Chibby is a nuclear antagonist of β-catenin (Takemaru et al. (2003) Nature 422(6934): 905-9). Reptin 52 is necessary for β-catenin transcriptional activation, while pontin52 inhibits β-catenin (Bauer et al. (2000) EMBO J. 19(22): 6121-30). Zebrafish harboring a gain of function reptin mutation, or injected with morpholino oligos against pontin, have enlarged hearts containing an excess of cardiomyocytes (Rottbauer et al. (2002) Cell 111(5): 661-72).

TCF is negatively regulated by phosphorylation by Nemo/NLK kinases. TAB1/TAK1 kinases stimulate the activity of NLK (Rocheleau et al. (1999) Cell 97(6): 717-26; Meneghini et al. (1999) Nature 399(6738): 793-7; Ishitani et al. (1999) Nature 399(6738): 798-802; Ishitani, et al. (2003) Mol Cell Biol 23(1): 131-9). NLK may also function in the Wnt/Ca pathway.

β-catenin also interacts directly with the transcription factor Pitx2, activating transcription (Kioussi et al. (2002) Cell 111(5): 673-85). β-catenin may also be regulated by HMG box factors, such as XSox17 (Zorn et al. (1999) Mol Cell 4(4): 487-98). Another HMG box protein, HBP1, acts as a co-repressor binding to TCF (Sampson et al. (2001) EMBO J. 20(16): 4500-11). Interestingly, HBP1 inhibition of TCF is relieved by inhibition of p38 (Xiu et al. (2003) Mol Cell Biol 23(23): 8890-901).

The methods and compositions of the present invention provide a novel solution for the problem of promoting cardiac cell proliferation, regeneration, and/or survival. In one embodiment, the methods and compositions of the invention promote cardiomyocyte proliferation and/or regeneration. In another embodiment, the methods and compositions of the invention promote proliferation, regeneration, and/or survival of fetal, postnatal, and adult cardiac cells, for example, cardiomyocytes. Furthermore, in certain embodiments, the compositions of the present invention are particularly advantageous for promoting cardiomyocyte proliferation and/or regeneration without inducing a hypertrophic response in the cardiomyocyte. The ability to induce proliferation and/or regeneration without inducing a hypertrophic response may be advantageous for certain in vivo applications of the methods and compositions of the invention.

The methods and compositions of the present invention function effectively despite the obvious complexity of the “canonical” Wnt signaling pathway. The methods and compositions of the present invention promote cardiac cell proliferation, regeneration, and/or survival (e.g., cardiomyocyte proliferation, regeneration, and/or survival) using any of a number of Wnt-related proteins and compositions, as described herein. Wnt-related proteins and compositions for use in the methods of the present invention promote cardiac cell (e.g., cardiomyocyte) proliferation, regeneration, and/or survival and furthermore promote Wnt signaling via the canonical Wnt signaling pathway. Exemplary Wnt-related compositions include Wnt3-related compositions such as Wnt3 and Wnt3A. Further exemplary Wnt-related compositions include Wnt polypeptides selected from any of Wnt1, Wnt2, Wnt2B, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, and Wnt16. We note that many Wnt polypeptides are thought to act via either the canonical or non-canonical Wnt signaling pathway depending on context (e.g., depending on the tissue or stage in embryonic or adult development). Thus, in the context of the present invention, when a polypeptide is said to promote Wnt signaling via the canonical Wnt signaling pathway all that is meant is that the polypeptide promotes signaling via the canonical Wnt signaling pathway in the context of the present invention (e.g., when promoting proliferation of cardiomyocytes). It is understood that the same polypeptide that promotes Wnt signaling via the canonical Wnt signaling pathway in promoting cardiomyocyte proliferation may promote Wnt signaling via either the canonical or non-canonical Wnt signaling pathway in other cell types or tissues. The present invention provides specific assays to readily identify Wnt polypeptides that signal via the canonical Wnt signaling pathway, specifically in cardiac cells such as cardiomyocytes. Accordingly, one of skill in the art can readily (i) identify Wnt polypeptide that can promote signaling via the canonical Wnt-signaling pathway in cardiac cells and (ii) test those Wnt polypeptides (including related polypeptides such as bioactive fragments, variants, and modified polypeptides) to assess whether they can promote proliferation, regeneration, and/or survival of cardiac cells. Using this approach, one of skill in the art can readily select from amongst all Wnt polypeptides (now known or later identified) to identify the Wnt polypeptides (including related polypeptides such as bioactive fragments, variants, and modified polypeptides) that have the following two characteristics: promote Wnt signaling via the canonical Wnt signaling pathway and promote cardiac cell proliferation, regeneration, and/or survival.

Additionally, the present invention provides a large number of agents that act at the cell surface to promote Wnt signaling via the canonical Wnt signaling pathway. Such methods and compositions can be used to promote cardiomyocyte proliferation. Exemplary methods and compositions include, but are not limited to, Wnt-related polypeptides, modified Wnt-related polypeptides, bioactive fragments of Wnt-related polypeptides, Wnt-related nucleic acids, LRP-related nucleic acids, LRP-related polypeptides, N-terminal deletions of LRP-related polypeptides, soluble extracellular fragments of LRP-related polypeptides, modified soluble extracellular fragments of LRP-related polypeptides, or anti-LRP-related antibodies.

(ii) Definitions

Unless defined otherwise, all technical and scientific terms have the same meaning as is commonly understood by one of skill in the art to which this invention belongs.

The articles “a” and “an” are used herein to refer to one or to more than one (i.e., to at least one) of the grammatical object of the article. By way of example, “an element” means one element or more than one element.

A “marker” is used to determine the state of a cell. Markers are characteristics, whether morphological or biochemical (enzymatic), particular to a cell type, or molecules expressed by the cell type. A marker may be a protein marker, such as a protein marker possessing an epitope for antibodies or other binding molecules available in the art. A marker may also consist of any molecule found in a cell, including, but not limited to, proteins (peptides and polypeptides), lipids, polysaccharides, nucleic acids and steroids. Additionally, a marker may comprise a morphological or functional characteristic of a cell. Examples of morphological traits include, but are not limited to, shape, size, and nuclear to cytoplasmic ratio. Examples of functional traits include, but are not limited to, the ability to adhere to particular substrates, ability to incorporate or exclude particular dyes, ability to migrate under particular conditions, and the ability to differentiate along particular lineages.

Markers may be detected by any method available to one of skill in the art. In addition to antibodies (and all antibody derivatives) that recognize and bind at least one epitope on a marker molecule, markers may be detected using analytical techniques, such as by protein dot blots, sodium dodecyl sulfate polyacrylamide gel electrophoresis (SDS-PAGE), or any other gel system that separates proteins, with subsequent visualization of the marker (such as Western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for an enzymatic product or intermediate, or the overall composition of a cell, such as the ratio of protein to lipid, or lipid to sugar, or even the ratio of two specific lipids to each other, or polysaccharides. In the case of nucleic acid markers, any known method may be used. If such a marker is a nucleic acid, PCR, RT-PCR, in situ hybridization, dot blot hybridization, Northern blots, Southern blots and the like may be used, coupled with suitable detection methods. If such a marker is a morphological and/or functional trait, suitable methods include visual inspection using, for example, the unaided eye, a stereomicroscope, a dissecting microscope, a confocal microscope, or an electron microscope.

“Differentiation” describes the acquisition or possession of one or more characteristics or functions different from that of the original cell type. A differentiated cell is one that has a different character or function from the surrounding structures or from the precursor of that cell (even the same cell). The process of differentiation gives rise from a limited set of cells (for example, in vertebrates, the three germ layers of the embryo: ectoderm, mesoderm and endoderm) to cellular diversity, creating all of the many specialized cell types that comprise an individual.

Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway. In many, but not all tissues, the process of differentiation is coupled with exit from the cell cycle. In these cases, the cells typically lose or greatly restrict their capacity to proliferate and such cells are commonly referred to as being “terminally differentiated. However, we note that the term “differentiation” or “differentiated” refers to cells that are more specialized in their fate or function than at a previous point in their development, and includes both cells that are terminally differentiated and cells that, although not terminally differentiated, are more specialized than at a previous point in their development.

“Muscle cells” are characterized by their principal role: contraction. Muscle cells are usually elongate and arranged in vivo in parallel arrays. The principal components of muscle cells, related to contraction, are the myofilaments. Two types of myofilaments can be distinguished: (1) those composed primarily of actin, and (2) those composed primarily of myosin. While actin and myosin can be found in many other cell types, enabling such cells, or portions, to be mobile, muscle cells have an enormous number of co-aligned contractile filaments that are used to perform mechanical work.

“Cardiac muscle” or “myocardium” consists of long fibers that, like skeletal muscle, are cross-striated. Cardiac muscle is composed of cells referred to as cardiomyocytes. In addition to the striations, cardiac muscle also contains special cross bands, the intercalated discs, which are absent in skeletal muscle. Also unlike skeletal muscle in which the muscle fiber is a single multinucleated protoplasmic unit, in cardiac muscle the fiber consists of mononucleated (sometimes binucleated) cells aligned end-to-end. Cardiac cells often anastomose and contain many large mitochondria. Usually, injured cardiac muscle is replaced with fibrous connective tissue, not cardiac muscle.

“Proliferation” refers to an increase in the number of cells in a population by means of cell division. Cell proliferation results from the coordinated activation of multiple signal transduction pathways, often in response to growth factors and other mitogens. Cell proliferation may also be promoted when cells are released from the actions of intra- or extracellular signals and mechanisms that block or down-regulate cell proliferation. An increase in cell proliferation can be assessed by an increase in DNA synthesis.

“Cardiomyocyte proliferation” refers to an increase in DNA synthesis in a population of cells, wherein the population of cells includes cardiomyocytes. The following are examples of cardiomyocyte proliferation within the meaning of the present application: (i) proliferation of a particular cardiomyocyte contacted with a Wnt-related composition; (ii) proliferation of a daughter cell (e.g., progeny) of a cardiomyocyte that was contacted with a Wnt-related composition; (iii) proliferation of a related cell adjacent to the cardiomyocyte contacted with a Wnt-related composition.

The Wnt gene family encodes secreted ligands that serve key roles in differentiation and development. This family comprises at least 15 vertebrate and invertebrate genes including the Drosophila segment polarity gene wingless. Wnt signaling is involved in a variety of developmental processes including early patterning, neural development, somite formation, cardiac development and kidney development, and inappropriate Wnt signaling can be involved in transformation of cells.

The Wnt signaling pathway is initiated via interaction of a Wnt polypeptide with a transmembrane receptor of the frizzled family. Wnt signals are transduced by either a canonical Wnt signaling pathway or a non-canonical Wnt signaling pathway. The compositions and methods of the present invention involve Wnt polypeptides that promote cardiomyocyte proliferation by promoting Wnt signaling via the canonical, β-catenin mediated Wnt signaling pathway. Intracellularly, transduction of the Wnt signal via the canonical Wnt signaling pathway is mediated by both positive and negative regulatory proteins. Positive regulators include disheveled, and the transcription factors β-catenin and Lef-1, and negative regulators include GSK3β. In addition to negative regulation intracellularly, Wnt signaling can be negatively regulated extracellularly by the activity of Frzb related polypeptides. This family of polypeptides, which includes FrzA, Frzb, and sizzled, comprises soluble polypeptides that resemble the ligand binding domain of the Wnt receptor. Wnt polypeptides can bind Frzb related polypeptides, however, such binding does not result in Wnt signal transduction.

The term “Wnt-related composition” refers to a composition comprising a Wnt-related polypeptide and/or a modified Wnt-related polypeptide. A “Wnt-related polypeptide” refers to a polypeptide comprising a Wnt amino acid sequence, a variant Wnt amino acid sequence, or a bioactive fragment thereof. Wnt-related polypeptides according to the invention also include modified Wnt-related polypeptides. Wnt-related polypeptides for use in the methods of the present invention promote Wnt signaling via the canonical Wnt signaling pathway. Preferred Wnt-related polypeptides of the invention are Wnt3 and Wnt3A. Other preferred Wnt-related polypeptides of the invention may be selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A/Wnt14, Wnt9B/Wnt15, Wnt10A, Wnt10B, Wnt11, and Wnt16. Specifically, one of skill in the art can select from amongst any of the foregoing Wnt-related polypeptides to identify the Wnt-related polypeptides that promote Wnt signaling via the canonical Wnt signaling pathway. Such Wnt polypeptides may be used in the methods and compositions of the present invention. We note that the term Wnt-related polypeptide is not meant to encompass non-Wnt polypeptides. Specifically, the term excludes polypeptides that fail to retain the basic structure or function of a Wnt polypeptide, or a bioactive fragment of a Wnt polypeptide. In certain embodiment, the Wnt-related polypeptides of the invention comprise an amino acid sequence at least 80%, 85%, 90%, 95%, 96%, 97%, 98%, 99%, or 100% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment of any of the foregoing. In any of the foregoing, a Wnt-related polypeptide or a Wnt-related composition for use in the methods of the invention retains one or more of the biological activities of the corresponding Wnt polypeptide. By way of example, a Wnt-related polypeptide retains one or more of the biological activities of native and/or unmodified Wnt polypeptide. Exemplary biological activities of a Wnt polypeptide include the following: (i) bind a frizzled receptor; (ii) promote Wnt signaling; (iii) promote expression, activity, nuclear localization, and/or stability of β-catenin. In the context of the present invention, said one or more biological activities include the ability to promote Wnt signaling via the canonical Wnt signaling pathway.

In certain embodiments, Wnt-related compositions refer to Wnt-related nucleic acid compositions. Such compositions comprise nucleic acid sequences encoding a Wnt-related polypeptide. The Wnt-related nucleic acid composition can be delivered, and the delivered Wnt-related nucleic acid composition encodes a Wnt-related polypeptide that promotes cardiomyocyte proliferation and/or regeneration. Wnt-related nucleic acid compositions comprise nucleic acid sequences identical to all or a portion of the nucleic acid sequences represented in SEQ ID NO: 1, SEQ ID NO: 3, SEQ ID NO: 5, SEQ ID NO: 7, SEQ ID NO:9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77, as well as nucleic acid sequences that hybridize under stringent conditions, including a wash step of 0.2×SSC at 65° C., to a nucleic acid sequence represented in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

There are at least 15 identified Wnt polypeptides. Non-limiting examples of nucleic acid and amino acid sequences corresponding to Wnt polypeptides are provided in Table 1. The Wnt-related polypeptides are characterized by one or more of the following biological functions: (i) bind to a frizzled receptor, (ii) promote Wnt signaling, and/or (iii) promote expression, activity, nuclear localization, and/or stability of β-catenin. Wnt-related polypeptides for use in the methods of the present invention promote Wnt signaling via the canonical Wnt signaling pathway.

In addition to full-length Wnt-related polypeptides, the invention contemplates the use of bioactive fragments of Wnt-related polypeptides that retain one or more of the biological activities of a full-length Wnt-related polypeptide. Exemplary bioactive fragment are bioactive fragments of SEQ ID NO: 2, SEQ ID: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78. Further exemplary bioactive fragments are fragments of a polypeptide at least 80% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78.

Additional exemplary Wnt-related nucleic acid and polypeptide sequences are known in the art and include, but are not limited to, Wnt1, Wnt2, Wnt3, Wnt5, Wnt8, and Wnt11. Further exemplary Wnt-related nucleic acids and polypeptides include, but are not limited to, Wnt2B, Wnt4, Wnt6, Wnt7A, Wnt7B, Wnt9A, Wnt9B, Wnt10A, Wnt10B, and Wnt16. Table 1 provides a list of exemplary mouse and human Wnt nucleic acid and amino acid sequences. The Wt-related polypeptides and nucleic acids for use in the methods of the present invention promote wnt signaling via the canonical wnt signaling pathway. In certain embodiments, Wnt polypeptide that promotes Wnt signaling via the canonical wnt signaling pathway are selected based on the ability to promote Wnt signaling via the canonical Wnt signaling pathway in a cardiac cell type, for example, in an in vitro assay indicative of signaling via the canonical Wnt signaling pathway.

The term “LRP-related composition” refers to a composition comprising an LRP-related polypeptide and/or a modified LRP-related polypeptide, or to a composition comprising an LRP-related nucleic acid that encodes an LRP-related polypeptide. An “LRP-related polypeptide” refers to a polypeptide comprising an LRP amino acid sequence, a variant LRP amino acid sequence, or a bioactive fragment thereof. LRP-related polypeptides according to the invention also include modified LRP-related polypeptides. Particularly preferred LRP-related polypeptides and nucleic acids of the invention are LRP5-related and LRP6-related polypeptides and nucleic acids. In certain embodiments, the LRP-related polypeptides of the invention comprise an amino acid sequence at least 80%, 85%, 90%, 95%, 96%, 97%, 98%, 99%, or 100% identical to any of SEQ ID NO: 80, SEQ ID NO: 82, SEQ ID NO: 84, SEQ ID NO: 86, or a bioactive fragment of any of the foregoing. In certain embodiments, the LRP-related nucleic acids of the invention comprise a nucleic acid sequence at least 80%, 85%, 90%, 95%, 96%, 97%, 98%, 99%, or 100% identical to any of SEQ ID NO: 79, SEQ ID NO: 81, SEQ ID NO: 83, SEQ ID NO: 85, or a bioactive fragment of any of the foregoing. In certain other embodiments, the LRP-related nucleic acids of the invention comprise a nucleic acid sequence that hybridizes under stringent conditions, including a wash step of 0.2×SSC at 65° C., to any of SEQ ID NO: 79, SEQ ID NO: 81, SEQ ID NO: 83, SEQ ID NO: 85. In any of the foregoing, an LRP-related polypeptides or compositions for use in the methods of the invention retain one or more of the biological activities of native and/or unmodified LRP polypeptides. Exemplary biological activities include the following: (i) promote Wnt signaling via the canonical Wnt signaling pathway; (ii) promote expression, activity, and/or stability of β-catenin. In the context of the present invention, said one or more biological activities include the ability to promote Wnt signaling via the canonical Wnt signaling pathway.

By “N-terminal deletions of LRP” or “N-terminal deletions of an LRP-related polypeptide” is meant deletions of all or a portion of the extracellular domain of an LRP-related polypeptide. Fragments of an LRP-related polypeptide comprising an N-terminal deletion retain the transmembrane domain and the intracellular domain. Such fragments are missing all or a portion of the extracellular domain of the native LRP-related polypeptide. Exemplary N-terminal deletions of LRP include N-terminal deletion of an LRP-related polypeptide represented in any of SEQ ID NO: 80, SEQ ID NO: 82, SEQ ID NO: 84, or SEQ ID NO: 86. Further exemplary N-terminal deletions are missing all or a portion of the following regions located in the extracellular domain of the protein: EGF repeats, LDLR repeats, or YWTD spacer regions. The invention further contemplates nucleic acids encoding N-terminal deletions of LRP.

“Soluble extracellular fragments of LRP-related polypeptides” and “modified soluble extracellular fragments of LRP-related polypeptides” are exemplary fragments of LRP-related polypeptides for use in the methods of the present invention. Such extracellular fragments can include all or a portion of the extracellular domain of an LRP-related polypeptide. Soluble extracellular fragments are of particular use due to their ease of administration and ease of modification (e.g., with one or more hydrophobic or hydrophilic moieties). Without being bound by theory, soluble extracellular fragments may function at the cell surface to promote Wnt signaling by competing with and relieving the inhibitory LRP dimerization that is endogenously relieved by binding of Wnt to a Wnt receptor.

By bioactive fragment is meant that a given portion of the protein maintains one or more of the functional attributes of the full length protein. In the context of the present invention, a bioactive fragment retains one or more of the biological functions of full length Wnt including, but not limited to, any of the following: retains the ability to promote Wnt signaling. Additional biological activities include, but are not limited to, (i) bind to a frizzled receptor, (ii) promote Wnt signaling via the canonical Wnt signaling pathway, and/or (iii) promote expression, activity, nuclear localization, and/or stability of β-catenin. The invention contemplates the use not only of bioactive fragments of Wnt, but also peptidomimetics (modified fragments). Furthermore, as outlined below, the invention contemplates modified Wnt-related polypeptides, and modified bioactive fragments thereof. Exemplary modified Wnt-related polypeptides and modified bioactive fragments thereof retain one or more of the biological activities of the corresponding native and/or unmodified Wnt.

Variants may be full length or other than full length. Variants of the nucleic acids or proteins of the invention include, but are not limited to, molecules comprising regions that are substantially identical to the nucleic acids or proteins of the invention. In various embodiments, the variants are at least about 70%, 75%, 80%, 85%, 90%, 95%, 96%, 97%, 98%, 99%, or greater than 99% identical to a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions (Ausubel et al., 1987). Variants for use in the methods and compositions of the present invention retain one or more of the biological activities of native and/or of unmodified Wnt

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wildtype polynucleotide sequence or any change in a wildtype protein sequence. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

A “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

In general, a “growth factor” is a substance that promotes cell growth and development by directing cell maturation and differentiation. Growth factors also mediate tissue maintenance and repair. Growth factors affect cell behavior by binding to specific receptors on the surface of cells. The binding of ligand to a growth factor receptor activates a signal transduction pathway that influences, for example, cell behavior. Growth factors typically exert an affect on cells at very low concentrations.

“Fibroblast growth factors” (Fgfs) belong to a class of growth factors consisting of a large family of short polypeptides that are released extracellularly and bind with heparin to dimerize and activate specific receptor tyrosine kinases (Fgfrs). Fgf signaling is involved in mammalian wound healing and tumor angiogenesis (Ortega et al., 1998; Zetter, 1998) and has numerous roles in embryonic development, including induction and/or patterning during organogenesis of the limb, tooth, brain, and heart (Crossley et al., 1996; Martin, 1998; Ohuchi et al., 1997; Peters and Balling, 1999; Reifers et al., 1998; Vogel et al., 1996; Zhu et al., 1996). Fgfs can easily be detected using either functional assays (Baird and Klagsbrun, 1991; Moody, 1993) or antibodies (Research Diagnostics; Flanders, N.J. or Promega, Wis.).

As used herein, the terms “transforming growth factor-beta” and “TGF-β” denote a family of structurally related paracrine polypeptides found ubiquitously in vertebrates, and prototypic of a large family of metazoan growth, differentiation, and morphogenesis factors (see, for review, Massague et al. (1990) Ann Rev Cell Biol 6:597-641; and Sporn et al. (1992) J Cell Biol 119:1017-1021). Included in this family are the “bone morphogenetic proteins” or “BMPs”, which refers to proteins isolated from bone, and fragments thereof and synthetic peptides which are involved in a variety of developmental processes. Preparations of BMPs, such as BMP-1, 2, 3, 4, 5, 6, and 7 are described in, for example, PCT publication WO 88/00205 and Wozney (1989) Growth Fact Res 1:267-280.

The term “agent” refers to compounds other than the Wnt-related compositions of the invention that can be used in combination with the Wnt-related compositions of the invention to further promote one or more of the activities of the Wnt-related compositions described herein. Agents include nucleic acids, peptides, polypeptide, and small organic molecules.

The term “agent that acts at the cell surface to promote Wnt signaling via the canonical Wnt signaling pathway” refers to nucleic acid, polypeptide, peptide, small molecule, or antibody-based agents that act at the cell surface (e.g., at the level of the interaction with a Wnt polypeptide and its receptor, or at the level of the presentation/interaction among receptors or receptor subunits). Such agents are distinguishable from agents that modulate Wnt signaling by acting intracellularly to promote or inhibit a protein involved in Wnt signal transduction. Exemplary agents include, but are not limited to, Wnt-related polypeptides, Wnt-related nucleic acids, LRP-related polypeptides, LRP-related nucleic acids, and anti-LRP antibodies.

The term “modified” refers to the derivatization of a polypeptide with one or more moieties by appending (e.g., attaching via covalent or non-covalent interactions) one or more moieties to one or more amino acid residues of that polypeptide. Exemplary modifications include hydrophobic moieties such as lipophilic moieties and fatty acid moieties, glycosylation, phosphorylation. Further exemplary modifications include hydrophilic modifications. In the context of the present invention, a preferred modified Wnt-related composition is a hydrophobically modified or hydrophilically modified Wnt-related composition (e.g., a composition comprising a modified Wnt-related polypeptide). We note that at least some investigators report the identification of a native form of Wnt3A that is modified with one palmitoyl group on Cys77 of the Wnt3A polypeptide (Note: Cys77 is bolded and underlined in the attached sequence listing to further indicate this modification). Modified polypeptides according to the present invention include, but are not limited to the following (i) Wnt-related polypeptides that are modified on Cys77 with one or more different hydrophobic moiety, or with one or more hydrophilic moiety; (ii) Wnt-related polypeptides that are not modified on Cys77 but are modified at one or more additional positions; (iii) Wnt-related polypeptides modified on Cys77 with more than one moiety; (iv) Wnt-related polypeptides modified on Cys77 with a palmitoyl moiety and further modified at one or more additional positions; and (v) Wnt-related polypeptides modified on Cys77 with a different moiety and further modified at one or more additional positions. For any of the foregoing, modified Wnt polypeptides, or bioactive fragments, retain one or more of the biological activities of the corresponding native and/or un-modified Wnt polypeptide. In the context of the present invention, the one or more biological activites include the ability to promote Wnt signaling via the canonical Wnt signaling pathway. A modified Wnt polypeptide may further possess one or more advantageous physiochemical properties in comparison to the corresponding native and/or un-modified Wnt polypeptide.

In addition to the aforementioned modified Wnt-related polypeptides, the invention contemplates modified LRP-related polypeptides, and bioactive fragments thereof. Exemplary modified LRP-related polypeptides retain the ability of un-modified LRP to promote Wnt signaling via the canonical Wnt signaling pathway. Further exemplary modified LRP-related polypeptides can be used to promote cardiomyocyte proliferation. The present invention contemplates that LRP-related polypeptides and bioactive fragments thereof can be modified with one or more hydrophobic or hydrophilic moieties using the same methods and compositions that can be used to modify Wnt-related compositions. Accordingly, throughout the present application references to methods and compositions for appending one or more moieties to a Wnt-related composition should be considered exemplary of the methods and compositions that can be used to modify LRP-related polypeptides.

The term “appended” refers to the addition of one or more moieties to an amino acid residue. The term refers, without limitation, to the addition of any moiety to any amino acid residue. The term includes attachment of a moiety via covalent or non-covalent interactions.

The term “N-terminal amino acid residue” refers to the first amino acid residue (amino acid number 1) of a polypeptide or peptide.

The term “C-terminal amino acid residue” refers to the last amino acid residue (amino acid number n, wherein n=the total number of residues in the peptide or polypeptide) of a polypeptide or peptide.

The term “hydrophobic” refers to the tendency of chemical moieties with nonpolar atoms to interact with each other rather than water or other polar atoms. Materials that are “hydrophobic” are, for the most part, insoluble in water. Natural products with hydrophobic properties include lipids, fatty acids, phospholipids, sphingolipids, acylglycerols, waxes, sterols, steroids, terpenes, prostaglandins, thromboxanes, leukotrienes, isoprenoids, retenoids, biotin, and hydrophobic amino acids such as tryptophan, phenylalanine, isoleucine, leucine, valine, methionine, alanine, proline, and tyrosine. A chemical moiety is also hydrophobic or has hydrophobic properties if its physical properties are determined by the presence of nonpolar atoms.

The term “lipophilic group”, in the context of being attached to a polypeptide, refers to a group having high hydrocarbon content thereby giving the group high affinity to lipid phases. A lipophilic group can be, for example, a relatively long chain alkyl or cycloalkyl (preferably n-alkyl) group having approximately 7 to 30 carbons. The alkyl group may terminate with a hydroxy or primary amine “tail”. To further illustrate, lipophilic molecules include naturally-occurring and synthetic aromatic and non-aromatic moieties such as fatty acids, esters and alcohols, other lipid molecules, cage structures such as adamantane and buckminsterfullerenes, and aromatic hydrocarbons such as benzene, perylene, phenanthrene, anthracene, naphthalene, pyrene, chrysene, and naphthacene.

The phrase “internal amino acid” means any amino acid in a peptide sequence that is neither the N-terminal amino acid nor the C-terminal amino acid.

The term “heteroatom” as used herein means an atom of any element other than carbon or hydrogen. Preferred heteroatoms are boron, nitrogen, oxygen, phosphorus, sulfur and selenium.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula: 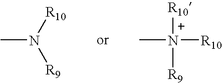
wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula: 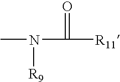
wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula: 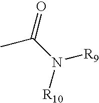
wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula: 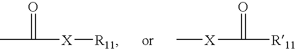
wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above.

Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group. The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula: 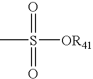
in which R₄₁ is an electron pair, hydrogen, alkyl, cycloalkyl, or aryl.

The term “sulfate” is art recognized and includes a moiety that can be represented by the general formula: 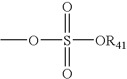
in which R₄₁ is as defined above.

The term “sulfonamido” is art recognized and includes a moiety that can be represented by the general formula: 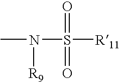
in which R₉ and R′₁₁ are as defined above.

The term “sulfamoyl” is art-recognized and includes a moiety that can be represented by the general formula: 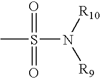
in which R₉ and R₁₀ are as defined above.

The term “sulfoxido” as used herein, refers to a moiety that can be represented by the general formula: 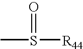
in which R₄₄ is selected from the group consisting of hydrogen, alkyl, alkenyl, alkynyl, cycloalkyl, heterocyclyl, aralkyl, or aryl.

A “phosphoryl” can in general be represented by the formula: 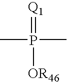
wherein Q₁ represented S or O, and R₄₆ represents hydrogen, a lower alkyl or an aryl.

When used to substitute, e.g., an alkyl, the phosphoryl group of the phosphorylalkyl can be represented by the general formula: 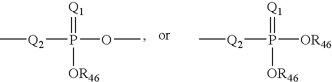
wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

A “phosphoramidite” can be represented in the general formula: 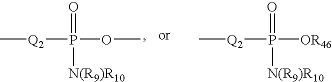
wherein R₉ and R₁₀ are as defined above, and Q₂ represents O, S or N.

A “phosphonamidite” can be represented in the general formula: 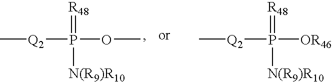
wherein R₉ and R₁₀ are as defined above, Q₂ represents O, S or N, and R₄₈ represents a lower alkyl or an aryl, Q₂ represents O, S or N.

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₇, m and R₇ being defined above.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed., Wiley: New York, 1991).

The term “amino acid side chain” is that part of an amino acid exclusive of the —CH(NH₂)COOH portion, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or H-(the side chain of glycine).

In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers.

A “reversed” or “retro” peptide sequence as disclosed herein refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman, M. and Chorev, M. Accounts of Chem. Res. 1979, 12, 423.

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide compounds of the invention can be formed by utilizing an appropriate amino acid mimetic moiety to link the two adjacent portions of the sequences depicted above utilizing a reversed peptide (reversed amide) bond. In case (a) above, a central residue of a diketo compound may conveniently be utilized to link structures with two amide bonds to achieve a peptidomimetic structure. In case (b) above, a central residue of a diamino compound will likewise be useful to link structures with two amide bonds to form a peptidomimetic structure.

The reversed direction of bonding in such compounds will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is preferably (D), and the configuration of the non-reversed portion is preferably (L). Opposite or mixed configurations are acceptable when appropriate to optimize a binding activity.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrastemal injection and infusion.

The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The phrase “effective amount” as used herein means that the amount of one or more agent, material, or composition comprising one or more agents as described herein which is effective for producing some desired effect in a subject.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

(iii) Exemplary Compositions and Methods

The present invention provides a variety of compositions comprising agents that act at the cell surface to promote Wnt signaling via the canonical Wnt signaling pathway. The invention further provides a variety of methods for using these agents to promote cardiomyocyte proliferation and regeneration, as well as methods for treating a number of diseases and conditions. Non-limiting examples of compositions comprising agents that may act at the cell surface to promote Wnt signaling via the canonical Wnt signaling pathway are described in detail below.

Polypeptides and peptide fragments: The present invention provides compositions comprising Wnt-related polypeptides, modified Wnt-related polypeptides, and bioactive fragments thereof. As outlined in detail herein, exemplary Wnt-related polypeptides include Wnt3A related polypeptides, modified Wnt3A related polypeptides, and bioactive fragments thereof. Further exemplary Wnt-related polypeptides may be selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A/Wnt14, Wnt9B, Wnt15, Wnt11, or Wnt16. In any of the foregoing, a Wnt-related polypeptide for use in the methods of the present invention promotes Wnt signaling via the canonical wnt signaling pathway in a cardiac cell type. In one embodiment, the Wnt polypeptide that promotes Wnt signaling via the canonical wnt signaling pathway is selected based on its ability to promote Wnt signaling via the canonical Wnt signaling pathway in a cardiac cell type, for example, in an in vitro assay indicative of signaling via the canonical Wnt signaling pathway.

Below we describe various polypeptides. These polypeptides are candidate agents that may be used in the methods and compositions of the present invention. Candidate agents useful in the methods of the present invention promote Wnt signaling via the canonical Wnt signaling pathway. The invention further contemplates that any of the polypeptides and polypeptide fragments described in detail below can be appended to produce a modified polypeptide or modified polypeptide fragment.

In certain embodiments, the composition comprises a Wnt-related polypeptide, or a bioactive fragment thereof. Such polypeptides or fragments can include either a wildtype peptide sequence or a variant sequence, and variant sequences can be readily constructed and tested to ensure that the variant sequence retains one or more of the biological activities of the native polypeptide. One of skill in the art can readily make variants comprising an amino acid sequence at least 60%, 70%, 75%, 80%, 85%, 90%, 95%, 96%, 97%, 98% or 99% identical to a particular polypeptide, and identify variants that activate Wnt signaling and retain one or more of the biological activities of the native polypeptide. To further illustrate, the present invention contemplates Wnt-related polypeptides comprising an amino acid sequence at least 60%, 70%, 75%, 80%, 85%, 90%, 95%, 96%, 97%, 98%, 99%, or 100% identical to a Wnt polypeptide selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A/Wnt14, Wnt9B, Wnt15, Wnt11, or Wnt16 (e.g., SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78). Furthermore, the invention contemplates Wnt-related polypeptides that differ from any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, or SEQ ID NO: 78, at from one-ten positions (e.g., one, two, three, four, five, six, seven, eight, nine, or ten positions). In one embodiment, the invention contemplates Wnt-related polypeptides that differ at Cys77—for example, polypeptides that differ at Cys77 of a polypeptide Wnt3A.

In any of the foregoing, the invention contemplates compositions comprising bioactive fragments of any of the foregoing Wnt-related polypeptides or modified Wnt-related polypeptides. Exemplary bioactive fragments include fragments of at least 25, 50, 60, 70, 80, 90, 100, 125, 150, 200, 250, 300, 325, 350, or greater than 350 amino acid residues of a Wnt polypeptide selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A/Wnt14, Wnt9B, Wnt15, Wnt11, or Wnt16 (e.g., of SEQ ID NO: 2, SEQ ID NO: 4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO: 20, SEQ ID NQ: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, or SEQ ID NO: 78) that retain the biological activity of the full-length polypeptide. These biological activities include, but are not limited to, the ability to bind a frizzled receptor, the ability to promote Wnt signaling, the ability to promote the expression, activity, nuclear localization, and/or stability of β-catenin. In the context of the present invention, the one or more biological activities retained by the variant polypeptide or the bioactive fragment should include the ability to promote Wnt signaling via the canonical Wnt signaling pathway.

The present invention contemplates a wide range of compositions and pharmaceutical compositions comprising Wnt-related polypeptides, modified Wnt-related polypeptides, and bioactive fragments thereof. Such polypeptides, modified polypeptides, bioactive fragments, compositions, and pharmaceutical compositions have a variety of uses which will be outlined in greater detail herein. Generally, however, the invention contemplates pharmaceutical compositions comprising one Wnt-related polypeptide (e.g., one Wnt-related polypeptide, one modified Wnt-related polypeptide, or one bioactive fragment), as well as pharmaceutical compositions comprising more than one Wnt-related polypeptide (e.g., two, three, four, five, or more than five Wnt-related polypeptides). Furthermore, the invention contemplates the use of compositions and pharmaceutical compositions administered alone, or in combination with one or more additional agents. Such additional agents include (i) agents that promote the binding of a Wnt-related polypeptide to a frizzled receptor, (ii) agents that promote cardiomyocyte proliferation, and (iii) agents that inhibit cardiomyocyte differentiation. Additionally, the invention contemplates administering Wnt-related polypeptides together with other compounds or therapies appropriate in light of the particular disease or condition being treated. Similarly, in methods of screening to identify or characterize additional modified Wnt-related polypeptides, the invention contemplates that putative modified polypeptides may be screened singly or in combination.

In addition to the polypeptides and fragments described in detail above, the present invention also pertains to isolated nucleic acids comprising nucleotide sequences that encode said polypeptides and fragments. The term nucleic acid as used herein is intended to include fragments as equivalents, wherein such fragments have substantially the same function as the full length nucleic acid sequence from which it is derived. Equivalent nucleotide sequences will include sequences that differ by one or more nucleotide substitutions, additions or deletions, such as allelic variants; and will, therefore, include sequences that differ from the nucleotide sequence of, for example, a wildtype Wnt (any of SEQ ID NO:1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77). Equivalent sequences include those that vary from a known wildtype or variant sequence due to the degeneracy of the genetic code. Equivalent sequences may also include nucleotide sequences that hybridize under stringent conditions (i.e., equivalent to about 20-27° C. below the melting temperature (Tₘ) of the DNA duplex formed in about 1M salt) to the nucleotide sequence of Wnt-related polypeptide. Further examples of stringent hybridization conditions include a wash step of 0.2×SSC at 65° C. For the foregoing examples of equivalents to the Wnt-related polypeptides of the present invention, one of skill in the art will recognize that an equivalent sequence encodes a polypeptide that retains one or more of the biological activities of native and/or un-modified Wnt. Specifically, the polypeptide retains one or more of the following biological activities: binds to a frizzled receptor; promotes Wnt signaling; promotes the expression, activity, nuclear localization, and/or stability of β-catenin.

In one example, the invention contemplates a Wnt-related polypeptide, modified Wnt-related polypeptide, or bioactive fragment thereof encoded or encodable by a nucleic acid sequence which hybridizes under stringent conditions, including a wash step of 0.2×SSC at 65° C., to a nucleic acid sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NQ: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77.

Equivalent nucleotide sequences for use in the methods described herein also include sequences which are at least 60% identical to a give nucleotide sequence. In another embodiment, the nucleotide sequence is at least 70%, 75%, 80%, 85%, 90%, 95%, 96%, 97%, 98%, 99%, or 100% identical to the nucleotide sequence of a native sequence that encodes a Wnt-related polypeptide and retains one or more of the biological activities of a native Wnt-related polypeptide.

Nucleic acids having a sequence that differs from nucleotide sequences which encode a particular Wnt-related polypeptide due to degeneracy in the genetic code are also within the scope of the invention. Such nucleic acids encode functionally equivalent peptides but differ in sequence from wildtype sequences known in the art due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist. One skilled in the art will appreciate that these variations in one or more nucleotides (up to about 3-5% of the nucleotides) of the nucleic acids encoding polypeptides having one or more of the biological activities of a native Wnt-related polypeptide may exist among individuals of a given species due to natural allelic variation.

In the context of the present invention, compositions comprising Wnt-related polypeptides can be administered as recombinant polypeptides or compositions comprising recombinant polypeptides. Furthermore, compositions of the invention comprising Wnt-related polypeptides can be administered as conditioned medium prepared from cells expressing and secreting a Wnt-related polypeptide. For example, condition medium from Wnt3A expressing and secreting L-cells (a commercially available mouse cell line—ATCC) can be used to provide an effective amount of a composition comprising a Wnt-related polypeptide.

The present invention further provides compositions comprising LRP-related polypeptides, modified LRP-related polypeptides, and bioactive fragments thereof. As outlined in detail herein, exemplary LRP-related polypeptides include LRP5 and LRP6-related polypeptides. Below we describe various polypeptides for use in the methods and compositions of the present invention. The invention contemplates that any of the polypeptides and polypeptide fragments described in detail below can be appended to produce a modified polypeptide or modified polypeptide fragment.

In certain embodiments, the composition comprises a LRP-related polypeptide, or a bioactive fragment thereof. Such polypeptides or fragments can include either a wildtype peptide sequence or a variant sequence, and variant sequences can be readily constructed and tested to ensure that the variant sequence retains one or more of the biological activities of the native polypeptide. One of skill in the art can readily make variants comprising an amino acid sequence at least 60%, 70%, 75%, 80%, 85%, 90%, 95%, 96%, 97%, 98% or 99% identical to a particular polypeptide, and identify variants that activate Wnt signaling and retain one or more of the biological activities of the native polypeptide. To further illustrate, the present invention contemplates LRP-related polypeptides comprising an amino acid sequence at least 60%, 70%, 75%, 80%, 85%, 90%, 95%, 96%, 97%, 98%, 99%, or 100% identical to a LRP polypeptide selected from any of LRP5 or LRP6 (e.g., SEQ ID NO: 80, SEQ ID NO: 82, SEQ ID NO: 84, SEQ ID NO: 86). Furthermore, the invention contemplates LRP-related polypeptides that differ from any of SEQ ID NO: 80, SEQ ID NO: 82, SEQ ID NO: 84, or SEQ ID NO: 86, at from one-ten positions (e.g., one, two, three, four, five, six, seven, eight, nine, or ten positions).

In any of the foregoing, the invention contemplates compositions comprising bioactive fragments of any of the foregoing LRP-related polypeptides or modified LRP-related polypeptides. Exemplary bioactive fragments include fragments of at least 25, 50, 60, 70, 80, 90, 100, 125, 150, 200, 250, 300, 325, 350, or greater than 350 amino acid residues of a LRP polypeptide selected from any of LRP5 or LRP6 (e.g., of SEQ ID NO: 80, SEQ ID NO: 82, SEQ ID NO: 84, SEQ ID NO: 86) that retain the biological activity of the full-length polypeptide. These biological activities include, but are not limited to, the ability to promote Wnt signaling, the ability to promote the expression, activity, and/or stability of β-catenin. In the context of the present invention, the one or more biological activities retained by the variant polypeptide or the bioactive fragment should include the ability to promote Wnt signaling via the canonical Wnt signaling pathway.

The present invention contemplates a wide range of compositions and pharmaceutical compositions comprising LRP-related polypeptides, modified LRP-related polypeptides, and bioactive fragments thereof. Such polypeptides, modified polypeptides, bioactive fragments, compositions, and pharmaceutical compositions have a variety of uses which will be outlined in greater detail herein. Generally, however, the invention contemplates pharmaceutical compositions comprising one LRP-related polypeptide (e.g., one LRP-related polypeptide, one modified LRP-related polypeptide, or one bioactive fragment), as well as pharmaceutical compositions comprising more than one LRP-related polypeptide (e.g., two, three, four, five, or more than five LRP-related polypeptides). Furthermore, the invention contemplates the use of compositions and pharmaceutical compositions administered alone, or in combination with one or more additional agents. Such additional agents include (i) agents that promote the binding of a Wnt-related polypeptide to a frizzled receptor, (ii) agents that promote cardiomyocyte proliferation, and (iii) agents that inhibit cardiomyocyte differentiation. Additionally, the invention contemplates administering LRP-related polypeptides together with other compounds or therapies appropriate in light of the particular disease or condition being treated. Similarly, in methods of screening to identify or characterize additional modified LRP-related polypeptides, the invention contemplates that putative modified polypeptides may be screened singly or in combination.

In addition to the polypeptides and fragments described in detail above, the present invention also pertains to isolated nucleic acids comprising nucleotide sequences that encode said polypeptides and fragments. The term nucleic acid as used herein is intended to include fragments as equivalents, wherein such fragments have substantially the same function as the full length nucleic acid sequence from which it is derived. Equivalent nucleotide sequences will include sequences that differ by one or more nucleotide substitutions, additions or deletions, such as allelic variants; and will, therefore, include sequences that differ from the nucleotide sequence of, for example, a wildtype LRP (any of SEQ ID NO: 79, SEQ ID NO: 81, SEQ ID NO: 83, SEQ ID NO: 85). Equivalent sequences include those that vary from a known wildtype or variant sequence due to the degeneracy of the genetic code. Equivalent sequences may also include nucleotide sequences that hybridize under stringent conditions (i.e., equivalent to about 20-27° C. below the melting temperature (Tₘ) of the DNA duplex formed in about 1M salt) to the nucleotide sequence of LRP-related polypeptide. Further examples of stringent hybridization conditions include a wash step of 0.2×SSC at 65° C. For the foregoing examples of equivalents to the LRP-related polypeptides of the present invention, one of skill in the art will recognize that an equivalent sequence encodes a polypeptide that retains one or more of the biological activities of native and/or un-modified LRP. Specifically, the polypeptide retains one or more of the following biological activities: promotes Wnt signaling; promotes the expression, activity, nuclear localization, and/or stability of β-catenin.

In one example, the invention contemplates a LRP-related polypeptide, modified LRP-related polypeptide, or bioactive fragment thereof encoded or encodable by a nucleic acid sequence which hybridizes under stringent conditions, including a wash step of 0.2×SSC at 65° C., to a nucleic acid sequence of any of SEQ ID NO: 79, SEQ ID NO: 81, SEQ ID NO: 83, or SEQ ID NO: 85.

Equivalent nucleotide sequences for use in the methods described herein also include sequences which are at least 60% identical to a given nucleotide sequence. In another embodiment, the nucleotide sequence is at least 70%, 75%, 80%, 85%, 90%, 95%, 96%, 97%, 98%, 99%, or 100% identical to the nucleotide sequence of a native sequence that encodes a LRP-related polypeptide and retains one or more of the biological activities of a native LRP-related polypeptide.

Nucleic acids having a sequence that differs from nucleotide sequences which encode a particular LRP-related polypeptide due to degeneracy in the genetic code are also within the scope of the invention. Such nucleic acids encode functionally equivalent peptides but differ in sequence from wildtype sequences known in the art due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist. One skilled in the art will appreciate that these variations in one or more nucleotides (up to about 3-5% of the nucleotides) of the nucleic acids encoding polypeptides having one or more of the biological activities of a native LRP-related polypeptide may exist among individuals of a given species due to natural allelic variation.

Peptidomimetics: In other embodiments, the invention contemplates that the Wnt-related polypeptide, modified Wnt-related polypeptide, LRP-related polypeptide, modified LRP-related polypeptide, or bioactive fragment thereof is a peptidomimetic (herein referred to interchangeably as a mimetic or a peptide mimetic). Preferable peptidomimetics retain one or more of the biological activities of a native polypeptide. Peptidomimetics are compounds based on, or derived from, peptides and proteins. The peptidomimetics of the present invention can be obtained by structural modification of the amino acid sequence of, for example, a known Wnt-related polypeptide using unnatural amino acids, conformational restraints, isosteric replacement, and the like. The subject peptidomimetics constitute the continuum of structural space between peptides and non-peptide synthetic structures. As used herein, the term peptide mimetic will apply to any polypeptide containing a structural modification at one or more positions. For example, a full-length Wnt-related polypeptide modified at one, two, three, four, or more than four positions is a peptide mimetic. Similarly, a Wnt-related polypeptide modified at every position is a peptide mimetic. Furthermore, a bioactive fragment of a Wnt-related polypeptide modified at one or more positions, or at every position, is a Wnt-related polypeptide.

Exemplary peptidomimetics can have such attributes as being non-hydrolyzable (e.g., increased stability against proteases or other physiological conditions which degrade the corresponding peptide), having increased specificity and/or potency, and having increased cell permeability for intracellular localization. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffrnan et al. in Peptides: Chemistry and Biologyy, G. R. Marshall ed., ESCOM Publisher: Leiden, Netherlands, 1988, p. 105), keto methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p 134). Also, see generally, Session III: Analytic and synthetic methods, in in Peptides: Chemistry and Biology, G. R. Marshall ed., ESCOM Publisher: Leiden, Netherlands, 1988)

In addition to a variety of sidechain replacements which can be carried out to generate the subject peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides. 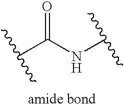
Examples of Surrogates

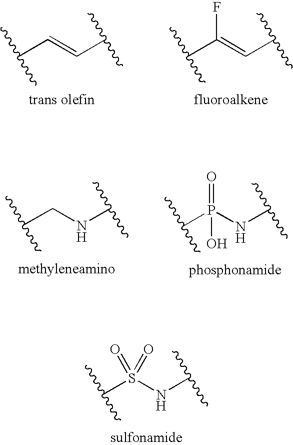

Additionally, peptidomimietics based on more substantial modifications of the backbone of a peptide can be used. Peptidomimetics which fall in this category include (i) retro-inverso analogs, and (ii) N-alkyl glycine analogs (so-called peptoids). 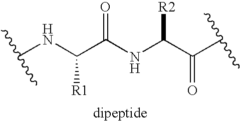
Examples of Analogs

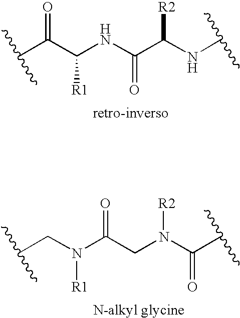

Furthermore, the methods of combinatorial chemistry are being brought to bear, e.g., PCT publication WO 99/48897, on the development of new peptidomimetics. For example, one embodiment of a so-called “peptide morphing” strategy focuses on the random generation of a library of peptide analogs that comprise a wide range of peptide bond substitutes.

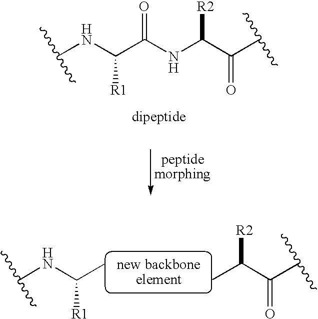

In an exemplary embodiment, the peptidomimetic can be derived as a retro-inverso analog of the peptide. Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching. The final product, or intermediates thereof, can be purified by HPLC.

In another illustrative embodiment, the peptidomimetic can be derived as a retro-enatio analog of the peptide. Retro-enantio analogs such as this can be synthesized using commercially available D-amino acids (or analogs thereof) and standard solid- or solution-phase peptide-synthesis techniques. For example, in a preferred solid-phase synthesis method, a suitably amino-protected (t-butyloxycarbonyl, Boc) residue (or analog thereof) is covalently bound to a solid support such as chloromethyl resin. The resin is washed with dichloromethane (DCM), and the BOC protecting group removed by treatment with TFA in DCM. The resin is washed and neutralized, and the next Boc-protected D-amino acid is introduced by coupling with diisopropylcarbodiimide. The resin is again washed, and the cycle repeated for each of the remaining amino acids in turn. When synthesis of the protected retro-enantio peptide is complete, the protecting groups are removed and the peptide cleaved from the solid support by treatment with hydrofluoric acid/anisole/dimethyl sulfide/thioanisole. The final product is purified by HPLC to yield the pure retro-enantio analog.

In still another illustrative embodiment, trans-olefin derivatives can be made for any of the subject polypeptides. A trans olefin analog can be synthesized according to the method of Y. K. Shue et al. (1987) Tetrahedron Letters 28:3225 and also according to other methods known in the art. It will be appreciated that variations in the cited procedure, or other procedures available, may be necessary according to the nature of the reagent used.

It is further possible to couple the pseudodipeptides synthesized by the above method to other pseudodipeptides, to make peptide analogs with several olefinic functionalities in place of amide functionalities.

Still another classes of peptidomimetic derivatives include phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in designing peptidomimetics. To illustrate, the peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic, heteroaromatic, or biheteroaromatic nucleus.

The subject peptidomimetics can be optimized by, e.g., combinatorial synthesis techniques combined with high throughput screening techniques, and furthermore can be tested to ensure that the peptidomimetic retains one or more of the biological activities of the native polypeptide. Any of the foregoing peptidomimetics can be modified with one or more hydrophobic and/or hydrophilic moieties, as described herein for other polypeptides. Exemplary modified polypeptide peptidomimetics retain one or more of the biological activities of the native polypeptide and additionally possess one or more advantageous physiochemical properties.

Hydrophobically Modified Polypeptides

In addition to providing Wnt-related compositions comprising polypeptides and bioactive fragments thereof, as described herein, the present invention recognizes that certain compositions comprising modified Wnt-related polypeptides and bioactive fragments thereof will have certain other advantages in comparison to their native and/or unmodified counter-parts. Such modified Wnt-related polypeptides (including full-length polypeptides and bioactive fragments) not only retain one or more of the biological activities of the corresponding native or un-modified Wnt, but may also possess one or more additional, advantageous physiochemical properties in comparison to a native and/or unmodified Wnt. Exemplary physiochemical properties include, but are not limited to, increased in vitro half-life, increased in vivo half-life, decreased immunogenicity, increased solubility, increased potency, increased bioavailability, and increased biodistribution. The present invention contemplates compositions comprising modified Wnt-related polypeptide. For example, the present invention contemplates modified Wnt3A-related polypeptides. Furthermore, the present invention contemplates modified Wnt-related polypeptides selected from any of Wnt1, Wnt2, Wnt 2B, Wnt3 Wnt3A, Wnt4, Wnt5A, Wnt5B, Wnt6, Wnt7A, Wnt7B, Wnt8A, Wnt8B, Wnt9A, Wnt9B, Wnt11, and Wnt16. Compositions comprising modified Wnt-related polypeptides area also referred to herein as modified Wnt-related compositions.

In one embodiment, the modified Wnt-related polypeptide is a hydrophobically modified Wnt-related polypeptide. The invention contemplates that a Wnt-related polypeptide may be appended with one or more moieties to produce a modified Wnt-related polypeptide. For example, a modified Wnt-related polypeptide may be appended with two, three, four, five, or more than five moieties. The moieties may be the same or may be different. When said one or more moieties are hydrophobic moieties, the modified Wnt-related polypeptide is also known as a hydrophobically modified Wnt-related polypeptide.

Furthermore, the invention contemplates that the one or more moieties (e.g., one or more independently selected hydrophobic moieties) may be appended to the N-terminal amino acid residue, the C-terminal amino acid residue, and/or one or more internal amino acid residues. When a modified Wnt-related polypeptide is appended with two or more moieities, the moieties may be appended to the same amino acid residue and/or to different amino acid residues. Additionally, as detailed above, the moieties may be the same or different.

The present invention provides modified Wnt-related polypeptides, and methods of using these modified Wnt-related polypeptides in vitro and in vivo. The modified Wnt-related polypeptides of the present invention should retain one or more of the biological activities of the corresponding native and/or un-modified Wnt. For example, the invention contemplates modified Wnt3A-related polypeptides that retain one or more of the biological activities of native and/or un-modified Wnt3A. Additionally, preferable modified Wnt-related polypeptides possess one or more advantageous physiochemical characteristics in comparison to the corresponding native and/or un-modified Wnt. For example, a modified Wnt3A-related polypeptide retains one or more biological activity of Wnt3A and possesses one or more advantageous physiochemical property in comparison to native and/or un-modified Wnt3A.

Accordingly, modified Wnt-related polypeptides not only provide additional possible compositions for manipulating Wnt signaling in vitro or in vivo, such modified Wnt-related polypeptides may also provide Wnt-related polypeptides with improved properties in comparison to the prior art. Exemplary modified Wnt-related polypeptides include hydrophobically modified Wnt-related polypeptides.

Modifying a polypeptide or peptide (i.e, adding or appending one or more hydrophobic moieties to an existing amino acid residue or substituting one or more hydrophobic moieties for an amino acid) can alter the physiochemical properties of the polypeptide in useful way. For example, such hydrophobically modified Wnt-related polypeptides may have increased biological activity, increased stability, increased in vivo or in vitro half-life, or decreased immunogenicity in comparison to a native and/or un-modified Wnt-related polypeptide.

The overall hydrophobic character of a polypeptide can be increased in any of a number of ways. Regardless of how the polypeptide is modified in order to increase its hydrophobicity, one of skill in the art will recognize that preferable modified Wnt-related polypeptides retain one or more of the biological activities of the corresponding native and/or un-modified Wnt. Additionally, particularly preferred modified polypeptides possess one or more advantageous physiochemical properties. In one embodiment, the modified Wnt-relatd polypeptide is a modified Wnt3A-related polypeptide.

Briefly, the hydrophobicity of a polypeptide can be increased by (a) chemically modifying an amino acid residue or (b) replacing an amino acid residue with one or more hydrophobic amino acid residues. By way of further example, a polypeptide can be chemically modified in any of a number of ways. A chemical moiety can be directly appended via a reactive amino acid residue (e.g., via reaction with a sulfhydryl and/or an alpha-amine of a cysteine residue or via reaction with another reactive amino acid residue). Such a reactive amino acid residue may exist in the native polypeptide sequence or such a reactive amino acid residue may be added to the native sequence to provide a site for addition of a hydrophobic moiety. Similarly, when the hydrophobicity of a polypeptide is increased by addition of hydrophobic amino acid residues, such additional hydrophobic amino acid residues may either replace amino acid residue of the native polypeptide, or such amino acid residue may be appended to the native amino acid residues.

Exemplary hydrophobic moieties may be appended to the N-terminal, C-terminal, and/or one or more internal amino acid residues. One class of hydrophobic moieties that may be appended to a Wnt-related polypeptide includes lipids such as fatty acid moieties and sterols (e.g., cholesterol). Derivatized proteins of the invention contain fatty acids which are cyclic, acyclic (i.e., straight chain), saturated or unsaturated, mono-carboxylic acids. Exemplary saturated fatty acids have the generic formula: CH3(CH2)n COOH. The table below lists examples of some fatty acids that can be conveniently appended to a Wnt-related polypeptide using conventional chemical methods.

### Exemplary Saturated and Unsaturated Fatty Acids

| | |
|---|---|
| Value of n | Common Name |

Saturated Acids: CH₃(CH₂)n COOH:

| | |
|---|---|
| 2 | butyric acid |
| 4 | caproic acid |
| 6 | caprylic acid |
| 8 | capric acid |
| 10 | lauric acid |
| 12 | myristic acid |
| 14 | palmitic acid |
| 16 | stearic acid |
| 18 | arachidic acid |
| 20 | behenic acid |
| 22 | lignoceric acid |

Unsaturated Acids:

| | |
|---|---|
| CH3CH═CHCOOH | crotonic acid |
| CH3(CH2)3CH═CH(CH2)7COOH | myristoleic acid |
| CH3(CH2)5CH═CH(CH2)7COOH | palmitoleic acid |
| CH3(CH2)7CH═CH(CH2)7COOH | oleic acid |
| CH3(CH2)3(CH2CH═CH)2(CH2)7COOH | linoleic acid |
| CH3(CH2)CH═CH)3(CH2)7COOH | linolenic acid |
| CH3(CH2)3(CH2CH═CH)4(CH2)3COOH | arachidonic acid |

Other lipids that can be attached to a Wnt-related polypeptide include branched-chain fatty acids and those of the phospholipid group such as the phosphatidylinositols (i.e., phosphatidylinositol 4-monophosphate and phosphatidylinositol 4,5-biphosphate), phosphatidycholine, phosphatidylethanolamine, phosphatidylserine, and isoprenoids such as farnesyl or geranyl groups.

There are a wide range of hydrophobic moieties with which a Wnt-related polypeptide can be derivatized. A hydrophobic group can be, for example, a relatively long chain alkyl or cycloalkyl (preferably n-alkyl) group having approximately 7 to 30 carbons. The alkyl group may terminate with a hydroxy or primary amine “tail”. To further illustrate, such molecules include naturally-occurring and synthetic aromatic and non-aromatic moieties such as fatty acids, esters and alcohols, other lipid molecules, cage structures such as adamantane and buckminsterfullerenes, and aromatic hydrocarbons such as benzene, perylene, phenanthrene, anthracene, naphthalene, pyrene, chrysene, and naphthacene.

Particularly useful as hydrophobic molecules are alicyclic hydrocarbons, saturated and unsaturated fatty acids and other lipid and phospholipid moieties, waxes, cholesterol, isoprenoids, terpenes and polyalicyclic hydrocarbons including adamantane and buckminsterfullerenes, vitamins, polyethylene glycol or oligoethylene glycol, (C1-C18)-alkyl phosphate diesters, —O—CH2—CH(OH)—O—(C12-C18)-alkyl, and in particular conjugates with pyrene derivatives. The hydrophobic moiety can be a lipophilic dye suitable for use in the invention including, but not limited to, diphenylhexatriene, Nile Red, N-phenyl-1-naphthylamine, Prodan, Laurodan, Pyrene, Perylene, rhodamine, rhodamine B, tetramethylrhodamine, Texas Red, sulforhodamine, 1,1′-didodecyl-3,3,3′,3′tetramethylindocarbocyanine perchlorate, octadecyl rhodamine B, and the BODIPY dyes available from Molecular Probes Inc.

Other exemplary lipophilic moieties include aliphatic carbonyl radical groups including 1- or 2-adamantylacetyl, 3-methyladamant-1-ylacetyl, 3-methyl-3-bromo-1-adamantylacetyl, 1-decalinacetyl, camphoracetyl, camphaneacetyl, noradamantylacetyl, norbornaneacetyl, bicyclo[2.2.2.]-oct-5-eneacetyl, 1-methoxybicyclo[2.2.2.]-oct-5-ene-2-carbonyl, cis-5-norbornene-endo-2,3-dicarbonyl, 5-norbornen-2-ylacetyl, (1R)-(−)-myrtentaneacetyl, 2-norbornaneacetyl, anti-3-oxo-tricyclo[2.2.1.0<2,6>]-heptane-7-carbonyl, decanoyl, dodecanoyl, dodecenoyl, tetradecadienoyl, decynoyl or dodecynoyl.

As outlined in detail above, the invention contemplates modified Wnt-related polypeptides containing one or more hydrophobic moieties, and further contemplates that said one or more moieties can be appended to the N-terminal amino acid residue, the C-terminal amino acid residue, and/or an internal amino acid residue. When the modified Wnt-related polypeptide is appended with two or more moieties, these moieties may be the same or may be different. Furthermore, such moieties may be appended to the same amino acid residue and/or to different amino acid residues.

The invention further contemplates that the hydrophobicity of a Wnt-related polypeptide may be increased by appending one or more hydrophobic amino acid residues to the polypeptide or by replacing one or more amino acid residue with one or more hydrophobic amino acid residues. For example, phenylalanine, isoleucine, and methionine are hydrophobic amino acid residues. Accordingly, appending one or more of these residues to a Wnt-related polypeptide would increase the hydrophobicity of the Wnt-related polypeptide. Similarly, replacing one or more of the amino acid residues of the native polypeptide with one or more of these amino acid residues would increase the hydrophobicity of the Wnt-related polypeptide. In one example, the substitution of a hydrophobic amino acid residue for a native residue may be a conservative substitution, and thus one of skill in the art would not expect the substitution to alter the function of the Wnt-related polypeptide. Further exemplary hydrophobic amino acid residues include tryptophan, leucine, valine, alanine, proline, and tyrosine.

The foregoing examples illustrate the varieties of modified Wnt-related polypeptide contemplated by the present invention. Any of these modified Wnt-related polypeptide can be synthesized using techniques well known in the art, and these modified Wnt-related polypeptide can be tested using in vitro and in vivo assays to identify modified compositions that (i) retain one or more of the biological activities of the corresponding native and/or un-modified Wnt polypeptide and, preferably (ii) possess one or more advantageous physiochemical characteristics in comparison to the native and/or unmodified Wnt polypeptide.

The present invention recognizes that certain native forms (e.g., major form or a minor form) of Wnt-related polypeptides may be hydrophibically modified. For example, some groups have reported a form of Wnt3A modified with a palmitoyl moiety on Cys77. The present invention contemplates hydrophobically modified polypeptides that further comprise (i) a palmitoyl moiety at Cys77; (ii) a moiety other than a palmitoyl moiety at Cys77; (iii) no modification at Cys77.

As outlined briefly above, any of a number of methods well known in the art can be used to modify a Wnt-related polypeptide (e.g., to append one or more moiety, such as a hydrophobic moiety, to one or more amino acid residue). Exemplary methods include, but are not limited to, the following: (i) derivatization of an amino acid residue; (ii) derivatization of a reactive amino acid residue; (iii) addition of a reactive amino acid residue to the native sequence, and derivatization of the added amino acid residue; (iv) replacement of an amino acid residue in the native sequence with a reactive amino acid residue, and derivatization of the reactive amino acid residue; (v) addition of a hydrophobic amino acid residue or hydrophobic peptide; and (vi) replacement of an amino acid residue in the native sequence with one or more hydrophobic amino acids or peptides.

If an appropriate amino acid is not available at a specific position, site-directed mutagenesis can be used to place a reactive amino acid at that site. Similarly, when synthesizing a Wnt-related polypeptide, an appropriate reactive amino acid can be added to the polypeptide (e.g., added to the N-terminus or C-terminus, or internally). Of course, any such variant sequences must be assessed to confirm that the variant retains one or more of the biological activities of the corresponding native and/or un-modified polypeptide. Reactive amino acids include cysteine, lysine, histidine, aspartic acid, glutamic acid, serine, threonine, tyrosine, arginine, methionine, and tryptophan, and numerous methods are well known in the art for appending moieties to any of these reactive amino acids. Furthermore, methodologies exist for appending various moieties to other amino acids, and one of skill in the art can readily select the appropriate techniques for appending a moiety to an amino acid residue.

There are specific chemical methods for the modification of many amino acids, including reactive amino acids. Therefore, a route for synthesizing a modified Wnt-related polypeptide would be to chemically attach a hydrophobic moiety to an amino acid in a Wnt-related polypeptide. Such amino acid may be a reactive amino acid. Such amino acid may exist in the native sequence or may be added to the native sequence prior to modification. If an appropriate amino acid is not available at the desired position, site-directed mutagenesis at a particular site can be used. Reactive amino acids would include cysteine, lysine, histidine, aspartic acid, glutamic acid, serine, threonine, tyrosine, arginine, methionine, and tryptophan. Thus the goal of creating a modified Wnt-related polypeptide could be attained by many chemical means and we do not wish to be restricted by a particular chemistry or site of modification. One of skill in the art can readily make a wide range of modified Wnt-related polypeptides using well-known techniques in chemistry, and one of skill in the art can readily test the modified Wnt-related polypeptides in any of a number of in vitro or in vivo assays to identify the modified Wnt-related polypeptides which retain one or more of the biological activities of the corresponding native and/or unmodified Wnt polypeptide. Furthermore, one of skill in the art can readily evaluate which modified Wnt-related polypeptides which retain one or more of the biological activities of the corresponding native and/or unmodified Wnt polypeptide also possess advantageous physiochemical properties.

The polypeptide can be linked to the hydrophobic moiety in a number of ways including by chemical coupling means, or by genetic engineering. To illustrate, there are a large number of chemical cross-linking agents that are known to those skilled in the art. One class of cross-linking agents are heterobifunctional cross-linkers, which can be used to link the polypeptides and hydrophobic moieties in a stepwise manner. Heterobifunctional cross-linkers provide the ability to design more specific coupling methods for conjugating to proteins, thereby reducing the occurrences of unwanted side reactions such as homo-protein polymers. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate]hexanoate (LC-SPDP).

Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl (DMP) are examples of useful homobifunctional cross-linking agents, and bis-[.beta.-(4-azidosalicylamido)ethyl]disulfide (BASED) and N-succinimidyl-6(4′-azido-2′-nitrophenyl-amino)hexanoate (SANPAH) are examples of useful photoreactive cross-linkers for use in this invention. For a recent review of protein coupling techniques, see Means et al. (1990) Bioconjugate Chemistry 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds.

The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules. For instance, SMPB has a span of 14.5 angstroms.

Preparing protein-protein conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using for instance, N-ethylmaleimide (see Partis et al. (1983) J. Pro. Chem. 2:263). Ellman's Reagent can be used to calculate the quantity of sulfhydryls in a particular protein (see for example Ellman et al. (1958) Arch. Biochem. Biophys. 74:443 and Riddles et al. (1979) Anal Biochem. 94:75).

The reaction buffer should be free of extraneous amines and sulfhydryls. The pH of the reaction buffer should be 7.0-7.5. This pH range prevents maleimide groups from reacting with amines, preserving the maleimide group for the second reaction with sulfhydryls.

The NHS-ester containing cross-linkers have limited water solubility. They should be dissolved in a minimal amount of organic solvent (DMF or DMSO) before introducing the cross-linker into the reaction mixture. The cross-linker/solvent forms an emulsion which will allow the reaction to occur.

The sulfo-NHS ester analogs are more water soluble, and can be added directly to the reaction buffer. Buffers of high ionic strength should be avoided, as they have a tendency to “salt out” the sulfo-NHS esters. To avoid loss of reactivity due to hydrolysis, the cross-linker is added to the reaction mixture immediately after dissolving the protein solution.

The reactions can be more efficient in concentrated protein solutions. The more alkaline the pH of the reaction mixture, the faster the rate of reaction. The rate of hydrolysis of the NHS and sulfo-NHS esters will also increase with increasing pH. Higher temperatures will increase the reaction rates for both hydrolysis and acylation.

Once the reaction is completed, the first protein is now activated, with a sulfhydryl reactive moiety. The activated protein may be isolated from the reaction mixture by simple gel filtration or dialysis. To carry out the second step of the cross-linking, the sulfhydryl reaction, the lipophilic group chosen for reaction with maleimides, activated halogens, or pyridyl disulfides must contain a free sulfhydryl. Alternatively, a primary amine may be modified with to add a sulfhydryl

In all cases, the buffer should be degassed to prevent oxidation of sulfhydryl groups. EDTA may be added to chelate any oxidizing metals that may be present in the buffer. Buffers should be free of any sulfhydryl containing compounds.

Maleimides react specifically with —SH groups at slightly acidic to neutral pH ranges (6.5-7.5). A neutral pH is sufficient for reactions involving halogens and pyridyl disulfides. Under these conditions, maleimides generally react with —SH groups within a matter of minutes. Longer reaction times are required for halogens and pyridyl disulfides.

The first sulfhydryl reactive-protein prepared in the amine reaction step is mixed with the sulfhydryl-containing lipophilic group under the appropriate buffer conditions. The conjugates can be isolated from the reaction mixture by methods such as gel filtration or by dialysis.

Exemplary activated lipophilic moieties for conjugation include: N-(1-pyrene)maleimide; 2,5-dimethoxystilbene-4′-maleimide, eosin-5-maleimide; fluorescein-5-maleimide; N-(4-(6-dimethylamino-2-benzofuranyl)phenyl)maleimide; benzophenone-4-maleimide; 4-dimethylaminophenylazophenyl-4′-maleimide (DABMI), tetramethylrhodamine-5-maleimide, tetramethylrhodamine-6-maleimide, Rhodamine Red™ C2 maleimide, N-(5-aminopentyl)maleimide, trifluoroacetic acid salt, N-(2-aminoethyl)maleimide, trifluoroacetic acid salt, Oregon Green™ 488 maleimide, N-(2-((2-(((4-azido-2,3,5,6-tetrafluoro)benzoyl)amino)ethyl)dithio)ethyl)maleimide (TFPAM-SS1), 2-(1-(3-dimethylaminopropyl)-indol-3-yl)-3-(indol-3-yl) maleimide (bisindolylmaleimide; GF 109203X), BODIPY.RTM. FL N-(2-aminoethyl)maleimide, N-(7-dimethylamino-4-methylcoumarin-3-yl)maleimide (DACM), Alexa™ 488 C5 maleimide, Alexa™ 594 C5 maleimide, sodium saltN-(1-pyrene)maleimide, 2,5-dimethoxystilbene-4′-maleimide, eosin-5-maleimide, fluorescein-5-maleimide, N-(4-(6-dimethylamino-2-benzofuranyl)phenyl)maleimide, benzophenone-4-maleimide, 4-dimethylaminophenylazophenyl-4′-maleimide, 1-(2-maleimidylethyl)-4-(5-(4-methoxyphenyl)oxazol-2-yl)pyridinium methanesulfonate, tetramethylrhodamine-5-maleimide, tetramethylrhodamine-6-maleimide, Rhodamine Red™ C2 maleimide, N-(5-aminopentyl)maleimide, N-(2-aminoethyl)maleimide, N-(2-((2-(((4-azido-2,3,5,6-tetrafluoro)benzoyl)amino)ethyl)dithio)ethyl)maleimide, 2-(1-(3-dimethylaminopropyl)—indol-3-yl)-3-(indol-3-yl) maleimide, N-(7-dimethylamino-4-methylcoumarin-3-yl)maleimide (DACM), 11H-Benzo[a]fluorene, Benzo[a]pyrene.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

The foregoing methods are merely provided to illustrate the techniques that one of skill in the art can readily employ in making a wide range of modified Wnt-related polypeptides. In one embodiment, the invention contemplates a wide range of modified Wnt-related polypeptides. Further methods are described in U.S. Pat. No. 6,444,793, which is hereby incorporated by reference in its entirety.

In addition to the aforementioned modified Wnt-related polypeptides, the invention contemplates modified LRP-related polypeptides, and bioactive fragments thereof. Exemplary modified LRP-related polypeptides retain the ability of unmodified LRP to promote Wnt signaling via the canonical Wnt signaling pathway. Further exemplary modified LRP-related polypeptides can be used to promote cardiomyocyte proliferation. The present invention contemplates that LRP-related polypeptides can be modified with one or more hydrophobic or hydrophilic moieties using the same methods and compositions that can be used to modify Wnt-related compostions. Accordingly, throughout the present application references to methods and compositions for appending one or more moieties to a Wnt-related composition should be considered exemplary of the methods and compositions that can be used to modify LRP-related polypeptides.

Hydrophilically Modified Polypeptides

In addition to providing Wnt-related compositions comprising polypeptides and bioactive fragments thereof, as described herein, the present invention recognizes that certain compositions comprising modified Wnt-related polypeptides and bioactive fragments thereof will have certain other advantages in comparison to their native and/or unmodified counter-parts. Such modified Wnt-related polypeptides (including full-length polypeptides and bioactive fragments) not only retain one or more of the biological activities of native or un-modified Wnt, but also possess one or more additional, advantageous physiochemical properties in comparison to a native and/or un-modified Wnt. Exemplary physiochemical properties include, but are not limited to, increased in vitro half-life, increased in vivo half-life, decreased immunogenicity, increased solubility, increased potency, increased bioavailability, and increased biodistribution. One class of preferred modified polypeptides include hydrophilically modified polypeptides such as polypeptides appended with one or more cyclodextran moieties, polypeptides appended with one or more PEG moieties, polypeptides appended with one or more laminin moieties, and polypeptides appended with one or more antibody moieties. One preferred class of modified polypeptides and compositions according to the present invention are pegylated polypeptides and compositions. A pegylated Wnt-related polypeptides is appended with a PEG containing moiety comprising one or more PEG [(poly(ethylene) glycol or (poly(ethylene) glycol derivative] moieties. An exemplary PEG moiety is represented in FIG. 1, and exemplary PEG containing moieties containing reactive groups for attachment to polypeptides are represented in FIGS. 2-14. In any of FIGS. 2-14, one of skill in the art will readily appreciate that the abbreviation PEG refers to any polyethylene glycol or polyethylene glycol related or derived moiety such as, for example, the PEG moiety represented in FIG. 1.

The invention provides compositions comprising modified Wnt-related polypeptides and methods for using these modified Wnt-related polypeptides. In one embodiment, the modified Wnt-related polypeptide is a pegylated Wnt polypeptide (e.g., the Wnt-related polypeptide is appended with one or more PEG containing moieties). Appending PEG containing moieties to polypeptides may be used to obtain modified compositions that retain one or more of the biological properties of the native or un-modified polypeptide, and further possess one or more advantageous physiochemical properties

The term “PEG containing moiety” and “PEG containing moiety comprising one or more PEG moiety” are used throughout this application to refer to the modified Wnt-related polypeptides of the invention. FIG. 1 provides a representation of a PEG containing moiety comprising one or more PEG moieties. As illustrated by the figure, PEG moieties may exist as a polymer of virtually any size, and the invention contemplates that PEG containing moieties comprising 1, 2, 3, 4, 5, 6, 8, 10, 20, 40, 50, 100, or greater than 100 PEG moieties can be appended to a Wnt-related polypeptide. FIGS. 2-14 provide representations of other exemplary PEG containing moieties (e.g., PEG containing moieties which further contain reactive groups for appending to a Wnt-related polypeptide). In any of FIGS. 2-14, the abbreviation PEG refers to any polyethylene glycol or polyethylene glycol derivative, as for example, provided in FIG. 1.

The polymer backbone is a water soluble, substantially non-immunogenic polymer, and is preferably poly(ethylene) glycol. However, as used throughout the specification, the term “PEG”, “PEG moiety”, and “PEG containing moiety” refer to poly(ethylene glycol) containing moieties, as well as other related polymers. Suitable polymer backbones include, but are not limited to, linear and branched poly(ethylene glycol), linear and branched poly(41kylene oxide), linear and branched poly(vinyl pyrrolidone), linear and branched poly(vinyl alcohol), linear and branched polyoxazoline, linear and branched poly(acryloylmorpholine), and derivatives thereof. Additionally, when the PEG containing moiety comprises more than one PEG moiety, the invention contemplates that the PEG moieties may be the same (e.g., each PEG moiety is polyethylene glycol) or that the PEG moieties may be different (e.g., one or more polyethylene glycol moiety and one or more polyvinyl alcohol moiety).

PEG moieties are useful in biological applications because they have properties that are highly desirable and are generally approved for biological applications in vivo and in vitro. PEG typically is clear, colorless, odorless, soluble in water, stable to heat, inert to many chemical agents, does not hydrolyze or deteriorate, and is generally nontoxic. Poly(ethylene) glycol and other PEG related polymers are considered to be biocompatible, which is to say that PEG is capable of coexistence with living tissues or organisms without causing harm. More specifically, PEG is non-immunogenic, which is to say that PEG does not tend to produce an immune response in the body. When attached to a molecule having some desirable function in the body, such as a biologically active agent, to form a conjugate, the PEG tends to mask the agent and can reduce or eliminate any immune response so that an organism can tolerate the presence of the agent. Accordingly, the conjugate is substantially non-toxic. PEG conjugates tend not to produce a substantial immune response or cause clotting or other undesirable effects.

PEG having the formula —CH₂ CH₂—(CH₂CH₂O)ₙ—CH₂ CH₂—, where n is from about 8 to about 4000, is one useful polymer in the practice of the invention. Preferably PEG having a molecular weight of from about 200 to about 100,000 Da is used as polymer backbone.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol.

Many other water soluble substantially non-immunogenic polymers than PEG are also suitable for the present invention. These other polymers can be either in linear form or branched form, and include, but are not limited to, other poly(alkylene oxides) such as poly(propylene glycol) (“PPG”), copolymers of ethylene glycol and propylene glycol and the like; poly(vinyl alcohol) (“PVA”) and the like. The polymers can be homopolymers or random or block copolymers and terpolymers based on the monomers of the above polymers, straight chain or branched.

Specific examples of suitable additional polymers include, but are not limited to, difunctional poly(acryloylmorpholine) (“PAcM”), and poly(vinylpyrrolidone) (“PVP”). PVP and poly(oxazoline) are well known polymers in the art and their preparation should be readily apparent to the skilled artisan. PAcM and its synthesis and use are described in U.S. Pat. Nos. 5,629,384 and 5,631,322. Although the molecular weight of each chain of the polymer backbone can vary, it is typically in the range of from about 100 to about 100,000, preferably from about 6,000 to about 80,000.

Those of ordinary skill in the art will recognize that the foregoing list for substantially water soluble non-immunogenic polymer backbone is by no means exhaustive and is merely illustrative, and that all polymeric materials having the qualities described above are contemplated.

In addition to PEG moieties, preferred PEG containing moieties of the invention also contain a reactive group to facilitate attachment of the PEG containing moiety to the Wnt-related polypeptide. The reactive group allows the PEG containing moiety to be readily appended to a free amine of an amino acid residue. For example, via the reactive group, a PEG containing moiety can be appended to the primary amine of the N-terminal amino acid residue of a Wnt-related polypeptide. Via the reactive group, a PEG containing moiety can be appended to an amine containing amino acid residue including an internal amino acid residue or a C-terminal amino acid residue. An amine containing amino acid residue may be naturally present in a particular polypeptide. However, if an amine containing amino acid residue is not present, an amine containing amino acid residue can be added to a polypeptide at either the N-terminus, C-terminus, or internally, and this added amine containing amino acid residue can supply a site for appending a PEG containing moiety. Following addition of an amine containing amino acid residue, the polypeptide should retain the function of the native polypeptide. Furthermore, if an amine containing amino acid residue is not present, an amine containing amino acid residue can be substituted for a residue already present in the polypeptide. Following substitution of an amine containing amino acid residue for an amino acid residue that does not contain a free amine, the polypeptide should retain the activity of the native polypeptide.

The reactive group (also referred to herein as the reactive moiety) is a moiety capable of reacting with a moiety in another molecule, e.g., a biologically active agent such as proteins, peptides, etc. Examples of suitable reactive moieties include, but are not limited to, active esters, active carbonates, aldehydes, isocyanates, isothiocyanates, epoxides, alcohols, maleimides, vinylsulfones, hydrazides, dithiopyridines, N-succinimidyl, and iodoacetamides. The selection of a free reactive moiety is determined by the moiety in another molecule to which the free reactive moiety is to react. For example, when the moiety in another molecule is a thiol moiety, then a vinyl sulfone moiety is preferred for the free reactive moiety of the activated polymer. On the other hand, an N-succinimidyl moiety is preferred to react to an amino moiety in a biologically active agent.

The invention contemplates any of a number of modified Wnt-related polypeptides. The modified Wnt-related polypeptides will vary with respect to the number and/or identity of the PEG moieties comprising the PEG containing moiety, and with respect to the reactive group through which the PEG containing moiety is appended to the Wnt-related polypeptide. Nevertheless, the present invention contemplates that any such pegylated Wnt-related polypeptide can be readily constructed and tested to identify modified Wnt-related polypeptides that retain one or more of the biological activities of native or un-modified Wnt and which possess one or more advantageous physiochemical property in comparison to native or unmodified Wnt. Particularly advantageous PEG containing moieties and methods for appending said PEG containing moieties to a Wnt-related polypeptide are further summarized in, for example, the following issued patents and publications. The disclosures of each of the following references are hereby incorporated by reference in their entirety: U.S. Pat. No. 6,664,331, U.S. Pat. No. 6,624,246, U.S. Pat. No. 6,610,281, WO03/070805, U.S. Pat. No. 6,602,952, U.S. Pat. No. 6,602,498, U.S. Pat. No. 6,541,543, U.S. Pat. No. 6,541,015, U.S. Pat. No. 6,515,100, U.S. Pat. No. 6,514,496, U.S. Pat. No. 6,514,491, U.S. Pat. No. 6,495,659, U.S. Pat. No. 6,461,603, U.S. Pat. No. 6,461,602, U.S. Pat. No. 6,436,386, U.S. Pat. No. 5,900,461, WO03/040211, WO03/000777, U.S. Pat. No. 6,448,369, U.S. Pat. No. 6,437,025, and Roberts et al. (2002) Advanced Drug Delivery Reviews 54: 459-476.

In addition, pegylated Wnt-related polypeptides according to the present invention may have any of the following properties. In certain embodiments, a pegylated Wnt-related polypeptide is modified with a moiety comprising one or more PEG (or PEG-related) moieties. Such one or more PEG moieties can be arranged linearly with respect to the Wnt-related polypeptide or can be arranged in a branched configuration. The PEG containing moiety may be covalently appended to the primary amine of the N-terminal amino acid residue of the Wnt-related polypeptide although the invention contemplates other well known methods for appending PEG moieties to polypeptides. Other preferred embodiments include appending one or more PEG containing moieties to an internal amino acid residue containing a free amine, appending one or more PEG containing moieties to a C-terminal amino acid residue containing a free amine, or appending one or more PEG containing moieties to a reactive lysine or cysteine residue (e.g., an N-terminal, internal, or C-terminal reactive lysine or cysteine residue). We note that certain polypeptides may not contain a convenient free amine for appending one or more PEG moieties. Accordingly, the invention further contemplates the addition or substitution of a free amine containing amino acid residue to a polypeptide to serve as a site of attachment for one or more PEG containing moiety. Following addition or substitution of an amino acid residue to the N-terminus, C-terminus, or internally, the variant polypeptide should retain one or more of the biological activities of the native polypeptide (e.g., addition or substitution of the free amine containing amino acid residue should not disrupt the activity of the polypeptide). For any of the foregoing, the invention contemplates that one or more PEG containing moieties can be appended to the same or to different amino acid residues.

The pegylated Wnt-related polypeptides according to the present invention can additionally be described in a number of ways. For example, the invention contemplates appending Wnt-related polypeptides with PEG containing moieties totaling approximately 5 kDa, 10 kDa, 20 kDa, 30 kDa, 40 kDa, 60 kDa, 80 kDa, or greater than 80 kDa (e.g., the PEG containing moiety increases the molecular weight of the Wnt-related polypeptide by approximately 5, 10, 20, 30, 40, 60, 80, or greater than 80 kDa).

Furthermore, the pegylated Wnt-related polypeptides of the invention can be described in terms of the polydispersity of the PEG containing moiety. In one embodiment, the polydispersity is approximately 1.01-1.02 MW/MN (molecular weight/molecular number). In another embodiment, the polydispersity is less than 1.05 MW/MN. In yet another embodiment, the polydispersity is greater than 1.05 MW/MN.

The present invention contemplates the attachment of PEG containing moieties (e.g., moieties comprising one or more PEG or PEG-related moieties) to Wnt-related polypeptides. For example, the present invention contemplates the attachment of PEG containing moieties to the primary amine of the N-terminal amino acid residue of a Wnt-related polypeptide. The present invention further contemplates the attachment of PEG containing moieties to any amine containing amino acid residue of a Wnt-related polypeptide (e.g., an N-terminal, C-terminal, or internal amine containing amino acid residue). Such attachment may be a covalant attachment, and such covalent attachment may occur via an active group of the PEG containing moiety. For example, attachment may occur via an active ester, an active aldehyde, or an active carbonate. Further examples of reactive groups used to covalently append a PEG containing moiety include but are not limited to, isocyanates, isothiocyanates, epoxides, alcohols, maleimides, vinylsulfones, hydrazides, dithiopyridines, and iodoacetamides.

In addition to the foregoing pegylated Wnt-related polypeptides, the invention contemplates Wnt-related polypeptides modified with other moieties that increase the hydrophilicity of the modified Wnt-related polypeptides. Such hydrophilic Wnt-related polypeptides retain one or more of the biological activities of unmodified or native Wnt, and preferably have one or more advantageous physiochemical properties in comparison to unmodified and/or native Wnt-related polypeptide. Exemplary physiochemical properties include, but are not limited to, increased in vitro half-life, increased in vivo half-life, decreased immunogenicity, increased solubility, increased potency, increased solubility, increased bioavailability, and increased biodistribution. Exemplary hydrophilic Wnt-related polypeptides include Wnt-related polypeptides appended with one or more cyclodextran moieties, or Wnt-related polypeptides that are otherwise appended with one or more glycosyl moieties. Other particularly preferred moieties with which a Wnt-related polypeptide can be appended include one or more albumin moieties or one or more antibody moieties.

In any of the foregoing, the invention contemplates modified Wnt-related polypeptides or bioactive fragments thereof, as well as mimetics of full-length Wnt or mimetics of a bioactive fragment of Wnt.

As outlined in detail above, the present invention contemplates a variety of modified Wnt-related polypeptides, wherein the modified Wnt-related polypeptide retains one or more of the biological activities of native or unmodified Wnt polypeptide and further possesses one or more advantageous physiochemical properties. By way of another example of modified Wnt-related polypeptides, and methods for using such polypeptides, the present invention contemplates modified Wnt-related polypeptides appended with one or more albumin moieties. As outlined in detail for pegylated Wnt-related polypeptides, albumin modified Wnt-related polypeptides can be modified with one or more albumin moieties and such albumin moieties can be appended to an N-terminal, C-terminal, and/or an internal amino acid residue. Detailed descriptions of albumin and exemplary methods that can be used to append albumin moieties to a Wnt-related polypeptide can be found in U.S. application 2004/0010134, the disclosure of which is hereby incorporated by reference in its entirety.

Additional modified Wnt-related polypeptides are also contemplated by the present invention and include Wnt-related polypeptides modified with one or more albumin moiety, Wnt-related polypeptides modified with one or more antibody moiety (e.g., IgG moiety, IgM moiety, IgE moiety, etc), and Wnt-related polypeptides otherwise modified so as to increase their hydrophilicity. A variety of methods can be used to append one or more moieties to a Wnt-related polypeptide, and exemplary methods are found in the following references which are hereby incorporated by reference in their entirety: U.S. application No. 2004/0010134, U.S. Pat. No. 6,664,331, U.S. Pat. No. 6,624,246, U.S. Pat. No. 6,610,281, WO03/070805, U.S. Pat. No. 6,602,952, U.S. Pat. No. 6,602,498, U.S. Pat. No. 6,541,543, U.S. Pat. No. 6,541,015, U.S. Pat. No. 6,515,100, U.S. Pat. No. 6,514,496, U.S. Pat. No. 6,514,491, U.S. Pat. No. 6,495,659, U.S. Pat. No. 6,461,603, U.S. Pat. No. 6,461,602, U.S. Pat. No. 6,436,386, U.S. Pat. No. 5,900,461, WO03/040211, WO03/000777, U.S. Pat. No. 6,448,369, U.S. Pat. No. 6,437,025, and Roberts et al. (2002) Advanced Drug Delivery Reviews 54: 459-476.

Classes of Modifications

The present invention contemplates compositions comprising modified Wnt-related polypeptides. In one embodiment, the modified Wnt polypeptide is a hydrophilically modified Wnt-related polypeptide. In another embodiment, the modified Wnt-related polypeptide is a pegylated Wnt polypeptide. The invention contemplates that a modified Wnt-related polypeptide may be appended with one or more moieties (or with a moiety containing one or more PEG moieties). The moieties may be the same or may be different, and the moieties may be arranged linearly or in a branched configuration.

Furthermore, the hydrophilically modified Wnt-related polypeptide may optionally contain a palmitoyl moiety on Cys77, as identified in some native preparations, or may contain a different (e.g., not a palmitoyl moiety) hydrophobic moiety on Cys77. In embodiments where the modified Wnt-related polypeptide contains a hydrophobic moiety on Cys77, the invention contemplates Wnt-related polypeptides that further comprise (i) one or more hydrophilic moieties on Cys77; (ii) one or more hydrophilic moieties on Cys77 and one or more hydrophilic moieties on one or more additional amino acid residues; (iii) one or more hydrophilic moieties on one or more additional amino acid residues other than Cys77.

However, such hydrophilically modified Wnt-related polypeptides are only one embodiment of the invention, and Wnt-related polypeptides that are hydrophilically modified but do not contain a hydrophobic modification on Cys77 or on any other amino acid residues are also contemplated.

The invention contemplates that the Wnt polypeptides can be modified by appending a moiety to the N-terminal amino acid residue (e.g., by appending a PEG containing moiety to the primary amine of the N-terminal amino acid residue). Furthermore, the invention contemplates that the Wnt-related polypeptides can be modified by appending a moiety to an internal amino acid residue or to the C-terminal amino acid residue (e.g., by appending a PEG containing moiety to an amine containing amino acid residue). Additionally, the invention contemplates addition or substitution of a free amine containing amino acid residue to a Wnt-related polypeptides to provide a site for attachment of one or more PEG containing moiety.

The present invention provides modified Wnt-related polypeptides, and methods of using these modified Wnt-related polypeptides in vitro and in vivo. The modified Wnt polypeptides of the present invention should retain one or more of the biological activities of unmodified and/or native Wnt. Additionally, preferable modified Wnt-related polypeptides possess one or more advantageous physiochemical characteristics in comparison to native and/or unmodified Wnt. Accordingly, modified Wnt-related polypeptides not only provide additional possible compositions for manipulating Wnt signaling in vitro or in vivo, such modified Wnt-related polypeptides may also provide Wnt-related polypeptides with improved properties in comparison to the prior art. Exemplary modified Wnt-related polypeptides include pegylated Wnt polypeptides.

The present invention contemplates appending Wnt-related polypeptides with any of a number of PEG containing moieties, as well as any of a number of methods for appending such PEG containing moieties to the primary amine of the N-terminal amino acid residue, an amine of an amine containing internal amino acid residues, and/or an amine of an amine containing C-terminal amino acid residue. Furthermore, the invention contemplates appending PEG containing moieties via reactive amino acid residues including cysteine residues.

Various PEG containing moieties are well known in the art. For example, several companies manufacture and market a variety of PEG containing reagents for use in pegylating peptides. In the earlier days of pegylation technology, pegylation occurred via reactive amino acid residues such as cysteines. Although powerful, such methodologies required either that the peptide of interest contain a cysteine residue, or required mutating or appending a cysteine residue to the peptide of interest. Such methodologies are extremely useful, and are well-known in the art. Given that Wnt-related polypeptides, for example Wnt3A contain a number of cysteine residues, methods of appending moieties via a cysteine residue offer a potentially powerful approach for appending moieties to Wnt-related polypeptide.

Additionally, the present invention describes pegylated Wnt-related polypeptides, wherein the PEG containing moiety is attached via a free amine (e.g., the primary amine of the N-terminal amino acid residue, a free amine of an internal amino acid reside, a free amine of a C-terminal amino acid residue, etc.).

Activated PEG containing moieties readily allow the conjugation of PEG containing moieties to primary amine of peptides. Thus, the methods and compositions of the present invention specifically contemplate PEG containing moieties comprising a reactive group (e.g., reactive PEG containing moieties), the invention further contemplates that attachment of the PEG containing moiety to the Wnt-related polypeptides occurs via the reactive group.

Preferable reactive PEG containing moieties readily react with polypeptides at physiological pH (e.g., 7.0, 7.5, 8.0, 8.5, 9.0, and 9.5) and at room temperature. In certain embodiment, the PEG containing moiety is capped with a methoxy PEG. Accordingly, the invention contemplates PEG containing moieties which may include methoxy PEG.

In one aspect, the PEG containing moiety is a lysine-active PEG (also referred to as an active ester containing PEG moiety). Such lysine active PEG containing moieties are particularly useful for either appending a PEG containing moiety to the primary amine of the N-terminal amino acid residue, as well as for appending a PEG containing moiety to an amino acid residue containing an imidazole group or a hydroxyl group (e.g., histidine, tyrosine). Exemplary active esters include, but are not limited to, N-hydroxylsuccinimide (NHS) active esters, succinimidyl propionate (SPA) active esters and, succinimidyl butanate (SBA) active esters. Examples of lysine active PEG containing moieties include, but are not limited to, PEG-N— hydroxylsuccinimide (PEG-NHS), succinimidyl ester of PEG propionic acid (PEG-SPA), and succinimidyl ester of PEG butanoic acid (PEG-SBA). These exemplary PEG containing moieties are illustrated in FIG. 2.

In another aspect, the PEG containing moiety is a PEG aldehyde (also referred to as a PEG thioester). FIG. 3 depicts an exemplary PEG thioester. PEG-thioester containing moieties are specifically designed for conjugation to the N-terminus, and preferable are designed for appending to a cysteine or a histidine.

In another aspect, the PEG containing moiety is a PEG double ester depicted in FIG. 4.

In another aspect, the PEG containing moiety is a PEG benzotriazole carbonate (PEG-BTC) (FIG. 5). Such PEG containing moieties are especially useful for producing modified proteins under mild conditions. The reaction of a PEG-BTC moiety with a polypeptide (e.g., a Wnt polypeptide) results in the attachment of PEG-BTC to the polypeptide via a stable urethane (carbamate) linkage.

In another aspect, the PEG containing moiety is an amine selective reagent such as PEG-ButyrALD (FIG. 6). Such selective reagents allow for more stable modified compositions than previously attainable. However, the invention contemplates the use of other PEG containing moieties bearing aldehyde groups. One specifically contemplated class of aldehyde bearing moieties reacts with primary amines in the presence of sodium cyanoborohydride and includes PEG aldehydes, PEG acetaldehydes, and PEG propionaldehydes.

In another aspect, the PEG containing moiety is a PEG acetaldehyde diethyl acetal (PEG-ACET) (FIG. 7). Such PEG containing moieties are particularly stable against aldol condensation.

In another aspect, the PEG containing moiety is a sulfhydryl-selective PEG. Exemplary sulfhydryl-selective PEGs include PEG-maleimide (PEG-MAL) (FIG. 8) and PEG-vinylsulfone (PEG-VS). Such PEG containing moieties are especially useful for reaction with thiol groups.

In addition to the foregoing PEG containing moieties comprising various reactive groups suitable for appending a PEG containing moiety to a Wnt-related polypeptide, the invention further contemplates PEG containing moieties that comprise both a suitable reactive group, and also another functional group designed to enhance the overall utility of the modified Wnt-related polypeptides.

In one aspect, the PEG containing moiety is protected with either a Boc or Fmoc protecting group (FIG. 9).

In another aspect, the PEG containing moiety is further modified with a detectable moiety. Such detectable moieties can be used to monitor the modified composition. Exemplary detectable moieties include fluorescent moieties such as rhodamine, fluorescein, and derivatives thereof, as well as detectable substrates such as biotin, alkaline phosphotase, and the like.

FIG. 10 shows two examples of PEG containing moieties containing both a reactive group and a detectable moiety: fluorescein-PEG-NHS and biotin-PEG-NHS. We note, however, that any of a range of detectable moieties, as well as any of a number of reactive groups can be readily employed to design related modified Wnt-related polypeptides.

The foregoing examples illustrate the varieties of modified Wnt-related polypeptides contemplated by the present invention. Any of these modified Wnt polypeptides can be synthesized using techniques well known in the art, and these modified Wnt-related polypeptides can be tested using in vitro and in vivo assays to identify modified polypeptides that (i) retain one or more of the biological activities of the corresponding native and/or unmodified Wnt polypeptide and, preferably (ii) possess one or more advantageous physiochemical characteristics in comparison to the corresponding native and/or unmodified Wnt polypeptide. In the context of the present invention, the one or more biological activites retain by the modified Wnt polypeptides includes the ability to promote Wnt signaling via the canonical Wnt signaling pathway.

In addition, one of skill in the art can readily select from amongst a great many additional PEG containing moieties and select appropriate PEG chemistries to append a PEG containing moiety to one or more of an N-terminal amino acid residue, an internal amino acid residue, or a C-terminal amino acid residue of a Wnt-related polypeptide. Examples of additional PEG containing moieties and PEG chemistries are described, for example, in Roberts et al. (2002) Advanced Drug Delivery Reviews 54: 459-476, U.S. Pat. No. 6,664,331, U.S. Pat. No. 6,624,246, U.S. Pat. No. 6,610,281, WO03/070805, U.S. Pat. No. 6,602,952, U.S. Pat. No. 6,602,498, U.S. Pat. No. 6,541,543, U.S. Pat. No. 6,541,015, U.S. Pat. No. 6,515,100, U.S. Pat. No. 6,514,496, U.S. Pat. No. 6,514,491, U.S. Pat. No. 6,495,659, U.S. Pat. No. 6,461,603, U.S. Pat. No. 6,461,602, U.S. Pat. No. 6,436,386, U.S. Pat. No. 5,900,461, WO03/040211, WO03/000777, U.S. Pat. No. 6,448,369, U.S. Pat. No. 6,437,025, the disclosures of which are hereby incorporated by reference in their entirety.

In addition to the aforementioned modified Wnt-related polypeptides, the invention contemplates modified LRP-related polypeptides, and bioactive fragments thereof. Exemplary modified LRP-related polypeptides retain the ability of unmodified LRP to promote Wnt signaling via the canonical Wnt signaling pathway. Further exemplary modified LRP-related polypeptides can be used to promote cardiomyocyte proliferation. The present invention contemplates that LRP-related polypeptides can be modified with one or more hydrophobic or hydrophilic moieties using the same methods and compositions that can be used to modify Wnt-related compostions. Accordingly, throughout the present application references to methods and compositions for appending one or more moieties to a Wnt-related composition should be considered exemplary of the methods and compositions that can be used to modify LRP-related polypeptides.

Additional Agents that Act at the Cell Surface to Promote Wnt Signaling

The foregoing examples of hydrophobically and hydrophilically modified polypeptides were meant to illustrate the modified polypeptides contemplated by the present invention. As should be clear from the examples provided herein, modified polypeptides of the invention can be appended with 1 or more, 2 or more, 3 or more, 4 or more, 5 or more, or more than 5 moieties. When a polypeptide is appended with more than one moiety, the moieties can be appended to the same amino acid residues or to different amino acid residues. When a polypeptide is appended with more than one moiety, the moieties are independently selected. The independent selection of moieties may include not only various hydrophobic moieties together to produce a hydrophobically modified polypeptide, or various hydrophilic moieties together to produce a hydrophilically modified polypeptide. The invention also contemplates appending a polypeptide with both hydrophobic and hydrophilic moieties to produce a mixed-modified polypeptide. Such a modified polypeptide can be readily evaluated to confirm that it retains one or more biological activities of the corresponding native and/or unmodified polypeptide, and further evulated to assess whether the modified polypeptide possess one or more advantageous physiochemical properties in comparison to the corresponding native and/or unmodified polypeptide.

Antibodies

Antibodies can be used as modulators of the activity of a particular protein. Antibodies can have extraordinary affinity and specificity for particular epitopes. For example, antibodies can act as inhibitors of the function of a particular protein by, for example, binding to a particular protein in such a way that the binding of the antibody to the epitope on the protein can interfere with the function of that protein. Such antibodies may inhibit the function of a protein by sterically hindering the proper protein-protein interactions or occupying active sites. Alternatively the binding of the antibody to an epitope on the particular protein may alter the conformation of that protein such that it is no longer able to properly function.

Antibodies that act as inhibitors of a particular protein may have any of a number of effects. If the function that the antibody inhibits is typically an activating function (e.g., the protein endogenously acts to promote cell proliferation or differentiation; the protein endogenously acts to promote signal transduction via a particular signaling pathway), then inhibition of the activity of this protein (e.g, antagonism of the endogenous function of that protein) will have a repressive effect on the cell or tissue (e.g., the antibody will inhibit cell proliferation or differentiation; the antibody will inhibit signal transduction via a particular signaling pathway). If, on the other hand, the function that the antibody inhibits is typically a repressive function (e.g., the protein endogenously acts to inhibit cell proliferation or differentiation; the protein endogenously acts to inhibit signal transduction via a particular signaling pathway), then inhibition of the activity of this protein will have an activating effect on the cell or tissue (e.g., the antibody will promote cell proliferation or differentiation; the antibody will promote signal transduction via a particular signaling pathway).

Monoclonal or polyclonal antibodies can be made using standard protocols (See, for example, Antibodies: A Laboratory Manual ed. by Harlow and Lane (Cold Spring Harbor Press: 1988)). A mammal, such as a mouse, a hamster, a rat, a goat, or a rabbit can be immunized with an immunogenic form of the peptide. Techniques for conferring immunogenicity on a protein or peptide include conjugation to carriers or other techniques well known in the art.

Following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a particular polypeptide and monoclonal antibodies isolated from a culture comprising such hybridoma cells.

The term antibody as used herein is intended to include fragments thereof which are also specifically reactive with a particular polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific and chimeric molecules having affinity for a particular protein conferred by at least one CDR region of the antibody.

Both monoclonal and polyclonal antibodies (Ab) directed against a particular polypeptides, and antibody fragments such as Fab, F(ab)₂, Fv and scFv can be used to modulate the activity of a particular protein. Such antibodies can be used either in an experimental context to further understand the role of a particular protein in a biological process, or in a therapeutic context.

In addition to the use of antibodies as agents, the present invention contemplate that antibodies raised against a particular protein can also be used to monitor the expression of that protein in vitro or in vivo (e.g., such antibodies can be used in immunohistochemical staining). In any of the foregoing, the invention contemplates that antibodies can be readily humanized to make them suitable for administration to human patients.

The present invention contemplates methods and compositions comprising agents that act at the cell surface to promote Wnt signaling via the canonical Wnt signaling pathway. Exemplary antibodies are anti-LRP antibodies. Exemplary anti-LRP antibodies include antibodies immunoreactive with all or a portion of an LRP-related polypeptide. Further exemplary anti-LRP antibodies include antibodies immunoreactive with all or a portion of an LRP-related polypeptide represented in SEQ ID NO: 80, SEQ ID NO: 82, SEQ ID NO: 84, or SEQ ID NO: 86.

Still further exemplary anti-LRP antibodies include antibodies immunoreactive with an N-terminal, extracellular fragment of an LRP-related polypeptide. Such exemplary anti-LRP antibodies include antibodies immunoreactive with one or more EGF repeat, one or more LDLR repeat, one or more YWTD spacer region, or a combination thereof (Liu et al. (2003) Mol. Cell Biology 23: 5825-5835).

The present invention contemplates that one class of agents that act at the cell surface to promote Wnt signaling via the canonical Wnt signaling pathway are anti-LRP antibodies. Without being bound by theory, anti-LRP antibodies immunoreactive with all or a portion of LRP5 or LRP6 (e.g., immunoreactive with all or a portion of the extracellular domain of LRP5 or LRP6) can be used to relieve repressive LRP-dimerization, and thereby promote Wnt signaling via the canonical Wnt signaling pathway. Such an antibody would relieve the repressive LRP-dimerization in much the same way that overexpression of fragments of LRP comprising an N-terminal deletion relieve the repression and constituitively activate Wnt signaling (Liu et al. (2003) Mol. Cell Biology 23: 5825-5835; Brennan et al. (2004) Oncogene).

(iv) Exemplary Expression Methods

The systems and methods described herein also provide expression vectors containing a nucleic acid encoding a Wnt-related polypeptide or an LRP-related polypeptide operably linked to at least one transcriptional regulatory sequence. Exemplary nucleic acids include, but are not limited to, a nucleic acid encoding a Wnt-related polypeptide, a nucleic acid encoding a bioactive fragment of a Wnt-related polypeptide, a nucleic acid encoding an LRP-related polypeptide, and a nucleic acide encoding an fragment comprising an N-terminal deletion of an LRP-related polypeptide. Accordingly, the invention contemplates delivery of a Wnt-related polypeptide, modified polypeptide, or bioactive fragment thereof, as well as delivery of a nucleic acid sequence encoding a Wnt-related polypeptide, or bioactive fragment thereof. The invention contemplates that delivery of either a composition comprising a nucleic acid sequence or delivery of a composition comprising a polypeptide can be used, for example, to (i) promote cardiac cell proliferation including, but not limited to, cardiomyocyte proliferation, (ii) promote cardiac cell regeneration including, but not limited to, cardiomyocyte regeneration, (iii) promote cardiac cell survival including, but not limited to, cardiomyocyte survival, (iv) treat any of a number of injuries and diseases that decrease cardiac function.

Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences may be used in these vectors to express nucleic acid sequences encoding the agents of this invention. Such useful expression control sequences, include, for example, a viral LTR, such as the LTR of the Moloney murine leukemia virus, the LTR of the Herpes Simplex virus-1, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs can be used to deliver nucleic acids encoding the subject polypeptides. Thus, another aspect of the invention features expression vectors for in vivo or in vitro transfection, viral infection and expression of a subject polypeptide in particular cell types. In one embodiment, such recombinantly produced polypeptides can be modified using standard techniques described herein, as well as other methodologies well known to one of skill in the art.

Expression constructs of the subject agents may be administered in biologically effective carriers, e.g. any formulation or composition capable of effectively delivering the recombinant gene to cells in vivo or in vitro. Approaches include insertion of the subject gene in viral vectors including recombinant retroviruses, adenovirus, adeno-associated virus, herpes simplex virus-1, lentivirus, mammalian baculovirus or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct, electroporation or CaPO₄ precipitation. One of skill in the art can readily select from available vectors and methods of delivery in order to optimize expression in a particular cell type or under particular conditions.

Retrovirus vectors and adeno-associated virus vectors have been frequently used for the transfer of exogenous genes. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes. Thus, recombinant retrovirus can be constructed in which part of the retroviral coding sequence (gag, pol, env) has been replaced by nucleic acid encoding one of the subject proteins rendering the retrovirus replication defective. The replication defective retrovirus is then packaged into virions through the use of a helper virus by standard techniques which can be used to infect a target cell. Protocols for producing recombinant retroviruses and for infecting cells in vitro or in vivo with such viruses can be found in Current Protocols in Molecular Biology, Ausubel, F. M. et al. (eds.) Greene Publishing Associates, (2000), and other standard laboratory manuals. Examples of suitable retroviruses include pBPSTR1, pLJ, pZIP, pWE and pEM which are known to those skilled in the art. Examples of suitable packaging virus lines for preparing both ecotropic and amphotropic retroviral systems include ψCrip, ψCre, ψ2, ψAm, and PA317.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein; or coupling cell surface receptor ligands to the viral env proteins. Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences which control expression of the gene of the retroviral vector such as tetracycline repression or activation.

Another viral gene delivery system which has been employed utilizes adenovirus-derived vectors. The genome of an adenovirus can be manipulated so that it encodes and expresses a gene product of interest but is inactivated in terms of its ability to replicate in a normal lytic viral life cycle. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they can be used to infect a wide variety of cell types, including airway epithelium, endothelial cells, hepatocytes, and muscle cells. Furthermore, the virus particle is relatively stable and amenable to purification and concentration, and as above, can be modified so as to affect the spectrum of infectivity.

Yet another viral vector system is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158: 97-129). It is also one of the few viruses that may integrate its DNA into non-dividing cells, and exhibits a high frequency of stable integration.

Another viral delivery system is based on herpes simplex-1 (HSV-1). HSV-1 based vectors have been shown to infect a variety of cells including post mitotic cells such as neuronal cells (Agudo et al. (2002) Human Gene Therapy 13: 665-674; Latchman (2001) Neuroscientist 7: 528-537; Goss et al. (2002) Diabetes 51: 2227-2232; Glorioso (2002) Current Opin Drug Discov Devel 5: 289-295; Evans (2002) Clin Infect Dis 35: 597-605; Whitley (2002) Journal of Clinical Invest 110: 145-151; Lilley (2001) Curr Gene Ther 1: 339-359).

The above cited examples of viral vectors are by no means exhaustive. However, they are provided to indicate that one of skill in the art may select from well known viral vectors, and select a suitable vector for expressing a particular protein in a particular cell type.

In addition to viral transfer methods, such as those illustrated above, non-viral methods can be used to express a subject polypeptide. Many nonviral methods of gene transfer rely on normal mechanisms used by cells for the uptake and intracellular transport of macromolecules. Exemplary gene delivery systems of this type include liposomal derived systems, poly-lysine conjugates, and artificial viral envelopes.

It may sometimes be desirable to introduce a nucleic acid directly to a cell, for example a cell in culture or a cell in an animal. Such administration can be done by injection of the nucleic acid (e.g., DNA, RNA) directly at the desired site. Such methods are commonly used in the vaccine field, specifically for administration of “DNA vaccines”, and include condensed DNA (U.S. Pat. No. 6,281,005).

In addition to administration of nucleic acids, the systems and methods described herein contemplate that polypeptides may be administered directly. Some proteins, for example factors that act extracellularly by contacting a cell surface receptor, such as growth factors, may be administered by simply contacting cells with said protein. For example, cells are typically cultured in media which is supplemented by a number of proteins such as FGF, TGFβ, insulin, etc. These proteins influence cells by simply contacting the cells.

In another embodiment, a polypeptide is directly introduced into a cell. Methods of directly introducing a polypeptide into a cell include, but are not limited to, protein transduction and protein therapy. For example, a protein transduction domain (PTD) can be fused to a nucleic acid encoding a particular agent, and the fusion protein is expressed and purified. Fusion proteins containing the PTD are permeable to the cell membrane, and thus cells can be directly contacted with a fusion protein (Derossi et al. (1994) Journal of Biological Chemistry 269: 10444-10450; Han et al. (2000) Molecules and Cells 6: 728-732; Hall et al. (1996) Current Biology 6: 580-587; Theodore et al. (1995) Journal of Neuroscience 15: 7158-7167).

Although some protein transduction based methods rely on fusion of a polypeptide of interest to a sequence which mediates introduction of the protein into a cell, other protein transduction methods do not require covalent linkage of a protein of interest to a transduction domain. At least two commercially available reagents exist that mediate protein transduction without covalent modification of the protein (Chariot™, produced by Active Motif, www.activemotif.com and Bioporter® Protein Delivery Reagent, produced by Gene Therapy Systems, www.genetherapysystems.com).

Briefly, these protein transduction reagents can be used to deliver proteins, peptides and antibodies directly to cells including mammalian cells. Delivery of proteins directly to cells has a number of advantages. Firstly, many current techniques of gene delivery are based on delivery of a nucleic acid sequence which must be transcribed and/or translated by a cell before expression of the protein is achieved. This results in a time lag between delivery of the nucleic acid and expression of the protein. Direct delivery of a protein decreases this delay. Secondly, delivery of a protein often results in transient expression of the protein in a cell.

As outlined herein, protein transduction mediated by covalent attachment of a PTD to a protein can be used to deliver a protein to a cell. These methods require that individual proteins be covalently appended with PTD moieties. In contrast, methods such as Chariot™ and Bioporter® facilitate transduction by forming a noncovalent interaction between the reagent and the protein. Without being bound by theory, these reagents are thought to facilitate transit of the cell membrane, and following internalization into a cell the reagent and protein complex disassociates so that the protein is free to function in the cell.

In another aspect, this application includes Wnt-related compositions which are polypeptides, modified polypeptides, or bioactive fragments. Recombinant polypeptides of the present invention include, but are not limited to Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, Wnt16, or bioactive fragments thereof. In one embodiment, the polypeptide is selected from Wnt3, Wnt 3A, or a bioactive fragment thereof. The invention further contemplates the use of variants of such proteins that promote Wnt signaling, wherein the variant retains one or more of the biological activities of native or un-modified Wnt polypeptide. Exemplary variants are at least 60% identical, more preferably 70% identical and most preferably 80% identical with SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment thereof. Additional preferred embodiments include recombinant polypeptides comprising an amino acid sequence at least 83%, 85%, 90%, 95%, 96%, 97%, 98%, or 99% identical to an amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, or a bioactive fragment thereof.

In any of the foregoing, the present invention specifically contemplates compositions comprising modified Wnt-related polypeptides, including modified Wnt3A-related polypeptides and bioactive fragments thereof. Preferred modified Wnt-related polypeptides include hydrophobically modified Wnt-related polypeptides and hydrophilically modified Wnt-related polypeptide. Such modified polypeptides retain one or more of the biological activities of the corresponding native and/or unmodified Wnt polypeptide. Exemplary biological activities include: (i) bind to a frizzled receptor; (ii) promote Wnt signaling. Exemplary biological activities of a modified Wnt3A polypeptide include: (i) bind to a frizzled receptor; (ii) promote Wnt signaling via β-catenin; (iii) promote the expression, activity, nuclear localization, and/or stability of β-catenin. In the context of the present invention, suitable compositions (e.g., polypeptides, modified polypeptides, variants, bioactive fragments thereof) retain one or more biological activities, wherein the one or more retained biological activities include the ability to promote Wnt signaling via the canonical Wnt signaling pathway.

The present invention contemplates that the modified polypeptides can be modified on the N-terminal amino acid residue, on the C-terminal amino acid residue, or on an internal amino acid residue. Furthermore the invention contemplates that the modified polypeptides can be modified with one or more moieties, two or more moieties, three or more moieties, four or more moieties, five or more moieties, or greater than five moieties. When the polypeptide is modified with two or more moieties, each moiety is independently selected, and may be the same as or different from any other moiety appended to that polypeptide. Furthermore, the moieties may be appended to the same amino acid residue and/or to different amino acid residues. Accordingly, the invention contemplates modified polypeptides that are modified one or more times on an N-terminal amino acid residue, C-terminal amino acid residue, and/or on one or more internal amino acid residue.

Additionally, the invention appreciates that a native form (either a predominant native form or a minor native form) of some Wnt polypeptides are modified. For example, some groups reported that a native form of Wnt3A is modified with a palmitoyl group on Cys77. Accordingly, the present invention contemplates modified polypeptides and methods of using modified polypeptides that include (i) modified polypeptides modified in the same manner as a native polypeptide; (ii) modified polypeptides including both a native modification and one or more additional modifications at the same position; (iii) modified polypeptides including both a native modification and one or more additional modifications at a different position; (iv) modified polypeptides modified with a different modification on the same position as the native polypeptide; (v) modified polypeptides modified with a different modification on the same position as the native polypeptide and modified on one or more additional positions.

This application also describes methods for producing the subject polypeptides. For example, a host cell transfected with a nucleic acid vector directing expression of a nucleotide sequence encoding the subject polypeptides can be cultured under appropriate conditions to allow expression of the peptide to occur. The polypeptide may be secreted and isolated from a mixture of cells and medium containing the recombinant polypeptide. Alternatively, the peptide may be expressed cytoplasmically and the cells harvested, lysed and the protein isolated. A cell culture includes host cells, media and other by-products. Suitable media for cell culture are well known in the art. The recombinant polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In one example, the recombinant polypeptide is a fusion protein containing a domain which facilitates its purification, such as a GST fusion protein. In another example, the subject recombinant polypeptide may include one or more additional domains which facilitate immunodetection, purification, and the like. Exemplary domains include HA, FLAG, GST, His, and the like. Further exemplary domains include a protein transduction domain (PTD) which facilitates the uptake of proteins by cells. Recombinantly expressed proteins can be modified using methods disclosed herein, as well as those well known to one of skill in the art.

This application also describes a host cell which expresses a recombinant form of the subject polypeptides. The host cell may be a prokaryotic or eukaryotic cell. Thus, a nucleotide sequence derived from the cloning of a protein encoding all or a selected portion (either an antagonistic portion or a bioactive fragment) of the full-length protein, can be used to produce a recombinant form of a polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known proteins, e.g. insulin, interferons, human growth hormone, IL-1, IL-2, and the like. Similar procedures, or modifications thereof, can be employed to prepare recombinant polypeptides by microbial means or tissue-culture technology in accord with the subject invention. Such methods are used to produce experimentally useful proteins that include all or a portion of the subject nucleic acids. For example, such methods are used to produce fusion proteins including domains which facilitate purification or immunodetection, and to produce recombinant mutant forms of a protein).

The recombinant genes can be produced by ligating a nucleic acid encoding a protein, or a portion thereof, into a vector suitable for expression in either prokaryotic cells, eukaryotic cells, or both. Expression vectors for production of recombinant forms of the subject polypeptides include plasmids and other vectors. For instance, suitable vectors for the expression of a polypeptide include plasmids of the types: pBR322-derived plasmids, pEMBL-derived plasmids, pEX-derived plasmids, pGEX-derived plasmids, pTrc-His-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae.

Many mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo, pBacMam-2, and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. For other suitable expression systems for both prokaryotic and eukaryotic cells, as well as general recombinant procedures, see Molecular Cloning A Laboratory Manual, 3rd Ed., ed. by Sambrook and Russell (Cold Spring Harbor Laboratory Press: 2001).

In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUWI), and pBlueBac-derived vectors (such as the β-gal containing pBlueBac III).

The present invention also makes available isolated polypeptides which are isolated from, or otherwise substantially free of other cellular and extracellular proteins. The term “substantially free of other cellular or extracellular proteins” (also referred to herein as “contaminating proteins”) or “substantially pure or purified preparations” are defined as encompassing preparations having less than 20% (by dry weight) contaminating protein, and preferably having less than 5% contaminating protein. Functional forms of the subject proteins can be prepared as purified preparations by using a cloned gene as described herein. By “purified”, it is meant, when referring to peptide or nucleic acid sequences, that the indicated molecule is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water and buffers can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

Isolated peptidyl portions of proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. Chemically synthesized proteins can be modified using methods described herein, as well as methods well known in the art.

The recombinant polypeptides of the present invention also include versions of those proteins that are resistant to proteolytic cleavage. Variants of the present invention also include proteins which have been post-translationally modified in a manner different than the authentic protein. Modification of the structure of the subject polypeptides can be for such purposes as enhancing therapeutic or prophylactic efficacy, or stability (e.g., ex vivo shelf life and resistance to proteolytic degradation in vivo). Such modified peptides, when designed to retain at least one activity of the naturally-occurring form of the protein, are considered functional equivalents of the polypeptides described in more detail herein. Such modified peptides can be produced, for instance, by amino acid substitution, deletion, or addition.

For example, it is reasonable to expect that, in some instances, an isolated replacement of a leucine with an isoleucine or valine, an aspartate with a glutamate, a threonine with a serine, or a similar replacement of an amino acid with a structurally related amino acid (e.g., isosteric and/or isoelectric mutations) may not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 5th ed. by Berg, Tymoczko and Stryer, WH Freeman and Co.: 2002). Whether a change in the amino acid sequence of a peptide results in a functional variant (e.g. functional in the sense that it acts to mimic or antagonize the wild-type form) can be determined by assessing the ability of the variant peptide to produce a response in cells in a fashion similar to the wild-type protein, or competitively inhibit such a response. Polypeptides in which more than one replacement has taken place can readily be tested in the same manner.

Advances in the fields of combinatorial chemistry and combinatorial mutagenesis have facilitated the making of polypeptide variants (Wissmanm et al. (1991) Genetics 128: 225-232; Graham et al. (1993) Biochemistry 32: 6250-6258; York et al. (1991) Journal of Biological Chemistry 266: 8495-8500; Reidhaar-Olson et al. (1988) Science 241: 53-57). Given one or more assays for testing polypeptide variants, one can assess whether a given variant retains one or more of the biological activities of the corresponding native polypeptide.

To further illustrate, the invention contemplates a method for generating sets of combinatorial mutants, as well as truncation mutants, and is especially useful for identifying potential variant sequences that retain one or more of the biological activities of a native Wnt polypeptide. The purpose of screening such combinatorial libraries is to generate, for example, novel variants.

In one aspect of this method, the amino acid sequences for a population of Wnt-related polypepitdes are aligned, preferably to promote the highest homology possible. Amino acids which appear at each position of the aligned sequences are selected to create a degenerate set of combinatorial sequences. In one example, the variegated library of variants is generated by combinatorial mutagenesis at the nucleic acid level, and is encoded by a variegated gene library. For instance, a mixture of synthetic oligonucleotides can be enzymatically ligated into gene sequences such that the degenerate set of potential sequences are expressible as individual polypeptides, or alternatively, as a set of larger fusion proteins (e.g. for phage display) containing the set of sequences therein.

The library of potential variants can be generated from a degenerate oligonucleotide sequence using a variety of methods. Chemical synthesis of a degenerate gene sequence can be carried out in an automatic DNA synthesizer, and the synthetic genes then ligated into an appropriate expression vector. One purpose of a degenerate set of genes is to provide, in one mixture, all the sequences encoding the desired set of potential variant sequences. The synthesis of degenerate oligonucleotides is known in the art.

A range of techniques are known for screening gene products of combinatorial libraries made by point mutations, and for screening cDNA libraries for gene products having a certain property. Such techniques will be generally adaptable for rapid screening of the gene libraries generated by the combinatorial mutagenesis of Wnt-related polypeptides. These techniques are also applicable for rapid screening of other gene libraries. One example of the techniques used for screening large gene libraries includes cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

(v) Methods of Screening

The present application describes methods and compositions for promoting cardiac cell proliferation and/or regeneration. In certain embodiments, the invention provides methods and compositions for promoting cardiomyocyte proliferation and/or regeneration. The present application further describes methods and compositions for treating a wide range of injuries and diseases of the cardiovascular system, including injuries and diseases that result in a decrease of myocardial function. One aspect of the present invention are compositions and methods of using Wnt-related polypeptides. In one embodiment, the invention provides various modified Wnt-related polypeptides that can be used in any of the methods of the present invention. Such modified Wnt-related polypeptides, including but not limited to modified Wnt3 polypeptides and modified Wnt3A polypeptides retain the biological activity of native and/or unmodified Wnt (e.g., Wnt3A), and may also possess one or more advantageous physiochemical activities in comparison to native and/or unmodified Wnt (e.g., Wnt3A). Modified Wnt polypeptides for use in the methods of the present invention retain at least one of the biological activities of the native or unmodified polypeptide, wherein the at least one retained biological activity includes the ability to promote Wnt signaling via the canonical Wnt signaling pathway.

In light of the importance of providing improved methods and compositions for treating the wide range of conditions that hamper functional performance of cardiac muscle, and in light of the finding that certain modified Wnt-related polypeptides retain the functional activity of native or unmodified Wnt, the present invention further provides screening methods to identify, characterize, and/or optimize modified Wnt-related polypeptides (e.g., Wnt3, Wnt3A, etc.). Exemplary modified Wnt-related polypeptides identified, characterized, and/or optimized by the methods of the present invention retain one or more of the following biological activities of the corresponding native Wnt polypeptide: (i) promote binding to a frizzled receptor; (ii) promote Wnt signaling. By way of example, the invention contemplates the identification, characterization, and/or optimization of modified Wnt3A polypeptides that retain one or more of the biological activities of native and/or unmodified Wnt3A: (i) promote binding to a frizzled receptor; (ii) promote Wnt signaling via the canonical Wnt signaling pathway; (iii) promote the expression, activity, nuclear localization, and/or stability of β-catenin. Additionally, modified polypeptides that retain one or more of the biological activities of the corresponding native and/or unmodified Wnt can be further screened to identify modified polypeptides that possess one or more advantageous physiochemical activities in comparison to the corresponding native and/or unmodified polypeptide. Modified Wnt polypeptides for use in the methods of the present invention retain at least one of the biological activities of the native or un-modified polypeptide, wherein the at least one retained biological activity includes the ability to promote Wnt signaling via the canonical Wnt signaling pathway.

The screening methods described herein can be used to identify Wnt-related polypeptides comprising one or more modifications appended to a native or variant Wnt amino acid sequence. The screening methods described herein can be used to identify and characterize a range of modified Wnt-related polypeptides. Exemplary Wnt-related polypeptides can be selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, Wnt16, as well as bioactive fragments of any of the foregoing. Based upon our knowledge of particular modifications that preserve the functional activity of Wnt3A, the present screening methods allow the identification of useful modifications of any Wnt polypeptide. Such modified Wnt-related polypeptides can then be formulated for their particular use. For example, modified polypeptides can be formulated for cardiovascular indications, as described herein. In contrast, modified Wnt1 polypeptides can be formulated for neuronal indications.

The invention contemplates any of a number of modified Wnt-related polypeptides, wherein the modification increases the hydrophilicity of the Wnt-related polypeptide. Exemplary modifications include PEG containing moieties. Further exemplary modifications include albumin moieties, cyclodextran moieties, antibody moieties, or combinations thereof. In any of the foregoing, preferable modified Wnt-related polypeptides identified, characterized, and/or optimized by the methods of the invention retain one or more of the biological activities of the corresponding native and/or un-modified Wnt polypeptide. Additionally, modified Wnt-related polypeptides so identified can be further examined to determine if the modified Wnt-related polypeptide possesses one or more advantageous, physiochemical property in comparison to the corresponding native and/or un-modified Wnt polypeptide.

The invention further contemplates any of a number of modified Wnt-related polypeptides, wherein the modification increases the hydrophobicity of the Wnt-related polypeptide. Exemplary modifications include sterols, fatty acids, hydrophobic amino acid residues, and hydrophobic peptides. In any of the foregoing, preferable modified Wnt-related polypeptides identified, characterized, and/or optimized by the methods of the invention retain one or more of the biological activities of the corresponding native and/or un-modified Wnt polypeptide. Additionally, modified Wnt-related polypeptides so identified can be further examined to determine if the modified Wnt-related polypeptide possesses one or more advantageous, physiochemical property in comparison to the corresponding native and/or un-modified Wnt polypeptide.

Furthermore, the invention contemplates any of a number of modified polypeptides containing a combination of hydrophilic and hydrophobic moieties. The screening methods of the invention are not biased based on modifications likely to retain biological activity or moieties likely to impart advantageous physiochemical properties. Accordingly, the screening methods of the invention provide the opportunity to identify, characterize, and/or optimize virtually any possible modification.

The screening methods contemplated include screening single candidate modified Wnt-related polypeptides, multiple modified Wnt-related polypeptides, and libraries of modified Wnt-related polypeptides. In many screening programs that test libraries of nucleic acids, polypeptides, chemical compounds and natural extracts, high throughput assays are desirable to increase the number of agents surveyed in a given period of time. Assays that are performed in cell-free systems, such as may be derived with purified or semi-purified proteins, are often preferred as “primary” screens in that they can be generated to permit rapid development and relatively easy detection of an alteration in a molecular target which is mediated by a test agent. Cell free systems include in vitro systems (preparations of proteins and agents combined in a test tube, Petri dish, etc.), as well as cell free systems such as those prepared from egg extracts or reticulocyte lysates. Moreover, the effects of cellular toxicity and/or bioavailability of the test agents can be generally ignored in such a system, the assay instead being focused primarily on the effect of the agent. Thus, in the context of the present invention, large numbers of candidate, modified Wnt-related polypeptides can be tested in a cell free assay to rapidly assess whether the modified polypeptide retains a biological activity of the corresponding native polypeptide. By way of specific example, modified Wnt-related polypeptides can be tested in a cell free assay to measure binding to a frizzled receptor.

The efficacy of the agent can be assessed by generating dose response curves from data obtained using various concentrations of the test agent. Moreover, a control assay can also be performed to provide a baseline for comparison. Such candidates can be further tested for efficacy in promoting Wnt signaling in vitro in Wnt responsive culture cells. Candidate modified Wnt-related polypeptides can be tested in a general Wnt responsive cell line, in a cardiac-derived Wnt-responsive cell line, or in a primary culture of cardiac-derived Wnt-responsive cells. Additional cell-based assays include measuring the biological activity of the modified Wnt-related polypeptide in comparison to the native and/or unmodified polypeptide using a reporter cell line. For example, a catenin report cell line allows rapid screening to identify modified polypeptides that retain the ability to promote the expression, activity, and/or stability of β-catenin.

The foregoing cell free and cell-based assays provide examples of the methods that can be used to rapidly screen modified Wnt-related polypeptides to identify, characterize, and/or optimize modified Wnt-related polypeptides that retain one or more of the biological activities of the corresponding native and/or unmodified Wnt polypeptide. Additionally, the modified Wnt-related polypeptides that retain one or more of the biological activities of the corresponding native and/or unmodified Wnt polypeptide can be further tested to determine whether it possesses one or more advantageous physiochemical property in comparison to the corresponding native and/or unmodified Wnt polypeptide.

Additionally, we note that methods of screening can be conducted in vivo in either wildtype or mutant animals. Exemplary mutant animals include animal models of cardiac disease, or Wnt homozygous or hemizygous mice. Exemplary wildtype animals include, but are not limited to, any non-human animal such as mice, rats, rabbits, cats, dogs, sheep, pigs, goats, cows, and non-human primates.

Regardless of the methodology used to identify, characterize, and/or optimize a modified Wnt-related polypeptide, such modified polypeptide will have a range of in vitro and in vivo applications. For example, modified polypeptides that retain the biological activity of the native polypeptide provide additional reagents for use in vitro and in vivo. Furthermore, certain modified polypeptides that retain the biological activity of the native and/or unmodified polypeptide also possess one or more advantageous physiochemical property in comparison to the native and/or unmodified polypeptide. These modified polypeptides represent a novel class of Wnt-related polypeptides that may be particularly well suited for therapeutic or laboratory use. Accordingly, the invention further contemplates the use of a modified Wnt-related polypeptide identified by the screening methods of the invention. Identified Wnt-related polypeptides may be used alone or in combination with other agents, or may be formulated in a pharmaceutically acceptable carrier.

Exemplary modified Wnt-related polypeptides can be tested using any of a number of well known assays to confirm that the modified Wnt-related polypeptide retain the ability to promote Wnt signaling via the canonical Wnt signaling pathway. Such assays include, but are not limited to, (i) the examination of β-catenin expression, nuclear localization, and/or stability in response to a Wnt polypeptide, (ii) the examination of GSK3β phosphorylation in response to a Wnt polypeptide, (iii) the examination of the expression of an endogenous downstream target gene in the canonical Wnt signaling pathway, (iv) the examination of the expression of a reporter construct responsive to signaling via the canonical Wnt signaling pathway.

(vi) Exemplary Injuries and Conditions

The methods and compositions of the present invention provide a treatment for any of a wide range of injuries and diseases that compromise the functional performance of cardiac tissue. Because the methods and compositions of the present invention promote, for example, cardiomyocyte or other cardiac cell proliferation, regeneration, and/or survival, and thus overcome the typical scarring response of cardiac tissue to myocardial damage, these methods and compositions help restore cardiac function independent of the cause of the original injury. Accordingly, the present invention has broad applicability to a wide range of conditions, including developmental disorders and congenital defects.

As outlined in detail throughout the application, the invention contemplates administration of any of the Wnt-related compositions of the invention alone, in combination with other Wnt-related compositions, or in combination with any of a number of agents. For example, one or more Wnt-related compositions can be administered consecutively or concurrently with any of the following: one or more agents that promote the binding of a Wnt-related polypeptide to a frizzled receptor; one or more agents that promote cardiomyocyte proliferation; one or more agents that inhibit cardiomyocyte differentiation. Furthermore, the Wnt-related compositions of the invention can be administered as part of a treatment regimen with other conventional therapeutics or procedures appropriate for the particular indication being treated.

By way of further example, the invention contemplates administration of any of the Wnt-related or LRP-related compositions alone or in combination with other Wnt-related or LRP-related compositions, as well as in combination with other agents or treatment regimens. By way of still further example, the invention contemplate administration of one or more agents (e.g., nucleic acid, peptide, polypeptide, antibody) that act at the cell surface to promote Wnt signaling via the canonical Wnt signaling pathway.

By way of non-limiting example, we provide a brief description of exemplary conditions that diminish the functional performance of cardiac tissue. The invention contemplates methods of treating any of these diseases, as well as other diseases that result in myocardial injury that diminishes cardiac function.

Myocardial infarction: Myocardial infarction is defined as myocardial cell death due to prolonged ischemia. Cell death is categorized pathologically as either coagulation or contraction band necrosis, or both, which usually evolves through necrosis, but can result to a lesser degree from apoptosis.

After the onset of myocardial ischemia, cell death is not immediate but takes a finite period to develop (as little as 15 min in some animal models, but even this may be an overestimate). It takes 6 hours before myocardial necrosis can be identified by standard macroscopic or microscopic postmortem examination. Complete necrosis of all myocardial cells at risk requires at least 4-6 hours or longer, depending on the presence of collateral blood flow into the ischemic zone, persistent or intermittent coronary artery occlusion and the sensitivity of the myocytes.

Infarcts are usually classified by size—microscopic (focal necrosis), small (<10% of the left ventricle), medium (10% to 30% of the left ventricle) or large (>30% of the left ventricle) as well as by location (anterior, lateral, inferior, posterior or septal or a combination of locations). The pathologic identification of myocardial necrosis is made without reference to morphologic changes in the epicardial coronary artery tree or to the clinical history.

The term MI in a pathologic context may be preceded by the words “acute, healing or healed.” An acute or evolving infarction is characterized by the presence of polymorphonuclear leukocytes. If the interval between the onset of infarction and death is brief (e.g., 6 h), minimal or no polymorphonuclear leukocytes may be seen. The presence of mononuclear cells and fibroblasts and the absence of polymorphonuclear leukocytes characterize a healing infarction. A healed infarction is manifested as scar tissue without cellular infiltration. The entire process leading to a healed infarction usually requires five to six weeks or more. Furthermore, reperfusion alters the gross and microscopic appearance of the necrotic zone by producing myocytes with contraction bands and large quantities of extravasated erythrocytes.

Infarcts are classified temporally according to the pathologic appearance as follows: acute (6 h to 7 days); healing (7 to 28 days), healed (29 days or more). It should be emphasized that the clinical and ECG timing of an acute ischemic event may not be the same as the pathologic timing of an acute infarction. For example, the ECG may still demonstrate evolving ST-T segment changes, and cardiac troponin may still be elevated (implying a recent infarct) at a time when, pathologically, the infarct is in the healing phase.

Myocardial necrosis results in and can be recognized by the appearance in the blood of different proteins released into the circulation due to the damaged myocytes: myoglobin, cardiac troponins T and I, creatine kinase, lactate dehydrogenase, as well as many others. Myocardial infarction is diagnosed when blood levels of sensitive and specific biomarkers, such as cardiac troponin and the MB fraction of creatine kinase (CK-MB), are increased in the clinical setting of acute ischemia. These biomarkers reflect myocardial damage but do not indicate its mechanism. Thus, an elevated value in the absence of clinical evidence of ischemia should prompt a search for other causes of cardiac damage, such as myocarditis.

The presence, absence, and amount of myocardial damage resulting from prolonged ischemia can be assessed by a number of different means, including pathologic examination, measurement of myocardial proteins in the blood, ECG recordings (ST-T segment wave changes, Q waves), imaging modalities such as myocardial perfusion imaging, echocardiography and contrast ventriculography. For each of these techniques, a gradient can be distinguished from minimal to small to large amounts of myocardial necrosis. Some clinicians classify myocardial necrosis as microscopic, small, moderate and large on the basis of the peak level of a particular biomarker. The sensitivity and specificity of each of these techniques used to detect myocardial cell loss, quantitate this loss and recognize the sequence of events over time, differ markedly. We note that the term myocardial necrosis refers to any myocardial cell death regardless of its cause. Although myocardial infarction is one cause of myocardial necrosis, many other conditions result in necrosis. The methods and compositions of the invention can be used to promote cardiomyocyte proliferation and/or regeneration, and thus improve cardiac function following myocardial infarction, as well as myocardial necrosis caused by any injury or condition.

Noncompaction of the ventricular myocardium: This rare condition, also known as “spongy myocardium,” is a congenital cardiomyopathy of children and adults resulting from arrested myocardial development during embryogenesis. Prior to formation of the epicardial coronary circulation at about 8 weeks of life, the myocardium is a meshwork of interwoven myocardial fibers that form trabeculae and deep trabecular recesses. The increased surface area permits perfusion of the myocardium by direct communication with the left ventricular cavity. Normally, as the myocardium undergoes gradual compaction, the epicardial coronary vessels form.

In this developmental disorder, echocardiography demonstrates a thin epicardium with extremely hypertrophied endocardium and prominent trabeculations with deep recesses. These features tend to be apically localized since compaction would normally proceed from base to apex, and from epicardium to endocardium.

Clinical presentation consists of congestive heart failure with depressed left ventricular systolic function, ventricular arrhythmias, arterial thromboemboli from thrombus formation within the inter-trabecular recesses, as well as restrictive physiology from endocardial fibrosis. The diagnosis can be made echocardiographically, and the entity may be associated with problems of cardiac rhythm. The methods and compositions of the present invention can be used to improve the impairments of the ventricular myocardium, and thus to help restore some of the diminished cardiac function.

The severity of noncompaction of the ventricular myocardium varies among patients, and patients with less severe disease may not present until later in life. In addition to patient populations presenting with only noncompaction of the ventricular myocardium, this disorder is also associated with more complex, multi-system syndromes. For example, noncompaction of the ventricular myocardium is also observed in Wolf-Parkinson-White syndrome and Roifman syndrome. Accordingly, the methods and compositions of the present invention may also be useful in ameliorating the noncompaction of the ventricular myocardium-related effects in these multi-system syndromes.

Congenital heart defects: Congenital heart defects are heart problems present at birth. They happen when the heart does not develop normally before birth. About 8 out of every 1,000 infants are born with one or more heart or circulatory problems. Doctors usually do not know the cause of congenital heart defects, but they do know of some conditions that increase a child's risk of being born with a heart defect. Such conditions include the following: (i) congenital heart disease in the mother or father; (ii) congenital heart disease in a sibling; (iii) diabetes in the mother; (iv) German measles, toxoplasmosis, or HIV infection in the mother; (v) mother's use of alcohol during pregnancy; (vi) mother's use of cocaine or other drugs during pregnancy; (vii) mother's use of certain over-the-counter and prescription medicines during pregnancy.

Congenital heart defects are often detected at birth, however certain defects are not diagnosed until later in life. In still other cases, the heart defect can be detected in utero—prior to birth. Given the broad range of congenital heart defects, as well as the variability in their onset and severity, effective methods of treatment previously needed to be designed for each particular condition. The present methods and compositions provide effective treatment option for this diverse class of disorders that decrease myocardial function. By way of example, congenital heart defects include atrial septal defects (ASD); ventricular septal defects (VSD); atrioventricular canal defects; patent ductus arteriosus; aortic Stenosis; pulmonary stenosis; Ebstein's anomaly; coarctation of the aorta; Tetralogy of Fallot; transposition of the great arteries; persistent truncus arteriosus; tricuspid atresia; pulmonary atresia; total anomalous pulmonary venous connection; and hypoplastic left heart syndrome.

Hypoplastic left heart syndrome: HLHS is an underdevelopment of the left side of the heart characterized by aortic valve atresia, hypoplastic ascending aorta, hypoplastic/atretic mitral valve, and endocardial fibroelastosis. Hypoplastic left heart syndrome is the most common cause of congenital heart failure in newborns, and is responsible for 25% of cardiac deaths occurring during the first week of life. If left untreated, this disorder has a 100% fatality rate.

The PDA usually closes a few days after birth, and separates the left and right sides of the heart. It is at this time that babies with undetected HLHS will exhibit problems as they experience a lack of blood flow to the body. They may look blue, have trouble eating, and breathe rapidly. If left untreated, this heart defect is fatal—usually within the first few days or weeks of life.

Currently, treatment for hypoplastic left heart syndrome requires one of two surgical procedures, and the patient must remain on the drug prostaglandin until surgery is performed. The present invention provides a novel, less invasive treatment option for this otherwise fatal disorder, and can be used alone or in combination with currently available surgical procedures.

Dilated Cardiomyopathy: DCM is an acquired disease characterized by the progressive loss of cardiac contractility. Although the causes of many forms of DCM are unknown, the causes of particular forms of DCM have been identified and include taurine deficiency, adriamycin, and parvovirus. As cardiac contractile function is progressively lost, there is a decrease in cardiac output. Increased blood volume and pressure within the chambers causes them to dilate, most dramatically evident in the left atrium and left ventricle. In response to the poor contractility and decreased cardiac output, the sympathetic nervous system and the renin-angiotensin-aldosterone axis are activated. As with degenerative valve disease, these compensatory mechanisms are initially beneficial, however their chronic activation becomes deleterious. Constant stimulation of the heart by the sympathetic nervous system causes ventricular arrhythmias and myocyte death, while constant activation of the renin-angiotensin-aldosterone axis causes excessive vasoconstriction and retention of sodium and water. The majority of cases exhibit signs of left-sided congestive heart failure, although right-sided signs (ascites) can also occur.

Infection and toxicity: The myocardium is affected by a variety of disease processes including the primary muscle disorders such as dilated cardiomyopathy and hypertrophic cardiomyopathy, degenerative and inflammatory diseases, neoplasia, and infarction. The myocardium is also sensitive to toxin exposure, including adriamycin, oleander, and fluoroacetate.

Myocarditis occurs in all species and may be caused by viral, bacterial, parasitic, and protozoal infection. Canine parvovirus, encephalomyocarditis virus, and equine infectious anemia are viruses with a propensity to cause myocarditis. Myocardial degeneration occurs in lambs, calves, and foals with white muscle disease, and in pigs with mulberry heart disease or hepatosis dietetica. Mineral deficiencies can also result in myocardial degeneration, including iron, selenium, and copper.

Common causes of myocarditis include the following: streptococcus, Salmonella, Clostridium, viral Equine influenza, Borrelia burgdorferi, and Strongylosis. Furthermore, vitamin E and selenium deficiency are known to cause myocardial necrosis.

Cardiac toxins include ionophore antibiotics such as monensin and salinomycin, cantharidin (blister beetle toxicosis), Cryptostegia grandiflora (rubber vine poisoning), and Eupatorium rugosum (white snake root poisoning). These diseases cause typical signs of congestive heart failure—exercise intolerance, tachycardia, and tachypnea.

Current treatment for toxicity and infection aim to stabilize the cardiac symptoms, while addressing the underlying infection or poisoning. However, this approach does not address the actual myocardial damage or necrosis that may result from infection or exposure to toxins. The present invention addresses such myocardial damage resulting from infection and toxicity.

DiGeorze syndrome: DiGeorge syndrome is a multi-system disorder characterized by a few specific cardiac malformations, a sub-set of facial attributes, and certain endocrine and immune anomalies. The cause of DiGeorge syndrome has been identified as a submicroscopic deletion of chromosome 22 in the DiGeorge chromosomal region. It is classified along with velo-cardio-facial syndrome (Shprintzen syndrome) and conotruncal anomaly face syndrome as a 22q11 microdeletion and is sometimes referred to by the simple name 22q11 syndrome.

People with DiGeorge syndrome may have the following congenital heart lesions: tetralogy of Fallot, interrupted aortic arch type B, truncus arteriosus, aberrant left subclavian artery, right infundibular stenosis, or ventricular septal defect. 74% of patients with 22q11 syndrome have conotruncal malformations. 69% of patients are found to have palatal abnormalities including velopharyngeal incompetence (VPI), submucosal cleft palate, and cleft palate. Given the large percentage of DiGeorge syndrome patients with significant cardiac malformation, the methods and compositions of the present invention may be used to help augment, improve, or restore diminished cardiac function.

The foregoing examples are merely illustrative of the broad range of diseases and injuries of vastly different mechanisms that can be treated using the methods and compositions of the present invention. Additionally, we note that although some of the foregoing conditions effect the vasculature, any condition that alters blood flow to or from the heart can damage cardiac tissue. Accordingly, the methods and compositions of the present invention can be used to treat diseases and injuries that primarily affect cardiac tissue, as well as diseases and injuries that affect cardiac tissue secondarily to a defect in the vasculature that alters blood flow or oxygenation of cardiac tissue.

(vii) Pharmaceutical Compositions and Methods of Administration

The present invention provides a large number of compositions comprising agents that act at the cell surface to promote Wnt signaling via the canonical Wnt signaling pathway. Such agents can be used alone or in combination with other agents that act at the cell surface to promote Wnt signaling via the canonical Wnt signaling pathway. Similarly, such agents can be used in combination with other, unrelated agents or with other therapeutic regimens appropriate for the particular application of the invention.

The invention further contemplates pharmaceutical compositions comprising agents that act at the cell surface to promote Wnt signaling via the canonical Wnt signaling pathway. By way of example, such compositions and pharmaceutical compositions include, but are not limited to, Wnt-related polypeptides, modified Wnt-related polypeptides, LRP-related nucleic acids, LRP-related polypeptides, and anti-LRP antibodies. Exemplary pharmaceutical compositions include pharmaceutical compositions comprising (i) a Wnt-related polypeptide, (ii) an active fragment of a Wnt-related polypeptide, (iii) a modified Wnt-related polypeptide, (iv) a modified active fragment of a Wnt-related polypeptide, (v) a Wnt-related nucleic acid, (vi) an LRP-related polypeptide, (vii) an LRP-related nucleic acid, (viii) a fragment of an LRP-related polypeptide comprising an N-terminal deletion, (ix) a nucleic acid encoding a fragment of an LRP-related polypeptide comprising an N-terminal deletion, (x) an anti-LRP antibody, formulated in a pharmaceutically acceptable carrier or excipient. Further exemplary pharmaceutical compositions include pharmaceutical compositions comprising one or more of the above referenced compositions. Still further exemplary pharmaceutical compositions include pharmaceutical compositions comprising one or more of the above referenced compositions, and one or more other agents. Such agents include, but are not limited to, agents that promote the binding of Wnt to a Wnt receptor, agents that promote proliferation of cardiomyocytes, agents that inhibit differentiation of cardiomyocytes, or agents used as a standard non-Wnt related method of treating a condition of cardiac tissue. Throughout this application, any of the foregoing examples of pharmaceutical compositions will be referred to interchangeably as “Wnt-related pharmaceutical compositions” or “Wnt-related compositions.” Wnt-related compositions and pharmaceutical compositions for use in the methods of the present invention retain the cardiac proliferative activity of the Wnt-related composition and furthermore retain at least one of the biological activities of the Wnt-related polypeptide. In the context of the present invention, the at least one biological activity includes the ability to promote Wnt signaling via the canonical Wnt signaling pathway. One of skill in the art will recognize that Wnt-related compositions are just one example of compositions comprising agents that act at the cell surface to promote Wnt signaling via the canonical Wnt signaling pathway. Accordingly, the invention contemplates that any such agent can be administered or used as described herein for Wnt-related compositions.

The pharmaceutical compositions of the present invention are formulated according to conventional pharmaceutical compounding techniques. See, for example, Remington's Pharmaceutical Sciences, 18th Ed. (1990, Mack Publishing Co., Easton, Pa.). Pharmaceutical formulations of the invention can contain the active polypeptide and/or agent, or a pharmaceutically acceptable salt thereof. These compositions can include, in addition to an active polypeptide and/or agent, a pharmaceutically acceptable excipient, carrier, buffer, stabilizer or other material well known in the art. Such materials should be non-toxic and should not interfere with the efficacy of the active agent. Preferable pharmaceutical compositions are non-pyrogenic. The carrier may take a wide variety of forms depending on the route of administration, e.g., intravenous, intravascular, oral, intrathecal, epineural or parenteral, transdermal, etc. Furthermore, the carrier may take a wide variety of forms depending on whether the pharmaceutical composition is administered systemically or administered locally, as for example, via a biocompatible device such as a catheter, stent, wire, or other intraluminal device. Additional methods of local administration include local administration that is not via a biocompatible device.

Illustrative examples of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like.

In one embodiment, the pharmaceutical composition is formulated for sustained-release. An exemplary sustained-release composition has a semi permeable matrix of a solid biocompatible polymer to which the composition is attached or in which the composition is encapsulated. Examples of suitable polymers include a polyester, a hydrogel, a polylactide, a copolymer of L-glutamic acid and ethyl-L-glutamase, non-degradable ethylene-vinyl acetate, a degradable lactic acid-glycolic acid copolymer, and poly-D+-hydroxybutyric acid.

Polymer matrices can be produced in any desired form, such as a film, or microcapsules.

Other sustained-release compositions include liposomally entrapped modified compositions. Liposomes suitable for this purpose can be composed of various types of lipids, phospholipids, and/or surfactants. These components are typically arranged in a bilayer formation, similar to the lipid arrangement of biological membranes. Liposomes containing the Wnt-related compositions of the present invention are prepared by known methods (see, for example, Epstein, et al. (1985) PNAS USA 82:3688-92, and Hwang, et al., (1980) PNAS USA, 77:4030).

Pharmaceutical compositions according to the invention include implants, i.e., compositions or device that are delivered directly to a site within the body and are, preferably, maintained at that site to provide localized delivery. For example, a preferred use for the methods and compositions of the present invention is to promote cardiac cell (e.g., cardiomyocyte) proliferation and/or regeneration. The compositions, including the pharmaceutical compositions described in the present application can be administered systemically, or locally. Locally administered compositions can be delivered, for example, to the pericardial sac, to the pericardium, to the endocardium, to the great vessels surrounding the heart (e.g., intravascularly to the heart), via the coronary arteries, or directly to the myocardium. When delivering to the myocardium to promote proliferation and repair damaged myocardium, the invention contemplates delivering directly to the site of damage or delivery to another site at some distance from the site of damage. Exemplary methods of administering compositions systemically or locally will be described in more detail herein.

The Wnt-related compositions, and pharmaceutical compositions thereof, of the invention also include implants comprising a Wnt-related composition attached to a biocompatible support. This combination of a biocompatible support and a Wnt-related composition can be used to deliver the Wnt-related composition in vivo. Preferable biocompatible supports include, without limitation, stents, wires, catheters, and other intraluminal devices. In one embodiment, the biocompatible support can be delivered intravascularly or intravenously.

The support can be made from any biologically compatible material, including polymers, such as polytetrafluorethylene (PFTE), polyethylene terphthalate, Dacronftpolypropylene, polyurethane, polydimethyl siloxame, fluorinated ethylene propylene (FEP), polyvinyl alcohol, poly(organo)phosphazene (POP), poly-1-lactic acid (PLLA), polyglycolic/polylactic acid copolymer, methacrylphosphorylcholine and laurylmethacrylate copolymer, phosphorylcholine, polycaprolactone, silicone carbide, cellulose ester, polyacrylic acid, and the like, as well as combinations of these materials. Metals, such as stainless steel, nitinol, titanium, tantalum, and the like, can also be employed as or in the support. The Wnt-related composition may be cross-linked or covalently attached to the biocompatible support. Alternatively, the Wnt-related composition may be formulated on, dissolved in, or otherwise noncovalently associated with the biocompatible support. In certain embodiments, the support is sufficiently porous to permit diffusion of Wnt-related compositions or products thereof across or out of the support. In other embodiments, the Wnt-related composition remains substantially associated with or attached to the support.

Supports can provide pharmaceutical compositions of the invention with desired mechanical properties. Those skilled in the art will recognize that minimum mechanical integrity requirements exist for implants that are to be maintained at a given target site.

Preferred intravascular implants, for example, should resist the hoop stress induced by blood pressure without rupture or aneurysm formation.

The size and shape of the support is dictated by the particular application. If the support is to be maintained at a vascular site, a tubular support is conveniently employed.

“Attachment” of Wnt-related compositions to support is conveniently achieved by adsorption of the compositions on a support surface. However, any form of attachment, e.g., via covalent or non-covalent bonds is contemplated. In one embodiment, the Wnt-related composition is prepared as a solution, preferably containing a carrier, such as bovine serum albumin (BSA). This solution is crosslinked using an agent such as glutaraldehyde, gamma irradiation, or a biocompatible epoxy solution and then applied to the surface of the support by coating or immersion.

Alternatively, Wnt-related compositions can be mechanically entrapped in a microporous support (e.g., PTFE). The Wnt-related composition solution employed for this method need not be crosslinked. After wetting the support (e.g., with 100% ethanol), the solution is forced into the pores of the support using positive or negative pressure. For tubular supports, a syringe containing the solution can be attached to the tube so that the solution is forced into the lumen of the tube and out through the tube wall so as to deposit the Wnt-related composition on internal and external support surfaces.

Wnt-related compositions can also be dissolved and suspended within a biocompatible polymer matrix, such as those described above, that can then be coated on a support or prosthetic device. Preferably, the polymerized matrix is porous enough to allow cellular interaction with the Wnt-related composition.

Wnt-related composition matrix/support assemblies intended for intravascular use may have the matrix attached to the outside surface of a tubular support. The matrix could also be attached to the interior of the support, provided the matrix was sufficiently firmly attached to the support. Loose matrix would predispose to intravascular flow disturbances and could result in thrombus formation.

In other embodiment, the Wnt-related composition is delivered via a biocompatible, intraluminal device, however, the Wnt-related composition is not crosslinked or otherwise desolved in the device. For example, the invention contemplates use of a catheter or other device to deliver a bolus of a Wnt-related composition. In such embodiments, the Wnt-related composition may not necessarily be associated with the catheter. The use of a catheter, or other functionally similar intraluminal device, allows localized delivery via the vasculature. For example, an intraluminal device can be used to deliver a bolus of Wnt-related composition directly to the myocardium, endocardium, or pericardium/pericardial space. Alternatively, an intraluminal device can be used to locally deliver a bolus of Wnt-related composition in the vasculature adjacent to cardiac tissue.

By way of illustration, intracardial injection catheters can be used to deliver the compositions of the invention directly to, for example, the myocardium or endocardium. Such catheters can be used, for example, in combination with imaging technology to deliver compositions directly into the myocardium. By way of specific example, the Stiletto™ injection system (Boston Scientific) includes two concentric fixed guide catheters and a spring loaded needle component. This and other similar injection catheters can be used for localized delivery to, for example, the myocardium or endocardium. Furthermore, such injection catheters can be used for delivery of agents into the pericardial sac. (Karmarkar et al. (2004) Magnetic Resonance in Medicine 51: 1163-1172; Naimark et al. (2003) Human Gene Therapy 14: 161-166; Bao et al. (2001) Catheter Cardiovasc Interv. 53: 429-434).

As outlined above, biocompatible devices for use in the various methods of delivery contemplated herein can be composed of any of a number of materials. The biocompatible devices include wires, stents, catheters, balloon catheters, and other intraluminal devices. Such devices can be of varying sizes and shapes depending on the intended vessel, duration of implantation, particular condition to be treated, and overall health of the patient. A skilled physician or cardiovascular surgeon can readily select from among available devices based on the particular application.

By way of further illustration, exemplary biocompatible, intraluminal devices are currently produced by several companies including Cordis, Boston Scientific, Guidant, and Medtronic (Detailed description of currently available catheters, stents, wires, etc., are available at www.cordis.com; www.medtronic.com; www.bostonscientific.com). One of skill in the art can readily select from amongst currently available or later designed devices to select a device appropriate for a particular application of the methods and compositions of the present invention.

The invention also provides articles of manufacture including pharmaceutical compositions of the invention and related kits. The invention encompasses any type of article including a pharmaceutical composition of the invention, but the article of manufacture is typically a container, preferably bearing a label identifying the composition contained therein.

The container can be formed from any material that does not react with the contained composition and can have any shape or other feature that facilitates use of the composition for the intended application. A container for a pharmaceutical composition of the invention intended for parental administration generally has a sterile access port, such as, for example, an intravenous solution bag or a vial having a stopper pierceable by a hypodermic injection needle.

Kits of the invention generally include one or more such articles of manufacture and preferably include instructions for use. Preferred kits include one or more devices that facilitate delivery of a pharmaceutical composition of the invention to a target site.

Modified Wnt-related compositions for use in the methods of the present invention, as well as modified Wnt-related compositions identified by the subject methods may be conveniently formulated for administration with a biologically acceptable medium, such as water, buffered saline, polyol (for example, glycerol, propylene glycol, liquid polyethylene glycol and the like) or suitable mixtures thereof. Exemplary modified Wnt-related compositions include hydrophobically modified, hydrophilically modified, and mixed-modified Wnt-related compositions. Such modified Wnt-related compositions may be modified with one or more moieties. Such one or more moieties may be appended to the N-terminal amino acid residue, the C-terminal amino acid residue, and/or one or more internal amino acid residue. When a modified Wnt-related composition is modified with more than one moiety, the invention contemplates that the moieties may be the same or different, and may be attached to the same amino acid residue or to different amino acid residues.

Throughout this section of the application, the term agent will be used interchangeably to refer to one or more Wnt-related compositions or modified Wnt-related compositions.

Optimal concentrations of the active ingredient(s) in the chosen medium can be determined empirically, according to procedures well known to medicinal chemists. As used herein, “biologically acceptable medium” includes solvents, dispersion media, and the like which may be appropriate for the desired route of administration of the one or more agents. The use of media for pharmaceutically active substances is known in the art. Except insofar as a conventional media or agent is incompatible with the activity of a particular agent or combination of agents, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Methods of introduction may also be provided by delivery via a biocompatible, device. Biocompatible devices suitable for delivery of the subject agents include intraluminal devices such as stents, wires, catheters, sheaths, and the like. However, administration is not limited to delivery via a biocompatible device. As detailed herein, the present invention contemplates any of number of routes of administration and methods of delivery. Furthermore, when an agent is delivered via a biocompatible device, the invention contemplates that the agent may be covalently linked, crosslinked to or otherwise associated with or dissolved in the device, or may not be so associated.

The agents identified using the methods of the present invention may be given orally, parenterally, or topically. They are of course given by forms suitable for each administration route. For example, they are administered in tablets or capsule form, by injection, inhalation, ointment, controlled release device or patch, or infusion.

The effective amount or dosage level will depend upon a variety of factors including the activity of the particular one or more agents employed, the route of administration, the time of administration, the rate of excretion of the particular agents being employed, the duration of the treatment, other drugs, compounds and/or materials used in combination with the particular agents employed, the age, sex, weight, condition, general health and prior medical history of the animal, and like factors well known in the medical arts.

The one or more agents can be administered as such or in admixtures with pharmaceutically acceptable and/or sterile carriers and can also be administered in conjunction with other compounds. These additional compounds may be administered sequentially to or simultaneously with the agents for use in the methods of the present invention.

Agents can be administered alone, or can be administered as a pharmaceutical formulation (composition). Said agents may be formulated for administration in any convenient way for use in human or veterinary medicine. In certain embodiments, the agents included in the pharmaceutical preparation may be active themselves, or may be a prodrug, e.g., capable of being converted to an active compound in a physiological setting.

Thus, another aspect of the present invention provides pharmaceutically acceptable compositions comprising an effective amount of one or more agents, formulated together with one or more pharmaceutically acceptable carriers (additives) and/or diluents. As described below, the pharmaceutical compositions of the present invention may be specially formulated for administration in solid or liquid form, including those adapted for the following: (1) delivery via a stent or other biocompatible, intraluminal device; (2) oral administration, for example, drenches (aqueous or non-aqueous solutions or suspensions), tablets, boluses, powders, granules, pastes for application to the tongue; (3) parenteral administration, for example, by subcutaneous, intramuscular or intravenous injection as, for example, a sterile solution or suspension; (4) topical application, for example, as a cream, ointment or spray applied to the skin; or (5) opthalamic administration, for example, for administration following injury or damage to the retina; (6) intramyocardial, intrapericardial, or intraendocardial administration; (7) intravascularly, intravenously, or via the coronary artiers. However, in certain embodiments the subject agents may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, one or more agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agent of the present invention. These salts can be prepared in situ during the final isolation and purification of the agents of the invention, or by separately reacting a purified agent of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the agents include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention. These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the agent which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent of the present invention as an active ingredient. An agent of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the agents of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Transdermal patches have the added advantage of providing controlled delivery of an agent of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the agent in a polymer matrix or gel.

Pharmaceutical compositions of this invention suitable for parenteral administration comprise one or more agents of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

For any of the foregoing, the invention contemplates administration to neonatal, adolescent, and adult patients, and one of skill in the art can readily adapt the methods of administration and dosage described herein based on the age, health, size, and particular disease status of the patient. Furthermore, the invention contemplates administration in utero to treat conditions in an affected fetus.

### EXEMPLIFICATIONS

The invention now being generally described, it will be more readily understood by reference to the following examples, which are included merely for purposes of illustration of certain aspects and embodiments of the present invention, and are not intended to limit the invention.

### Example 1

### Preparation of Neonatal Rat Cardiomyocyte Cultures

Neonatal rat cardiomyocytes were isolated from postnatal day 2 Wistar rat pups. Rat pups were anesthetized by hypothermia in ice water for 10 min and euthanized by decapitation. Hearts were isolated and placed in PBS-G (KCl 2 g/L; KH₂PO₄ 2 g/L; NaCl 80 g/L; Na₂HPO₄.7H₂0 21.6 g/L; D-glucose 10 g/L) on ice. The atria were removed and ventricles were washed in PBS-G and cut into pieces smaller than 2 millimeters. PBS-G containing 119.6 units/ml collagenase type 2 and 0.2 mg/ml pancreatin was warmed to 37° C. Amounts of collagenase were adjusted for batch variations in units/mg activity. Ventricles were dissociated in collagenase/pancreatin solution for 15 minutes on a rotator at 37° C. Tissue was dispersed gently by pipetting and allowed to settle for 5 min at room temperature. Cell suspension from first dissociation was discarded and replaced with fresh collagenase/pancreatin solution, and incubated at 37° C. for 15 minutes on an orbital shaker. Tissue was again dispersed and allowed to settle. The cell supension was transferred to a new tube and incubated an additional 5 min at 37° C., and the digestion was stopped by addition of an equal volume of DMEM/20% NCS and stored on ice. Digestion of the ventricular tissue was repeated 3 additional times and supernatants were collected and stored on ice in equal volumes of DMEM/20% NCS.

Cells from all the fractions were collected at 200 RCF for 6 min at room temperature. Cell pellets were resuspended in 2 ml ADS buffer (6.8 g/L NaCl, 0.4 g/L KCl, 1.0 g/L D-glucose, 1.5 g/L NaH₂PO₄, 4.75 g/L HEPES, 0.1 MgSO₄.7H₂O). One ml of cell suspension was applied to two Percoll gradients set up in the following manner. The top Percoll gradient (density 1.059 g/ml) was generated by mixing Percoll stock with ADS buffer in a 9:11 ratio, and the bottom Percoll gradient (density 1.082 g/ml) was made by mixing Percoll stock and ADS buffer in a 13:7 ratio. Four ml Top Percoll was added to a 15 ml conical tube, and three ml Bottom Percoll was laid below it by placing a pipet tip at the bottom of the tube, then carefully withdrawing it as the solution was delivered. The gradients were centrifuged at 3000 rpm for 30 consecutive minutes at room temperature. Cardiac non-myocytes were located above the top percoll buffer while cardiac myocytes were located at the interface of the top and bottom percoll layers. Blood and undissociated tissue were found at the bottom of the tube. Cardiac myocytes were collected after the top percoll was aspirated, and resuspended in DMEM/10% FBS, then centrifuged at 200 RCF for 6 min. Pelleted myocytes were resuspended in plating medium containing DMEM with 25 mM HEPES, 5% horse serum, 4 mM glutamine, penicillin and streptomycin. Four volumes of DMEM/10% FBS were added and the cell suspension was passed through a 40 uM cell strainer. Cells were pre-plated in 10 cm tissue culture plates for 1 hour at 37° C. Unattached cells were collected from the medium and centrifuged at 200 RCF for 6 min. Pelleted myocytes were resuspended in plating medium containing 10 uM AraC at a density of 100000 cells/ml and 200 ul/well were distributed into 96 well plates which had previously been coated with 0.1% gelatin/12.5 ug/ml fibronectin at 37° C. for more than 4 hours. Plated cells were grown for at least 48 hours at 37° C. prior to use in further experiments.

### Example 2

### A Wnt-Related Composition Promotes Cardiomyocyte Proliferation

Neonatal rat cardiomyocytes were prepared and cultured as outlined in example 1. As summarized in FIG. 15, administration of recombinant, mouse Wnt3A protein to the neonatal rat cardiomyocytes resulted in an increase in proliferation, as measured by incorporation of BrdU. Wnt3A protein was administered in increasing concentrations (from left, 0.06, 0.4, 2.3, 14, 83, and 500 ng/ml), and resulted in a statistically significant, dose dependent increase in cardiomyocyte proliferation. FIG. 16 shows the results of additional experiments that confirmed that recombinant Wnt3A promoted cardiomyocyte proliferation in a dose dependent manner, and that higher doses of Wnt3A promoted cardiomyocyte proliferation at levels comparable to serum.

Briefly, the experiments summarized in FIG. 15 and FIG. 16 were conducted as follows. Neonatal rat cardiomyocytes were prepared and cultured as outlined in example 1. Cells were grown for 48 hours at 37° C., and then washed 3 times with neonatal base medium containing DMEM, 25 mM HEPES, 4 mM glutamine, penicillin and streptomycin (neonatal base medium). Care was taken to leave 25 ul in the well with each wash to avoid drying the cells. Cells were left in 75 ul neonatal base medium. A 2× stock of base medium containing the desired additive was added in equal volumes to the wells. For example, in the experiments summarized in FIG. 15, recombinant mouse Wnt3A (R&D Systems) was added to the 2× stock base solution. 24 hours after the addition of stimulation medium (base medium+Wnt3A), 15 ul of a solution of 100 uM 5-bromo-2′-deoxyuridine (BrdU) was added to each well. After an additional 24 hours the cells were fixed in 3.7% formaldehyde.

Following fixation, immunocytochemistry to detect incorporation of BrdU was performed. Cells were washed 3× with PBS and treated with 4M HCl/1% Triton X-100 in H₂O for 5 minutes to denature the nuclear DNA. The acid was washed off with 4 washes of PBS. Cells were blocked for immunohistochemistry with 5% Goat serum in PBS/0.2% Tween-20 for one hour. A solution of primary antibodies containing rat anti-BrdU clone OBT0300 (Accurate Chemical) diluted 1:250 and mouse anti-tropomyosin clone CH1 supernatant (Developmental Studies Hybridoma Bank) diluted 1:100 was applied at room temperature or 37° C. for 2 hours. Cells were washed three times in PBS/0.2% Tween-20 (PBS-T) and incubated in Goat anti-mouse Alexa 488 and Goat anti-rat Alexa 594 (Molecular Probes) each diluted 1:200 for 1 hour. Nuclei were counterstained with DAPI (400 ug/ml).

Detection and quantitation of DNA synthesis in cardiomyocytes was performed as follows. Immunocytochemistry was visualized using an Axon Imagexpress automated image analyzer and software. An Axon Imagexpress software script written by Axon Instruments for the purpose of detecting overlapping red and blue nuclei surrounded by green cytoplasmic stain was applied to the acquired images. This software separately identified nuclei (blue, stained with DAPI) that were or were not BrdU positive (red, rat anti-BrdU antibody-Alexa 594 goat anti-rat antibody pair). Furthermore, it separately identified each class of nucleus by whether it was surrounded by tropomyosin stain indicative of a cardiomyocyte (green, mouse anti-tropomyosin CH1-Alexa 488 goat anti-mouse antibody pair) within a 5 uM ring drawn around the red nuclei. Thresholds were set appropriately for each plate such that overall background for each stain was not counted as positive.

Data from the Imagexpress script was imported to Microsoft Excel and percent of total cardiomyocytes that were BrdU positive was plotted for each condition. Images were exported from Imagexpress files into Adobe Photoshop. Adjustments of color and contrast were made simultaneously on all images shown in each figure.

### Example 3

### A Wnt-Related Composition Promotes Cardiomyocyte Proliferation

Neonatal rat cardiomyocytes were prepared and cultured as outlined in example 1. As summarized in FIG. 17, administration of conditioned medium from mouse L-cells expressing Wnt3A (L-Wnt3A cells available from ATCC) stimulated proliferation of neonatal rat cardiomyocytes, as measured by BrdU incorporation. In contrast, administration of conditioned medium from the parental mouse L-cells (non-Wnt expressing cells available from ATCC) did not promote cardiomyocyte proliferation.

Briefly, the experiment summarized in FIG. 17 was conducted as follows. Neonatal rat cardiomyocytes were prepared and cultured as outlined in example 1. Cells were grown for 48 hours at 37° C., and then washed 3 times with neonatal base medium containing DMEM, 25 mM HEPES, 4 mM glutamine, penicillin and streptomycin (neonatal base medium). Care was taken to leave 25 ul in the well with each wash to avoid drying the cells. Cells were left in 75 ul neonatal base medium. A 2× stock of base medium containing the desired additive was added in equal volumes to the wells. For example, in the experiment summarized in FIG. 17, conditioned medium from either Wnt3A expressing L-cells or from the non-expressing parental cell line were added to the 2× stock base solution. 24 hours after the addition of stimulation medium (base medium+conditioned medium), 15 ul of a solution of 100 uM 5-bromo-2′-deoxyuridine (BrdU) was added to each well. After an additional 24 hours the cells were fixed in 3.7% formaldehyde.

Following fixation, immunocytochemistry to detect incorporation of BrdU was performed. Cells were washed 3× with PBS and treated with 4M HCl/1% Triton X-100 in H₂O for 5 minutes to denature the nuclear DNA. The acid was washed off with 4 washes of PBS. Cells were blocked for immunohistochemistry with 5% Goat serum in PBS/0.2% Tween-20 for one hour. A solution of primary antibodies containing rat anti-BrdU clone OBT0300 (Accurate Chemical) diluted 1:250 and mouse anti-tropomyosin clone CH1 supernatant (Developmental Studies Hybridoma Bank) diluted 1:100 was applied at room temperature or 37° C. for 2 hours. Cells were washed three times in PBS/0.2% Tween-20 (PBS-T) and incubated in Goat anti-mouse Alexa 488 and Goat anti-rat Alexa 594 (Molecular Probes) each diluted 1:200 for 1 hour. Nuclei were counterstained with DAPI (400 ug/ml).

Detection and quantitation of DNA synthesis in cardiomyocytes was performed as follows. Immunocytochemistry was visualized using an Axon Imagexpress automated image analyzer and software. An Axon Imagexpress software script written by Axon Instruments for the purpose of detecting overlapping red and blue nuclei surrounded by green cytoplasmic stain was applied to the acquired images. This software separately identified nuclei (blue, stained with DAPI) that were or were not BrdU positive (red, rat anti-BrdU antibody-Alexa 594 goat anti-rat antibody pair). Furthermore, it separately identified each class of nucleus by whether it was surrounded by tropomyosin stain indicative of a cardiomyocyte (green, mouse anti-tropomyosin CH1-Alexa 488 goat anti-mouse antibody pair) within a 5 uM ring drawn around the red nuclei. Thresholds were set appropriately for each plate such that overall background for each stain was not counted as positive.

Data from the Imagexpress script was imported to Microsoft Excel and percent of total cardiomyocytes that were BrdU positive was plotted for each condition. Images were exported from Imagexpress files into Adobe Photoshop. Adjustments of color and contrast were made simultaneously on all images shown in each figure.

### Example 4

### Wnt3A is the Active Factor in Wnt3A-L-Cell Supernatant

To confirm that Wnt3A was responsible for the stimulation of cardiomyocyte proliferation observed in FIG. 17, we co-administered supernatant from Wnt3A expressing mouse L-cells and the Wnt antagonist dkk (recombinant dkk obtained from R&D Systems). As summarized in FIG. 18, co-administration of 200 ng/ml of recombinant dkk abolished the cardiomyocyte proliferative activity of the Wnt3A-L-cell supernatant. This, in combination with experiments indicating that medium from the parental L-cell line did not promote cardiomyocyte proliferation, confirmed that Wnt3A is the active cardiomyocyte proliferative factor in L-cell conditioned medium.

We note that, as indicated in FIG. 18, although dkk abolished Wnt3A induced cardiomyocyte proliferation, 100 ng/ml of the Wnt inhibitors FRP2 and FRP3 did not. This result is consistent with the different mechanisms by which dkk and FRP inhibit Wnt signaling. Nevertheless, the inhibition of Wnt3A induced cardiomyocyte proliferation by dkk supports the conclusion that Wnt3A is the active cardiomyocyte proliferative factor in the conditioned medium.

The experiments were conducted and analyzed as detailed above in examples 2 and 3.

### Example 5

### A Wnt-Related Composition Promotes Cardiomyocyte Proliferation and Promotes Wnt Signaling

The results summarized in FIG. 19 demonstrate that a Wnt-related composition that promoted cardiomyocyte proliferation in neonatal rat cardiomyocytes also promotes Wnt signaling. Specifically, the Wnt-related composition stabilizes β-catenin. The increase in expression and/or stability of β-catenin indicated that Wnt3A promoted cardiomyocyte proliferation via the canonical Wnt signaling pathway. This is in contrast to administration of serum (FCS) which promotes proliferation, but does not promote Wnt signaling.

Briefly, neonatal rat cardiomyocytes were treated with either serum or with recombinant Wnt3A (e.g., 167 ng/ml or 500 ng/ml) in neonatal base medium for 48 hours. The treated cells were fixed, permeablilized with 0.5% Triton-X-100 for 15 minutes, and blocked in 5% goat serum in PBS-T for one hour. Mouse anti-β-catenin antibody (BD-Pharmingen) diluted 1:250 were incubated on cells for one hour. Cells were washed 3 times in PBS-T and incubated one hour in Alexa 594 goat anti-mouse antibody (Molecular Probes) at a dilution of 1:200. As shown in FIG. 19, administration of recombinant Wnt3A, but not serum, resulted in stabilization of β-catenin—as assessed by increased detection and nuclear localization of β-catenin. This result indicated that Wnt3A promoted cardiomyocyte proliferation and promoted Wnt signaling via the canonical Wnt signaling pathway.

### Example 6

### A Wnt-Related Composition Promotes Cardiomyocyte Proliferation, but Does Not Produce a Hypertrophic Response

One limitation of many stimuli that induce cardiomyocyte proliferation is that those stimuli also produce hypertrophy. Such hypertrophy is inconsistent with the production of cells capable of functionally replacing damaged cardiomyocytes. Accordingly, preferable Wnt-related polypeptides and compositions should promote cardiomyocyte proliferation without inducing a hypertrophic response.

The experiments summarized in FIGS. 20 and 21 demonstrated that Wnt-related compositions promoted cardiomyocyte proliferation but did not produce a hypertrophic response. Briefly, neonatal rat cardiomyocytes were treated with serum or phenylephrine (two agents known to induce hypertrophy), or were treated with 150 ng/ml recombinant Wnt3A. Following treatment, the cells were analyzed by immunocytochemistry for expression of atrial naturietic factor (ANF) (rabbit anti-ANF/Peninsula laboratories/Bachem) or expression of tropomyosin (CH1 antibody available from Developmental Hybridoma).

FIG. 20 shows that ANF expression increased in cells treated with hypertrophic stimuli, but not in cells treated with Wnt3A. FIG. 21 shows the dramatic change in cardiomyocyte cell size and shape following treatment with hypertrophic stimuli, but not following treatment with Wnt3A. Accordingly, the experiments summarized in FIGS. 20-21 indicated that Wnt-related compositions promote cardiomyocyte proliferation without inducing a hypertrophic response.

### Example 7

### A Wnt-Related Composition Promotes Cardiomyocyte Proliferation and Promotes Wnt Signaling Via the Canonical Wnt Signaling Pathway

The results summarized in FIG. 22 provide further evidence that a Wnt-related composition that promoted cardiomyocyte proliferation in neonatal rat cardiomyocytes also promotes Wnt signaling via the canonical Wnt signaling pathway. Specifically, the cardiomyocyte-proliferative activity of a Wnt-related composition is phenocopied by administration of lithium chloride (LiCl), a known activator of the canonical Wnt signaling pathway.

Briefly, neonatal rat cardiomyocytes were treated with either serum, with recombinant Wnt3A (e.g., 150 ng/ml, 50 ng/ml, or 17 ng/ml), or with LiCl (e.g., 10 mM, 5 mM, or 2.5 mM) in neonatal base medium for 48 hours. Cardiomyocyte proliferation was assessed, as outlined in detail above. As shown in FIG. 22, administration of Wnt3A or LiCl promoted cardiomyocyte proliferation. Given that LiCl is a known activator of the canonical Wnt signaling pathway, this result further indicated that Wnt-related compositions promote cardiomyocyte proliferation and promote Wnt signaling via the canonical Wnt signaling pathway.

### Example 8

### A Wnt-Related Composition Promotes Cardiomyocyte Proliferation and Promotes Wnt Signaling

The results summarized in Example 5 indicate that Wnt signaling via the canonical Wnt signaling pathway can promote cardiomyocyte proliferation. The specific example provided aboved demonstrated that Wnt3A can promote cardiomyocyte proliferation and can promote Wnt signaling via the canonical Wnt signaling pathway, as assessed by promoting the stability of β-catenin. The invention further contemplates that other Wnt-related polypeptides can function to promote Wnt signaling via the canonical Wnt signaling pathway, and can also promote cardiomyocyte proliferation. Furthermore the invention contemplates a wide range of methods for assessing whether a particular Wnt-related polypeptide promotes Wnt signaling via the canonical Wnt signaling pathway.

By way of example, any Wnt-related composition, modified Wnt-related composition, or bioactive fragment thereof can be tested. Such compositions can be tested in cultures of rat neonatal cardiomyocytes, as outlined in detail above. Alternatively or in addition to, such compositions can be tested in cultures of neonatal cardiomyocytes derived from other animals, in cultures of fetal or adult cardiomyocytes derived from mouse, rats, humans, etc., or in transformed cardiac cell lines. Suitable Wnt-related compositions for testing in these and other assays include compositions comprising a modified or un-modified Wnt polypeptide selected from any of Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, Wnt16, or a bioactive fragment of any of the foregoing. Such assays allow one to select Wnt-related compositions for use in the methods of the present invention (e.g., allow the selection of a Wnt-related composition that promotes cardiomyocyte proliferation and that promotes Wnt signaling via the canonical Wnt signaling pathway).

Many suitable assays indicative of Wnt signaling via the canonical Wnt signaling pathway are known in the art. Example 5 outlined one assay based on the stability of β-catenin. Briefly, an antibody immunoreactive with β-catenin was used to show increased stability and nuclear localization of β-catenin following treatment of cells with Wnt3A. Such an assay can be used to identify additional Wnt polypeptides that promote Wnt signaling via the canonical Wnt signaling pathway and that promote cardiomyocyte proliferation.

By way of further example, the ability of a Wnt-related composition to promote Wnt signaling via the canonical Wnt signaling pathway can be measured by examining the phosphorylation states of GSK3β, or by examing the expression of downstream target genes activated in response to canonical Wnt signaling via β-catenin. Such downstream genes include TCF, siamois, and other targets known in the art. The activation of these genes in response to a Wnt polypeptide indicates that the Wnt polypeptide promotes Wnt signaling via the canonical Wnt signaling pathway. The expression of endogenous Wnt responsive genes can be readily measured using Northern blot, RT-PCR, in situ hybridization, and other commonly employed molecular biological techniques. Additionally, Wnt signaling via the canonical Wnt signaling pathway can be assayed in cells comprising a reporter construct responsive to Wnt signaling via the canonical Wnt signaling pathway. Such reporter constructs include β-catenin/TCF dependent reporter constructs including TOPFlash, FOPFlash, and SuperTOPFlash (Korinek et al. (1997) Science 275: 1784-1787; Veeman et al. (2003) Current Biology 13: 680).

### Example 9

### A Composition Comprising an Agent that Acts at the Cell Surface to Promote Wnt Signaling via the Canonical Wnt Signaling Pathway Promotes Cardiomyocyte Proliferation

The results summarized in Example 5 indicate that Wnt signaling via the canonical Wnt signaling pathway can promote cardiomyocyte proliferation. The specific example provided above demonstrated that Wnt3A can act at the cell surface to promote cardiomyocyte proliferation and to promote Wnt signaling via the canonical Wnt signaling pathway, as assessed by promoting the stability of β-catenin. The invention further contemplates that compositions comprising other agents can act at the cell surface to both promote Wnt signaling via the canonical Wnt signaling pathway and to promote cardiomyocyte proliferation. Furthermore the invention contemplates a wide range of methods for assessing whether a particular composition that promotes cardiomyocyte proliferation promotes Wnt signaling via the canonical Wnt signaling pathway.

Exemplary agents that act at the cell surface to promote Wnt signalnig via the canonical Wnt signaling pathway include, but are not limited, to nucleic acid agents, peptide agents, polypeptide agents, antibody agents, and small molecule agents. Agents can be readily tested to determine whether the agent (i) promotes cardiomyocyte proliferation and (ii) promotes Wnt signaling via the canonical Wnt signaling pathway. Such agents include any Wnt-related composition, modified Wnt-related composition, bioactive fragment of a Wnt-related composition, LRP-related nucleic acid, LRP-related polypeptide, fragment of an LRP-related polypeptide comprising an N-terminal deletion, anti-LRP antibody, a nucleic acid encoding a fragment of an LRP-related polypeptide comprising an N-terminal deletion, a soluble extracellular fragment of an LRP-related polypeptide, or a modified soluble extracellular fragment of an LRP-related polypeptide.

Such compositions can be tested in cultures of rat neonatal cardiomyocytes, as outlined in detail above. Alternatively or in addition to, such compositions can be tested in cultures of neonatal cardiomyocytes derived from other animals, in cultures of fetal or adult cardiomyocytes derived from mice, rats, humans, etc., or in transformed cardiac cell lines.

Many suitable assays indicative of Wnt signaling via the canonical Wnt signaling pathway are known in the art. Example 5 outlined one assay based on the stability of β-catenin. Briefly, an antibody immunoreactive with β-catenin was used to show increased stability and nuclear localization of β-catenin following treatment of cells with Wnt3A. Such an assay can be used to identify additional compositions that act at the cell surface to promote Wnt signaling via the canonical Wnt signaling pathway and that promote cardiomyocyte proliferation.

By way of further example, the ability of an agent to promote Wnt signaling via the canonical Wnt signaling pathway can be measured by examining the phosphorylation states of GSK3β, or by examing the expression of downstream target genes activated in response to canonical Wnt signaling via β-catenin. Such downstream genes include TCF, siamois, and other targets known in the art. The activation of these genes in response to an agent indicates that the agent promotes Wnt signaling via the canonical Wnt signaling pathway. The expression of endogenous Wnt responsive genes can be readily measured using Northern blot, RT-PCR, in situ hybridization, and other commonly employed molecular biological techniques. Additionally, Wnt signaling via the canonical Wnt signaling pathway can be assayed in cells comprising a reporter construct responsive to Wnt signaling via the canonical Wnt signaling pathway. Such reporter constructs include β-catenin/TCF dependent reporter constructs including TOPFlash, FOPFlash, and SuperTOPFlash (Korinek et al. (1997) Science 275: 1784-1787; Veeman et al. (2003) Current Biology 13: 680).

### Example 10

### Agents that Promote Wnt Signaling Via the Canonical Pathway in Cardiac Cells can be Readily Identified

The present invention provides methods and compositions that both promote Wnt signaling via the canonical wnt signaling pathway and that promote proliferation, regeneration, and/or survival of cardiac cells. Given that some Wnt polypeptides have been shown to signal either via the canonical Wnt signaling pathway or via the noncanonical Wnt signaling pathway depending on the expression of particular frizzled receptors in particular cell types, it would be useful to have a method of easily testing the various Wnt-related polypeptides, modified polypeptides, and bioactive fragments thereof to identify the Wnt-related polypeptides (or other agents that act at the cell surface to promote Wnt signaling) that promote Wnt signaling via the canonical wnt signaling pathway specifically (though not necessarily exclusively) in cardiac cells types. In this way, Wnt-related polypeptides can be easily classified by whether they can promote wnt signaling via the canonical wnt signaling pathway in cardiac cell types. The present invention provides such a method.

We have used a nuclear beta-catenin assay to readily identify Wnt-related polypeptides that promote Wnt signaling via the canonical Wnt signaling pathway in cardiac cells. Specifically, and by way of example, we conducted a nuclear beta-catenin assay in rat neonatal cardiomyocytes to identify Wnt-related polypeptides that promote Wnt signaling via the canonical Wnt signaling pathway in cardiomyocytes. Such an assay conducted in neonatal or adult cardiac cells including, but not limited to, cardiomyocytes can be used to identify candidate agents that are capable of promoting Wnt signaling via the canonical Wnt signaling pathway in cardiac cells.

Briefly, neonatal cardiomyocytes were prepared and plated as outlined in detail above. Cells were washed two times with serum-free medium and were incubated in medium containing either a recombinant Wnt-related polypeptide, or some control factor. Cells were incubated for 16 hours, were fixed in 4% Formaldehye for 10 minuntes at room temperature, and were permeabilized with 0.2% Triton X-100 for 5 minutes. To detect activated beta-catenin (dephosphorylated beta-catenin; indicates active Wnt signaling via the canonical pathway), cells were incubated overnight with mouse monoclonal antibody to beta-catenin (BD Transduction Laboratories, Catalog# 610153) in Tris-buffered saline/3% BSA and then for 2 hours with goat anti-mouse IgG conjuaged with Alexa Fluor 594 (Molecular Probes). Although beta-catenin is not typically detectable by immunocytochemistry in the absence of active Wnt signaling, the nuclei were counterstained with 4′,6-diamidino-2-phenylindole (DAPI) to specifically confirm that the detected beta-catenin expression was localized to the nucleus. Nuclear localization of beta-catenin, for example in response to a Wnt protein, indicates active Wnt signaling via the canonical Wnt signaling pathway. Images and analysis were done using “Imagexpress” software.

Exemplary results of these experiments are summarized in FIG. 23. Neonatal cardiomyocytes were cultured under one of the following conditions: medium alone (negative control), 10 mM LiCl, 2% fetal calf serum (FCS), 10% fetal calf serum (FCS), 25 ng/ml recombinant mouse Wnt5A (Catalog# 645-WN/CF), 50 ng/ml recombinant mouse Wnt5A (Catalog# 645-WN/CF), 100 ng/ml recombinant mouse Wnt5A (Catalog# 645-WN/CF), 25 ng/ml recombinant mouse Wnt3A (Catalog # 1324-WN/CF), 50 ng/ml recombinant mouse Wnt3A (Catalog # 1324-WN/CF), or 100 ng/ml recombinant mouse Wnt3A (Catalog # 1324-WN/CF). Following culture under one of the foregoing conditions, cells were analyzed by immunocytochemistry for expression of beta-catenin protein, and nuclear localization was verified by counterstaining the cells with DAPI. The results of these experiments are indicated as the percentage of cells with nuclear beta-catenin staining. As summarized in FIG. 23, 10 mM LiCl (a known, intracellular activator of the canonical Wnt signaling pathway) promoted beta-catenin nuclear localization (e.g., promoted Wnt signaling via the canonical Wnt signaling pathway) in neonatal cardiomyocytes in comparison to medium alone (negative control), or in comparison to 2% or 10% serum. Recombinant Wnt3A at concentrations of 25 ng/ml-100 ng/ml promoted beta-catenin nuclear localization (e.g., promoted Wnt signaling via the canonical Wnt signaling pathway) in neonatal cardiomyocytes. In contrast, recombinant Wnt5A at concentrations of 25 ng/ml-100 ng/ml did not promote beta-catenin nuclear localization in neonatal cardiomyocytes.

This assay provides a mechanism to rapidly assess which Wnt-related polypeptides can promote Wnt signaling via the canonical Wnt signaling pathway specifically in one or more cardiac cell type. The exemplary methods detailed above can be readily modified, for example, to assess conditioned medium from Wnt expressing cells such as Wnt-expressing L cells. Furthermore, the method can be readily modified to assess Wnt signaling via the canonical Wnt signaling pathway in other cardiac cell types. By way of nonlimiting example, the ability of a particular Wnt-related polypeptide or other composition to promote Wnt signaling via the canonical Wnt signaling pathway can be evaluated in cultures of unfractionated adult cardiac cell preparations, in adult cardiomyocyte preparations, or in fractioned neonatal or adult cardiac cell preparations.

One of skill in the art can readily use this methodology to categorize Wnt-related polypeptides, as well as other agents that act at the cell surface to promote Wnt signaling, that activate Wnt signaling via the canonical Wnt signaling pathway specifically in cardiac cells. One of skill in the art can readily test the various Wnt-related polypeptides, thereby identify which of the Wnt-related polypeptides can promote signaling via the canonical wnt signaling pathway in cardiac cells. Alternatively, one of skill in the art can prioritize which Wnt-related polypeptides to assess based on knowledge of which Wnt polypeptides have been shown to signal via the canonical Wnt signaling pathway in other cell types. We note however, evidence that a particular polypeptide can signal via the canonical wnt signaling pathway in other cell types is not necessarily predictive of whether it can signal via the canonical wnt signaling pathway in cardiac cell types.

By way of example of how one of skill in the art might prioritize assessing which Wnt-related polypeptides signal via the canonical wnt signaling pathway in cardiac cells, the following wnt polypeptides have been shown to signal via the canonical pathway in one or more non-cardiac cells types: Wnt1, Wnt2, Wnt2B, Wnt3, Wnt3A, Wnt6, Wnt7a, Wnt8a, Wnt8b, Wnt9a, Wnt9b, Wnt10a, Wnt10b, and Wnt16. (Fahnert et al. (2004) Journal of Biological Chemistry 279(46): 47520-7; Shimizu et al. (1997) Cell Growth Differ 8: 1349-1358; Katoh et al. (2001) Biochem Biophys Res Commun 289: 1093-1098; Nakamura et al. (2003) PNAS 100(10): 5834-9; Lu et al. (2004) PNAS 101: 3118-3123; Caricasole et al. (2003) Journal of Biological Chemistry 278: 37024-37031; Veeman et al. (2003) Developmental Cell 5: 367-377; Qian et al. (2003) Genomics 81: 34-46; Longo et al. (2004) Journal of Biological Chemistry 279(34): 35503-9; Guo et al. (2004) Genes and Development 18: 2404-2417). In one approach, one of skill in the art could first assess each of these Wnt polypeptides, as well as related variants and modified polypeptides, to determine whether they signal via the canonical wnt signaling pathway in one or more cardiac cell types. The following Wnt polypeptides have been shown to signal via the non-canonical signaling pathway in one or more non-cardiac cell types: Wnt4, Wnt5a, Wnt5b, Wnt7b, and Wnt11. (Matsui et al. (2005) Genes and Development 19: 164-175; Liang et al. (2003) Cancer Cell 4: 349-360; Kuhl et al. (2000) Trends Genet 16: 279-283; Veeman et al. (2003) Developmental Cell 5: 367-377; Kim et al. (2004) Nat Cell Biol 6: 1212-1220). Although evidence that these polypeptides signal via the non-canonical wnt signaling pathway does not conclusively indicate that they cannot signal canonically in cardiac cell types, one of skill in the art could decide to analyze these polypeptides, as well as related variants and modified polypeptides, secondarily.

This and other assays that indicate active Wnt signaling via the canonical Wnt signaling pathway can be conducted in one or more cardiac cell types. Using such approaches, one can readily select from amongst known Wnt polypeptides, modified Wnt polypeptides, and bioactive fragments thereof, and identify polypeptides that promote Wnt signaling via the canonical Wnt signaling pathway in one or more cardiac cell type. Such Wnt-related polypeptides that promote Wnt signaling via the canonical wnt signaling pathway in one or more cardiac cell types may be used in the methods and compositions of the invention. For example, Wnt-related polypeptides known to promote Wnt signaling via the canonical Wnt signaling pathway in other cell types (e.g., Wnt1, Wnt2, Wnt2B, Wnt3, Wnt3A, Wnt6, Wnt7A, Wnt8a, Wnt8b, Wnt9a, Wnt9b, Wnt10a, Wnt10b, and Wnt16) can be tested for the ability to promote Wnt signaling via the canonical Wnt signaling pathway in cardiac cell types. In this way, Wnt-related polypeptides for use in the methods of the invention can be readily selected from amongst all known Wnt-related polypeptides.

### Example 11

### A Wnt-Related Composition Promotes Proliferation and/or Survival of Adult Cardiac Cells

We assessed the affect of a Wnt-related composition on various adult cardiac cell populations. As part of this and other analysis, we made a stable cell line expressing increased levels of Wnt3A. This cell line, referred to as LWW60, was made by transfecting Wnt3A expressing L cells (purchased from ATCC), and selecting for stable cells expressing Wnt3A. Briefly, Wnt3A expressing L-cells were grown to 80% confluence, and transfected with a plasmid containing and expressing Wnt3A and the selectable marker hygromycin. Transfectants were selected in hygromycin containing media, and tested for protein production and bioactivity.

Whole hearts were obtained from adult mice (greater than 5 weeks of age). Cardiac tissue was minced and washed three times to remove blood. Cardiac cells were dissociated with pancreatin and collagenase for 15 minutes at 37° C. and washed. Following dissociation the unfractionated cells were either plated, or fractionated into a Sca1+ and Sca1− population prior to plating. These three cardiac cell populations (unfractionated cell preparation; Sca1+cell preparation; Sca1− cell preparation) were cultured in the presence of various Wnt-related compositions. Specifically, the cardiac cell preparations were cultured for 3-4 days in either L-cell conditioned medium alone; LWW60 conditioned medium; or culture medium containing purified Wnt3A protein. Following 3-4 days in culture, the cardiac cells were washed with PBS and trypsinized, and viable cells were counted.

As summarized in FIGS. 24 and 25, a dramatically increased number of viable adult cardiac cells were present following culture in the presence of a Wnt-related composition in comparison to controls. FIG. 24 summarizes the results of experiments in which unfractioned adult cardiac cells were cultured for 4 days in the presence of either control medium or in the presence of LWW60 conditioned medium. Viable cell count was substantially increased in the presence of LWW60 conditioned medium. This difference is likely the result of increased cell proliferation and possibly increased cell survival in adult cardiac cells in the presence of LWW60 conditioned medium.

FIG. 25 summarizes the results of experiments examining the effects of LWW60 conditioned medium (also referred to as Lwnt/wnt CM) or recombinant Wnt3A (50ng/ml) on various adult cardiac cell populations. FIG. 25a shows that LWW60 conditioned medium increased the number of viable cells in both unfractioned (whole heart) preparations and in Sca1+cells preparations. Furthermore, FIG. 25a shows that 50 ng/ml of recombinant Wnt3A also increased the number of viable cells in both unfractioned (whole heart) preparations and in Sca1+cells preparations. We note that overall cell viability is decreased in the Sca1+preparations. This is largely an artifact of the Sca1+antibody-based selection procedure which appears to isolate a substantial number of dead and dying cells. Notably however and despite this limitation of the selection protocol, the Wnt-related compositions still increase viable cell counts in these Sca1+preparations. FIG. 25b shows that LWW60 conditioned medium dramatically increased the number of viable cells in Sca1− cell preparations from adult cardiac tissue.

### Example 12

### Combinatorial Affect on Cardiac Proliferation of a Wnt-Related Composition and IGF-1

We examined the effect of contacting neonatal cardiomyocytes with both Wnt3a and IGF-1, and these results aree summarized in FIGS. 26 and 27. Briefly, neonatal cardiomyocytes were prepared and cultured as outlined in detail above. Cells were cultured in the presence of either recombinant Wnt3A protein alone (at doses of 100 ng/ml, 33.3 ng/ml, 11.1 ng/ml, or 3.7 ng/ml), recombinant IGF-1 protein alone (at does of 1 ng/ml, 3 ng/ml, or 9 ng/ml), or both recombinant Wnt3A protein and recombinant IGF-1 protein. Following culture, cardiomyocyte proliferation was assessed, as described in detail above.

FIGS. 26 and 27 summarize the results of the same experiments. However, for illustrative purposes, the two figures present the results differently. Briefly, these experiments demonstrated that a combination of an agent that promotes Wnt signaling via the canonical Wnt signaling pathway (e.g., Wnt3A) and IGF-1 promoted cardiac cell proliferation, specifically cardiomyocyte proliferation. Furthermore, the effect of the combination of the two proteins on cardiac cell proliferation is at least additive, and even synergistic, in comparison to the effect on cardiac cell proliferation of either protein alone.

These results indicate that combinations of a Wnt-related polypeptide that promotes Wnt signaling via the canonical Wnt signaling and particular growth factors can be used to promote cardiac cell proliferation, and furthermore that the combination of these factors may act additively or synergistically to promote cardiac cell proliferation. These results suggest, and the invention contemplates, that Wnt-related polypeptides that promote Wnt signaling via the canonical Wnt signaling pathway can be administered in combination with other agents to promote cardiac cell proliferation additively or synergistically. By way of example, IGF1 is exemplary of a particular class of agents that may be used in combination with wnt polypeptides to promote cardiac cell proliferation. Specifically, IGF1 is known to signal, at least in part, by activating the Akt/PI3 kinase pathway. Accordingly, the invention contemplates that other factors that, like IGF1, activate the Akt/PI3K pathway can be combined with a Wnt polypeptide that activates Wnt signaling via the canonical Wnt signaling pathway to promote cardiac cell proliferation. In certain embodiments, this combination of a Wnt polypeptide and a factor that activates the Akt/PI3K pathway act additively or synergistically to promote cardiac cell proliferation. Exemplary factors that, like IGF 1, activate the Akt/PI3 kinase pathway include, but are not limited to, IGF2, insulin, hepatocyte growth factor, interleukin-6, and interleukin-7.

Exemplary Nucleic Acid and Amino Acid Sequences Referenced Herein

TABLE 1

| | |
|---|---|
| SEQ ID NO: 1 | Human Wnt1 nucleic acid sequence |
| | (NM_005430) |
| SEQ ID NO: 2 | Human Wnt1 amino acid sequence |
| | (NM_005430) |
| SEQ ID NO: 3 | Mouse Wnt1 nucleic acid sequence |
| | (NM_021279) |
| SEQ ID NO: 4 | Mouse Wntl amino acid sequence |
| | (NM_021279) |
| SEQ ID NO: 5 | Human Wnt2 nucleic acid sequence |
| | (NM_003391) |
| SEQ ID NO: 6 | Human Wnt2 amino acid sequence |
| | (NM_003391) |
| SEQ ID NO: 7 | Mouse Wnt2 nucleic acid sequence |
| | (NM_023653) |
| SEQ ID NO: 8 | Mouse Wnt2 amino acid sequence |
| | (NM_023653) |
| SEQ ID NO: 9 | Human Wnt2B1/Wnt13, transcript variant 1, |
| | nucleic acid sequence (NM_004185) |
| SEQ ID NO: 10 | Human Wnt2B1/Wnt13, transcript variant 1, |
| | amino acid sequence (NM_004185) |
| SEQ ID NO: 11 | Human Wnt2B2/Wnt13, transcript variant 2, |
| | nucleic acid sequence (NM_024494) |
| SEQ ID NO: 12 | Human Wnt2B2/Wnt13, transcript variant 2, |
| | amino acid sequence (NM_024494) |
| SEQ ID NO: 13 | Mouse Wnt2B/Wnt13 nucleic acid sequence |
| | (NM_009520) |
| SEQ ID NO: 14 | Mouse Wnt2B/Wnt13 amino acid sequence |
| | (NM_009520) |
| SEQ ID NO: 15 | Human Wnt3 nucleic acid sequence |
| | (NM_030753) |
| SEQ ID NO: 16 | Human Wnt3 amino acid sequence |
| | (NM_030753) |
| SEQ ID NO: 17 | Mouse Wnt3 nucleic acid sequence |
| | (NM_009521) |
| SEQ ID NO: 18 | Mouse Wnt3 amino acid sequence |
| | (NM_009521) |
| SEQ ID NO: 19 | Human Wnt3A nucleic acid sequence |
| | (NM_033131) |
| SEQ ID NO: 20 | Human Wnt3A amino acid sequence |
| | (NM_033131) |
| SEQ ID NO: 21 | Mouse Wnt3A nucleic acid sequence |
| | (NM_009522) |
| SEQ ID NO: 22 | Mouse Wnt3A amino acid sequence |
| | (NM_009522) |
| SEQ ID NO: 23 | Human Wnt4 nucleic acid sequence |
| | (NM_030761) |
| SEQ ID NO: 24 | Human Wnt4 amino acid sequence |
| | (NM_030761) |
| SEQ ID NO: 25 | Mouse Wnt4 nucleic acid sequence |
| | (NM_009523) |
| SEQ ID NO: 26 | Mouse Wnt4 amino acid sequence |
| | (NM_009523) |
| SEQ ID NO: 27 | Human Wnt5A nucleic acid sequence |
| | (NM_003392) |
| SEQ ID NO: 28 | Human Wnt5A amino acid sequence |
| | (NM_003392) |
| SEQ ID NO: 29 | Mouse Wnt5A nucleic acid sequence |
| | (NM_009524) |
| SEQ ID NO: 30 | Mouse Wnt5A amino acid sequence |
| | (NM_009524) |
| SEQ ID NO: 31 | Human Wnt5B nucleic acid sequence |
| | (NM_030775) |
| SEQ ID NO: 32 | Human Wnt5B amino acid sequence |
| | (NM_030775) |
| SEQ ID NO: 33 | Mouse Wnt5B nucleic acid sequence |
| | (NM_009525) |
| SEQ ID NO: 34 | Mouse Wnt5B amino acid sequence |
| | (NM_009525) |
| SEQ ID NO: 35 | Human Wnt6 nucleic acid sequence |
| | (NM_006522) |
| SEQ ID NO: 36 | Human Wnt6 amino acid sequence |
| | (NM_006522) |
| SEQ ID NO: 37 | Mouse Wnt6 nucleic acid sequence |
| | (NM_009526) |
| SEQ ID NO: 38 | Mouse Wnt6 amino acid sequence |
| | (NM_009526) |
| SEQ ID NO: 39 | Human Wnt7A nucleic acid sequence |
| | (NM_004625) |
| SEQ ID NO: 40 | Human Wnt7A amino acid sequence |
| | (NM_004625) |
| SEQ ID NO: 41 | Mouse Wnt7A nucleic acid sequence |
| | (NM_009527) |
| SEQ ID NO: 42 | Mouse Wnt7A amino acid sequence |
| | (NM_009527) |
| SEQ ID NO: 43 | Human Wnt7B nucleic acid sequence |
| | (NM_058238) |
| SEQ ID NO: 44 | Human Wnt7B amino acid sequence |
| | (NM_058238) |
| SEQ ID NO: 45 | Mouse Wnt7B nucleic acid sequence |
| | (NM_009528) |
| SEQ ID NO: 46 | Mouse Wnt7B amino acid sequence |
| | (NM_009528) |
| SEQ ID NO: 47 | Human Wnt5A nucleic acid sequence |
| | (NM_031933) |
| SEQ ID NO: 48 | Human Wnt8A amino acid sequence |
| | (NM_031933) |
| SEQ ID NO: 49 | Mouse Wnt8A nucleic acid sequence |
| | (NM_009290) |
| SEQ ID NO: 50 | Mouse Wnt8A amino acid sequence |
| | (NM_009290) |
| SEQ ID NO: 51 | Human Wnt8B nucleic acid sequence |
| | (NM_003393) |
| SEQ ID NO: 52 | Human Wnt8B amino acid sequence |
| | (NM_003393) |
| SEQ ID NO: 53 | Mouse Wnt8B nucleic acid sequence |
| | (NM_011720) |
| SEQ ID NO: 54 | Mouse Wnt8B amino acid sequence |
| | (NM_011720) |
| SEQ ID NO: 55 | Human Wnt9A (previously Wnt14) nucleic acid |
| | sequence (NM_003395) |
| SEQ ID NO: 56 | Human Wnt9A (previously Wnt14) amino acid |
| | sequence (NM_003395) |
| SEQ ID NO: 57 | Mouse Wnt9A (previously Wnt14) nucleic acid |
| | sequence (NM_139298) |
| SEQ ID NO: 58 | Mouse Wnt9A (previously Wnt14) amino acid |
| | sequence (NM_139298) |
| SEQ ID NO: 59 | Human Wnt9B (previously Wnt15) nucleic acid |
| | sequence (NM_003396) |
| SEQ ID NO: 60 | Human Wnt9B (previously Wnt15) amino acid |
| | sequence (NM_003396) |
| SEQ ID NO: 61 | Mouse Wnt9B (previously Wnt15) nucleic acid |
| | sequence (NM_011719) |
| SEQ ID NO: 62 | Mouse Wnt9B (previously Wnt15) amino acid |
| | sequence (NM_11719) |
| SEQ ID NO: 63 | Human Wnt10A nucleic acid sequence |
| | (NM_025216) |
| SEQ ID NO: 64 | Human Wnt10A amino acid sequence |
| | (NM_025216) |
| SEQ ID NO: 65 | Mouse Wnt10A nucleic acid sequence |
| | (NM_009518) |
| SEQ ID NO: 66 | Mouse Wnt10A amino acid sequence |
| | (NM_009518) |
| SEQ ID NO: 67 | Human Wnt10B nucleic acid sequence |
| | (NM_003394) |
| SEQ ID NO: 68 | Human Wnt10B amino acid sequence |
| | (NM_003394) |
| SEQ ID NO: 69 | Mouse Wnt10B nucleic acid sequence |
| | NM_011718) |
| SEQ ID NO: 70 | Mouse Wnt10B amino acid sequence |
| | (NM_011718) |
| SEQ ID NO: 71 | Human Wnt11 nucleic acid sequence |
| | (NM_004626) |
| SEQ ID NO: 72 | Human Wnt11 amino acid sequence |
| | (NM_004626) |
| SEQ ID NO: 73 | Mouse Wnt11 nucleic acid sequence |
| | NM_009519) |
| SEQ ID NO: 74 | Mouse Wnt11 amino acid sequence |
| | (NM_009519) |
| SEQ ID NO: 75 | Human Wnt16, transcript variant 1, nucleic |
| | acid sequence (NM_057168) |
| SEQ ID NO: 76 | Human Wnt16, transcript variant 1, amino |
| | acid sequence (NM_057168) |
| SEQ ID NO: 77 | Human Wnt16, transcript variant 2, nucleic |
| | acid sequence (NM_016087) |
| SEQ ID NO: 78 | Human Wnt16, transcript variant 2, amino |
| | acid sequence (NM_016087) |
| SEQ ID NO: 79 | Human LRP5 nucleic acid sequence |
| | (NM_002335) |
| SEQ ID NO: 80 | Human LRP5 amino acid sequence |
| | (NM_002335) |
| SEQ ID NO: 81 | Mouse LRP5 nucleic acid sequence |
| | (NM_008513) |
| SEQ ID NO: 82 | Mouse LRP5 amino acid sequence |
| | (NM_008513) |
| SEQ ID NO: 83 | Human LRP6 nucleic acid sequence |
| | (NM_002336) |
| SEQ ID NO: 84 | Human LRP6 amino acid sequence |
| | (NM_002336) |
| SEQ ID NO: 85 | Mouse LRP6 nucleic acid sequence |
| | (NM_008514) |
| SEQ ID NO: 86 | Mouse LRP6 amino acid sequence |
| | (NM_008514) |

All publications, patents and patent applications are herein incorporated by reference in their entirety to the same extent as if each individual publication, patent or patent application was specifically and individually indicated to be incorporated by reference in its entirety.

### Equivalents

Those skilled in the art will recognize, or be able to ascertain using no more than routine experimentation, many equivalents to the specific embodiments of the invention described herein. Such equivalents are intended to be encompassed by the following claims.

1. A method of promoting neonatal or adult cardiac cell proliferation, comprising contacting said cell with a composition comprising an agent that acts at the cell surface to promote signaling via the canonical Wnt signaling pathway, wherein said cell is contacted with an amount of said composition effective to promote neonatal or adult cardiac cell proliferation.

2. A method of promoting neonatal or adult cardiac cell regeneration, comprising contacting said cell with a composition comprising an agent that acts at the cell surface to promote signaling via the canonical Wnt signaling pathway, wherein said cell is contacted with an amount of said composition effective to promote neonatal or adult cardiac cell regeneration.

3. The method of claim 1, comprising contacting said cell with a composition comprising a Wnt-related polypeptide, or a bioactive fragment thereof, wherein said cell is contacted with an amount of said composition effective to promote cardiac cell proliferation, and wherein said Wnt-related polypeptide, or bioactive fragment thereof, promotes Wnt signaling via the canonical Wnt signaling pathway.

4. The method of claim 2, comprising administering a composition comprising a Wnt-related polypeptide, or a bioactive fragment thereof, wherein said composition is administered in an amount effective to promote cardiac cell regeneration, and wherein said Wnt-related polypeptide, or bioactive fragment thereof, promotes Wnt signaling via the canonical Wnt signaling pathway.

5. The method of claim 3, wherein said Wnt-related polypeptide is a Wnt polypeptide selected from Wnt3, Wnt3A, or a bioactive fragment thereof.

6. The method of claim 4, wherein said Wnt-related polypeptide is a Wnt polypeptide selected from Wnt3, Wnt3A, or a bioactive fragment thereof.

7. The method of claim 1, wherein said cardiac cell is a cardiomyocyte.

8. The method of claim 2, wherein said cardiac cell is a cardiomyocyte.

9. The method of claim 1, wherein said composition promotes cardiac cell proliferation, and wherein said composition does not induce a hypertrophic response.

10. The method of claim 2, wherein said composition promotes cardiac cell regeneration, and wherein said composition does not induce a hypertrophic response.

11. The method of claim 1, wherein said composition further comprises one or more agents that promote binding of a Wnt polypeptide to a Wnt receptor.

12. The method of claim 11, wherein said agent is selected from heparin or heparin sulfate.

13. The method of claim 1, wherein said composition further comprises one or more additional agents that promote cardiac cell proliferation.

14. The method of claim 13, wherein said one or more additional agents is selected from insulin, an insulin-like growth factor, or a fibroblast growth factor family member.

15. The method of claim 1, wherein said composition further comprises one or more agents that inhibit cardiac cell differentiation.

16. The method of claim 15, wherein said agent is selected from a p38 inhibitor.

17. The method of claim 3, wherein said Wnt-related polypeptide comprises a Wnt polypeptide, or bioactive fragment thereof, that is modified with one or more moieties to produce a modified Wnt polypeptide, or bioactive fragment thereof, and wherein said modified Wnt polypeptide, or bioactive fragment thereof, promotes Wnt signaling via the canonical Wnt signaling pathway.

18. The method of claim 17, wherein said one or more moieties are appended to an N-terminal amino acid residue, a C-terminal amino acid residue, and/or an internal amino acid residue.

19. The method of claim 18, wherein said one or more moieties are hydrophobic moieties.

20. The method of claim 18, wherein said one or more moieties are hydrophilic moieties.

21. The method of claim 19, wherein said one or more hydrophobic moieties are independently selected from any of a sterol, a fatty acid, a hydrophobic amino acid residue, or a hydrophobic peptide.

22. The method of claim 20, wherein said one or more hydrophilic moieties are independently selected from any of a PEG containing moiety, cyclodextran, or albumin.

23. The method of claim 4, wherein said composition is administered to the myocardium, pericardium, or endocardium via a syringe, catheter, stent, wire, or other intraluminal device.

24. A method of treating a condition characterized by cardiac cell injury or death, comprising administering a composition comprising an agent that acts at the cell surface to promote signaling via the canonical Wnt signaling pathway, wherein said composition is administered in an amount effective to treat said condition characterized by cardiac cell injury or death.

25. The method of claim 24, comprising administering a composition comprising a Wnt polypeptide, or a bioactive fragment thereof, wherein said composition is administered in an amount effective to treat said condition characterized by cardiac cell injury or death, and wherein said Wnt polypeptide or bioactive fragment thereof promotes Wnt signaling via the canonical Wnt signaling pathway.

26. The method of claim 25, comprising administering a composition comprising a Wnt3 polypeptide, a Wnt3A polypeptide, or a bioactive fragment thereof, wherein said composition is administered in an amount effective to treat said condition characterized by cardiac cell injury or death, and wherein said Wnt3 polypeptide, Wnt3A polypeptide, or bioactive fragment thereof promotes Wnt signaling via the canonical Wnt signaling pathway.

27. The method of claim 26, wherein said condition characterized by cardiac cell injury or death is myocardial damage from myocardial infarction, and wherein said composition is administered in an amount effective to treat said myocardial damage.

28. The method of claim 26, comprising administering a composition comprising a Wnt polypeptide, or a bioactive fragment thereof, wherein said composition is administered in an amount effective to treat said myocardial damage, and wherein said Wnt polypeptide, or bioactive fragment thereof, promotes Wnt signaling via the canonical Wnt signaling pathway.

29. The method of claim 28, comprising administering a composition comprising a Wnt3 polypeptide, a Wnt3A polypeptide, or a bioactive fragment thereof, wherein said composition is administered in an amount effective to treat said myocardial damage, and wherein said Wnt polypeptide, Wnt3A polypeptide, or bioactive fragment thereof promotes Wnt signaling via the canonical Wnt signaling pathway.

30. The method of claim 26, wherein said condition characterized by cardiac cell injury or death is selected from any of myocardial infarction; atherosclerosis; coronary artery disease; obstructive vascular disease; dilated cardiomyopathy; heart failure;
myocardial necrosis; valvular heart disease; non-compaction of the ventricular myocardium; hypertrophic cardiomyopathy; cancer or cancer-related conditions such as structural defects resulting from cancer or cancer treatments.

31. The method of claim 28, wherein said injury results from myocarditis, exposure to a toxin, exposure to an infectious agent, or from a mineral deficiency.

32. The method of claim 24, wherein said composition is administered systemically.

33. The method of claim 24, wherein said composition is administered to the myocardium, pericardium, or endocardium via a syringe, catheter, stent, wire, or other intraluminal device.

34. The method of claim 25, wherein said Wnt polypeptide, or bioactive fragment thereof, is modified with one or more moieties to produce a modified Wnt polypeptide, or bioactive fragment thereof, and wherein said modified Wnt polypeptide, or bioactive fragment thereof, promotes Wnt signaling via the canonical Wnt signaling pathway.

35. A method of treating a developmental disorder of cardiac cells, comprising administering a composition comprising an agent that acts at the cell surface to promote Wnt signaling via the canonical Wnt signaling pathway, wherein said composition is administered in an amount effective to promote proliferation of cardiac cells, thereby treating said developmental disorder.

36. The method of claim 35, comprising administering a composition comprising a Wnt polypeptide or a bioactive fragment thereof, wherein said composition is administered in an amount effective to promote proliferation of cardiac cells, thereby treating said developmental disorder, and wherein said Wnt polypeptide or bioactive fragment thereof promotes Wnt signaling via the canonical Wnt signaling pathway.

37. The method of claim 36, comprising administering a composition comprising a Wnt3 polypeptide, a Wnt3A polypeptide, or a bioactive fragment thereof, wherein said composition is administered in an amount effective to promote proliferation of cardiac cells, thereby treating said developmental disorder, and wherein said Wnt3 polypeptide, Wnt3A polypeptide, or bioactive fragment thereof promotes Wnt signaling via the canonical Wnt signaling pathway.

38. The method of claim 36, wherein said developmental disorder is selected from any of non-compaction of the ventricular myocardium; congenital heart disease; DiGeorge syndrome; or hypoplastic left heart syndrome.

39. The method of claim 36, wherein said composition is administered in utero.

40. The method of claim 36, wherein said composition is administered systemically.

41. The method of claim 36, wherein said composition is administered to the myocardium, pericardium, or endocardium via a syringe, catheter, stent, wire, or other intraluminal device.

42. The method of claim 36, wherein said Wnt polypeptide, or bioactive fragment thereof, is modified with one or more moieties to produce a modified Wnt polypeptide, or bioactive fragment thereof, and wherein said modified Wnt polypeptide, or bioactive fragment thereof, promotes Wnt signaling via the canonical Wnt signaling pathway.

43. Use of a Wnt polypeptide, or bioactive fragment thereof, in the manufacture of a medicament for promoting cardiac cell proliferation and/or regeneration, wherein said Wnt polypeptide, or bioactive fragment thereof, promotes Wnt signaling via the canonical Wnt signaling pathway, and wherein said Wnt polypeptide, or bioactive fragment thereof, is modified with one or more moieties to produce a modified Wnt polypeptide or bioactive fragment thereof.

44. A modified polypeptide, comprising a Wnt-related polypeptide, or bioactive fragment thereof, appended with one or more moieties to produce a modified Wnt-related polypeptide, or bioactive fragment thereof, wherein said modified Wnt-related polypeptide, or bioactive fragment thereof, promotes Wnt signaling via the canonical Wnt signaling pathway.

45. The modified polypeptide of claim 44, wherein said Wnt-related polypeptide, or bioactive fragment thereof, is appended with two or more moieties to produce a modified Wnt-related polypeptide, or bioactive fragment thereof, wherein said modified Wnt-related polypeptide, or bioactive fragment thereof, promotes Wnt signaling via the canonical Wnt signaling pathway.

46. The modified polypeptide of claim 44, wherein said one or more moieties are appended to an N-terminal amino acid residue, a C-terminal amino acid residue, and/or an internal amino acid residue.

47. The modified polypeptide of claim 46, wherein said one or more moieties are hydrophobic moieties.

48. The modified polypeptide of claim 46, wherein said one or more moieties are hydrophilic moieties.

49. The modified polypeptide of claim 47, wherein said one or more hydrophobic moieties are independently selected from any of a sterol, a fatty acid, a hydrophobic amino acid residue, or a hydrophobic peptide.

50. The modified polypeptide of claim 48, wherein said one or more hydrophilic moieties are independently selected from any of a PEG containing moiety, cyclodextran, or albumin.

51. The modified polypeptide of claim 50 formulated in a pharmaceutically acceptable carrier.

52. The modified polypeptide of claim 50 attached to a biocompatible device or dissolved in a biocompatible matrix.
